(19) United States
(12) Patent Application Publication
(10) Pub. No.: US 2005/0084913 A1
(43) Pub. Date: Apr. 21, 2005

(54) Novel tumor-associated antigens

(75) Inventors: Juha Punnonen, Belmont, CA (US); Doris Apt, San Jose, CA (US); Margaret Neighbors, San Jose, CA (US); Steven R. Leong, Berkeley, CA (US)

Correspondence Address: MAXYGEN, INC.;INTELLECTUAL PROPERTY DEPARTMENT, 515 GALVESTON DRIVE, RED WOOD CITY, CA, 94063, US

(73) Assignee: Maxygen, Inc.

(21) Appl. No.: 10/828,559

(22) Filed: Apr. 19, 2004

Related U.S. Application Data
(60) Provisional application No. 60/464,780, filed on Apr. 22, 2003.

(51) Int. Cl. C12Q 1/68; G01N 33/53; G01N 33/567; C07K 16/28

(52) U.S. Cl. 435/7.2; 530/388.22

(57) ABSTRACT

The invention provides novel polypeptides, including novel tumor-associated antigens, and related nucleic acids, vectors, cells, and antibodies. The invention also provides compositions comprising such polypeptides, nucleic acids, vectors, cells, and antibodies, and methods of producing and using the same.

### CROSS REFERENCE TO RELATED APPLICATION

This application claims priority to and benefit of U.S. Provisional Patent Application No. 60/464,780, filed Apr. 22, 2003, the disclosure of which is incorporated herein by reference in its entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

This invention pertains to novel polypeptides, which include novel tumor-associated antigens, and nucleic acids encoding tumor-associated antigens, and related vectors, cells, compositions, antibodies, and methods of use and production.

### BACKGROUND OF THE INVENTION

Cancer is a leading cause of death in all industrialized nations, where life expectancy continues to rise. For example, cancer is the second leading cause of death in the United States, accounting for almost 500,000 deaths each year. More than 1,000,000 new cases of cancer are diagnosed in the U.S. annually. The American Cancer Society estimates the lifetime risk that an American will develop cancer is 1 in 2 for men and 1 in 3 for women. It is expected that cancer mortality will continue to increase in all industrialized areas of the world.

Common types of cancer in the industrialized world include lung cancer, colorectal cancers, melanomas, breast cancer, and ovarian cancer. Currently, the most effective forms of therapy against these types of cancer are radiation treatment, chemotherapy, and surgery. These forms of treatment are expensive and can have a significant negative impact on patient quality of life. Moreover, tumors in many cancer cases are not susceptible to removal using current surgical techniques and some patients have other conditions that eliminate the possibility of using radiation therapy and/or chemotherapy. Unfortunately, even after apparent complete removal of an identified tumor, survival rates in many cancer cases remain low. For example, less than one-third of lung cancer patients presently survive more than five years after surgical tumor removal.

Almost all forms of cancer continue to be refractory to conventional forms of treatment despite many years of therapeutic experience. It has been proposed that some of the shortcomings of conventional cancer treatments may be overcome by causing a patient's immune system to generate a response to cancer-associated cells through the administration of an immunogenic polypeptide or DNA vaccine. However, such cancer “vaccine”development has been slow, and no effective vaccine currently exists for any form of cancer. Moreover, several aspects of cancer vaccines currently in development may limit their efficacy. For example, antigens currently being developed as cancer vaccines are generally “self” antigens that are typically expressed at low levels on the normal cells of the host. Because the immune system is typically tolerant against such self antigens, the immune responses induced by cancer vaccines are often sub-optimal. In addition, in the case of DNA vaccines, in vivo expression levels of naturally occurring antigen-encoding DNAs are often low and may not stimulate a sufficient systemic immune response necessary to treat disseminated disease.

In view of these and other issues, there remains a need for therapies to treat cancer and prevent the recurrence of the disease. In particular, compositions and methods useful for inducing an immune response(s) against tumor-associated or cancer-associated cells and treating tumors and cancers are needed. The invention includes such compositions and methods. These and other advantages of the invention, as well as additional inventive features, will be apparent from the description of the invention provided herein.

### SUMMARY OF THE INVENTION

In one aspect, the invention provides an isolated, recombinant or non-naturally occurring polypeptide that comprises a polypeptide sequence having at least about 96% amino acid sequence identity to a polypeptide sequence selected from the group consisting of SEQ ID NOS: 1, 9, 12, and 92. Some such polypeptides typically have an ability to induce or enhance an immune response against a mammalian epithelial cell adhesion molecule (EpCAM) or an antigenic or immunogenic fragment or subsequence thereof. Some such polypeptides have an ability to induce or promote an immune response against human EpCAM (“hEpCAM”) or an antigenic fragment thereof.

In another aspect, the invention provides an isolated, recombinant or non-naturally occurring polypeptide that comprises a polypeptide sequence having at least about 96% sequence identity to the polypeptide sequence of SEQ ID NO:5. Such polypeptide typically has an ability to induce or enhance an immune response against a mammalian EpCAM (“mEpCAM”), particularly hEpCAM, or an antigenic or immunogenic fragment thereof.

In yet another aspect, the invention provides an isolated, recombinant or non-naturally occurring polypeptide that comprises a polypeptide sequence having at least about 96% sequence identity to a polypeptide sequence selected from the group consisting of SEQ ID NOS:4, 13, 32, and 78. Some such polypeptides typically have an ability to induce or enhance an immune response against mEpCAM, especially hEpCAM, or an antigenic or immunogenic fragment thereof.

Also provided is an isolated, recombinant or non-naturally occurring polypeptide that comprises a polypeptide sequence having at least about 96% sequence identity to a polypeptide sequence selected from the group consisting of SEQ ID NOS:6, 14, and 34. Some such polypeptides are capable of inducing or enhancing an immune response against mEpCAM (e.g., hEpCAM), or an antigenic or immunogenic fragment thereof.

One aspect of the invention pertains to an isolated or non-naturally occurring polypeptide comprising a polypeptide sequence that has at least about 97% amino acid sequence identity to an amino acid sequence corresponding to amino acid residues 81-265, amino acid residues 82-265, amino acid residues 24-265, or amino acid residues 1-265 of the sequence of SEQ ID NO:4, wherein said polypeptide has an ability to induce an immune response against human EpCAM.

The invention further provides isolated, recombinant, or non-naturally occurring nucleic acid vectors that comprise at least one nucleic acid of the invention or encode at least one polypeptide of the invention, including any of those described above. Also included are viral vectors, viruses and virus-like particles (VLP) that comprise at least one polynucleotide or polypeptide of the invention as described above and in further detail below.

In one aspect, the invention provides an isolated, recombinant or non-naturally occurring nucleic acid comprising a nucleotide sequence that has at least about 80% nucleotide sequence identity to a nucleotide sequence selected from the group consisting of SEQ ID NOS:16, 19-23, 26-28, 33, 35, and 79. Some such nucleic acids encode a polypeptide that induces an immune response against hEpCAM or an antigenic fragment thereof.

In another aspect, the invention provides an isolated or recombinant nucleic acid comprising a nucleotide sequence that has at least about 85% nucleotide sequence identity to a nucleotide subsequence of SEQ ID NO: 19, said subsequence comprising about nucleotide residues 241-795 of SEQ ID NO: 19. Also included in an isolated or non-naturally occurring nucleic acid comprising a nucleotide sequence has, or comprises a subsequence that has, at least about 85% nucleotide sequence identity to a subsequence comprising nucleotide residues 70-795 of SEQ ID NO: 19, wherein said nucleic acid optionally encodes a polypeptide that induces an immune response against EpCAM or an antigenic fragment thereof. Some such nucleic acids encode a polypeptide that induces an immune response against hEpCAM or an antigenic fragment thereof.

In another aspect, the invention provides a nucleic acid encoding a polypeptide having an ability to induce an immune response against human EpCAM, said nucleic acid comprising a nucleotide sequence selected from the group consisting of the group of:
(a) a nucleotide sequence having at least about 96% sequence identity to an amino acid subsequence of SEQ ID NO:4 corresponding to amino acids 81-265, amino acids 82-265, amino acids 22-265, amino acids 24-265, or amino acids 1-265 of the polypeptide sequence of SEQ ID NO:4, or a complementary nucleotide sequence thereof;
(b) a nucleotide sequence comprising nucleotides 64-795, nucleotides 67-795, nucleotides 70-795, nucleotides 73-795, nucleotides 241-795, or 1-795 of the nucleotide sequence of SEQ ID NO: 19, or a complementary nucleotide sequence or any thereof;
(c) a nucleotide sequence selected from the group consisting of SEQ ID NOS: 16, 20-23, 26-28, 33, 35, and 79, or a complementary nucleotide sequence of any thereof; and
(d) a nucleotide sequence that hybridizes under at least stringent conditions over substantially the entire length of the nucleotide sequence of (a), (b), or (c).

In another aspect, the invention provides a nucleic vector comprising at least one nucleic acid of the invention. Also provided are non-nucleic acid vectors, such as viral vectors, that comprise at least one nucleic acid or polypeptide of the invention.

In another aspect, the invention provides a composition comprising a population of antibodies against hEpCAM or an antigenic fragment thereof. Also provided is a monoclonal antibody that specifically binds to hEpCAM or an antigenic fragment thereof. Typically, such antibodies are produced in a subject in vivo in response to a polypeptide of the invention.

The invention additionally provides cells comprising one or more polypeptides, nucleic acids, vectors, and/or antibodies of the invention. Also provided are compositions that comprise one or more polypeptides, nucleic acids, vectors, antibodies, and cells of the invention. For example, in a particular aspect, the invention provides a composition comprising a polypeptide of the invention and a pharmaceutically acceptable carrier.

The polypeptides, nucleic acids, vectors, antibodies, cells, and compositions of the invention are useful in a number of respects, including in therapeutic or prophylactic treatment therapies and/or vaccines for a variety of tumors and cancers, including those associated with expression or over-expression of human EpCAM. Some such polypeptides, nucleic acids, vectors, antibodies, cells, and compositions on the invention are useful in inducing specific immune responses against EpCAM, including an EpCAM-specific antibody response, a T cell proliferation or activation response (e.g., EpCAM-specific CD8+ response), and/or cytokine responses (e.g., enhanced production of cytokines, such as IFN-γ and/or IL-5). The polypeptides, nucleic acids, vectors, antibodies, cells, and compositions of the invention may also be useful in diagnostic assays as described in greater detail below.

In one aspect, the invention includes a method of inducing an immune response to hEpCAM or hEpCAM-associated cells (e.g., ne plastic EpCAM-overexpressing cells) in a subject, including a mammalian (e.g., a human). The method comprises administering an effective amount of one of the aforementioned polypeptides, nucleic acids, vectors, cells, vaccines, and/or antibodies of the invention to the subject, such that at least one immune response to hEpCAM or hEpCAM-associated cells results. Such methods can be used in the therapeutic or prophylactic treatment of a variety of cancers, including, but not limited to, colon, rectal, colorectal, breast, prostate, cervical, ovarian, lung, pancreatic, head and/or neck cancers or other EpCAM/KSA-expressing cancers. Treatment methods include methods for reducing the progression or re-occurrence of a cancer or tumor or metastatic disease associated with an EpCAM malignancy or EpCAM over-expressing caner or tumor.

Polypeptides, nucleic acids, vectors, antibodies, vaccines, cells, and compositions of the invention are also useful in modulating binding of EpCAM to a ligand and/or serving as diagnostic tools for the detection of tumors or cancers associated with EpCAM-expressing or EpCAM-overexpressing cells. Methods for modulating binding between EpCAM and a ligand (including, e.g., EpCAM:EpCAM interactions, where an EpCAM molecule acts as a ligand through binding to another EpCAM molecule) and methods for detecting tumors or cancers associated with EpCAM-expressing or EpCAM-overexpressing cells are contemplated.

The invention provides isolated, synthetic, and/or recombinant polypeptides that induce at least one immune response to a mammalian EpCAM polypeptide or an antigenic fragment thereof. Mammalian EpCAM polypeptides include human EpCAM, the tumor-associated calcium signal transducer 1 (TACST1), which is a murine ortholog of EpCAM (GenBank Accession No. AAH05618), and the human EpCAM-homolog described in International (Int'l) Patent Application WO 01/22920 (see SEQ ID NO:2 shown therein)). Antigenic fragments include subsequences of hEpCAM, such as a polypeptide comprising the signal peptide, propeptide domain, and extracellular domain of human EpCAM, but lacking transmembrane and cytoplasmic domains of hEpCAM.

Among other uses, the polypeptides of the invention, and nucleic acids encoding such polypeptides, are capable of inducing an immune response(s) to mammalian EpCAM and/or EpCAM-associated cells, such as tumor cells that overexpress mammalian EpCAM, including human EpCAM. In this sense, the invention provides a novel group or family of tumor-associated antigens (TAgs). The polypeptides of the invention constitute non-self antigens that are useful for inducing or enhancing EpCAM/KSA-specific immunity in a subject, including EpCAM-specific B cell immunity (EpCAM-specific antibody responses) and/or T cell immunity (EpCAM-specific CD8 CTL responses) for the therapeutic and/or prophylactic treatment of EpCAM/KSA-expressing tumors in mammals, including humans. Administration of such polypeptide or nucleic acid encoding such polypeptide induces a specific antibody or cell-mediated immune response against such tumor(s). Such polypeptides and nucleic acids encoding such polypeptides are particularly useful in tumor-specific vaccines and compositions for the therapeutic or prophylactic treatment of tumors associated with expression or over expression of mammalian EpCAM, including hEp CAM. Such vaccines and compositions may further include at least one adjuvant, at least one immunomodulatory polypeptide or at least one polynucleotide encoding a immunomodulatory polypeptide, or at least one costimulatory polypeptide or at least one polynucleotide encoding a costimulatory polypeptide.

The invention also provides novel isolated, recombinant or non-naturally occurring nucleic acids encoding such immunogenic polypeptides, novel recombinant, isolated, or non-naturally occurring antibodies that react with and/or are generated in response to such immunogenic polypeptides, cells comprising such polypeptides or nucleic acids, vectors comprising such nucleic acids or encoding such polypeptides of the invention, and methods of producing and using such immunogenic polypeptides, nucleic acids, vectors, cells, and antibodies. The nucleic acids, antibodies, and cells of the invention also are useful in inducing an immune response to EpCAM, an antigenic fragment thereof, and/or EpCAM-associated cells. Other uses of the novel polypeptides, nucleic acids, antibodies, and cells of the invention are described below. While the several aspects of the invention can be discussed separately herein, it is to be understood that any feature or features of a particular aspect can apply to any other aspect, unless explicitly stated or contradicted by context.

In another aspect, the invention provides an RNA polynucleotide, said RNA polynucleotide comprising a DNA sequence selected from the group of SEQ ID NOS: 16, 19-23, 26-28, 33, 35, 79, and 94, or a complementary nucleotide sequence of any thereof, in which each thymine nucleotide residue in the DNA sequence is replaced with a uracil nucleotide residue. The invention includes any RMA polynucleotide that can be derived from any DNA sequence of the invention. A cDNA can serve as the template for transcription of RNA polynucleotide. Some such RNA polynucleotides are typically capable of encoding a polypeptide that induces an immune response against a mammalian EpCAM, or an antigenic fragment thereof.

Additional aspects of the invention are described below.

### BRIEF DESCRIPTION OF THE FIGURES

FIG. 1 illustrates exemplary antigen-specific antibody ELISA assays.

FIG. 2 is a graph of antibody concentration (ng/mL) versus absorbance at 450 nanometers (nm) for complexes resulting from the binding of antibodies expressed by hybridomas generated in response to TAg-25 polypeptide (SEQ ID NO:4) to human sEpCAM antigen (SEQ ID NO:40) using human sEpCAM-coated ELISA plates.

FIG. 3 is a graph of EC50 values obtained by subjecting sera drawn from mice injected intramuscularly (i.m.) or subcutaneously (s.c.) with either TAg-25 polypeptide (SEQ ID NO:4) or human sEpCAM polypeptide (SEQ ID NO:40) to an ELISA antibody assay using human sEpCAM-coated ELISA plates or TAg-25-coated ELISA plates. Immunization with TAg-25 polypeptide induces human EpCAM-specific antibodies in vivo in mice.

FIG. 4 illustrates an exemplary monocistronic mammalian plasmid vector of the invention. A restriction map of the vector is shown. This expression vector comprises a polynucleotide sequence that encodes TAg-25 polypeptide (SEQ ID NO:4) and is referred to as a “pMaxVax_{TAg-25}”vector. In constructing this vector, the polynucleotide sequence encoding TAg-25 polypeptide (e.g., SEQ ID NO:19) is cloned in the restriction sites XbaI and NotI in the polylinker of the vector. An exemplary polynucleotide sequence that encodes TAg-25 polypeptide is shown in SEQ ID NO: 19. Additional restriction sites (BamH1, ClaI, EcoRI, HindIII, KpnI, NotI, SmaI) are shown in the figure. Resulting fragment sizes after restriction digest and gel electrophoresis can be calculated from the positions given in parentheses adjacent respective restriction sites. Additional plasmid vectors comprising at least one polynucleotide of the invention are also contemplated. Such polynucleotides typically encode a recombinant or non-naturally occurring polypeptide that induces an immune response against EpCAM or an antigenic fragment thereof.

FIG. 5 illustrates an exemplary bicistronic mammalian plasmid vector of the invention that encodes TAg-25 polypeptide and a CD28 binding protein (CD28BP). A restriction map of the vector is shown. This expression vector is referred to as a pMaxVax_{TAg-25:CD28BP-15} vector. A polynucleotide encoding the CD28BP polypeptide, which is included in the first expression cassette, is operably linked to a first CMV promoter (or variant thereof) and a first BGH polyA sequence; the polynucleotide encoding TAg-25, which is included in the second expression cassette, is operably linked to a second CMV promoter (or variant thereof) and a second BGH polyA sequence. The unique restriction sites BamH1 and KpnI in the polylinker of the vector were used to clone the CD28BP-encoding polynucleotide into the first expression cassette. The unique restriction sites NgoMI, AccI and NheI were used to clone the TAg-25-encoding polynucleotide into the second expression cassette. Additional restriction sites are shown. Resulting fragment sizes after restriction digest and gel electrophoresis can be calculated from the positions given in parentheses adjacent respective restriction sites.

FIG. 6 shows two photographs of two Western blots. The first Western blot was obtained by subjecting supernatant from mammalian cells transfected with a monocistronic DNA plasmid vector comprising a polynucleotide sequence encoding either (1) TAg-25 polypeptide, or (2) sEpCAM, to SDS PAGE and blotting and probing the blot with an antibody against human sEpCAM. The second Western blot was obtained by subjecting supernatant from mammalian cells transfected with a bicistronic plasmid vector comprising a polynucleotide sequence encoding either (1) a TAg polypeptide and a costimulatory polypeptide (e.g., human B7-1 or a CD28BP polypeptide), or (2) human sEpCAM and a costimulatory polypeptide, to SDS PAGE and blotting and probing the blot with an antibody against human sEpCAM.

FIG. 7 is a graph of OD values resulting from anti-human sEpCAM antibody ELISA assays using serum obtained from mice injected i.m. with a plasmid vector comprising a polynucleotide sequence encoding either TAg-25 or sEpCAM. Each mouse was injected with the respective vector 3 times. OD values obtained via ELISA assay after each injection are shown.

FIG. 8 provides absorbance values resulting from ELISA assays using plates coated with either human sEpCAM or TAg-25. Sera was obtained from cynomolgus monkeys, each of which had been injected i.d. or i.m. on days 22 and 43 with: (1) a pMaxVax DNA vector comprising a polynucleotide sequence encoding TAg-25 (e.g., SEQ ID NO: 19); (2) a pMaxVax DNA vector comprising a polynucleotide sequence encoding human sEpCAM antigen (e.g., SEQ ID NO:93), or (3) a control (null or empty) pMaxVax DNA vector that does not encode any antigen. Individual diluted serum samples were placed on respective antigen-coated plates, allowing formation of labeled antigen-antibody complexes. Absorbance of labeled complex formed on each plate was measured at 450 nm. Immunization of cynomolgous monkeys with TAg-25 encoding DNA expression vector induced antibodies that cross-react with or bind human EpCAM.

FIG. 9A shows the results of T cell proliferation assays performed on murine lymphocytes obtained from mice injected i.m. with a DNA plasmid vector comprising a polynucleotide sequence encoding TAg-25 polypeptide or an empty “control” vector and restimulated with TAg-25-his-tagged fusion protein, baculovirus-expressed sEpCAM, or cRPMI medium. FIG. 9B shows the results of T cell proliferation assays performed on murine lymphocytes obtained from mice injected i.m. with TAg-25-his-tagged fusion protein or BSA and restimulated with TAg-25-his-tagged fusion protein or sEpCAM-his-tagged fusion protein. Results of T cell proliferation assays performed on murine lymphocytes obtained from mice receiving no protein injection (“untreated mice”) are also shown. “CPM” refers to counts per minute.

FIGS. 10A and 10B show interferon gamma (“IFN-γ” or “IFN-g”) and interleukin-5 (“IL-5”) concentrations (picograms/milliliter (pg/mL)) in culture supernatants of murine lymphocytes obtained from mice immunized with a pMaxVax DNA plasmid vector comprising a polynucleotide sequence (e.g., SEQ ID NO: 19) encoding TAg-25 polypeptide and restimulated with human sEpCAM polypeptide (SEQ ID NO:40). A pMaxVaxₙᵤₗₗ vector was used as a control for the DNA vector immunizations, and BSA was used as a control for the protein immunizations.

FIG. 11 is a table showing results of four immunizations of cynomolgus macaque monkeys with a pMaxVax DNA plasmid encoding either sEpCAM or TAg-25 polypeptide. Serum obtained from each monkey was analyzed for the presence of antibodies specific to sEpCAM or to TAg-25 polypeptide.

FIG. 12 is a graph of optical density values based on antibody ELISA assays versus reciprocal serum dilution using supernatant obtained from cynomolgus monkeys immunized with pMaxVax DNA expression vector encoding sEpCAM or TAg-25 or a saline-treated control. Each monkey was immunized with 1 mg/dose on days 0, 22, 43, and 64 for a total of 4 doses as shown in FIG. 11. Immunization of a mammal with TAg-25 encoding DNA expression vector induced production of a mean titer level of antibodies against human sEpCAM (i.e., human sEpCAM-specific antibodies) that was about equal to the mean titer level of antibodies against human sEpCAM induced by immunization with a human sEpCAM-encoding DNA expression vector. Immunization of a human with DNA vector encoding TAg-25 or another polypeptide of the invention is expected similarly to induce production of antibodies against human EpCAM expressed in vivo on tissues or cells.

FIG. 13 is a graph showing EC50 values based on antigen-specific antibody ELISA assays using the supernatant obtained from 10 different cynomolgus monkeys The 10 monkeys were divided into three groups of 2, 2, and 6 monkeys, respectively, for immunization. Monkeys in the first group of two monkeys were immunized with a 1 mg dose of a DNA vector encoding sEpCAM (pMaxVax_{sEpCAM}) or TAg-25 (pMaxVax_{Tag-25}) in phosphate-buffered saline (PBS) on days 0, 22, 43 and 64 for a total of 4 doses. Monkeys in the second group were immunized with 1 mg of an empty control vector (pMaxVaxₙᵤₗₗ) in PBS on days 0, 22, 43 and 64 for a total of 4 doses. Monkeys in the third group of 6 monkeys were immunized with a 1 mg dose of pMaxVax_{sEpCAM} or pMaxVax_{Tag-25} vector in PBS on days 0, 22, 43 and 64 for a total of 4 doses as shown along the X axis. Subsequently, each of the monkeys in the second and third groups were immunized with 100 ug of TAg-25 protein in 1.5% alum on days 126 and 154 for a total of two protein boost doses. The results indicate that of the various immunization protocols, the protocols comprising immunization with TAg-25 protein boost (2 times) induced production of the highest titers of specific antibodies against human sEpCAM irrespective of whether or not the animals had first received 4 doses of pMaxVaxₙᵤₗₗ, pMaxVax_{sEpCAM} or pMaxVax_{Tag-25} vector in PBS on days 0, 22, 43 and 64 (as shown in FIG. 13). Immunization of a human with a solution of TAg-25 or another antigenic polypeptide of the invention in saline with, if desired, an adjuvant (e.g., 1.5% alum) is similarly expected to induce high titers of antibodies specifically against human EpCAM.

FIG. 14 is a graph showing IFN-gamma spot forming cells (SFC) as determined by IFN-gamma ELISPOT (amount of cells making IFN-γ in a total of 2×10⁵ cells/well) for each of the immunization protocols for the three groups of monkeys described in FIG. 13. The most potent CD8+ T cell proliferation was induced in cells from animals immunized first with pMaxVax_{sEpCAM} or pMaxVax_{Tag-25} vector in PBS on days 0, 22, 43 and 64 followed by immunization with TAg-25 protein boost (2 times) (FIG. 14). A human sEpCAM-specific CD8+ T cell proliferation response was induced by restimulating the cells with a mixture of the following human EpCAM-derived peptides (comprising 9-11 amino acid residues in length), wherein the mixture comprised a final concentration of 10 μg/mL of each peptide in sterile supplemented DMEM: peptide₁₇₄₋₁₈₄ (YQLDPKFITSI); peptide₁₈₄₋₁₉₂ (ILYENNVIT); peptide₁₈₄₋₁₉₃ (ILYENNVITI); and peptide₂₆₃₋₂₇₁ (GLKAGVIAV). Each such peptide comprises a predicted CTL epitope of human EpCAM. The numerical subscripts indicate the positions of the amino acid residues of the peptide sequence in the polypeptide sequence of human EpCAM (see, e.g., SEQ ID NO:41). For example, the peptide₁₇₄₋₁₈₄ (YQLDPKFITSI) comprises amino acid residues 174-184 of hEpCAM, inclusive. Supplemented DMEM is described in Example 1 (referred to as “growth medium” therein). This peptide mixture is referred to in FIG. 14 as “pep mix.” The peptide₁₇₄₋₁₈₄ (YQLDPKFITSI), peptide₁₈₄₋₁₉₂ (ILYENNVIT), and peptide₁₈₄₋₁₉₃ (ILYENNVITI) are also predicted CTL epitopes of TAg-25 and of other antigenic polypeptides of the invention that include these peptide sequences. The peptide₂₆₃₋₂₇₁ (GLKAGVIAV) is a predicted epitope of a polypeptide of the invention comprising a sequence that comprises, e.g., the ECD of TAg-25 and a transmembrane domain (see, e.g., sequences set forth in SEQ ID NOS:6-8) and a predicted epitope of other antigenic polypeptides of the invention that comprise a polypeptide sequence including at least ECD and TMD domains. There was no detectable proliferation made by cells restimulated with the irrelevant MAGE peptide, which is referred to in FIG. 14 as “Irr Pep.” The 4-amino acid sequence of MAGE peptide is deemed “irrelevant” because this peptide sequence is not found as a subsequence within the polypeptide sequence of human EpCAM (SEQ ID NO:41), sEpCAM (SEQ ID NO:40), or TAg-25 (SEQ ID NO:4). Use of this “irrelevant” sequence confirmed that cell proliferation would not be caused by restimulation with peptide sequences not found within EPCAM. Immunization of a human with at least one dose of a DNA vector encoding TAg-25 or another polypeptide of the invention (“DNA priming”) followed by at least one protein boost comprising a solution of TAg-25 or another antigenic polypeptide of the invention in saline with, if desired, an adjuvant (e.g., 1.5% alum) is similarly expected to induce a CD8+ T cell response specifically reactive against human EpCAM.

FIG. 15 is a schematic illustrating an antigen-specific IFN-γ ELISPOT assay.

FIG. 16 shows an exemplary schedule for DNA immunizations i.m. or i.d. of monkeys with an expression vector encoding TAg-25 antigen of the invention with or without wild-type human B7-1 protein or CD28BP-15 protion four times for 3 weeks (2 mg DNA total). DNA immunizations were followed by i.d. administration to each animal of 100 microgram TAg-2 polypeptide in 2 mg alum twice every four weeks. TAg-25 and CD28BP can be delivered via separate DNA vectors (monocistronic vectors) or delivered together on one DNA vectors (bicistronic vector). The polypeptide and nucleic acid sequences of CD28BP-15 are shown as SEQ ID NO:66 and SEQ ID NO: 19, respectively, in Int'l Patent App. No. PCT/US01/19973 (published as WO 02/00717), filed Jun. 22, 2001, and Int'l Patent App. No. PCT/US02/19898, filed Jun. 21, 2002.

FIG. 17 shows exemplary results of the TAg-25 and/or CD28BP-15 immunizations of cynomolgous monkeys as described in FIG. 16. FIG. 17 shows that CD28BP-15 enhanced EpCAM-specific CD8+ T cell proliferation in such monkeys. Restimulation was performed using standard procedures and a mixture of EpCAM-specific peptides comprising from 9-11 amino acids.

FIG. 18 shows exemplary results of the TAg-25 and/or CD28BP-15 immunizations of cynomolgous monkeys as described in FIGS. 16-17. Administration of CD28BP-15 increased the number of monkeys exhibiting EpCAM-specific IFNγ responses. The number of animals exhibiting antigen-specific CD4 T cell responses (number of animals that are positive when restimulated with TAg-25) and CD4 and CD8 T cell responses (number of animals that are positive for restimulation with both TAg-25 and the mixture of EpCAM-specific peptides. 10 spots above background was considered positive.

FIG. 19 illustrates exemplary results of the immunizations of cynomolgous monkeys as described in FIGS. 16-18 (4^{th} DNA immunization). An EpCAM-specific T cell response was associated with an induction of EpCAM-specific antibodies.

FIG. 20 illustrates exemplary results of the immunizations described in FIG. 16. A DNA prime immunization (using, e.g., TAg-25/CD28BP-encoding DNA vector) followed by one or more protein boosts (using, e.g., TAg-25 protein) enhanced the mean EpCAM-specific antibody titers.

### DETAILED DESCRIPTION OF THE INVENTION

The present invention relates to a novel group of polypeptides that exhibit an ability to induce an immune response against an antigen associated with a tumor. In one aspect, the invention provides a novel family of polypeptides referred to herein as tumor-associated antigens (“TAg”). Such polypeptides are typically characterized by an ability to generate an immune response against an antigenic polypeptide associated with a tumor cell or tissue. In a particular aspect, such polypeptides are capable of inducing at least one type of immune response against an epithelial cell adhesion molecule (“EpCAM”) or an antigenic fragment thereof. Nucleic acids of the invention include those that encode such polypeptides; such a nucleic acid is typically referred to as a “TAg nucleic acid.”

EpCAM is variously is known as GA733-2, epithelial cell glycoprotein 40 (“EGP40” or “GP40”), EPG2, the KS ¼ antigen (“KSA”), or EpCAM/KSA. Unless otherwise noted, the term “EpCAM” is generally used throughout to refer to the EpCAM protein, not the nucleic acid encoding EpCAM. Mammalian EpCAM is a cell surface glycoprotein antigen associated with a variety of tumors and malignant neoplasma. Mammalian EpCAM polypeptides include human EpCAM, the tumor-associated calcium signal transducer 1 (TACST1), which is a murine ortholog of EPCAM (GenBank Accession No. AAH05618), and the human EpCAM-homolog described in International Patent Application WO 01/22920 (see SEQ ID NO:2 shown therein)).

Human EpCAM is a human cell surface glycoprotein antigen associated with carcinomas of various origins, including colorectal, pancreatic, head, neck, ovarian, lung, cervical, prostate, and breast carcinomas. See, e.g., Herlyn et al., J. Immunol. Meth. 73:157-167 (1984); Gottlinger et al., Int. J. Cancer 38:47-53 (1986); Litinov et al., Cell Adhes. Commun. 2(5):417-428 (1994); Balzar et al., J. Mol. Med. 77(10):669-712 (1999); Int'l J. Cancer 87:548 (2000); and J. Urol. 162:1462 (1999). Malignant cell proliferation is often always associated with EpCAM expression at some stage of tumor development and high levels of EpCAM expression negatively correlate with cell differentiation. High levels of EpCAM expression have been shown to correlate with poor survival among breast cancer patients (see, e.g., Spizzo et al., Int. J. Cancer 98(6):883-888 (2002) and Gastl et al., Lancet 356:1981-1982 (2000)). Anti-EpCAM therapy has been found to reduce micrometastases in bone marrow (Kirchneer et al., Ann. Oncol. 13:1044-1048 (2002)).

EpCAM is an antigen often associated With malignant tumors (see, e.g., Ross et al., Biochem. Biophys. Res. Comm., 135:297-303 (1986)). Several independently derived mAbs, including GA733, CO17-IA, M77, M79, MH99, AUA1, MOC 31, KS 1/4, HEA 125, VULD, K931, GZ1, GZ2, GZ20, and 323/A3, have been used to isolate EPCAM (see, e.g., Herlyn et al., supra; Herlyn et al., Proc. Natl. Acad. Sci. USA 75:1438-1482 (1979), Herlyn et al., Hybridoma 5:S3-S10 (1986), Edwards et al., Cancer Res. 48:1306-1317 (1986), Strassburg et al., Cancer Res. 52(4):815-21 (1992), Gottlinger et al., supra, and Balzar et al., supra).

EpCAM mediates Ca₂⁺-independent homotypic cell-cell adhesions and binds through its first cysteine-rich domain (previously referred to as an epidermal growth factor (EGF)-like domain (see, e.g., Balzar et al., 1999, supra—compare with Chong and Speicher, J. Biol. Chem. 276(8):5804-5813 (2001)). It is believe that EpCAM molecules are capable of binding one another; thus, a ligand for EpCAM may comprise another EPCAM molecule.

The polypeptide and nucleic acid sequences of wild-type (WT) human EpCAM have been determined (see, e.g., U.S. Pat. No. 5,348,887 and Strnad et al., Cancer Res., 49:314-17 (1989)). The polypeptide and nucleotide sequences of hEpCAM are set forth herein in SEQ ID NOS:41 and 42, respectively. Experimental evidence indicates that hEpCAM is a type I membrane protein that is 265 amino acids in length and comprises a signal peptide, propeptide, extracellular domain, transmembrane domain, and intracellular anchor (e.g., typically a cytoplasmic domain). Human EpCAM includes an amino-terminal signal peptide comprising a sequence of about 23 amino acids is followed by a 242-amino acid residue extracellular domain comprising 12 cysteine residues and 3 potential N-glycosylation loci, a 23-amino acid residue transmembrane domain, and a highly charged 26 residue intracellular anchor or cytoplasmic domain (see, e.g., Szala et al., Proc. Natl. Acad. Sci. USA 87:3542-3546 (1990), Perez et al., J. Immunol. 142:3662-67 (1989), Strnad et al., Cancer Res. 49:314-17 (1989), and Simon et al., Proc. Natl. Acad. Sci. USA 87:2755-59 (1990)). It is believed that the signal peptide of hEpCAM is proteolytically cleaved from the full-length polypeptide upon processing and expression. There is also some evidence that the ECD of hEpCAM is subject to proteolytic cleavage at about Arg₈₀ of hEpCAM, resulting in a “mature” domain and a propeptide, wherein the propeptide is about 57 amino acid residues in length. The mature domain of hEpCAM typically comprises the ECD, transmembrane domain, and cytoplasmic domain. The mature domain may be bound or covalently linked to a cell membrane in vivo. EpCAM-derived polypeptides and uses of EpCAM and such EpCAM-derived polypeptides are further described in U.S. Pat. No. 5,738,867, European Patent Application 0 609 292, and European Patent Application 0 857 176.

As used herein, “sEpCAM” refers to a polypeptide comprising the signal peptide, propeptide, and extracellular domain of WT full-length or membrane-bound human EPCAM sEpCAM differs from full-length or membrane-bound human EpCAM in that sEpCAM lacks the transmembrane domain and cytoplasmic domain (or other intracellular anchor). sEpCAM is believed to comprise the most important antigenic and immunogenic regions or domains of full-length or membrane bound hEpCAM. Cells transfected with a nucleic acid comprising a nucleotide sequence that encodes sEpCAM will typically secrete the sEpCAM polypeptide. Upon delivery to a host, a secreted sEpCAM may be more accessible to antigen-presenting cells in lymph nodes and other lymphoid organs than full-length or membrane-bound human EpCAM. Secreted forms include a polypeptide subsequence of hEpCAM comprising the PP and ECD of hEpCAM, and a polypeptide subsequence comprising the SP, PP, and ECD of hEpCAM. An sEpCAM-encoding nucleic acid is a nucleic acid that encodes the signal peptide, propeptide and extracellular domain of full-length or membrane-bound hEpCAM.

There are a number of antigenic or immunogenic fragments or subsequences of hEpCAM. These include, but are not limited to, a polypeptide comprising the extracellular domain (ECD) of hEpCAM; a polypeptide comprising the ECD and propeptide (PP) of hEpCAM; a polypeptide comprising the signal peptide (SP), PP, and ECD of hEpCAM; a polypeptide comprising the SP, PP, ECD, and transmembrane domain (TMD) of hEpCAM; a polypeptide comprising the SP, PP, ECD, TMD, and cytoplasm ic domain (CD) of hEpCAM; a polypeptide comprising the PP, ECD, and TMD of hEpCAM; a polypeptide comprising the PP, ECD, TMD; and. CD of hEpCAM; and a polypeptide comprising the ECD and TMD of hEpCAM; a polypeptide comprising the ECD, TMD, and CD of hEpCAM (referred to as the “mature domain”); and secreted forms of hEpCAM.

As noted above, tumor cells are among the cells that are typically associated with EpCAM or that overexpress EpCAM. In humans and likely in other mammalian species, EpCAM is expressed on numerous tumor cells, including particular cells or tissues associated with breast, lung, colon, colorectal, and prostate tumors and thus EpCAM constitutes a self protein. Because EpCAM is a self protein, a host is typically tolerant of EpCAM. One approach to treating tumors associated with self proteins, such as EpCAM, is the administration of “non-self” tumor antigens that induce cross-reactivity against self tumor antigens. With such approach, immunological tolerance can be broken in vivo.

In one aspect, the polypeptides and nucleic acids of the invention are capable of inducing an immune response against an antigenic polypeptide associated with tumor cells or tissues. The novel group or family of tumor-associated antigens (“TAgs”) of the invention includes non-self antigens designed to break immunological tolerance against EPCAM in mammals and/or to induce anti-tumor immunological responses in mammals, particularly humans. Among other uses, the polypeptides and nucleic acids of the invention are useful for inducing or enhancing EpCAM/KSA-specific immunity in a subject, including EpCAM-specific B cell immunity (EpCAM-specific antibody responses) and/or T cell immunity (EpCAM-specific CD8 CTL responses). In one aspect, administration of a TAg polypeptide or Tag-encoding nucleic acid induces various specific antibody or cell-mediated immune responses against such tumor(s). In humans, such immune responses include human EpCAM-specific antibodies (B cell immunity), antigen-specific CD8T cells (T cell immunity), and specific cytokine responses (e.g., IFN-γ and IL-5). The TAg polypeptides and TAg-encoding nucleic acids of the invention are particularly useful in methods for the therapeutic and/or prophylactic treatment of EpCAM/KSA-expressing tumors in mammals, including humans. Such methods are described in greater detail below. TAg polypeptides and nucleic acids are also useful in tumor-specific vaccines and compositions for the treatment of tumors associated with expression or overexpression of mammalian EPCAM, including hEpCAM. Vaccination formats, including those comprising DNA vaccination and protein boosting using TAg molecules of the invention are provided. If desired, a TAg polypeptide or nucleic acid is administered with a costimulatory molecule to further augment the immune response as described in greater detail below.

In other aspects, the invention provides vectors, cells, compositions, and vaccines that comprise at least one TAg polypeptide or TAg-polypeptide-encoding nucleic acid, or any combination thereof. Additionally, the invention provides methods of treating tumors and cancers and related diseases that utilize such polypeptides or nucleic acids. Also included are diagnostic assays for detecting the presence of EpCAM or an antigenic fragment thereof. Details of these and other aspects of the invention are provided below.

Definitions

It is also to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific examples of appropriate materials and methods are described herein.

As used in this specification and the appended claims, the singular forms “a”, “an” and “the” are to be construed to cover both singular and plural referents unless the content or context clearly dictates otherwise. Thus, for example, reference to “polypeptide” includes two or more such polypeptides. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. The headings provided in the description of the invention are included merely for convenience and are not intended to be limiting in the scope of the disclosure.

The terms “nucleic acid,” “polynucleotide,” “polynucleotide sequence,” and “nucleotide sequence” are used to refer to a polymer of nucleotides (A,C,T,U,G, etc. or naturally occurring or artificial nucleotide analogues), e.g., DNA or RNA, or a representation thereof, e.g., a character string, etc, depending on the relevant context. The terms “nucleic acid” and “polynucleotide” are used interchangeably herein; these terms are used in reference to DNA, RNA, or other novel nucleic acid molecules of the invention, unless otherwise stated or clearly contradicted by context. A given polynucleotide or complementary polynucleotide can be determined from any specified nucleotide sequence. A nucleic acid may be in single- or double-stranded form.

The terms “protein,” “polypeptide,” “amino acid sequence,” and “polypeptide sequence” are used to refer to a polymer of amino acids (a protein, polypeptide, etc.) or a character string representing an amino acid polymer, depending on context. The terms “protein,” “polypeptide,” and “peptide” are used interchangeably herein. Given the degeneracy of the genetic code, one or more nucleic acids, or the complementary nucleic acids thereof, that encode a specific amino acid sequence or polypeptide sequence can be determined from the amino acid or polypeptide sequence.

The term “isolated,” when applied to a nucleic acid or polypeptide, typically refers to a nucleic acid or polypeptide that (1) is produced (e.g., replicated or cloned) or exists in a cell and thereafter rendered at least substantially free of other cellular components, such as biomolecules (e.g., a nucleic acid or polypeptide that is rendered essentially free of such other cellular biomolecules by purification and/or enrichment of a composition containing the nucleic acid or polypeptide, respectively); (2) is the dominant component in a composition or preparation and which may be (though not necessarily) the only detectable in a composition or preparation; and/or (3) is rendered present in a desired (i.e., approximately set) amount in a particular composition by purification, enrichment, synthesis, or other suitable technique. In particular, an isolated nucleic acid usually refers a nucleotide sequence that is not immediately contiguous with one or more nucleotide sequences with which it is normally immediately contiguous (i.e., at the 5′ and/or 3′ end) in the sequence from which it is obtained and/or derived. For example, an isolated gene is separated from open reading frames which flank the gene and encode a protein other than the gene of interest. An isolated nucleic acid or polypeptide comprises at least about 70% or 75%, typically at least about 80% or about 85%, or preferably at least about 90%, 95%, or more of a composition or preparation (e.g., percent by weight or volume).

An isolated nucleic acid or polypeptide can be obtained by application of any suitable isolation technique. For example, an isolated polypeptide can be obtained by expressing a nucleic acid encoding the polypeptide in a host cell in a medium, such that the polypeptide is present, and isolating the polypeptide by separating the polypeptide from other cellular biomolecules (e.g., other cellular polypeptides, lipids, glycoproteins, nucleic acids, etc.). Alternatively, an isolated polypeptide can be obtained by synthesizing the polypeptide through chemical synthesis techniques under conditions and at levels where the synthesized polypeptide is either the dominant polypeptide species in a composition (e.g., a library of polypeptides) or at least present in a predominant concentration with respect to other polypeptides and biomolecules in the composition. A polypeptide isolated from a cell culture from which it is expressed can subsequently be mixed in a composition such that it is no longer the dominant polypeptide species in the composition. Nucleic acids may be similarly isolated by suitable techniques.

The invention provides compositions that exhibit essential homogeneity with respect to polypeptide and/or nucleic acid content, such that contaminant polypeptide or nucleic acid species cannot be detected in the composition by conventional detection methods. Purity and homogeneity are typically determined using analytical chemistry techniques, such as polyacrylamide gel electrophoresis or high performance liquid chromatography. The term “purified,” as applied to nucleic acids or polypeptides, generally denotes a nucleic acid or polypeptide that is essentially free from other components as determined by standard analytical techniques (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” Particularly, it means that the nucleic acid or polypeptide is at least about 50% pure, usually at least about 75% or 80% pure, more preferably at least about 85% or 90% pure, and most preferably at least about 99% pure (e.g., percent by weight on a molar basis).

In a related sense, the invention provides methods of enriching compositions for such molecules. A composition is enriched for a molecule when there is a substantial increase in the concentration of the molecule after application of a purification or enrichment technique. A substantially pure polypeptide or polynucleotide will typically comprise at least about 55%, 60%, 70%, 80%, 90%, 95%, or at least about 99% percent by weight. (on a molar basis) of all macromolecular species in a particular composition.

A nucleic acid or polypeptide is “recombinant” when it is artificial or engineered, or derived from an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide or polypeptide that does not appear in nature, for example, a variant of a naturally-occurring polynucleotide or polypeptide, respectively, is recombinant. A recombinant polynucleotide or recombinant polypeptide may include one or more nucleotides or amino acids, respectively, from more than one source nucleic acid or polypeptide, which source nucleic acid or polypeptide can be a naturally-occurring nucleic acid or polypeptide, or can itself have been subjected to mutagenesis or other type of modification.

An “immunogen” refers generally to a substance capable of provoking or altering an immune response, and includes, but is not limited to, e.g., immunogenic proteins, polypeptides, and peptides; antigens and antigenic peptide fragments thereof; nucleic acids having immunogenic properties or encoding polypeptides having such properties.

An “immunomodulator” or “immunomodulatory” molecule, such as an immunomodulatory polypeptide or nucleic acid, modulates an immune response. By “modulation” or “modulating” an immune response is intended that the immune response is altered. For example, “modulation” of or “modulating” an immune response in a subject generally means that an immune response is stimulated, induced, inhibited, decreased, increased, enhanced, or otherwise altered in the subject. Such modulation of an immune response can be assessed by means known to those skilled in the art, including those described below. An “immunostimulator” is a molecule, such as a polypeptide or nucleic acid, that stimulates an immune response.

An immune response generally refers to the development of a cellular or antibody-mediated response to an agent, including, e.g., an antigen, immunogen, an immunomodulator, immunostimulator, or nucleic acid encoding any such agent. An immune response includes production of at least one or a combination of cytotoxic T lymphocytes (CTLs), B cells, antibodies, or various classes of T cells that are directed specifically to antigen-presenting cells expressing the antigen of interest.

A “subsequence” or “fragment” is any portion of the entire sequence.

Numbering of an amino acid or nucleotide polymer corresponds to numbering of a selected amino acid polymer or nucleic acid when the position of a given monomer component (amino acid residue, nucleotide residue, etc.) of the polymer corresponds to the same residue position (or equivalent residue position) in a selected reference polypeptide or polynucleotide.

An “antigen” refers to a substance that is capable of inducing an immune response (e.g., humoral and/or cell-mediated) in a host, including, but not limited to, eliciting the formation of antibodies in a host, or generating a specific population of lymphocytes reactive with that substance. Antigens are typically macromolecules (e.g., proteins and polysaccharides) that are foreign to the host.

An “adjuvant” refers to a substance that enhances an immune response. For example, an adjuvant may enhance an antigen's immune-stimulating properties or the pharmacological effect(s) of a compound or drug. An adjuvant may comprise an oil, emulsifier, killed bacterium, aluminum hydroxide, or calcium phosphate (e.g., in gel form), or any combination of one or more thereof. Examples of adjuvants include “Freund's Complete Adjuvant,” “Freund's incomplete adjuvant,” Alum, and the like. Freund's Complete Adjuvant is an emulsion of oil and water containing an immunogen, an emulsifying agent and mycobacteria. Freund's Incomplete Adjuvant is the same, but without mycobacteria. Other adjuvants include BCG adjuvants, DETOX, cytokines (such as, e.g., interleukin-12 (IL-12)), co-stimulatory molecules (such as, e.g., B7-1 (CD80) or B7-2 (CD86)), and haptens, such as dinitrophenyl (DNP). An adjuvant is typically administered to a subject (e.g., via injection intramuscularly or subcutaneously) in an amount sufficient to enhance an immune response.

A “vector” is a composition or module for facilitating cell transduction or transfection by a selected nucleic acid, or expression of the nucleic acid in the cell. Vectors include, e.g., plasmids, cosmids, viruses, YACs, bacteria, poly-lysine, etc.

A “signal peptide” is an amino acid sequence that is translated in conjunction with a polypeptide and directs the polypeptide to the secretory system.

An “expression vector” is a nucleic acid construct or sequence, generated recombinantly or synthetically, with a series of specific nucleic acid elements that permit transcription of a particular nucleic acid in a host cell. The expression vector can be part of a plasmid, virus, or nucleic acid fragment. The expression vector typically includes a nucleic acid to be transcribed operably linked to a promoter.

The term “expression” includes any step involved in the production of the polypeptide including, but not limited to, transcription, post-transcriptional modification, translation, post-translational modification, and/or secretion.

A “host cell” includes any cell type that is susceptible to transformation with a nucleic acid.

“Substantially the entire length of a polynucleotide sequence” or “substantially the entire length of a polypeptide sequence” refers to at least about 50%, generally at least about 60%, 70%, or 75%, usually at least about 80% or 85%, and preferably at least about 90%, 91%, 92%, 93%, 94%, 95%, 96%, 97%, 98%, 99%, 99.5% or more of the length of a polynucleotide sequence or polypeptide sequence, respectively.

“Naturally occurring” as applied to an object refers to the fact that the object can be found in nature as distinct from being artificially produced by man. Non-naturally occurring as applied to an object means the object cannot be found in nature.

The term “synthetic” in reference to an entity or object means an entity or object produced at least in part by an artificial process, in particular, an object not of natural origin.

A “variant” of a polypeptide refers to a polypeptide comprising a polypeptide sequence that differs in one or more amino acid residues from the polypeptide sequence of a parent or reference polypeptide, usually in at least about 1, 2, 3, 4, 5, 6, 7, 8, 9, 10, 11, 12, 13, 14, 15, 20, 23, 25, 30, 40, 50, 75, 100 or more amino acid residues. A polypeptide variant may differ from a parent or reference polypeptide by, e.g., deletion, addition, or substitution of one or more amino acid residues of the parent or reference polypeptide, or any combination of such deletion(s), addition(s), and/or substitution(s). A “variant” of a nucleic acid refers to a nucleic acid comprising a nucleotide sequence that differs in one or more nucleic acid residues from the nucleotide sequence of a parent or reference nucleic acid, usually in at least about 1, 2, 3, 4, 5, 6, 7, 8, 9, 10, 11, 12, 13, 14, 15, 17, 20, 21, 24, 27, 30, 33, 36, 39, 40, 45, 50, 60, 66, 75, 90, 100, 120, 150, 225 or more nucleic acid residues. A nucleic acid variant may differ from a patent or reference nucleic acid, by e.g., deletion, addition, or substitution of one or more nucleic acid residues parent or reference nucleic acid, or any combination of such deletion(s), addition(s), and/or substitution(s).

The term “encoding” refers to the ability of a nucleotide sequence to code for one or more amino acids. The term does not require a start or stop codon. An amino acid sequence can be encoded in any one of six different reading frames provided by a polynucleotide sequence and its complement.

The term “subject” as used herein includes, but is not limited to, an organism, including mammals and non-mammals. A mammal includes, a human, non-human primate (e.g., baboon, orangutan, monkey), mouse, pig, cow, goat, cat, rabbit, rat, guinea pig, hamster, horse, monkey, and sheep. A non-mammal includes a non-mammalian invertebrate and non-mammalian vertebrate, such as a bird (e.g., a chicken or duck) or a fish.

The term “pharmaceutical composition” refers to a composition suitable for pharmaceutical use in a subject, including an animal or human. A pharmaceutical composition typically comprises an effective amount of an active agent and a carrier. The carrier is typically pharmaceutically acceptable carrier.

The term “effective amount” means a dosage or amount of a molecule or composition sufficient to produce a desired result. The desired result may comprise an objective or subjective improvement in the recipient of the dosage or amount. For example, the desired result may comprise a measurable or testable induction, promotion, enhancement or modulation of an immune response in a subject to whom a dosage or amount of a particular antigen or immunogen (or composition thereof) has been administered. An amount of an immunogen sufficient to produce such result also can be described as an “immunogenic” amount.

A. “prophylactic treatment” is a treatment administered to a subject who does not display signs or symptoms of, or displays only early signs or symptoms of, a disease, pathology, or disorder, such that treatment is administered for the purpose of preventing or decreasing the risk of developing the disease, pathology, or disorder. A prophylactic treatment functions as a preventative treatment against a disease, pathology, or disorder. A “prophylactic activity” is an activity of an agent that, when administered to a subject who does not display signs or symptoms of, or who displays only early signs or symptoms of, a pathology, disease, or disorder, prevents or decreases the risk of the subject developing the pathology, disease, or disorder. A “prophylactically useful” agent refers to an agent that is useful in preventing or decreasing development of a disease, pathology, or disorder.

A “therapeutic treatment” is a treatment administered to a subject who displays symptoms or signs of pathology, disease, or disorder, in which treatment is administered to the subject for the purpose of diminishing or eliminating those signs or symptoms. A “therapeutic activity” is an activity of an agent that eliminates or diminishes signs or symptoms of pathology, disease or disorder when administered to a subject suffering from such signs or symptoms. A “therapeutically useful” agent means the agent is useful in decreasing, treating, or eliminating signs or symptoms of a disease, pathology, or disorder.

The term “gene” broadly refers to any nucleic acid segment (e.g., DNA) associated with a biological function. Genes include coding sequences and/or regulatory sequences required for their expression. Genes also include non-expressed DNA nucleic acid segments that, e.g., form recognition sequences for other proteins (e.g., promoter, enhancer, or other regulatory regions). Genes can be obtained from a variety of sources, including cloning from a source of interest or synthesizing from known or predicted sequence information, and may include sequences designed to have desired parameters.

Generally, the nomenclature used hereafter and the laboratory procedures in cell culture, molecular genetics, molecular biology, nucleic acid chemistry, and protein chemistry described below are those well known and commonly employed by those of ordinary skill in the art. Standard techniques, such as described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”) and CURRENT PROTOCOLS IN MOLECULAR BIOLOGY, F. M. Ausubel et al., eds., Current Protocols, a joint venture between Greene Publishing Associates, Inc. and John Wiley & Sons, Inc. (1994, supplemented through 1999) (hereinafter “Ausubel”), are used for recombinant nucleic acid methods, nucleic acid synthesis, cell culture methods, and transgene incorporation, e.g., electroporation, injection, gene gun, impressing through the skin, and lipofection. Generally, oligonucleotide synthesis and purification steps are performed according to specifications. The techniques and procedures are generally performed according to conventional methods in the art and various general references that are provided throughout this document. The procedures therein are believed to be well known to those of ordinary skill in the art and are provided for the convenience of the reader.

As used herein, an “antibody” refers to a protein comprising one or more polypeptides substantially or partially encoded by immunoglobulin genes or fragments of immunoglobulin genes. The term antibody (abbreviated “Ab”) is used to mean whole antibodies and binding fragments thereof. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (e.g., antibody) structural unit comprises a tetramer. Each tetramer is composed of two identical pairs of polypeptide chains, each pair having one “light” (about 25 KDa) and one “heavy” chain (about 50-70 KDa). The N-terminus of each chain defines a variable region of about 100 to 110 or more amino acids primarily responsible for antigen recognition. The terms variable light chain (VL) and variable heavy chain (VH) refer to these light and heavy chains, respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of a Fab fragment which itself is a light chain joined to VH-CH1 by a disulfide bond. The F(ab)′2 may be reduced under mild conditions to break the disulfide linkage in the hinge region thereby converting the (Fab′)₂ dimer into an Fab′ monomer. The Fab′ monomer is essentially a Fab fragment with part of the hinge region. The Fc portion of the antibody molecule corresponds largely to the constant region of the immunoglobulin heavy chain, and is responsible for the antibody's effector function (see FUNDAMENTAL IMMUNOLOGY, W. E. Paul, ed., Raven Press, N.Y. (1993) for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody also includes antibody fragments either produced by the modification of whole antibodies or synthesized de novo using recombinant DNA methodologies. Antibodies also include single-armed composite monoclonal antibodies, single chain antibodies, including single chain Fv (sFv) antibodies in which a variable heavy and a variable light chain are joined together (directly or through a peptide linker) to form a continuous polypeptide, as well as diabodies, tribodies, and tetrabodies (Pack et al. (1995) J. Mol. Biol. 246:28; Biotechnol. 11:1271; Biochem. 31:1579), polyclonal antibodies, chimeric and humanized antibodies, fragments produced by an Fab expression library, and the like.

The term “epitope” refers to an antigenic determinant capable of specific binding to a part of an antibody. Epitopes usually consist of chemically active surface groupings of molecules such as amino acids or sugar side chains and usually have specific 3-dimensional structural characteristics, as well as specific charge characteristics. An epitope may comprise a short peptide sequence (e.g., 3-20 amino acid residues). Conformational and nonconformational epitopes are distinguished in that the binding to the former but not the latter is lost in the presence of denaturing solvents.

A “specific binding affinity” between two molecules, e.g., a ligand and a receptor, means a preferential binding of one molecule for another. The binding of molecules is typically considered specific if the binding affinity is about 1×10² M⁻¹ to about 1×10⁹ M⁻¹ (i.e., about 10⁻²-10⁻⁹ M) or greater.

An “antigen-binding fragment” of an antibody is a peptide or polypeptide fragment of the antibody that binds or selectively binds an antigen. An antigen-binding site is formed by those amino acids of the antibody that contribute to, are involved in, or affect the binding of the antigen. See Scott, T. A. and Mercer, E. I., CONCISE ENCYCLOPEDIA: BIOCHEMISTRY AND MOLECULAR BIOLOGY (de Gruyter, 3d ed. 1997), and Watson, J. D. et al., RECOMBINANT DNA (2d ed. 1992)[hereinafter “Watson, Recombinant DNA”].

A nucleic acid is “operably linked” with another nucleic acid sequence when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “cytokine” includes, e.g., interleukins, interferons, chemokines, hematopoietic growth factors, tumor necrosis factors and transforming growth factors. In general these are small molecular weight proteins that regulate maturation, activation, proliferation, and differentiation of cells of the immune system.

The term “nucleic acid construct” or “polynucleotide construct” means a nucleic acid molecule, either single- or double-stranded, which is isolated from a naturally occurring gene or which has been modified to contain segments of nucleic acids in a manner that would not otherwise exist in nature. The term nucleic acid construct is synonymous with the term “expression cassette” when the nucleic acid construct contains the control sequences required for expression of a coding sequence of the present invention.

The term “control sequence” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, a control sequence include a promoter, and transcriptional and translational stop signals. The control sequences may be provided with linkers for the purpose of introducing specific restriction sites facilitating ligation of the control sequences with the coding region of the nucleotide sequence encoding a polypeptide.

When used herein the term “coding sequence” is intended to cover a nucleotide sequence, which directly specifies the amino acid sequence of its protein product. The boundaries of the coding sequence are generally determined by an open reading frame, which usually begins with the ATG start codon.

The term “screening” describes, in general, a process that identifies optimal molecules of the present invention, including polypeptides having an ability to induce an immune response against EpCAM or a fragment thereof. Several properties of the respective molecules can be used in selection and screening, for example, an ability of a respective molecule to induce an immune response in a test system. Selection is a form of screening in which identification and physical separation are achieved simultaneously by expression of a selection marker, which, in some genetic circumstances, allows cells expressing the marker to survive while other cells die (or vice versa). Screening markers include, for example, luciferase, beta-galactosidase and green fluorescent protein, reaction substrates, and the like. Selection markers include drug and toxin resistance genes, and the like. Because of limitations in studying primary immune responses in vitro, in vivo studies are particularly useful screening methods. In some such studies, a genetic vaccine or vector that comprises one or more polynucleotide sequences of the invention, or a polypeptide of the invention, is first introduced to test animals, and an induced immune response is subsequently studied by analyzing the type of immune responses (Ab production, T cell proliferation, cytokine production), or by studying the quality or strength of the induced immune response using lymphoid cells derived from the immunized animal. In the case of novel TAg antigens of the invention, various properties of the antigen can be used in selection and screening, including expression, folding, stability, ability to induce an immune response against a mammalian EpCAM or antigenic fragment thereof, and presence of epitopes by comparison with epitopes of related antigens. Although spontaneous selection can and does occur in the course of natural evolution, in the present methods, selection is performed by man.

Various additional terms are defined or otherwise characterized herein.

Polypeptides of the Invention

In one aspect, the invention provides polypeptides that are capable of inducing an immune response. In a particular aspect, the invention provides a novel group or family of tumor-associated antigenic polypeptides or “TAg polypeptides.” Such polypeptides are typically characterized by an ability to generate an immune response against an antigenic polypeptide that is associated with or overexpressed by tumor cells or tissues. In a one aspect, such polypeptides are capable of inducing at least one type of immune response against an EpCAM or an antigenic fragment thereof. For example, TAg polypeptides of the invention are capable of inducing an immune response against cells or tissues that are associated with or express EpCAM. In one aspect, the invention provides polypeptides that have the ability to induce an immune response against a mammalian EpCAM (“mEpCAM”) polypeptide or antigenic fragment thereof or a related self-antigen or mEpCAM homolog, and/or against cells or tissues that are associated with or express hEpCAM. In a particular aspect, the invention provides polypeptide that are capable of inducing an immune response against hEpCAM, or an antigenic fragment thereof, and/or against cells or tissues that are associated with or express hEpCAM. The immune response may include humoral and/or cellular response(s) against a mEpCAM, particularly hEpCAM. In one aspect, the invention provides a TAg polypeptide that is capable of inducing a mEpCAM-specific antibody response, a mEpCAM-specific T cell proliferative response, and/or production of one or more cytokines. Some such TAg polypeptides specifically bind antibodies to mEpCAM or hEpCAM.

Polypeptides Comprising Extracellular Domains

In another aspect, the invention provides an isolated, recombinant or non-naturally occurring polypeptide that comprises a polypeptide sequence having at least about 75, 80, 85, 86, 87, 88, 90, 91, 92, 93, 94, 95, 96, 97, 98, 99, or 100% sequence identity to a polypeptide sequence selected from the group consisting of SEQ ID NOS: 1, 9, 12, and 92. Such polypeptides comprise extracellular domains. Some such polypeptides typically have an ability to induce or enhance an immune response against a mammalian EpCAM or an antigenic or immunogenic fragment or subsequence thereof. Some such polypeptides have an ability to induce or promote an immune response against hEpCAM. Some such polypeptides bind antibodies to mEpCAM or hEpCAM.

In some embodiments, such ECD polypeptides further comprise one or more additional polypeptides selected from among a signal peptide, propeptide, transmembrane domain, and/or a cytoplasmic domain, including, e.g., a novel recombinant or non-naturally occurring signal peptide, propeptide, transmembrane domain, and/or cytoplasmic domain of the invention as described in detail below, or a known signal peptide, propeptide, transmembrane domain, and/or cytoplasmic domain of human EpCAM, a homolog of human or other mammalian EpCAM (e.g., GenBank Accession No. XP_{—}067815), or an ortholog of human or other mammalian EpCAM (see, e.g., International Patent Applications WO 00/37503 and 01/88188), or a variant of any thereof. Such polypeptide of the invention or nucleic acid encoding any such polypeptide typically has the ability to induce at least one immune response in a mammalian host. Such polypeptides usually are capable of inducing an immune response against human EpCAM or an antigenic fragment thereof. Such immune responses include, e.g., the ability to induce or promote: (1) production of antibodies that bind mEpCAM or hEpCAM or an antigenic or immunogenic fragment thereof, (2) T cell proliferation and/or T cell activation, and/or (3) production of one or more cytokines, such as one or more interleukins (IL) and/or interferons (IFN).

In another aspect, the invention provides an isolated, recombinant or non-naturally occurring polypeptide comprising a polypeptide sequence that has at least about 80, 85, 86, 87, 88, 90, 91, 92, 93, 94, 95, 96, 97, 98, 99, or 100% sequence identity to a polypeptide sequence selected from the group consisting of SEQ ID NOS:4, 13, 32, and 78. Preferable, such polypeptide is capable of inducing an immune response against mEpCAM or hEpCAM or an antigenic fragment of either. A preferred polypeptide of the invention, referred to as tumor-associated antigen 25 (abbreviated “TAg-25” polypeptide or “TAg-25” antigen), comprises the polypeptide sequence shown in SEQ ID NO:4. The TAg-0.25 polypeptide includes a signal peptide, propeptide, and extracellular domain.

Another aspect of the invention pertains to an isolated, recombinant or non-naturally occurring polypeptide that comprises a first polypeptide having a sequence with at least about 80, 85, 90, 91, 92, 93, 94, 95, 96, 97, 98, 99, or 100% amino acid sequence identity to a polypeptide sequence selected from the group consisting of SEQ ID NOS: 1, 9, 12, and 92 and a second polypeptide comprising a polypeptide sequence having at least about 80, 85, 90, 91, 92, 93, 94, 95, 96, 97, 98, 99, or 100% sequence identity to a polypeptide sequence selected from the group consisting of SEQ ID NOS:2 and 38. Some such polypeptides are capable of inducing an immune response against mEpCAM or hEpCAM or an antigenic fragment thereof. The polypeptide sequence of SEQ ID NO: 1 corresponds to the ECD of TAg-25 polypeptide, and the sequence of SEQ ID NO:2 corresponds to the propeptide of TAg-25 polypeptide. Typically, the second polypeptide is fused to the N-terminus of said first polypeptide, forming a fusion protein. Some such isolated, recombinant or non-naturally occurring polypeptides further comprise a signal peptide fused to the N-terminus, thereby forming a fusion polypeptide comprising a signal peptide, propeptide, and ECD. The signal peptide typically comprises an amino acid sequence that has at least about 85, 90, 91, 92, 93, 94, 95, 96, 97, 98, 99, or 100% sequence identity to a polypeptide sequence selected from the group consisting of SEQ ID NOS:3 and 37.

In another aspect, the invention provides an isolated, recombinant or non-naturally occurring polypeptide, which polypeptide comprises a polypeptide sequence that has at least about 80, 85, 90, 91, 92, 93, 94, 95, 96, 97, 98, 99, or 100% amino acid sequence identity to the polypeptide sequence of SEQ ID NO:5. In a preferred embodiment, the polypeptide is capable of inducing an immune response against hEpCAM or an antigenic fragment thereof. Some such polypeptides can further comprise a signal peptide, transmembrane domain, and/or a cytoplasmic domain. The C-terminus of a signal peptide is fused to the N-terminus of the polypeptide; the N-terminus of a TMD is fused to the C-terminus of the polypeptide. The N-terminus of a CD may be fused to the C-terminus of the TMD. A variety of signal peptide sequences can be employed, including either those set forth in SEQ ID NOS:3 and 37. A variety of TM and/or CD sequences can also be used, including a TMD and/or CD sequence derived from EpCAM, a homolog of EpCAM (e.g., GenBank Accession No. XP_{—}067815), or an ortholog of EpCAM (see, e.g., International Patent Applications WO 00/37503 and 01/88188).

In yet a further aspect, the invention provides an isolated, recombinant or non-naturally occurring polypeptide comprising a polypeptide sequence that has at least about 80, 85, 90, 91, 92, 93, 94, 95, 96, 97, 98, 99, or 100% sequence identity to a polypeptide sequence selected from the group consisting of SEQ ID NOS:1, 4-10, 12-14, 32, 34, 78, and 92. Some such polypeptides have the ability to induce at least one type of immune response against mEpCAM or hEpCAM or an antigenic fragment thereof. Such immune response includes the ability to induce production of antibodies that specifically bind mEpCAM or hEpCAM or an antigenic or immunogenic fragment thereof, ability to induce T cell proliferation and/or T cell activation, and/or the ability to induce production of one or more cytokines (e.g., including IL and/or IFN). Such immune responses can be measured using techniques well known to those of skill and as described in further detail below.

One aspect of the invention pertains to an isolated, recombinant or non-naturally occurring polypeptide comprising a polypeptide sequence that has at least about 80, 85, 90, 91, 92, 93, 94, 95, 96, 97, 98, 99, or 100% sequence identity to an amino acid subsequence of the polypeptide sequence of SEQ ID NO:4, which amino acid subsequence comprises or consists essentially of amino acid residues 81-265 (i.e., residue 81 through and inclusive of residue 265), 82-265, 22-265, 23-265, 24-265, or 1-265 of SEQ ID NO:4, wherein the resultant polypeptide has an ability to induce at least one type of immune response against hEpCAM or an antigenic fragment thereof. As noted above, such immune responses include the ability to induce or promote production of antibodies that specifically bind hEpCAM or an antigenic fragment thereof, induce or promote T cell proliferation and/or T cell activation, and/or induce or promote production of one or more cytokines, including an IFN and/or IL.

Also provided is an isolated, recombinant or non-naturally occurring polypeptide comprising an amino acid sequence that has at least about 85, 90, 91, 92, 93, 94, 95, 96, 97, 98, 99, or 100% sequence identity to the polypeptide sequence of SEQ ID NO:4, wherein said amino acid sequence further comprises a substitution of at least one amino acid residue in the polypeptide sequence of SEQ ID NO:4 at an amino acid position selected from the group consisting of Ala₆, Leu₉, Glu₄₅, Ile₈₂, Ala₁₁₄, Glu₁₅₂, Ser₁₅₅, His₁₆₃, Met₁₉₆, Asp₂₀₅, Arg₂₃₄, and Leu₂₃₉, wherein the polypeptide preferably induces an immune response against hEpCAM or an antigenic fragment thereof, including inducing or promoting production of antibodies that specifically bind mEpCAM or hEpCAM or an antigenic fragment thereof, inducing or promoting T cell proliferation and/or T cell activation, and/or inducing or promoting production of at least one cytokine. As will be discussed further herein, the position of the substitution or substitutions in the context of the amino acid sequence of the resultant polypeptide can vary relative to the position of the substituted amino acid(s) in the sequence of SEQ ID NO:4 due, e.g., but not limited to, the presence of one or more deletions, additions, and/or substitutions of amino acid residues in the sequence of the resultant polypeptide that do not occur in the SEQ ID NO:4 sequence, or a combination of such additions, deletions, and/or substitutions.

Novel and/or immunogenic amino acid sequences of the invention that have a length and sequence identity similar to SEQ ID NO:4 (i.e., that have at least about 70% sequence identity to SEQ ID NO:4 and about 265 amino acids in length) typically comprise a signal peptide, propeptide and extracellular domain (ECD).

The polypeptides represented by SEQ ID NOS: 13, 32, and 78 are exemplary of polypeptides that comprise a signal peptide, propeptide and ECD. Polypeptides comprise a signal peptide, propeptide and ECD, but that do not include a transmembrane domain and/or cytoplasmic domain are typically excreted from a cell upon expression, e.g., following transfection of the cell with a nucleic acid encoding the polypeptide. Such polypeptides may be termed “soluble” polypeptides, since they do not typically remain bound or anchored to a cell membrane.

SEQ ID NOS: 1-3 represent amino acid sequence segments of the polypeptide sequence of SEQ ID NO:4, which correspond essentially to subsequences of the polypeptide sequence of SEQ ID NO:4 that are typically generated by the proteolytic cleavage of the sequence of SEQ ID NO:4 (e.g., at least in particular cells). Thus, for example, a polypeptide comprising or consisting of SEQ ID NO:4, when such a polypeptide is expressed in a mammalian cell, may be subject to proteolytic cleavage resulting in polypeptides comprising or consisting essentially of one or more of the polypeptide sequences shown in SEQ ID NO: 1, SEQ ID NO:2, and SEQ ID NO:3. In one aspect, the polypeptide comprises a fusion protein comprising the polypeptide sequences of SEQ ID NO:3, SEQ ID NO:2, and SEQ ID NO: 1 fused together in such order N terminal to C terminal, e.g., with the C-terminus of the polypeptide sequence of SEQ ID NO:3 fused to the N-terminus of the polypeptide sequence of SEQ ID NO: 1, the N terminus of the polypeptide sequence of SEQ ID NO:2 fused to the C-terminus of the polypeptide sequence of SEQ ID NO:2, and the N-terminus of the polypeptide sequence of SEQ ID NO: 1 fused to the C-terminus of the sequence of SEQ ID NO:2. SEQ ID. NO:1, for example, represents the largest predominant fragment of a polypeptide consisting of SEQ ID NO:4 obtainable from a culture of mammalian cells transformed with a nucleic acid that expresses SEQ ID NO:4. As such, polypeptide sequences provided by the invention that have a similar composition and length similar to the sequence set forth in SEQ ID NO: 1 (i.e., that are at least about 80% identical to SEQ ID NO: 1 and are that about 185 amino acids in length) can conveniently be referred to as mature extracellular domain polypeptides, since they do not include a signal peptide or propeptide. A TAg polypeptide (e.g., TAg-25, SEQ ID NO:4) may be processed in vivo such that cellular proteases cleave and degrade the signal peptide and, ultimately, the propeptide, thereby leaving a “mature ECD” TAg polypeptide. Fully mature polypeptides typically do not include signal peptides and propeptides. SEQ ID NO: 12 is an example of such a “mature ECD” polypeptide of the invention. A processed TAg polypeptide may, however, further include a TMD fused to the C-terminus of the polypeptide; optionally, a processed TAg polypeptide may further include a CD fused to the C-terminus of the TMD.

A mature domain of a. TAg polypeptide comprises an ECD, transmembrane domain, and cytoplasmic domain. Exemplary TAg polypeptides comprising a mature domain are represented by the polypeptide sequences of SEQ ID NOS:7 and 10. Each of these TAg polypeptides comprises an ECD, transmembrane domain, and cytoplasmic domain and is capable of inducing an immune response against hEpCAM or an antigenic fragment thereof. Exemplary nucleic acids that encode a TAg mature domain are represented by the nucleotide sequences set forth in SEQ ID NOS:22 and 28.

SEQ ID NO:3, which comprises amino acid residues 1-23 of the polypeptide sequence of SEQ NO:4, corresponds to the predicted signal peptide of TAg-25 polypeptide (SEQ ID NO:4). The sequence of SEQ ID NO:3 is predicted to be cleaved from TAg-25 polypeptide upon expression of the polypeptide in mammalian cells. Alternatively, TAg-25 polypeptide can be proteolytically cleaved at an alternative position, such that a smaller signal peptide is removed. Thus, e.g., TAg-25 can be subject to cleavage of a signal peptide after amino acid 22 or amino acid 21 of the sequence of SEQ ID NO:4. In this case, the signal peptide would comprise amino acid residues 1-22 or 1-21 of the SEQ ID NO:4 sequence, respectively.

The polypeptide sequence of SEQ ID NO:2 corresponds to a propeptide of TAg-25, corresponding to amino acid residues 24-80 of SEQ ID NO:4. This propeptide is typically proteolytically cleaved from TAg-25 in mammalian cells. For sake of convenience, amino acid sequences of the invention that are of similar length and composition as SEQ ID NO:2 (i.e., that are about 57 amino acids in length and at least about 70% identical to SEQ ID NO:2) may be referred to as “propeptide” sequences. For example, the invention provides amino acid sequence variants of SEQ ID NO:2, which are described elsewhere herein, that can be described as propeptides.

Polypeptides of the invention may be subject to cell-type specific proteolytic cleavage. Thus, a polypeptide comprising a polypeptide sequence selected from the group consisting of SEQ ID NOS:4, 13, 32, and 78, which polypeptides comprise signal peptide, propeptide, and ECD, can be subject to cellular proteolytic cleavage as described above, in some cell systems, typically resulting in the production of two subsequences—a signal peptide and a propeptide/ECD subsequence, or a three subsequence—signal peptide, a propeptide, and an ECD. However, in other cell systems, such polypeptides may not be subject to significant amounts of proteolytic cleavage.

Sequence Identity and Sequence Similarity

One aspect of the invention relates to a polypeptide comprising an extracellular domain, which typically comprises one or more antigenic or immunogenic regions or subsequences, which include, e.g., one or more epitopes (e.g., B cell and/or T cell epitopes). For example, in a particular aspect, the invention provides an isolated, recombinant or synthetic polypeptide comprising a polypeptide sequence that has at least about 90%, 95%, 96%, 97%, 98%, or 99% identity to the polypeptide sequence of SEQ ID NO: 1. Such polypeptides are able to induce at least one type of immune response, as described above, to hEpCAM and antigenic fragments thereof, including, e.g., sEpCAM, the extracellular domain of hEpCAM, and/or mature domain of hEpCAM. Moreover, such polypeptides can be used to induce or promote an immune response to hEpCAM-associated cells, such as tumor-associated cells that overexpress EpCAM in a mammal, including, e.g., a human. Further features of such polypeptides are provided elsewhere herein.

With regard to nucleic acid sequences, the term “sequence identity” means that two nucleic acid sequences are identical (i.e., on a nucleotide-by-nucleotide basis) over a window of comparison. A percentage of nucleotide sequence identity (or percentage of nucleotide sequence similarity) is calculated by comparing two optimally aligned nucleic acid sequences over the window of comparison, determining the number of positions at which the identical residues occur in both nucleotide sequences to yield the number of matched positions, dividing the number of matched positions by the total number of positions in the window of comparison (i.e., the window size), and multiplying the result by 100 to yield the percentage of sequence identity (or percentage of sequence similarity). With regard to amino acid sequences, the term “sequence identity” likewise means that two amino acid sequences are identical (on an amino acid-by-amino acid basis) over a window of comparison. The percentage of amino acid sequence identity (or percentage of nucleotide sequence similarity) is similarly calculated by comparing two optimally aligned amino acid sequences over the window of comparison, determining the number of positions at which the identical amino acid residues occur in both amino acid sequences to yield the number of matched positions, dividing the number of matched positions by the total number of positions in the window of comparison, and multiplying the result by 100 to yield the percentage of sequence identity (or percentage of sequence similarity). Maximum correspondence can be determined by using one of the sequence algorithms described herein (or other algorithms available to those of ordinary skill in the art) or by visual inspection. The terms “percent identity,” “percent identical,” “percentage of sequence identity, and “percent sequence identity” are used interchangeably.

The term “identity” as used herein is to be considered synonymous with “overall identity,” in contrast to the phrase “local sequence identity,”which measures the identity of a portion or subsequence of a first (standard) sequence to a portion or subsequence of a second sequence in an optimal local sequence alignment. Local sequence identity normally is obtained using algorithms such as those incorporated in the LALIGN or LFASTA programs, which are known in the art.

Optimal alignment is the alignment that provides the highest level of identity between the aligned sequences. In obtaining the optimal alignment, gaps can be introduced, and some amount of non-identical sequences and/or ambiguous sequences can be ignored to obtain an alignment that provides the highest level of identity between the aligned sequences. The introduction of gaps and/or the ignoring of non-homologous/ambiguous sequences are associated with a “gap penalty,” unless otherwise stated herein. In other words, a gap between two sequences will reduce the level of identity by one residue or nucleotide base.

Alignment and comparison of relatively short sequences (less than about 30 residues) is typically straightforward, and identity between relatively short amino acid or nucleic acid sequences can be easily determined by visual inspection. Comparison of longer sequences can require more sophisticated methods to achieve optimal alignment of two sequences. Analysis with an appropriate algorithm, typically facilitated through computer software, commonly is used to determine identity between longer sequences. When using a sequence comparison algorithm, test and reference sequences typically are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. A number of mathematical algorithms for rapidly obtaining the optimal alignment and calculating identity between two or more sequences are known and incorporated into a number of available software programs. Examples of such programs include the MATCH-BOX; MULTAIN, GCG, FASTA, and ROBUST programs for amino acid sequence analysis, and the SIM, GAP, NAP, LAP2, GAP2, and PIPMAKER programs for nucleotide sequences. Suitable software analysis programs for both amino acid and polynucleotide sequence analysis include the ALIGN, CLUSTALW (e.g., version 1.6 and later versions thereof, such as version W 1.8 available from European Bioinformatics Institute, Cambridge, UK), and BLAST programs (e.g., BLAST 2.1, BL2SEQ, and later versions thereof). Select examples are further described in the following paragraphs.

For amino acid sequence analysis and amino acid alignments, a weight matrix, such as the BLOSUM matrixes (e.g., the BLOSUM45, BLOSUM50, BLOSUM62, and BLOSUM80 matrixes—as described in, e.g., Henikoff and Henikoff, Proc. Natl. Acad. Sci. USA 89:10915-10919 (1992)), Gonnet matrixes (e.g., the Gonnet40, Gonnet80, Gonnet120, Gonnet160, Gonnet250, and Gonnet350 matrixes), or PAM matrixes (e.g., the PAM30, PAM70, PAM120, PAM160, PAM250, and PAM350 matrixes), are used in determining identity. BLOSUM matrixes, such as the BLOSUM50 and BLOSUM62 matrixes are commonly used. In the absence of availability of such weight matrixes (e.g., in nucleic acid sequence analysis and with some amino acid analysis programs), a scoring pattern for residue/nucleotide matches and mismatches can be used (e.g., a +5 for a match and −4 for a mismatch pattern).

The ALIGN program produces an optimal global (overall) alignment of the two chosen protein or nucleic acid sequences using a modification of the dynamic programming algorithm described by Myers and Miller CABIOS 4:11-17 (1988). The ALIGN program typically, although not necessary, is used with weighted end-gaps. If gap opening and gap extension penalties are available, they are often set between about −5 to −15 and 0 to −3, respectively, more preferably about −12 and −0.5 to −2, respectively, for amino acid sequence alignments, and −10 to −20 and −3 to −5, respectively, more commonly about −16 and −4, respectively, for nucleic acid sequence alignments. The ALIGN program and principles underlying it are further described in, e.g., Pearson et al., Proc. Natl. Acad. Sci. USA 85:2444-48 (1988), and Pearson et al., Meth. Enzymol. 18:63-98 (1990).

Alternatively, and particularly for multiple sequence analysis (i.e., comparison of more than three sequences), the CLUSTALW program (described in, e.g., Thompson et al. Nucl. Acids Res. 22:4673-4680 (1994)) can be used. CLUSTALW is an algorithm suitable for multiple DNA and amino acid sequence alignments is the CLUSTALW program (Thompson, J. D. et al. (1994) Nucl. Acids Res. 22:4673-4680). CLUSTALW performs multiple pairwise comparisons between groups of sequences and assembles them into a multiple alignment based on homology. In one aspect, Gap open and Gap extension penalties are set at 10 and 0.05, respectively. Alternatively or additionally, the CLUSTALW program is run using “dynamic” (versus “fast”) settings. Typically, nucleotide sequence analysis with CLUSTALW is performed using the BESTFIT matrix, whereas amino acid sequences are evaluated using a variable set of BLOSUM matrixes depending on the level of identity between the sequences (e.g., as used by the CLUSTALW version 1.6 program available through the San Diego Supercomputer Center (SDSC) or version W 1.8 available from European Bioinformatics Institute, Cambridge, UK). Preferably, the CLUSTALW settings are set to the SDSC CLUSTALW default settings (e.g., with respect to special hydrophilic principles of operation are further described in, e.g., Higgins et al., CABIOS 8(2): 189-91 (1992), Thompson et al., Nucleic Acids Res. 22:4673-80 (1994), and Jeanmougin et al., Trends Biochem. Sci. 2:403-07 (1998).

In an alternative format, the identity or percent identity between a particular pair of aligned amino acid sequences refers to the percent amino acid sequence identity that is obtained by ClustalW analysis (e.g., version W 1.8)), counting the number of identical matches in the alignment and dividing such number of identical matches by the greater of (i) the length of the aligned sequences, and (ii) 96, and using the following default ClustalW parameters to achieve slow/accurate pairwise alignments Gap Open Penalty: 10; Gap Extension Penalty:0.10; Protein weight matrix:Gonnet series; DNA weight matrix IUB; Toggle Slow/Fast pairwise alignments=SLOW or FULL Alignment.

Another useful algorithm for determining percent identity or percent similarity is the FASTA algorithm, which is described in Pearson et al., Proc Natl. Acad. Sci. USA 85:2444 (1988). See also, Pearson, Methods Enzymol. 266:227-258 (1996). Typical parameters used in a FASTA alignment of DNA sequences to calculate percent identity are optimized, BL50 Matrix 15: −5, k-tuple 2; joining penalty=40, optimization=28; gap penalty −12, gap length penalty=−2; and width=16.

Other suitable algorithms include the BLAST and. BLAST 2.0 algorithms, which facilitate analysis of at least two amino acid or nucleotide sequences, by aligning a selected sequence against multiple sequences in a database (e.g., GenSeq), or, when modified by an additional algorithm such as BL2SEQ, between two selected sequences. Software for performing BLAST analyses is publicly available through the National Center for Biotechnology Information (NCBI) (http://www.ncbi.nlm.nih.gov/). The BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) can be used with a word length (W) of 11, an expectation (E) of 10, M=5, N=−4 and a comparison of both strands. For amino acid sequences, the BLASTP program (e.g., BLASTP 2.0.14; Jun.-29-2000) can be used with a word length of 3 and an expectation (E) of 10. The BLOSUM62 scoring matrix (see Henikoff & Henikoff, (1989) Proc. Natl. Acad. Sci. USA 89:10915) uses alignments (B) of 50, expectation (E) of 10, M=5, N=−4, and a comparison of both strands. Again, as with other suitable algorithms, the stringency of comparison can be increased until the programidentifies only sequences that are more closely related to those in the sequence listings herein (e.g., sequences having at least about 80, 90, 95, 96, 97% or more % sequence identity to a sequence selected from SEQ ID NOS:19, 27, 33, and 79; or sequences having at least about 80, 90, 95, 96, 97% or more % sequence identity to a sequence selected from SEQ ID NOS:4, 13, 32, or 78.

The BLAST algorithm also performs a statistical analysis of the similarity or identity between two sequences (see, e.g., Karlin & Altschul, (1993) Proc. Natl. Acad. Sci. USA 90:5873-5787). One measure of similarity or identity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

BLAST program analysis also or alternatively can be modified by low complexity filtering programs such as the DUST or SEG programs, which are preferably integrated into the BLAST program operations (see, e.g., Wootton et al., Comput. Chem. 17:149-63 (1993), Altschul et al., Nat. Genet. 6:119-29 (1991), Hancock et al., Comput. Appl. Biosci. 10:67-70 (1991), and Wootton et al., Meth Enzymol. 266:554-71 (1996)). In such aspects, if a lambda ratio is used, useful settings for the ratio are between 0.75 and 0.95, more preferably between 0.8 and 0.9. If gap existence costs (or gap scores) are used in such aspects, the gap existence cost typically is set between about −5 and −15, more typically about −10, and the per residue gap cost typically is set between about 0 to −5, more preferably between 0 and −3 (e.g., −0.5). Similar gap parameters can be used with other programs as appropriate. The BLAST programs and principles underlying them are further described in, e.g., Altschul et al. (1990) J. Mol. Biol. 215:403-10, Karlin and Altschul (1990) Proc. Natl. Acad. Sci. USA 87:2264-68 (as modified by Karlin and Altschul (1993) Proc. Natl. Acad. Sci. USA 90:5873-77), and Altschul et al. (1997) Nucl. Acids Res. 25:3389-3402.

Another example of a useful algorithm is incorporated in PILEUP software. The PILEUP program creates a multiple sequence alignment from a group of related sequences using progressive, pair-wise alignments to show relationship and percent sequence identity or percent sequence similarity. PILEUP uses a simplification of the progressive alignment method of Feng & Doolittle (1987) J. Mol. Evol. 35:351-360, which is similar to the method described by Higgins & Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity (or percent sequence similarity) relationship using specified parameters. Exemplary parameters for the PILEUP program are: default gap weight (3.00), default gap length weight (0.10), and weighted end gaps. PILEUP is a component of the GCG sequence analysis software package, e.g., version 7.0 (see, e.g., Devereaux et al. (1984) Nucl. Acids Res. 12:387-395).

Other useful algorithms for performing identity analysis include the local homology algorithm of Smith and Waterman (1981) Adv. Appl. Math. 2:482, the homology alignment algorithm of Needleman and Wunsch (1970) J. Mol. Biol. 48:443, and the search Computerized implementations of these algorithms (e.g., GAP, BESTFIT, FASTA and TFASTA) are provided in the Wisconsin Genetics Software Package Release 7.0, Genetics Computer Group, 575 Science Dr., Madison, Wis.

Several additional commercially available software suites incorporate the ALIGN, BLAST, and CLUSTALW programs and similar functions, and may include significant improvements in settings and analysis. Examples of such programs include the GCG suite of programs and those available through DNASTAR, Inc. (Madison, Wis.), such as the Lasergene® and Protean® programs. A preferred alignment method is the Jotun Hein method, incorporated within the MegaLine™ DNASTAR package (MegaLine™ Version 4.03) used according to the manufacturer's instructions and default values specified in the program.

As applied to polypeptides, the term substantial identity or substantial similarity means that two polypeptide sequences, when optimally aligned, such as by the programs BLAST, GAP or BESTFIT using default gap weights (described in detail below) or by visual inspection, share at least about 60 percent, 70 percent, or 80 percent sequence identity or sequence similarity, preferably at least about 90 percent amino acid residue sequence identity or sequence similarity, more preferably at least about 95 percent sequence identity or sequence similarity, or more (including, e.g., about 96, 97, 98, 98.5, 99, or more percent amino acid residue sequence identity or sequence similarity). Similarly, as applied in the context of two nucleic acids, the term substantial identity or substantial similarity means that the two nucleic acid sequences, when optimally aligned, such as by the programs BLAST, GAP or BESTFIT using default gap weights (described in detail below) or by visual inspection, share at least about 60 percent, 70 percent, or 80 percent sequence identity or sequence similarity, preferably at least about 90 percent amino acid residue sequence identity or sequence similarity, more preferably at least about 95 percent sequence identity or sequence similarity, or more (including, e.g., about 96, 97, 98, 98.5, 99, or more percent nucleotide sequence identity or sequence similarity).

It will be understood by one of ordinary skill in the art, that the above discussion of search and alignment algorithms also applies to identification and evaluation of polynucleotide sequences, with the substitution of query sequences comprising nucleotide sequences, and where appropriate, selection of nucleic acid databases.

In one aspect, the present invention provides homologue nucleic acids having at least about 70, 75, 80, 85, 90, 91, 92, 93, 94, 95, 96, 97, 98, 99, 99.5 or 100% sequence identity or sequence similarity with the nucleic acid sequence selected from the group of SEQ ID NOS:16-28, 32, 33-35, and 79 or a fragment thereof, such as a fragment encoding an antigenic polypeptide that induces an immune response against hEpCAM, or an antigenic fragment thereof, or a cell or tissue expressing hEpCAM. In another aspect, the present invention provides homologue polypeptides having at least about 70, 75, 80, 85, 90, 91, 92, 93, 94, 95, 96, 97, 98, 99, 99.5 or 100% sequence identity or sequence similarity with a polypeptide sequence selected from the group of SEQ ID NOS:1-15, 32, 34, 78, 80, and 92, or a fragment thereof, such as an antigenic fragment that induces an immune response, including an immune response against hEpCAM, or an antigenic fragment thereof, or a cell or tissue expressing hEpCAM.

In yet another aspect, the present invention provides TAg homologue polypeptides that are substantially identical or substantially similar over at least about 150, 160, 170, or 180 contiguous amino acids of at least one of SEQ ID NOS: 1, 9, 12, and 92, wherein some such polypeptides induce an immune response against hEpCAM or a cell or tissue expressing hEpCAM. In another aspect are provided TAg homologue polypeptides that are substantially identical or substantially similar over at least about 200, 210, 220, or 230 contiguous amino acids of at least one of SEQ ID NOS:4, 13, 32, and 78, wherein some such polypeptides induce an immune response against hEpCAM or a cell or tissue expressing hEpCAM. Also included are TAg homologue polypeptides that are substantially identical or substantially similar over at least about 225, 240, 250, or 260 contiguous amino acids of at least one of SEQ ID NOS:4, 13, 32, and 78, wherein some such polypeptides induce an immune response against hEpCAM or a cell or tissue expressing hEpCAM.

Preferably, amino acid residue positions that are not identical differ by conservative amino acid substitutions. Conservative amino acid substitution refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Advantageously, many polypeptides (and nucleic acids) of the invention described above and throughout this application typically are capable of generating an immune response in vivo in a mammalian host including a primate and, more particularly, a human. Alternatively, an immune response can be generated in a tissue culture or other population of cells comprising a number of immune system cells under conditions suitable for such cells to exhibit an immune response. The measurement of such an immune response can be in vivo (e.g., an indication of a reduction of progression of an EpCAM-associated cancer) or in vitro (e.g., the result of an ELISA assay or T cell proliferation assay using sera of a mammalian host treated with the polypeptide of the present aspect). Examples of immune responses to EpCAM resulting from such polypeptides and other polypeptides of the invention, and the detection of such responses, are described in further detail elsewhere herein and throughout. The immune response to a mammalian EpCAM which is induced by a polypeptide of the invention (or a nucleic acid of the invention) can be measured by any suitable technique. For example, an increase in the amount of antibodies produced that bind to EpCAM, typically determined by measuring the optical density (OD) values in an ELISA antibody assay, and/or increased proliferation of EpCAM-reactive T cells in reaction to a polypeptide of the invention. The immune response induced by a polypeptide of the invention can be compared to the immune response induced by a mammalian EpCAM, such as hEpCAM, or antigenic fragment thereof, such as an antigenic fragment comprising at least the ECD and optionally the PP of hEpCAM.

Sequence Variations

The invention includes polypeptides that comprise conservatively modified variations of any polypeptide sequence of the invention described herein. In a particular aspect, such polypeptide variants include conservatively modified variations of a polypeptide sequence selected from the group of SEQ ID NOS: 1, 4-10, 12-14, 32, 34, 78, and 92.

A conservative amino acid residue substitution typically involves exchanging a member within one functional class of amino acid residues for a residue that belongs to the same functional class (identical amino acid residues are considered functionally homologous or conserved in calculating percent functional homology).

Conservative substitution tables providing functionally similar amino acids are well known in the art. Table 1 sets forth exemplary functional classes of amino acids and members of those classes that would constitute “conservative substitutions” for one another.

TABLE 1
Amino Acid Residue Classes

| Amino Acid Class | Amino Acid Residues |
|---|---|
| Acidic Residues | ASP and GLU |
| Basic Residues | LYS, ARG, and HIS |
| Hydrophilic Uncharged Residues | SER, THR, ASN, and GLN |
| Aliphatic Uncharged Residues | GLY, ALA, VAL, LEU, and ILE |
| Non-polar Uncharged Residues | CYS, MET, and PRO |
| Aromatic Residues | PHE, TYR, and TRP |

An alternative set of conservative amino acid substitutions, delineated by six conservation groups, is provided in Table 2.

TABLE 2
Alternative Amino Acid Residue Substitution Groups

| | | | |
|---|---|---|---|
| 1 | Alanine (A) | Serine (S) | Threonine (T) |
| 2 | Aspartic acid (D) | Glutamic acid (E) | |
| 3 | Asparagine (N) | Glutamine (Q) | |
| 4 | Arginine (R) | Lysine (K) | |
| 5 | Isoleucine (I) | Leucine (L) | Methionine (M) |
| 6 | Phenylalanine (F) | Tyrosine (Y) | Tryptophan (W) |

More conservative substitutions exist within the above-described amino acid residue classes, which also or alternatively can be suitable. Conservation groups for substitutions that are more conservative include: valine-leucine-isoleucine, phenylalanine-tyrosine, lysine-arginine, alanine-valine, and asparagine-glutamine. Thus, for example, the invention provides a polypeptide comprising an amino acid sequence that has at least about 90, 95, 96, 97, 98, or 99% identity to SEQ ID NO: 1 that differs from SEQ ID NO: 1 by mostly (e.g., at least 50%), if not entirely by such more conservative amino acid substitutions.

Additional groups of amino acids substitutions that also can be suitable can be determined using the principles described in, e.g., Creighton (1984) PROTEINS: STRUCTURE AND MOLECULAR PROPERTIES (2d Ed. 1993), W.H. Freeman and Company. In some aspects, at least about 33%, at least about 50%, at least about 65%, or more (e.g., at least about 90, 95, 96, 97% or more) of the substitutions in the amino acid sequence variant (as compared to SEQ ID NO: 1), comprise substitutions of amino acid residues in a polypeptide sequence of the invention (SEQ ID NOS: 1, 4-10, 12-14 and 92) with residues that are within the same functional homology class (as determined by any suitable classification system, such as those described above) as the amino acid residues of the polypeptide sequence (SEQ ID NOS: 1, 4-8, 78 and 92, respectively) that they replace.

Conservatively substituted variations of a polypeptide sequence of the present invention include substitutions of a small percentage, typically less than 5%, more typically less than 4%, 3%/, 2%, or 1%, of the amino acids of the sequence, with a conservatively selected amino acid of the same conservative substitution group.

One aspect of the invention pertains to a chimeric antigenic polypeptide comprising an antigenic polypeptide sequence having at least about 90, 91, 92, 93, 94, 95, 96, 97, 98, or 99% amino acid sequence identity to a polypeptide sequence selected from the group of SEQ ID NOS:1, 4-10, 12-14, 32, 34, 78, and 92, and wherein such polypeptide induces and/or promotes an immune response against hEpCAM or antigenic fragment thereof. The immune response induced against hEpCAM can be any type of immune response, which can be manifested in any detectable manner. For example, the polypeptide can induce a cellular immune response (e.g., a cytotoxic or T cell immune response), a humoral (e.g., an antibody-associated and/or antibody-mediated) immune response, or both. Immune responses include an ability to induce and/or enhance an immune response against hEpCAM, an ability to induce and/or enhance a hEpCAM-specific T cell proliferative response, an ability to induce or enhance production of at least one cytokine, and/or an ability to bind anti-hEpCAM antibodies. Standard methods for evaluating such immune responses are known to those of skill in the art, and selected methods are described below. Also provided are polypeptide variants of such an antigenic polypeptide, wherein the amino acid sequence of the polypeptide variant differs from the respective antigenic polypeptide sequence by one or more conservative amino acid residue substitutions, although non-conservative substitutions are sometimes permissible or even preferred (examples of such non-conservative substitutions are discussed further herein). For example, the sequence of the polypeptide variant can vary from such anti genic polypeptide sequence by one or more substitutions of amino acid residues in the antigenic polypeptide sequence with one or more amino acid residues having similar weight (i.e., a residue that has weight homology to the residue in the respective polypeptide sequence that it replaces). The weight (and correspondingly the size) of amino acid residues of a polypeptide can significantly impact the structure of the polypeptide. Weight-based conservation or homology is based on whether a non-identical corresponding amino acid is associated with a positive score on one of the weight-based matrices described herein (e.g., the BLOSUM50 matrix and preferably the PAM250 matrix). Similar to the above-described functional amino acid classes, naturally occurring amino acid residues can be divided into weight-based conservations groups (which are divided between “strong” and “weak” conservation groups). The eight commonly used weight-based strong conservation groups are Ser Thr Ala, Asn Glu Gln Lys, Asn His Gln Lys, Asn Asp Glu Gln, Gln His Arg Lys, Met Ile Leu Val, Met Ile Leu Phe, His Tyr, and Phe Tyr Trp. Weight-based weak conservation groups include Cys Ser Ala, Ala Thr Val, Ser Ala Gly, Ser Thr Asn Lys, Ser Thr Pro Ala, Ser Gly Asn Asp, Ser Asn Asp Glu Gln Lys, Asn Asp Glu Gln His Lys, Asn Glu Gln His Arg Lys, Phe Val Leu Ile Met, and His Phe Tyr. Some versions of the CLUSTAL W sequence analysis program provide an analysis of weight-based strong conservation and weak conservation groups in the output of an alignment, thereby offering a convenient technique for determining weight-based conservation (e.g., CLUSTAL W provided by the SDSC, which typically is used with the SDSC default settings). In some aspects, at least about 33%, at least about 50%, at least about 65%, or more. (e.g., at least about 90%) of the substitutions in such polypeptide variant comprise substitutions wherein a residue within a weight-based conservation replaces an amino acid residue of the antigenic polypeptide sequence that is in the same weight-based conservation group. In other words, such a percentage of substitutions are conserved in terms of amino acid residue weight characteristics.

The sequence of a polypeptide variant can differ from the antigenic polypeptide sequence by one or more substitutions with one or more amino acid residues having a similar hydropathy profile (i.e., that exhibit similar hydrophilicity) to the substituted (original) residues of the antigenic polypeptide. A hydropathy profile can be determined using the Kyte & Doolittle index, the scores for each naturally occurring amino acid in the index being as follows: I (+4.5), V (+4.2), L (+3.8), F (+2.8), C (+2.5), M (+1.9); A (+1.8), G (−0.4), T (−0.7), S (−0.8), W (−0.9), Y (−1.3), P (−1.6), H (−3.2); E (−3.5), Q (−3.5), D (−3.5), N (−3.5), K (−3.9), and R (−4.5) (see, e.g., U.S. Pat. No. 4,554,101 and Kyte & Doolittle, J. Molec. Biol. 157:105-32 (1982) for further discussion). Examples of typical amino acid substitutions that retain similar or identical hydrophilicity include arginine-lysine substitutions, glutamate-aspartate substitutions, serine-threonine substitutions, glutamine-asparagine substitutions, and valine-leucine-isoleucine substitutions. Algorithms and software, such as the GREASE program available through the SDSC, provide a convenient way for quickly assessing the hydropathy profile of an amino acid sequence. Because a substantial proportion (e.g., at least about 33%), if not most (at least 50%) or nearly all (e.g., about 65, 80, 90, 95, 96, 97, 98, 99%) of the amino acid substitutions in the sequence of a polypeptide variant often will have a similar hydropathy score as the amino acid residue that they replace in the antigenic (reference) polypeptide sequence, the sequence of the polypeptide variant is expected to exhibit a similar GREASE program output as the antigenic polypeptide sequence. For example, in a particular aspect, a polypeptide variant of SEQ ID NO: 1 is expected to have a GREASE program (or similar program) output that is more like the GREASE output obtained by inputting the polypeptide sequence of SEQ ID NO: 1 than that obtained using a non-human ortholog of EpCAM, such as TACST1 (i.e., GenBank Accession No. AAH05618) (which can be determined by visual inspection or computer-aided comparison of the graphical (e.g., graphical overlay/alignment) and/or numerical output provided by subjecting the test variant sequence and SEQ ID NO: 1 to the program).

The conservation of amino acid residues in terms of functional homology, weight homology, and hydropathy characteristics, also apply to other polypeptide sequence variants provided by the invention, including, but not limited to, e.g., polypeptide sequence variants of a polypeptide sequence selected from the group consisting of SEQ ID NOS:2, 3, 11, 15, 80, which are discussed further herein.

In a particular aspect, the invention includes at least one such polypeptide variant comprising an amino acid sequence that differs from an antigenic polypeptide sequence selected from the group of SEQ ID NOS:1, 4-10, 12-14, 32, 34, 78, and 92, wherein the amino acid sequence of the variant has at least one such amino acid residue substitution selected according to weight-based conservation or homology or similar hydropathy profile as discussed above.

Such polypeptide variants described above typically induce at least one type of immune response against hEpCAM as described previously and in greater detail below in the Examples.

Polypeptides Comprising Selected Epitopes

Polypeptides of the invention that have an ability to induce an immune response against a mEpCAM, such as hEpCAM, or antigenic fragment thereof typically include one or more antigenic determinants (e.g., epitopes), such as those described further below and set forth in the sequence listing. Some such epitopes are cross-reactive with mEpCAM or hEpCAM. For example, in one aspect, the invention provides an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:1, 4-10, 12-14, 32, 34, 78, and 92, wherein said polypeptide includes as a subsequence within its polypeptide sequence at least one antigenic determinant (e.g., epitope) that is identical to a peptide sequence selected from the group consisting of SEQ ID NOS:47-64. Such polypeptides induce at least one type of immune response of a TAg polypeptide against hEpCAM as described above. Thus, for example, the invention provides an isolated or recombinant polypeptide comprising a polypeptide sequence having at least about 95, 96, 97, 98, or 99% sequence identity to SEQ ID NO: 1, which polypeptide includes as a subsequence within its sequence at least one peptide sequence selected from the group of SEQ ID NOS:47-64.

As some of the peptide sequences in the group consisting of SEQ ID NOS:47-64 share one or more residues in common with other peptide sequences in this group, a polypeptide of the invention may include more than one of these peptide sequences, although such sequences are not discrete with respect to (i.e., are not separate from) one another. Two peptide sequences are “discrete” sequences in a polypeptide sequence of the invention if none of their respective amino acid residues overlap with one another in the polypeptide sequence. For example, SEQ ID NO:56 only differs from SEQ ID NO:54 by the addition of an N-terminal. Gln residue. As such, a polypeptide of the invention that comprises the peptide sequence of SEQ ID NO:56 will typically also comprise the peptide sequence of SEQ ID NO:54; the sequences overlap and share substantial sequence identity. In this instance, the peptide sequence of SEQ ID NO:54 would not be a discrete or separate peptide sequence, but would comprise a subsequence of the peptide sequence of SEQ ID NO:56. Alternatively or in addition, polypeptide of the invention can also comprise at least two peptide sequences selected from the group consisting of SEQ ID NOS:47-64, wherein each peptide sequence is present as discrete peptide sequence within the sequence of the polypeptide. The polypeptide can advantageously include at least three peptide sequences, at least four peptide sequences, at least five peptide sequences, or more that are selected from the group consisting of SEQ ID NOS:47-64, which peptide sequences are present as discrete peptide sequences (e.g., the peptide sequences do not overlap with one another) within the sequence of the polypeptide.

One particular aspect of the invention provides an isolated or recombinant polypeptide variant of the polypeptide sequence set forth in SEQ ID NO:1 in which a serine residue is inserted at about position 149 of SEQ ID NO: 1. An example of such a polypeptide variant is the polypeptide sequence of SEQ ID NO: 12. The sequence of SEQ ID NO: 12 differs from the sequence of SEQ ID NO: 1 by further comprises an insertion of a serine residue between Ser₁₄₈ and Lys₁₄₉ in the sequence of SEQ ID NO:1. Such polypeptides induce at least one type of immune response of a TAg polypeptide against hEpCAM as described above.

In another aspect, the invention provides an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:6-8, 10, and 34, wherein said polypeptide includes as a subsequence within its sequence at least one antigenic determinant that is identical to a peptide sequence selected from the group consisting of SEQ ID NOS:65-70. In a particular aspect, the invention provides an isolated or recombinant polypeptide having at least about 96, 97, 98, or 99% sequence identity to a polypeptide sequence selected from the group of SEQ ID NOS:6-8, wherein said polypeptide comprises as a subsequence within its sequence at least one peptide sequence selected from the group consisting of SEQ ID NOS:65-70.

Also provided is an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:4-6, 13-14, 32, 34, and 78, wherein said polypeptide includes as a subsequence within its sequence at least one peptide sequence selected from the group consisting of SEQ ID NOS:71-73. Also provided is an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:4, 6, 13-14, 32, 34, and 78, wherein said polypeptide includes as a subsequence within its sequence at least one peptide sequence selected from the group consisting of SEQ ID NOS:74-76.

Also provided is an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:4, 6, 13-14, 32, 34, and 78, wherein said polypeptide includes as a subsequence within its sequence at least one peptide sequence selected from the group consisting of SEQ ID NOS:47-64, at least one peptide sequence selected from the group consisting of SEQ ID NOS:71-73, and at least one peptide sequence selected from the group consisting of SEQ ID NOS:74-76.

In another aspect, the invention includes an isolated or recombinant polypeptide comprising a polypeptide sequence having at least about 90, 91, 92, 93, 94, 95, 96, 97, 98, or 99% sequence identity to a polypeptide sequence selected from the group of SEQ ID NOS:6, 14, and 34, wherein said polypeptide includes as a subsequence within its sequence at least one peptide sequence selected from the group consisting of SEQ ID NOS:47-64, at least one peptide sequence selected from the group consisting of SEQ ID NOS:65-70, at least one peptide sequence selected from the group consisting of SEQ ID NOS:71-73, and at least one peptide sequence selected from the group consisting of SEQ ID NOS:74-76, and optionally including the peptide sequence of SEQ ID NO:77.

In another aspect, the invention provides is an isolated or recombinant polypeptide comprising a polypeptide sequence having at least about 90, 91, 92, 93, 94, 95, 96, 97, 98, or. 99% sequence identity to a polypeptide sequence selected from the group of SEQ ID NOS:6-8, 10, 14, and 34, wherein said polypeptide includes as a subsequence within its sequence at least one peptide sequence selected from the group consisting of SEQ ID NOS:47-64, at least one peptide sequence selected from the group consisting of SEQ ID NOS:65-70, and optionally including the peptide sequence of SEQ ID NO:77.

All such polypeptides comprising one or more of such peptide sequences (i.e., epitopes) described above typically induce at least one type of immune response against hEpCAM or antigenic fragment thereof as described previously and below in the Examples.

Polypeptides Comprising Propeptides (PP) and/or Extracellular Domains (ECD)

In another aspect, the invention also provides an isolated, recombinant or non-naturally occurring polypeptide comprising a propeptide and an immunogenic ECD. Such PP/ECD polypeptides typically induce at least one type of immune response against hEpCAM or an antigenic fragment thereof as described previously and in detail below. For example, the invention provides an immunogenic polypeptide comprising: (1) a first polypeptide (i.e., ECD) comprising a polypeptide sequence is selected from the group of SEQ ID NOS: 1, 7-10, 12, and 92 or any one of the above-described amino acid sequence variants of SEQ ID NOS: 1, 7-10, 12, 92, and (2) a second polypeptide (i.e., propeptide) comprising a polypeptide sequence having at least about 80, 85, 90, 91, 92, 93, 94, 95, 96, 97, 98, 99, or 100% identity to the polypeptide sequence of SEQ ID NO:2 or SEQ ID NO:38. An exemplary ECD sequence is a polypeptide sequence selected from the group consisting of SEQ ID NOS:1, 9, 12, and 92. An exemplary PP/ECD polypeptide is the polypeptide sequence of SEQ ID NO: 5.

Typically, the propeptide sequence is fused to the ECD sequence. The first and second polypeptide sequences can have any suitable relationship to one another in the polypeptide (e.g., with respect to bonding and/or positioning in the polypeptide). Typically, the propeptide (second polypeptide) is positioned N-terminal to the ECD (first polypeptide). Commonly, the C-terminus of the propeptide sequence will be positioned at (such that the propeptide sequence is fused to by a normal peptide bond) or near (e.g., within about 10 amino acid residues of) the N-terminus of the ECD polypeptide sequence. The resulting immunogenic polypeptide comprising the first and second polypeptides (propeptide and ECD) is commonly subject to proteolytic cleavage when expressed in mammalian cells, especially primate cells, and most especially human cells; either in vitro, in vivo, or both. An Arg Arg Ala or similar amino acid motif (e.g., an Arg Arg Ile or Arg Arg Met motif) near the junction of the sequences of the first and second polypeptides in the immunogenic polypeptide sequence may act as a protease cleavage signal in this respect in many mammalian cell systems. Further characteristics of such motifs and predicted protease cleavage features of such polypeptides are provided elsewhere herein. Such polypeptides typically induce at least one type of immune response against hEpCAM or an antigenic fragment thereof as described previously and below.

The propeptide may comprise any suitable amino acid sequence that fulfills the requisite level of amino acid sequence identity to SEQ ID NO:2 or SEQ ID NO:38 and that imparts one or more desired biological functional and/or structural qualities to the polypeptide. For example, the propeptide may itself be immunogenic and/or may enhance or induce an immune response to hEpCAM. A polypeptide comprising such a propeptide of the invention and an ECD of the invention may have an ability to induce an immune response against hEpCAM that differs from (e.g., is greater than) that induced by an ECD polypeptide. A propeptide of the invention may be able to induce an immune response against hEpCAM independently. In some polypeptides provided by the invention, the EpCAM-specific immune response induced by a propeptide of the invention is greater than that induced by an ECD polypeptide of the invention.

The invention includes an isolated or recombinant propeptide comprising a polypeptide sequence having at least about 80, 85, 90, 91, 92, 93, 94, 95, 96, 97, 98, 99, or 100% identity to the polypeptide sequence of SEQ ID NO:2. Some such propeptides comprise a polypeptide sequence that further includes within said polypeptide sequence at least one peptide sequence selected from the group consisting of SEQ ID NOS:71-73. Such a propeptide may comprise a polypeptide sequence that further includes the peptide sequence of (1) SEQ ID NO:74 or SEQ ID NO:76, (2) SEQ ID NO:73, and (3) SEQ ID NO:71, wherein these peptide sequences are arranged in a N-to-C-terminal order with respect to one another in the propeptide sequence; the peptide sequence of SEQ ID NO:74 or SEQ ID NO:76 may overlap with the sequence of SEQ ID NO:73 in part.

In another aspect, the invention provides a propeptide comprising a polypeptide sequence that falls within the sequence pattern Gln Xaa₁ Xaa₂ Cys Val Cys Xaa₃ Asn Tyr Lys Leu Xaa₄ Xaa₅ Xaa₆ Cys Xaa₇ Xaa₈ Asn Xaa₉ Xaa₁₀ Xaa₁₁ Xaa₁₂ Cys Gln Cys Thr Ser Xaa₁₃ Gly Xaa₁₄ Gln Asn Thr Val Ile Cys Ser Lys Leu Ala Xaa₁₅ Met Lys Ala Glu Met Xaa₁₆ Xaa₁₇ Ser Lys Xaa₁₈ Gly Arg (SEQ ID NO:81), wherein each Xaa represents any suitable amino acid residue. Usually, the amino acid residues at the variable (i.e., Xaa) positions in a sequence falling within this sequence pattern are selected from the amino acid residues set forth in Table 3:

TABLE 3

| Position | Selected Residues |
|---|---|
| Xaa₁ | E, R, or K |
| Xaa₂ | D or E |
| Xaa₃ | D, E, or N |
| Xaa₄ | A or T |
| Xaa₅ | S, T, or V |
| Xaa₆ | N, R, or S |
| Xaa₇ | F, S, or Y |
| Xaa₈ | E, L, or V |
| Xaa₉ | E or N |
| Xaa₁₀ | N or Y |
| Xaa₁₁ | G or R |
| Xaa₁₂ | E or Q |
| Xaa₁₃ | V or Y |
| Xaa₁₄ | A or T |
| Xaa₁₅ | A or S |
| Xaa₁₆ | A or V |
| Xaa₁₇ | N or T |
| Xaa₁₈ | G or H |
| Xaa₁₉ | L or S |

In more particular aspects, the propeptide comprises a polypeptide sequence that falls within the sequence pattern Gln Xaa₁ Xaa₂ Cys Val Cys Glu Asn Tyr Lys Leu Ala Val Xaa₃ Cys Xaa₄ Xaa₅ Asn Xaa₆ Xaa₇ Xaa₈ Xaa₉ Cys Gln Cys Thr Ser Xaa₁₀ Gly Xaa₁₁ Gln Asn Thr Val Ile Cys Ser Lys Leu Ala Val Met Lys Ala Glu Met Xaa₁₂ Xaa₁₃ Ser Lys Xaa₁₄ Gly Arg (SEQ ID NO:82), wherein each Xaa represents any suitable amino acid residue. Commonly the amino acid residues in the variable positions in this sequence pattern are selected from the amino acid residues in Table 4.

TABLE 4

| Position | Selected Residues |
|---|---|
| Xaa₁ | E, R, or K |
| Xaa₂ | D or E |
| Xaa₃ | N, R, or S |
| Xaa₄ | F, S, or Y |
| Xaa₅ | E, L, or V |
| Xaa₆ | E or N |
| Xaa₇ | G or R |
| Xaa₈ | E or Q |
| Xaa₉ | V or Y |
| Xaa₁₀ | A or T |
| Xaa₁₁ | A or V |
| Xaa₁₂ | N or T |
| Xaa₁₃ | G or H |
| Xaa₁₄ | L or S |

The propeptide also comprises a subsequence within the immature form of certain TAg polypeptides, such as, e.g., TAg-25 (SEQ ID NO:4), TAg-21 (SEQ ID NO:13), TAG-18 (SEQ ID NO:32). The propeptide is typically subject to proteolytic cleavage. For some polypeptides of the invention that initially comprise (e.g., upon initial expression in a cell) a signal peptide, propeptide and ECD, the signal peptide and propeptide portion are similarly cleaved and degraded by cellular proteases after expression of the polypeptide, e.g., in vivo or ex vivo. A fully processed polypeptide that does not include a signal peptide or propeptide may be referred to as a “mature” polypeptide. In some instances, a “mature” polypeptide may refer to a polypeptide comprising only an ECD. However, the term “mature” polypeptide is also used in reference to a polypeptide that comprises an ECD and a TM, and optionally further includes a CD. The term “mature domain” typically refers to a polypeptide comprising an ECD, CD, and TMD. As with hEpCAM, the mature domain of TAg polypeptides of the invention typically includes an ECD, CD and TMD. An exemplary polypeptide comprising a mature domain is the polypeptide sequence of SEQ ID NO:7.

In another aspect, the invention provides an isolated or recombinant polypeptide that induces and/or promotes an immune response against human EpCAM comprising the polypeptide sequence of SEQ ID NO:5. Such a polypeptide usually undergoes proteolytic cleavage within the sequence of SEQ ID NO:5 when the polypeptide is expressed in eukaryotic cells, particularly in human or primate cells (either in vivo or in vitro); such cleavage results in a propeptide (e.g., the polypeptide sequence of SEQ ID NO:2) or a similar sequence (e.g., a polypeptide sequence that is about 1-3 amino acids longer or shorter in length than the sequence of SEQ ID NO:2 at the C-terminus thereof), and a relatively stable polypeptide (the “mature” ECD) that comprises the polypeptide sequence of SEQ ID NO: 1 or a similar polypeptide sequence. The propeptide is usually subsequently degraded.

In another aspect, the invention provides an isolated or recombinant chimeric polypeptide that induces and/or promotes an immune response against hEpCAM or an antigenic fragment thereof that comprise a polypeptide sequence having at least about 96, 97, 98 or 99% sequence identity to the polypeptide sequence of SEQ ID NO:5. Such chimeric polypeptides comprise a polypeptide sequence that includes as a subsequence(s) at least one epitope peptide sequence selected from the group consisting of SEQ ID NOS:47-64 and 71-73. Usually, the polypeptide sequence of such a chimeric polypeptide includes at least 2, at least 3, at least 4, at least 5, or more epitope peptide sequences selected from the group consisting of SEQ ID NOS:47-64 and 71-73. As discussed above, many such peptide sequences overlap in terms of residues or motifs, such that the isolated or recombinant polypeptide may comprise several of these peptide sequences as overlapping, but not discrete subsequences.

Some such chimeric polypeptides may comprise a polypeptide sequence that includes as discrete subsequences (unless otherwise noted) with said polypeptide sequence at least 2, 3, 4, 5, 6, 7, 8, or preferably 9 epitope peptide sequences selected from the group consisting of: (1) SEQ ID NO:71 or SEQ ID NO:72; (2) SEQ ID NO:47 and/or SEQ ID NO:63 or SEQ ID NO:64; (3) SEQ ID NO:59 or SEQ ID NO:60; (4) SEQ ID NO:57 or SEQ ID NO:58; (5) SEQ ID NO:48; (6) SEQ ID NO:49 or any one of SEQ ID NOS:50-53 (wherein the sequence of any of SEQ ID NOS:50-53 can overlap with the sequence of SEQ ID NO:48); (7) any one of SEQ ID NOS:54-56, (8) SEQ ID NO:61 or SEQ ID NO:62; and (9) any one of SEQ ID NOS:65-70; wherein the two or more peptide sequences are positioned with respect to one another in the polypeptide sequence of the chimeric polypeptide in N-terminal to C-terminal order in the order designated above from (1) to (9). The sequence of such chimeric polypeptide can include as subsequences any suitable combination of at least two of these 9 peptide sequences.

Such a chimeric polypeptide above may further comprise a functional signal peptide, including, e.g., the signal peptide sequence of SEQ ID NO:3 or SEQ ID NO:37 or any signal peptide described further herein. Such chimeric polypeptide also or alternatively may comprise a suitable transmembrane domain as described further herein (e.g., any sequence selected from the group of SEQ ID NOS: 15, 45, and 80) alone, or in combination with a suitable cytoplasmic domain as described further herein (e.g., the polypeptide sequence of SEQ ID NO:46).

Such chimeric polypeptides can also or alternatively can comprise a polypeptide sequence comprising: (1) a first cysteine-rich domain according to the sequence pattern Cys Xaa Cys Xaa₍₈₎ Cys Xaa₍₇₎ Cys Xaa Cys Xaa₍₁₀₎ Cys (SEQ ID NO:84), (2) a cysteine-rich domain (similar to a thyroglobulin type 1A motif or domain) according to the sequence pattern Cys Xaa₍₃₂₎ Cys Xaa₍₁₀₎ Cys Xaa₍₅₎ Cys Xaa Cys Xaa₍₁₆₎ Cys (SEQ ID NO:85), or (3) a first cysteine-rich domain according to SEQ ID NO:84 and second cysteine-rich domain according to SEQ ID NO:85 (i.e., sequence patterns (1) and (2)), wherein Xaa represents any suitable amino acid sequence and subscripted parentheticals refer to numbers of residues occurring at a particular position (e.g., Xaa₍₈₎=Xaa Xaa Xaa Xaa Xaa Xaa, Xaa Xaa). In a particular aspect, such chimeric polypeptide comprises a polypeptide sequence that comprises a first cysteine-rich domain according to the sequence pattern Cys Val Cys Glu Asn Tyr Lys Leu Ala Val Xaa Cys Xaa₍₇₎ Cys Xaa Cys Xaa₍₁₀₎ Cys (SEQ ID NO:86), a second cysteine-rich domain according to the sequence pattern Cys Xaa₍₁₃₎ Arg Arg Xaa• Xaa₍₆₎ Gln Asn Asn Asp Gly Leu Tyr Asp Pro Asp Cys Asp Glu Ser Gly Leu Phe Lys Xaa₍₃₎ Cys Xaa₍₃₎ Ala Thr Cys Trp Cys Val Asn Thr Ala Xaa₍₁₂₎ Cys (SEQ ID NO:87), wherein Xaa• is preferably an Ala, Ile, or Met residue.

In another aspect, such chimeric polypeptide comprises a polypeptide sequence that comprises twelve cysteines, characterized by 1-4,2-6, 3-5 disulfide bonds in a first domain (i.e., Cys₁-Cys₄, Cys₂-Cys₆, Cys₃-Cys₅ bonding—wherein the subscripted numbers reference the numbering of the cysteines in the amino acid sequence from N-terminus to C-terminus) and 1-2, 3-4, 5-6 Cys-Cys bonding in a second domain (e.g., Cys₇-Cys₈, Cys₉-Cys₁₀, and Cys₁₁-Cys₁₂ bonding), which second domain is similar to the thyroglobulin type 1A domain of insulin-like growth factor-binding proteins 1 and 6. These cysteine-rich regions normally occur in the chimeric polypeptide in a similar position to the cysteine-rich regions of SEQ ID NO:5, as applicable (e.g., a Cys₁-Cys₄ bond, normally corresponds to a cysteine at about position 27 forming a disulfide bond with a cysteine at about position 46 in an amino acid sequence variant of SEQ ID NO:4). Alternatively, the first cysteine-rich domain (i.e., the portion of the amino acid sequence comprising Cys₁, Cys₂, Cys₃, Cys₄, Cys₅, and Cys₆) can be characterized by 1-3, 2-4, 5-6 pattern of cysteine-cysteine bonding. Techniques for the analysis of disulfide bonding and glycosylation are provided in, e.g., Chong and Speicher, J. Biol. Chem. 276(8):5804-5813 (2001).

In another aspect, such chimeric polypeptide comprises a polypeptide sequence comprising a relatively cysteine-rich region comprising a sequence falling within the sequence pattern Cys Xaa Cys Xaa₍₈₎ Cys Xaa₍₇₎ Cys Xaa Cys Xaa₍₁₀₎ Cys Xaa₍₆₎ Cys Xaa₍₃₂₎ Cys Xaa₍₁₀₎ Cys Xaa₍₅₎ Cys Xaa Cys Xaa₍₁₆₎ Cys (SEQ ID NO:88). Such polypeptide sequence can further comprise SEQ ID NO:71, SEQ ID NO:47, and/or SEQ ID NO:59 or SEQ ID NO:60. In another aspect, such chimeric polypeptide comprises a polypeptide sequence comprising the sequence pattern Cys Val Cys Glu Asn Tyr Lys Leu Ala Val Xaa Cys Xaa₍₇₎ Cys Xaa Cys Xaa₍₁₀₎ Cys Xaa₍₆₎ Cys Xaa₍₁₃₎ Arg Arg Xaa• Xaa₍₆₎ Gln Asn Asn Asp Gly Leu Tyr Asp Pro Asp Cys Asp Glu Ser Gly Leu Phe Lys Xaa₍₃₎ Cys Xaa₍₃₎ Ala Thr Cys Trp Cys Val Asn Thr Ala Xaa₍₁₂₎ Cys (SEQ ID NO:89), wherein Xaa represents any suitable amino acid residue and Xaa• typically is an Ala, Ile, or Met residue. It is expected that the twelve cysteine residues in this cysteine-rich region form six disulfide bonds according to a 1-3, 2-4, 5-6, 7-8, 9-10, and 11-12. Such a polypeptide sequence is expected to undergo proteolytic cleavage in or near the Arg Arg Xaa• motif, forming a propeptide sequence that comprises the portion of the sequence N-terminal to the cleavage site and a mature polypeptide portion C-terminal to the proteolytic cleavage site. As such, the invention includes a truncated chimeric polypeptide that induces an immune response to EPCAM comprising a polypeptide sequence having at least about 97, 98, or 99% identity to the sequence of SEQ ID NO: 1, formed by such cleavage, and which polypeptide sequence is characterized by two disulfide bonds and/or more particularly by a sequence according to the sequence pattern Arg Xaa• Xaa₍₆₎ Gln Asn Asn Asp Gly Leu Tyr Asp Pro Asp Cys Asp Glu Ser Gly Leu Phe Lys Xaa₍₃₎ Cys Xaa₍₃₎ Ala Thr Cys Trp Cys Val Asn Thr Ala Xaa₍₁₂₎ Cys (SEQ ID NO:90), wherein the four C-terminal cysteine residues form disulfide bonds according to a 1-2, 3-4 bonding pattern. A corresponding propeptide, such as the polypeptide sequence of SEQ ID NO:2, is similarly provided.

In another aspect, such chimeric polypeptides may comprise a polypeptide sequence that differs from that of SEQ ID NO:5 by at least one substitution in the sequence of SEQ ID NO:5 of a functionally conservative amino acid residue and/or of a residue that retains the weight and/or hydropathy characteristics of the substituted amino acid residue.

Polypeptides Further Comprising Signal Peptides

Polypeptides of the invention that comprise at least an extracellular domain and optionally a propeptide may, if desired, further include a functional “signal sequence” or “signal peptide.” For example, a polypeptide comprising a polypeptide sequence selected from the group consisting of SEQ ID NOS:5, may further include a signal peptide. An exemplary signal peptide comprises a polypeptide sequence that has at least about 85, 90, 91, 92, 93, 94, 95, 96, 97, 98 or 99% sequence identity to the polypeptide sequence of SEQ ID NO:3 or 37, and that serves to target a polypeptide of the invention comprising at least an ECD and optionally a propeptide to the ER, secretory pathway, and/or to be secreted from the cell in which it is expressed. Thus, for example, in one aspect, the invention provides a polypeptide comprising a polypeptide sequence that comprises a first polypeptide sequence selected from the group of SEQ ID NOS: 1, 5, 7, 8, 9, 10, 12, and 92 and a second polypeptide sequence that is a signal peptide. Many such polypeptides of the invention induce at least one type of immune response against hEpCAM or an antigenic fragment thereof as described previously and below. Additionally, the novel signal peptides of the invention are useful with other polypeptide molecules for signal functioning.

Several types of functional signal peptides, and principles related to the identification and generation of such sequences, are known in the art. Generally, a signal peptide directs the organelle trafficking and/or secretion of at least a portion of an associated polypeptide upon expression in a host cell (e.g., an animal cell). For example, a signal peptide can direct a polypeptide with which it is associated to the endoplasmic reticulum (ER), golgi, and/or other secretory-related organelles, vesicles, or structures of a host cell. A signal peptide also or alternatively can direct an associated polypeptide to the nucleus or other organelle, to a cell membrane in which at least a portion of the polypeptide becomes translocated or through which the polypeptide is secreted. As mentioned above, the signal peptide comprises a subsequence of the immature (i.e., not fully processed) form of certain TAg polypeptides, such as, e.g., TAg-25 (SEQ ID NO:4), TAg-21 (SEQ ID NO: 13), TAG-18 (SEQ ID NO:32). The signal peptide normally is subsequently removed and degraded by cellular proteases, yielding a more mature form of such TAg polypeptide.

In some instances, the polypeptide can comprise a signal peptide that targets a secreted polypeptide to a cell other than the cell the protein is expressed in and secreted from. In this respect, the polypeptide can include an intracellular targeting sequence (or “Sorting signal”) that directs the polypeptide to an endosomal and/or lysosomal compartment(s) or other compartment rich in MHC II to promote CD4+ and/or CD8+ T cell presentation and response, such as a lysosomal/endosomal-targeting sorting signal derived from lysosomal associated membrane protein 1 (e.g., LAMP-1—see, e.g., Wu et al. Proc. Natl. Acad. Sci. USA 92:1161-75 (1995) and Ravipraskash et al., Virology 290:74-82 (2001)), a portion or homolog thereof (see, e.g., U.S. Pat. No. 5,633,234), or other suitable lysosomal, endosomal, and/or ER targeting sequence (see, e.g., U.S. Pat. No. 6,248,565). In some aspects, it may desirable for the intracellular targeting sequence to be located near or adjacent to a selected or predicted epitope within the polypeptide (e.g., at least one peptide sequence selected from the group consisting of SEQ ID NOS:47-64), thereby increasing the likelihood of T cell presentation of immunogenic fragments of the polypeptide.

A polypeptide that comprises at least an ECD (SEQ ID NO:1; SEQ ID NOS:7, 8, 10) or propeptide/ECD (SEQ ID NO:5) of the invention typically further includes a signal peptide that directs the polypeptide to the ER and secretory pathway and thereafter to be secreted from the cell in which it is expressed. The polypeptide can comprise any suitable ER-targeting sequence. Many ER-targeting sequences are known in the art. Examples of such signal peptide sequences are described in U.S. Pat. No. 5,846,540. Commonly employed heterologous ER/secretion signal peptide sequences include the yeast alpha factor signal sequence and mammalian viral signal sequences such as herpes virus gD signal sequence. Further examples of signal peptide sequences are described in, e.g., U.S. Pat. Nos. 4,690,898, 5,284,768, 5,580,758, 5,652,139, and 5,932,445. Suitable signal peptide sequences can be identified using skill known in the art. For example, the SignalP program (described in, e.g., Nielsen et al. Protein Engineering 10:1-6 (1997)), which is publicly available through the Center for Biological Sequence Analysis at http://www.cbs.dtu.dk/services/SignalP, or similar sequence analysis software capable of identifying signal-sequence-like domains can be used. Related techniques for identifying suitable signal peptides are provided in Nielsen et al., Protein Eng. 10(1):1-6 (1997). Sequences can be manually analyzed for features commonly associated with signal peptide sequences, as described in, e.g., European Patent Application 0 621 337, Zheng and Nicchitta J. Biol. Chem. 274(51):36623-30 (1999), and Ng et al., J. Cell Biol. 134(2):269-78 (1996). Generally, such a signal peptide will comprise predominantly hydrophobic amino acid residues. By directing the polypeptide into the secretory pathway, the signal peptide facilitates glycosylation of one or more portions of the polypeptide and/or the formation of disulfide bonds between the various cysteine residues of the immunogenic amino acids of the polypeptide (e.g., between the cysteines of SEQ ID NO: 1). The signal sequence also or alternatively will typically direct the polypeptide to be secreted from, or embedded (translocated) in the membrane of, a cell in which it is expressed.

Also provided are functional signal peptide sequences related to SEQ ID NO:3 (i.e., polypeptide variants of the polypeptide sequence of SEQ ID NO:3) that differ from the sequence of SEQ ID NO:3 by functionally conservative amino acid substitutions and/or substitutions in which weight homology and/or hydropathy is conserved (as described above). A polypeptide variant of the sequence of SEQ ID NO:3 can be characterized as falling within the sequence pattern Met Ala Xaa₁ Pro Xaa₂ Xaa₃ Leu Ala Xaa₄ Gly Leu Leu Leu Ala Xaa₅ Xaa₆ Thr Ala Thr Xaa₇ Ala Ala Ala (SEQ ID NO:83), wherein Xaa represents any amino acid residue. Commonly, the variable amino acid residues in the variable positions comprised within this sequence pattern will correspond to the residues set forth in Table 5.

TABLE 5

| Position | Selected Residues |
|---|---|
| Xaa₁ | G or P |
| Xaa₂ | K or Q |
| Xaa₃ | A or V |
| Xaa₄ | F or L |
| Xaa₅ | A or V |
| Xaa₆ | A or V |
| Xaa₇ | F or L |

Preferably, the polypeptide comprises a signal peptide that promotes, enhances, and/or induces an immune response to EpCAM. For example, the polypeptide can comprise a signal peptide that enhances an EpCAM-specific immune response in a subject induced by; for example, the polypeptide sequence of SEQ ID NO: 1, SEQ ID NO:2, SEQ ID NO:5, or SEQ ID NO:7 or a variant thereof. In one aspect, the signal peptide comprises a recombinant or non-naturally occurring polypeptide sequence having at least about 95, 96, 97, 98, or 99% identity to the polypeptide sequence of SEQ ID NO:3, wherein said polypeptide sequence of the signal peptide also includes a subsequence(s) comprising the peptide sequence of SEQ ID NO:75 or SEQ ID NO:74, or both SEQ ID NO:75 and SEQ ID NO:74 (usually in an N-to-C terminal order with respect to one another in the recombinant or non-naturally occurring polypeptide).

Polypeptides Further Comprising Transmembrane Domains

In another aspect, the invention provides an isolated or recombinant polypeptide comprising an ECD, propeptide/ECD, or signal peptide/propeptide/ECD of the invention as described above, and further comprising a functional transmembrane sequence (transmembrane portion), such that at least a portion of the polypeptide will be fixedly associated with (e.g., translocated in) the membrane of a eukaryotic cell (typically an animal cell, and more typically a mammalian cell) upon expression of the polypeptide therein. Any transmembrane sequence that causes the polypeptide to associate with the surface of the cell from which it is expressed for a detectable period of time and allows the polypeptide to induce an immune response to EpCAM is suitable. Such polypeptides typically induce at least one type of immune response against hEpCAM or an antigenic fragment thereof as described previously and further below in the Examples. Additionally, the novel transmembrane domain sequences of the invention are useful in other contexts and with other polypeptides where a transmembrane domain is desired.

The selection of a suitable transmembrane domain sequence may take into account other factors, such as secondary, tertiary, and/or quaternary structure of the transmembrane domain. Suitable transmembrane domain sequences, principles related to their selection, and nucleic acids encoding such sequences for the production of a fusion protein comprising, e.g., an ECD, propeptide/ECD, or signal peptide/propeptide/ECD of the invention, as described above, and such a transmembrane domain are known in the art. Briefly, a transmembrane domain typically comprises one or more alpha helix domains of about 20 amino acids, which alpha helix domain is comprised primarily of hydrophobic amino acids (beta-sheet and beta-barrel transmembrane domains also are known). A feature of particular transmembrane sequences is the ability for the polypeptide to act as a cell adhesion molecule (CAM), similar to EpCAM.

The transmembrane domain can be located in any suitable portion of the polypeptide. Normally, the transmembrane portion will be positioned near or adjacent to the C-terminus of the ECD (e.g., SEQ ID NO: 1) or a partially processed mature form, such as one which includes a propeptide (propeptide/ECD; e.g., SEQ ID NO:5), although the polypeptide can comprise additional intervening sequences (e.g., a flexible linker) positioned between the polypeptide sequence corresponding to the ECD (or PP/ECD partially processed mature form), and the polypeptide sequence corresponding to the transmembrane domain.

In one aspect, the invention provides a TAg polypeptide, such as TAg-25, TAg-18, or TAg-21, comprising a transmembrane domain sequence that is a predicted or confirmed transmembrane domain of, or derived from, a polypeptide that is expressed on epithelial and/or cancerous cells in a mammalian host. For example, the transmembrane domain sequence of the CEA cell adhesion molecule 1 (CEACAM1), a cadherin, a prostate-specific membrane antigen (PSMA), MUC1 (or related epithelial cell cancer-associated antigen), a VEGF receptor, an integrin receptor (e.g., anb3), a member of the CD44 protein family, or TROP-2 (GA733-1—see, e.g., U.S. Pat. No. 5,185,254) can be used to form a fusion protein with TAg-25 (SEQ ID NO:4).

Other potentially suitable transmembrane domains include the transmembrane domains of homologs and orthologs of EpCAM, such as the murine tumor-associated calcium signal transducer 1, murine lymphocyte antigen 74 (GenBank Accession No. NP_{—}032558) (see also Bergsagel et al., J. Immunol. 148(2):590-6 (1992)), GA733-1, and EGP-314 (GenBank Accession No. CAA04498—see, e.g., Wurfel et al., Oncogene 18(14):2323-2334 (1999)), or the transmembrane domain of a mammalian EpCAM, such as hEpCAM (see SEQ ID NO:45). Such domains can be predicted by comparison with the transmembrane domain of EpCAM (amino acids 266-291) or by bioinformatic analysis of these sequences (e.g., by TMPred, which is available at http://www.ch.embnet.org/software/TMPRED_form.html, and TMAP, which is available at http://www.mbb.ki.se/tmap/index.html, and GREASE).

In some aspects, the invention provides a recombinant immunogenic polypeptide of the invention comprising an ECD, propeptide/ECD, or signal peptide/propeptide/ECD as described above may further comprise a transmembrane domain, wherein the transmembrane domain (TMD) comprises a polypeptide sequence having at least about 70, 80, 90, 91, 92, 93, 94, 95, 96, 97, 98, or 99% sequence identity to a polypeptide sequence selected from the group consisting of SEQ ID NOS:15, 45, and 80. Typically, the fusion of such a TMD to the C-terminus of an ECD, propeptide/ECD, or signal peptide/propeptide/ECD of the invention is such that the resultant recombinant polypeptide upon cellular expression is bound to the cell membrane for at least a detectable period of time by the TMD. The polypeptide sequence of some such TMDs further includes the epitope peptide sequence of SEQ ID NO:77. Also provided are polypeptide variants of such TMDs. Such variants usually differ from the above-described TMD sequences by the substitution of one or more amino acid residues in the above-described TMD sequences with one or more functionally conservative amino acid residues and/or one or more amino acid residues that retain (i.e., conserve) weight and/or hydropathy characteristics as the substituted residues.

In alternative aspects, the invention provides a polypeptide that comprises a transmembrane sequence that has at least about 90% sequence identity (e.g., about 91-99% sequence identity) to SEQ ID NO:80. Such polypeptides can comprise SEQ ID NO: 1 or an amino acid sequence variant thereof, SEQ ID NO:2 or an amino acid sequence variant thereof, a mature domain of hEpCAM, a mature domain of an EpCAM homolog or ortholog, or combinations of portions thereof. The mature form of hEpCAM is a polypeptide comprising the ECD, TMD, and CD of hEpCAM. Particular sequence variants of the sequence of SEQ ID NO:80 comprise: (1) the substitution of Cys₄ of the sequence of SEQ ID NO:80 with an Ile residue, or (2) the deletion of Ile₁₂ of the sequence of SEQ ID NO:80, which Ile₁₂ deletion is typically associated with an insertion of a Val residue or functionally homologous residue between Val₁₀ and Met₁₁ of the sequence of SEQ ID NO:80.

Polypeptides Further Comprising Cytoplasmic Domains

In another aspect, the invention provides an isolated or recombinant polypeptide comprising an ECD/TM, propeptide/ECD/TM, signal peptide/propeptide/ECD/TM of the invention as described above and further comprising a functional cytoplasmic domain that serves as an intracellular anchor, such that the resultant polypeptide remains bound to the cell membrane of a eukaryotic cell (typically an animal cell, and more typically a mammalian cell) upon expression of the polypeptide therein or is not secreted. Such polypeptides typically induce at least one type of immune response against hEpCAM or an antigenic fragment thereof as described herein and in the Examples below.

The cytoplasmic domain can comprise any suitable amino acid sequence. Normally, the cytoplasmic domain is positioned at or near the C-terminus of the transmembrane domain of the isolated or recombinant polypeptide described above. The cytoplasmic domain is usually highly charged. Commonly, the cytoplasmic domain comprises mostly positive residues (e.g., about 9 positively charged amino acid residues and about 4 negatively charged amino acid residues). Typically, the cytoplasmic domain comprises a polypeptide sequence having at least about 80, 90, 91, 92, 93, 94, 95, 96, 97, 98, or 99% sequence identity to the polypeptide sequence of SEQ ID NO: 11 or SEQ ID NO:46. In one aspects, the isolated or recombinant polypeptide comprises a cytoplasmic domain comprising the sequence of SEQ ID NO:46. Polypeptide variants of the sequences of SEQ ID NOS: 11 and 46 are also provided; such variants commonly differ from the sequence of SEQ ID NO: 11 or SEQ ID NO:46 by one or more functionally conservative amino acid substitutions and/or one or more substitutions with amino acid residues that retain the hydropathy and/or weight characteristics of the substituted amino acid residues of the polypeptide sequence of SEQ ID NO: 11 or SEQ ID NO:46, respectively. Particular variants of the sequence SEQ ID NO: 11 comprise a substitution at Arg₁₉ of the sequence of SEQ ID NO: 11 and/or a deletion of one or more of the three C-terminal amino acids of the sequence of SEQ ID NO: 11.

Polypeptides Comprising SP/PP/ECDs

In yet a further aspect, the invention provides a recombinant or chimeric polypeptide comprising a polypeptide sequence comprising a signal peptide (SP), propeptide (PP) and extracellular domain (ECD) of the invention, which polypeptide induces or enhances an immune response against hEpCAM or an antigenic fragment thereof. In one aspect, the invention provides a recombinant or chimeric polypeptide comprising a polypeptide sequence having at least about 97, 98, or 99% sequence identity to the polypeptide sequence of SEQ ID NO:4 (termed TAg-25), which polypeptide induces or enhances an immune response against hEpCAM or an antigenic fragment thereof. In a preferred aspect, the invention provides a polypeptide that consists of the polypeptide sequence of SEQ ID NO:4. Such novel TAg-25 polypeptides are at least as immunogenic as human EpCAM. In particular, such antigenic polypeptides induce production of hEpCAM-specific antibodies, induce T cell proliferation and/or T cell activation, and induce production of IFN-γ and IL-5. Furthermore, such TAg polypeptides are capable of specifically binding antibodies to human EpCAM. Such TAg polypeptides are useful in therapeutic and/or prophylactic methods described further herein, including, e.g., as compositions and vaccines against EpCAM-associated tumors and metastatic diseases, and in diagnostic assays described in further detail below. Some such chimeric polypeptides comprise a polypeptide sequence that includes as a subsequence(s) at least one epitope peptide sequence selected from the group consisting of SEQ ID NOS:47-64 and 71-76. Usually, the polypeptide sequence of such a chimeric polypeptide includes at least 2, at least 3, at least 4, at least 5, or more epitope peptide sequences selected from the group consisting of SEQ ID NOS:47-64 and 71-76. As discussed above, many such peptide sequences overlap in terms of residues or motifs, such that the isolated or recombinant polypeptide may comprise several of these peptide sequences as overlapping, but not discrete subsequences.

Some such chimeric polypeptides may comprise a polypeptide sequence that includes as discrete subsequences (unless otherwise noted) with said polypeptide sequence at least 2, 3, 4, 5, 6, 7, 8, 9 or preferably 10 epitope peptide sequences selected from the group consisting of: (1) SEQ ID NO:74 or SEQ ID NO:75; (2) SEQ ID NO:71 or SEQ ID NO:72; (3) SEQ ID NO:47 and/or SEQ ID NO:63 or SEQ ID NO:64; (4) SEQ ID NO:59 or SEQ. ID NO:60; (5) SEQ ID NO:57 or SEQ ID NO:58; (6) SEQ ID NO:48; (7) SEQ ID NO:49 or any one of SEQ ID NOS:50-53 (wherein the sequence of any of SEQ ID NOS:50-53 can overlap with the sequence of SEQ ID NO:48); (8) any one of SEQ ID NOS:54-56; (9) SEQ ID NO:61 or SEQ ID NO:62; and (10) any one of SEQ ID NOS:65-70; wherein the two or more peptide sequences are positioned with respect to one another in the polypeptide sequence of the chimeric polypeptide in N-terminal to C-terminal order in the order designated above from (1) to (10). The sequence of such chimeric polypeptide can include as subsequences any suitable combination of at least two of these 10 peptide sequences. Furthermore, such chimeric polypeptide may comprise a sequence that differs from that of SEQ ID NO:4 by one or more substitutions of functionally conservative amino acids or one or more substitutions wherein the weight and/or hydropathy characteristics of the substituted amino acid residues are retained.

Such chimeric polypeptide also or alternatively may comprise a suitable transmembrane domain as described elsewhere herein. (e.g., any sequence selected from the group of SEQ ID NOS: 15, 45, and 80) and, optionally, a suitable cytoplasmic domain as described elsewhere herein (e.g., the sequence of SEQ ID NO:46).

The polypeptide sequence of SEQ ID NO:4 comprises a signal peptide domain, propeptide domain, and extracellular domain (which is similar to the mature extracellular (ECD) domain of a type I membrane protein). The ECD of the sequence of SEQ ID NO:4 comprises from about amino acid residue 81 to about amino acid residue 265.

Another aspect of the invention pertains to an isolated or recombinant polypeptide that induces or enhances an immune response against hEpCAM or an antigenic fragment thereof, wherein said polypeptide comprises a polypeptide sequence that has at least about 96, 97, 98, or 99% sequence identity to an ECD sequence comprising about amino acid residues 81-265 of SEQ ID NO:4. Some such isolated or recombinant polypeptides comprise a polypeptide sequence that differs from said ECD sequence by one or more, but less than all, of the following amino acid substitutions: (1) the substitution of Ile₈₂ of the sequence of SEQ ID NO:4 with an Ala or Met residue; (2) the substitution of Ala ₁₁₄ of the sequence of SEQ ID NO:4 with a Ser residue; (3) the substitution of Glu₁₅₂ of the sequence of SEQ ID NO:4 with an Ala residue; (4) the substitution of Ser₁₅₅ of the sequence of SEQ ID NO:4 with a Gln or Lys residue; (5) the substitution of His₁₆₃ of the sequence of SEQ ID NO:4 with a Gln or Arg residue; (6) the substitution of Met₁₉₆ of the sequence of SEQ ID NO:4 with a Val residue; (7) the substitution of ASp₂₀₅ of the sequence of SEQ ID NO:4 with an Asn residue; (8) the substitution of Arg₂₃₄ of the sequence of SEQ ID NO:4 with a Thr residue; and (9) the substitution of Leu₂₃₉ of the sequence of SEQ ID NO:4 with a Gln or Pro residue. The position(s) in the sequence at which the one or more substitutions occur can vary with respect to the position of the substituted amino acid residue of the sequence of SEQ ID NO:4 due to the deletion and/or addition of one or more amino acid residues occurring in the ECD sequence of SEQ ID NO:4. Some such polypeptides may comprise a sequence that differs from said ECD sequence by one or more conservative substitutions in terms of function, weight, and or hydropathy of the substituted amino acid residues.

In another aspect, the invention provides an isolated recombinant polypeptide comprising SEQ ID NO:9 or SEQ ID NO: 12, wherein said polypeptide is capable of inducing an immune response against hEpCAM or an antigenic fragment thereof. In a particular aspect, the invention provides a polypeptide consisting essentially of or consisting of SEQ ID NO:1, SEQ ID NO:9, or SEQ ID NO:12.

An amino acid subsequence of the polypeptide sequence of SEQ ID NO:4 comprising amino acid residues 24-265 is expected to include a propeptide and ECD (“PP/ECD”). As such, the invention provides an isolated or recombinant chimeric polypeptide that induces an immune response against EPCAM, which polypeptide comprises a polypeptide sequence that has at least about 97, 98, or 99% sequence identity to the amino acid residues 24-265 subsequence of the polypeptide sequence of SEQ ID NO:4 (i.e., a PP/ECD sequence of SEQ ID NO:4). Some such chimeric polypeptides comprise a polypeptide sequence that differs from the sequence of SEQ ID NO:4 by the substitution of Glu₄₅ of the sequence of SEQ ID NO:4 with an Ala residue. Some such chimeric polypeptides comprise a polypeptide sequence that differs from the sequence of SEQ ID NO:4 in at least the substitution of Glu₄₅ of the sequence SEQ ID NO:4 with an Asp residue. Some such chimeric polypeptides comprise a polypeptide sequence that differs from the sequence of SEQ ID NO:4 in at least that Glu₄₅ of the sequence SEQ ID NO:4 is substituted with an Asn, Gln, Glu, or Lys residue.

In another aspect, the invention further provides a chimeric polypeptide that induces an immune response against hEpCAM or an antigenic fragment thereof, said polypeptide comprising a polypeptide sequence that has at least about 95, 96, 97, 98, or 99% identity to SEQ ID NO:4, wherein the polypeptide sequence differs from that of SEQ ID NO:4 by the substitution of Ala₆ of SEQ ID NO:4 with a Val residue, the substitution of Leu₉ of SEQ ID NO:4 with a Phe residue, or both, wherein the position in the amino acid sequence at which the substitution or substitutions occur can vary with respect to the position of the substituted amino acid residue of SEQ ID NO:4 due to the deletion and/or of one or more amino acid residues occurring in SEQ ID NO:4.

Also provided are immunogenic fragments of the sequence of SEQ ID NO:4 that have an ability to induce an immune response against hEpCAM or an antigenic fragment thereof. For example, the invention provides a polypeptide comprising a polypeptide sequence that has at least about 96, 97, 98, 99, or 100% sequence identity to an amino acid sequence corresponding to amino acid residues 81-265, amino acid residues 82-265, amino acid residues 24-265 or amino acid residues 1-265 of the sequence of SEQ ID NO:4, wherein said chimeric polypeptide has an ability to induce an immune response against hEpCAM or an antigenic fragment thereof. Also provided is a chimeric polypeptide comprising a sequence corresponding to about residues 1-21, 22-106, 1-106, 107-122, 22-122, 1-122, 123-152, 22-152, 1-152, 153-182, 22-182, 123-182, 1-182, 123-192, 153-192, 22-192, 1-192, 22-249, 122-249, 153-249, 182-249, 192-249, 123-265, 153-265, 182-265, or 193-265 of SEQ ID NO:4, which polypeptide preferably induces an immune response against mEpCAM.

In another aspect, the invention provides a chimeric polypeptide comprising the polypeptide sequence of SEQ ID NO:4 or an immunogenic fragment thereof, wherein said polypeptide induces at least one type of immune response as described further herein against human EpCAM, or an antigenic fragment thereof, and further comprising a polypeptide sequence corresponding to a functional transmembrane domain, such as are described elsewhere herein. For example, such a chimeric polypeptide may comprise a TMD having at least about 95, 96, 98, 98, 99, or 100% sequence identity to the sequence of SEQ ID NO:45. The resultant chimeric polypeptide comprises a signal peptide, propeptide, ECD, and TMD and thus has the form SP/PP/ECD/TM. Such SP/PP/ECD/TM polypeptides can further include a cytoplasmic domain as further described elsewhere herein. For example, such polypeptides can include a cytoplasmic domain that has at least about 95, 96, 98, 98, 99, or 100% sequence identity to the sequence of SEQ ID NO:46.

In another aspect, the invention provides a chimeric polypeptide comprising a polypeptide sequence having at least about 95, 96, 97, 98, 99 or 100% sequence identity to the polypeptide sequence of SEQ ID NO:6, which chimeric polypeptide induces an immune response against a mammalian EpCAM (e.g., hEpCAM) or an antigenic fragment thereof, as described elsewhere herein. Such chimeric polypeptide typically acts as a type I transmembrane protein and comprises the following domains: signal peptide (about residues 1-23 of the sequence of SEQ ID NO:6), propeptide (about residues 24-80 of the sequence of SEQ ID NO:61), extracellular domain (about residues 81-265 of the sequence of SEQ ID NO:6), transmembrane domain (about residues 266-288 of the sequence of SEQ ID NO:6), and cytoplasmic domain (about residues 289-314 of the sequence of SEQ ID NO:6).

Many polypeptides of the invention that have an ability to induce at least one type of immune response against a mammalian EpCAM or antigenic fragment thereof as described elsewhere herein comprise an immunogenic polypeptide sequence that has at least about 96, 97, 98, 99% or more sequence identity to the sequence of SEQ ID NO:1, SEQ ID NO:4, SEQ ID NO:5, or SEQ ID NO:6, and have structure substantially similar to the structure of a polypeptide consisting essentially or consisting of the polypeptide sequence of SEQ. ID NO: 1, SEQ ID NO:4, SEQ ID NO:5, or SEQ ID NO:6, respectively. By a substantially similar structure, it is meant that the polypeptide retains a similar secondary structure (i.e., in terms of secondary structure domains and turns), a similar tertiary structure, a similar quaternary structure, or a combination thereof. The determination of a substantially similar secondary structure can readily be performed by computer analysis of the subject and reference sequences using programs such as GOR4, PELE, and/or CHOFAS, available through the SDSC. For example, polypeptides having an above-specified sequence identity with the polypeptide sequence of SEQ ID NO:4 will typically comprise a predicted beta sheet sequence at about residues 56-61 followed by an alpha helix domain at about residues 63-71 and a predicted beta sheet at about residues 248-252. Polypeptides having an above-specified sequence identity with the polypeptide sequence of SEQ ID NO: 1 will typically comprise one or more beta sheets in a region within (or consisting of) about residues 24-40, an alpha helix domain at about residues 80-95, a predicted beta sheet at about residues 110-115, alpha helix domains at about residues 129-137 and 145-146, and a predicted beta sheet region at about residues 168-172.

Polypeptides having an above-specified sequence identity with the polypeptide sequence of SEQ ID NO:4 or SEQ ID NO: 1 also or alternatively will typically comprise a sequence that is recognized as a Thyroglobulin type-1 repeat signature pattern (pfam00086.4, thyroglobulin_{—}1: PSSM-Id:654) when the sequence is compared to the National Center for Biotechnology Information (NCBI) Conserved Domain Database (CDD), which conveniently is automatically performed when using default settings for the NCBI blastp program. A Thyroglobulin type-1 repeat motif in such a variant typically will comprise a sequence according to the sequence pattern Cys Xaa Val Glu Arg Xaa₍₆₎ Ser Xaa₍₈₎ Glu Gly Ala Leu Xaa₍₄₎ Gly Leu Tyr Xaa Pro Xaa Cys Asp Glu Xaa Gly Xaa₍₂₎ Lys Xaa₍₂₎ Gln Cys Xaa₍₆₎ Cys Trp Cys Val Asp Xaa₍₂₎ Gly Xaa₍₆₎ Asp Xaa₍₃₎ Glu (SEQ ID NO:91).

Several other suitable techniques for determining whether a polypeptide sequence shares substantial structural similarity with a target sequence are known in the art. For example, software programs include the MAPS program and the TOP program (described in Lu, Protein Data Bank Quarterly Newsletter, #78:10-11 (1996), and Lu, J. Appl. Cryst. 33:176-183 (2000)) can be used to determine structural similarity of two polypeptides. A polypeptide sequence will desirably exhibit low topological diversity in such contexts (e.g., a topical diversity of less than about 20, preferably less than about 15, and more preferably less than about 10), but some structurally diverse polypeptides can be suitable. As another example, the structural similarity of polypeptides can be compared using the PROCHECK program (described in, e.g., Laskowski, J. Appl. Cryst. 26:283-291 (1993)), the MODELLER program, or commercially available programs incorporating such features. Alternatively still, structure predictions can be compared by way of a sequence comparison using a program such as the PredictProtein server (available at http://dodo.cpmc.columbia.edu/predictprotein/). Additional examples of techniques for analyzing protein structure that can be applied to determine structural similarity are described in, e.g., Yang and Honig, J. Mol. Biol. 301(3):665-78 (2000), Aronson et al., Protein Sci. 3(10):1706-11 (1994), Marti-Remon et al., Annu. Rev. Biophys. Biomol. Struct. 29:291-325 (2000), Halaby et al., Protein Eng. 12(7):563-71 (1999), Basham, Science 283:1132 (1999), Johnston et al., Crit. Rev. Biochem. Mol. Biol. 29(1):1-68 (1994), Moult, Curr. Opin. Biotechnol. 10(6):583-6 (1999), Benner et al., Science 274:1448-49 (1996), and Benner et al., Science 273:426-8 (1996), as well as International Patent Application WO 00/45334.

Protein Modifications

Polypeptides of the invention described herein can be further modified in a variety of ways by, e.g., post translational modification and/or synthetic modification or variation. For example, polypeptides of the invention may be suitably glycosylated, typically via expression in a mammalian cell. In one aspect, the invention provides glycosylated polypeptides induce an immune response against human EpCAM or an antigenic fragment thereof as described elsewhere herein, wherein said glycosylated polypeptides comprise the polypeptide sequence of SEQ ID NO: 1, SEQ ID NO:4, SEQ ID NO:5, or SEQ ID NO:6. Some such glycosylated polypeptides of the sequence of SEQ ID NO:4 or SEQ ID NO:6, for example, comprise an N-linked glycosylated Asn residue at about residue 74, at about residue 111, or both. Some such glycosylated polypeptides of the sequence of SEQ ID NO:4 or SEQ ID NO:6 additionally or alternatively comprise the peptide sequence Asn Gly Ser Lys at about residues 74-77, wherein the Asn residue is partially glycosylated, and the peptide sequence Asn Gly Thr Ala at about residues 111-114, wherein the Asn residue is completely glycosylated (being associated with a carbohydrate complex of about 890-1260 Da), both Asn residues being subject to N-linked glycosylation. Glycosylated polypeptides with similar glycosylation patterns can be readily determined for SEQ ID NOS: 1 and 5, by optimal alignment with the sequences of SEQ ID NOS:4 and 6. For example, the invention provides a glycosylated polypeptide comprising the sequence of SEQ ID NO: 1, wherein for the peptide sequence Asn Gly Ser Lys at about position 52 of the sequence, wherein the Asn is at least partially glycosylated by N-linked glycosylation.

The polypeptide sequence of SEQ ID NO:4 or SEQ ID NO:5 is typically subject to glycosylation after expression in a suitable host cell, such that about 1-3 glycans are added to the sequence. Such glycosylation can add about 2-4 kDa (e.g., about 3.8 kDa) to the weight of the polypeptide. Polypeptides of the invention may be subject to heterogeneity in terms of glycosylation. Thus, for example, recombinant or chimeric polypeptides consisting of the sequence of SEQ ID NO:4 expressed in a cell culture, can exhibit a weight of about 38 kDa, about 40 kDa, about 42 kDa, or about 45 kDa (e.g., about 37-46 kDa) due to such heterogeneous glycosylation. Polypeptides comprising or consisting of smaller immunogenic amino acid sequences of the invention (e.g., SEQ ID NO: 1 or another ECD polypeptide sequence) usually have lower apparent and actual molecular weights (e.g., about 32-36 kDa), which weights can vary due to differences in glycosylation and cleavage of an immunogenic portion (e.g., ECD) from one or more other portions or domains, such as a propeptide and/or signal peptide.

A polypeptide comprising the polypeptide sequence of SEQ ID NO:4, when expressed in a eukaryotic cell, isolated by SDS PAGE, normally has an apparent molecular weight of about 30-40 kDa, more usually about 32-36 kDa, which is expected to correspond to the weight of the predominant polypeptide species after proteolytic cleavage and other processing (e.g., glycosylation) of the immature polypeptide has occurred. In some instances, a polypeptide comprising the polypeptide sequence of SEQ ID NO:4 is subject to multiple points of proteolytic cleavage, resulting in several polypeptides having different apparent molecular weights within such a range. As mentioned above, proteolytic cleavage also can be cell type-dependent for such polypeptides.

The polypeptides of the invention can be subject to any number of additional forms suitable of post translational and/or synthetic modification or variation. For example, the invention provides protein mimetics of the polypeptides of the invention. Peptide mimetics are described in, e.g., U.S. Pat. No. 5,668,110 and the references cited therein.

In another aspect, a polypeptide of the invention can be modified by the addition of protecting groups to the side chains of one or more the amino acids of the fusion protein. Such protecting groups can facilitate transport of the fusion peptide through membranes, if desired, or through certain tissues, for example, by reducing the hydrophilicity and increasing the lipophilicity of the peptide. Examples of suitable protecting groups include ester protecting groups, amine protecting groups, acyl protecting groups, and carboxylic acid protecting groups, which are known in the art (see, e.g., U.S. Pat. No. 6,121,236). Synthetic fusion proteins of the invention can take any suitable form. For example, the fusion protein can be structurally modified from its naturally occurring configuration to form a cyclic peptide or other structurally modified peptide.

Polypeptides of the invention also can be linked to one or more nonproteinaceous polymers, typically a hydrophilic synthetic polymer, e.g., polyethylene glycol (PEG), polypropylene glycol, or polyoxyalkylene, as described in, e.g., U.S. Pat. Nos. 4,179,337, 4,301,144, 4,496,689, 4,640,835, 4,670,417, and 4,791,192, or a similar polymer such as polyvinylalcohol or polyvinylpyrrolidone (PVP).

As discussed above, polypeptides of the invention can commonly be subject to glycosylation. Polypeptides of the invention can further be subject to (or modified such that they are subjected to) other forms of post-translational modification including, e.g., hydroxylation, lipid or lipid derivative-attachment methylation, myristylation, phosphorylation, and sulfation. Other post-translational modifications that a polypeptide of the invention can be rendered subject to include acetylation, acylation, ADP-ribosylation, amidation, covalent attachment of flavin, covalent attachment of a heme moiety, covalent attachment of a nucleotide or nucleotide derivative, covalent attachment of phosphotidylinositol, cross-linking, cyclization, disulfide bond formation, demethylation, formylation, GPI anchor formation, iodination, oxidation, proteolytic processing, prenylation, racemization, selenoylation, arginylation, and ubiquitination. Other common protein modifications are described in, e.g., Creighton, supra, Seifteretal., Meth Enzymol. 18:626-646 (1990), and Rattan et al., Ann. NY Acad. Sci. 663:48-62 (1992). Post-translational modifications for polypeptides expressed from nucleic acids in host cells vary depending what kind of host or host cell type the peptide is expressed in. For instance, glycosylation often does not occur in bacterial hosts such as E. coli and varies considerably in baculovirus systems as compared to mammalian cell systems. Accordingly, when glycosylation is desired (which usually is the case for most polypeptides of the present invention), a polypeptide should be expressed (produced) in a glycosylating host, generally a eukaryotic cell (e.g., a mammalian cell or an insect cell). Modifications to the polypeptide in terms of post-translational modification can be verified by any suitable technique, including, e.g., x-ray diffraction, NMR imaging, mass spectrometry, and/or chromatography (e.g., reverse phase chromatography, affinity chromatography, or GLC).

The polypeptide also or alternatively can comprise any suitable number of non-naturally occurring amino acids (e.g., β amino acids) and/or alternative amino acids (e.g., selenocysteine), or amino acid analogs, such as those listed in the MANUAL OF PATENT EXAMINING PROCEDURE § 2422 (7th Revision—2000), which can be incorporated by protein synthesis, such as through solid phase protein synthesis (as described in, e.g., Merrifield, Adv. Enzymol. 32:221-296 (1969) and other references cited herein). A polypeptide of the invention can further be modified by the inclusion of at least one modified amino acid. The inclusion of one or more modified amino acids may be advantageous in, for example, (a) increasing polypeptide serum half-life, (b) reducing polypeptide antigenicity, or (c) increasing polypeptide storage stability. Amino acid(s) are modified, for example, co-translationally or post-translationally during recombinant production (e.g., N-linked glycosylation at N-X-S/T motifs during expression in mammalian cells) or modified by synthetic means. Non-limiting examples of a modified amino acid include a glycosylated amino acid, a sulfated amino acid, a prenlyated (e.g., farnesylated, geranylgeranylated)amino acid, an acetylated amino acid, an acylated amino acid, a PEG-ylated amino acid, a biotinylated amino acid, a carboxylated amino acid, a phosphorylated amino acid, and the like. References adequate to guide one of skill in the modification of amino acids are replete throughout the literature. Example protocols are found in Walker (1998) Protein Protocols on CD-ROM Humana Press, Towata, N.J. Preferably, the modified amino acid is selected from a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent.

Recently, the production of fusion proteins comprising a prion-determining domain has been used to produce a protein vector capable of non-Mendelian transmission to progeny cells (see, e.g., Li et al., J. Mol. Biol. 301(3):567-73 (2000)). The inclusion of such prion-determining sequences in a fusion protein comprising immunogenic amino acid sequences of the invention is contemplated, ideally to provide a hereditabie protein vector comprising the fusion protein that does not require a change in the host's genome.

The invention further provides polypeptides having the above-described characteristics that further comprise additional amino acid sequences that impact the biological function (e.g., immunogenicity, targeting, and/or half-life) of the polypeptide. For example, in one aspect the invention provides a polypeptide comprising an immunogenic polypeptide sequence of the invention (including, e.g., but not limited to, SEQ ID NO: 1, SEQ ID NO:2, SEQ ID NO:4, SEQ ID NO:5, or SEQ ID NO:6 or variant thereof as described herein) and the polypeptide sequence of an Interleukin, such as Interleukin-2 (IL-2), or a fragment thereof that enhances the ability of the polypeptide to generate an immune response to a mammalian EpCAM.

In another aspect, the invention provides a chimeric or recombinant fusion protein comprising an immunogenic polypeptide sequence of the invention (including, e.g., but not limited to, SEQ ID NO: 1, SEQ ID NO:2, SEQ ID NO:4, SEQ ID NO:5, or SEQ ID NO:6 or variant thereof as described herein) and a cytokine-like factor or modified cytokine factor, such as the factors described in International Patent Applications WO 02/36628, WO 01/51510, WO 01/40257, WO 01/36001, WO 01/25438, and WO01/15736. Such cytokine-like and modified cytokine peptides also can form a separate part of a composition (or be co-administered with) a polypeptide of the invention, be encoded by a nucleic acid of the invention (i.e., in combination with an immunogenic polypeptide of the invention in separate expression cassettes), or be encoded by a nucleic acid vector or viral vector that is administered with a novel biomolecule of the invention.

Fusion proteins and complex polypeptides comprising a first polypeptide comprising at least one immunogenic polypeptide sequence of the invention (including, e.g., but not limited to, SEQ ID NO:1, SEQ ID NO:2, SEQ ID NO:4, SEQ ID NO:5, or SEQ ID NO:6 or variant thereof as described herein) and a second polypeptide comprising a cytokine (e.g., IL-2) are generated in view of structural considerations. Thus, considerations with respect to multimerization are taken into account in generating such fusion proteins. For example, a fusion protein comprising a first polypeptide consisting essentially of the sequence of SEQ ID NO:4 or SEQ ID NO: 1 fused to a second polypeptide consisting essentially of a TNF-α amino acid sequence will take into account the trimerization of TNF-α as important to the function of the TNF-α sequence. As such, linker sequences (discussed elsewhere herein) may be used to provide sufficient space and/or flexibility between the EpCAM immunogenic portion and cytokine portion of the fusion protein. Also, a nucleic acid construct encoding the fusion protein is designed such that necessary multimerization domains are retained.

Another feature of the invention is a polypeptide comprising an immunogenic polypeptide of the invention and further comprising a targeting sequence other than, or in addition to, a signal sequence. For example, the polypeptide can comprise a sequence that targets a receptor on a particular cell type (e.g., a monocyte, dendritic cell, or associated cell) to provide targeted delivery of the polypeptide to such cells and/or related tissues. Signal sequences are described above, and include membrane localization/anchor sequences (e.g., stop transfer sequences, GPI anchor sequences), and the like.

In another aspect, the invention provides polypeptides, such as fusion proteins, that comprise an immunogenic polypeptide sequence as described above (e.g., a sequence selected from the group of SEQ ID NOS:1, 4-10, 12-14, 32, 34, 78, and 92 or a variant thereof) and one or more additional cancer antigens or immunogenic polypeptide fragments thereof (e.g., one or more epitopes from carcinoembryonic antigen (CEA)). For example, a polypeptide comprising an immunogenic amino acid sequence of the invention can further comprise MUC1, MUC2, MUC3, MUC4, MUC5AC, MUC5B, MUC7, prostate-specific membrane antigen (PSMA), HER-2/neu, and human chorionic gonadotropin-beta. Other cancer antigens, cancer vaccines, and related principles that can be used for selection of additional amino acid sequences that can be components of such a fusion protein are described in, e.g., Moingenon, Vaccine 19:1305-1326 (2001), Mellstedt, Ann. NY Acad. Sci. (2000) 910:254-61; discussion 261-2, Finn and Forni, Curr. Opin. Immunol. (2002) 14(2):172-7, Bitton et al., Oncol Rep. (2002) 9(2):267-76, Zhu and Stevenson, Curr. Opin. Mol Ther. (2002) 4(1):41-8, Weber, Cancer Invest. (2002) 20(2):208-21, Kaufman et al., Expert Opin. Biol. Ther. (2002) 2(4):395-408, Reilly et al., Methods Mol. Med. (2002) 69:233-57, Monzavi-Karbassi et al., Hybrid Hybridomics (2002) 21(2):103-9. Kumatomo et al., J. Dermatol. (2001) 28(11):658-62, Wang et al., Expert Opin. Biol. Ther. (2001) 1(2):277-90, Brossart et al., Exp. Hematol. (2001) 29(11):1247-55, Zeh et al., Trends Mol. Med. (2001) 7(7):307-13, Van Tedeelo et al., Leukemia (2001) 15(4):545-58, Cohen, Trends Mol. Med. (2001) 7(4):175-9, Mosca, Surgery (2001) 129(3):248-54, Maxwell-Armstrong et al., Br. J. Surg. (1998) 85(2):149-54, and Basak et al., Ann. NY Acad. Sci. (2000) 910:237-52; discussion 252-3.

Another possible advantageous fusion partner for an immunogenic polypeptide of the invention (e.g., a sequence selected from the group of SEQ ID NOS:1, 4-10, 12-14, 32, 34, 78, and 92 or a variant thereof) is an immunogenic heat shock protein (HSP) or portion thereof, such as HSP65, HSP70, HSP110, and gp96 (see, e.g., U.S. Pat. No. 6,335,183).

Also provided is a fusion protein comprising an immunogenic polypeptide of the invention (e.g., a sequence selected from the group of SEQ ID NOS:1, 4-10, 12-14, 32, 34, 78, and 92 or a variant thereof) and a receptor amino acid sequence, such that the polypeptide acts as a chimeric immune receptor (CIR—see, e.g., Patel et al.—Cancer Gene Ther. (2000) 7(8): 1127-34 for discussion of similar CIR molecules).

A particularly useful fusion partner for an immunogenic polypeptide of the invention is a peptide fragment or peptide portion that facilitates purification of the polypeptide (“polypeptide purification subsequence”). Several types of suitable polypeptide purification subsequences are known in the art. Examples of such fusion partners include histidine-tryptophan modules that allow purification on immobilized metals, such as a hexa-histidine peptide or other a polyhistidine sequence, a sequence encoding such a tag is incorporated in the pQE vector available from QIAGEN, Inc. (Chatsworth, Calif.), a sequence which binds glutathione (e.g., glutathione-S-transferase (GST)), a hemagglutinin (HA) tag (corresponding to an epitope derived from the influenza hemagglutinin protein; Wilson et al., Cell 37:767 (1984)), maltose binding protein sequences, the FLAG epitope utilized in the FLAGS extension/affinity purification system (Immunex Corp, Seattle, Wash.)—commercially available FLAG epitopes also are available through Kodak (New Haven, Conn.), thioredoxin (TRX), avidin, and the like. Other purification-facilitating epitope tags have been described in the art (see, e.g., Whitehorn et al., Biotechnology 13:1215-19 (1995)). In particular aspect, the polypeptide comprises an e-his tag, which comprises a polyhistidine sequence and an anti-e-epitope sequence (Pharmacia Biotech Catalog), which e-his tags can be made by standard techniques. The inclusion of a protease-cleavable polypeptide linker sequence between the purification domain and the immunogenic amino acid sequence or immunogenic amino acid sequence/signal sequence portion of the polypeptide is useful to facilitate purification of an immunogenic fragment of the fusion protein. Histidine residues facilitate purification on IMIAC (immobilized metal ion affinity chromatography (IMAC), as described in Porath et al. Protein Expression and Purification 3:263-281 (1992)) while the enterokinase cleavage site provides a method for separating the polypeptide from the fusion protein pGEX vectors (Promega; Madison, Wis.) conveniently can be used to express foreign polypeptides as fusion proteins with glutathione S-transferase (GST). Additional examples of such sequences and the use thereof for protein purification are described in, e.g., Int'l Patent Appn Publ. No. WO 00/15823. After expression of the polypeptide and isolation thereof by such fusion partners or otherwise as described above, protein refolding steps can be used, as desired, in completing configuration of the mature polypeptide.

A fusion protein of the invention also can include one or more additional peptide fragments or peptide portions which promote detection of the fusion protein. For example, a reporter peptide fragment or portion (e.g., green fluorescent protein (GFP), β-galactosidase, or a detectable domain thereof) can be incorporated in the fusion protein. Additional marker molecules that can be conjugated to the polypeptide of the invention include radionuclides, enzymes, fluorophores, small molecule ligands, and the like. Such detection-promoting fusion partners are particularly useful in fusion proteins used in diagnostic techniques discussed elsewhere herein.

In another aspect, an immunogenic polypeptide of the invention can comprise a fusion partner that promotes stability of the polypeptide, secretion of the polypeptide (other than by signal targeting), or both. For example, the polypeptide can comprise an immunoglobulin (Ig) domain, such as an IgG polypeptide comprising an Fc hinge, a CH2 domain, and a CH3 domain, that promotes stability and/or secretion of the polypeptide.

The fusion protein peptide fragments or peptide portions can be associated in any suitable manner. Typically and preferably, the various polypeptide fragments or portions of the fusion protein are covalently associated (e.g., by means of a peptide or disulfide bond). The polypeptide fragments or portions can be directly fused (e.g., the C-terminus of the immunogenic amino acid sequence can be fused to the N-terminus of a purification sequence or heterologous immunogenic sequence). The fusion protein can include any suitable number of modified bonds, e.g., isosteres, within or between the peptide portions. Alternatively or additionally, the fusion protein can include a peptide linker between one or more polypeptide fragments or portions that includes one or more amino acid sequences not forming part of the biologically active peptide portions. Any suitable peptide linker can be used. Such a linker can be any suitable size. Typically, the linker is less than about 30 amino acid residues, preferably less than about 20 amino acid residues, and more preferably about 10 or less than 10 amino acid residues. Typically, the linker predominantly comprises or consists of neutral amino acid residues. Suitable linkers are generally described in, e.g., U.S. Pat. Nos. 5,990,275, 6,010,883, 6,197,946, and European Patent Application 0 035 384. If separation of peptide fragments or peptide portions is desirable a linker that facilitates separation can be used. An example of such a linker is described in U.S. Pat. No. 4,719,326. “Flexible” linkers, which are typically composed of combinations of glycine and/or serine residues, can be advantageous. Examples of such linkers are described in, e.g., McCafferty et al., Nature 348:552-554 (1990), Huston et al., Proc. Natl. Acad. Sci. USA 85:5879-5883 (1988), Glockshuber et al., Biochemistry 29:1362-1367 (1990), and Cheadle et al., Molecular Immunol. 29:21-30 (1992), Bird et al., Science 242:423-26 (1988), and U.S. Pat. Nos. 5,672,683, 6,165,476, and 6,132,992.

The use of a linker also can reduce undesired immune response to the fusion protein created by the fusion of the two peptide fragments or peptide portions, which can result in an unintended MHC I and/or MHC II epitope being present in the fusion protein. In addition to the use of a linker, identified undesirable epitope sequences or adjacent sequences can be PEGylated (e.g., by insertion of lysine residues to promote PEG attachment) to shield identified epitopes from exposure. Other techniques for reducing immunogenicity of the fusion protein of the invention can be used in association with the administration of the fusion protein include the techniques provided in U.S. Pat. No. 6,093,699.

Making Polypeptides

Recombinant methods for producing and isolating polypeptides of the invention are described below. In addition to recombinant production, the polypeptides may be produced by direct peptide synthesis using solid-phase techniques (see, e.g., Stewart et al. (1969) Solid-Phase Peptide Synthesis, WH Freeman Co, San Francisco; Merrifield (1963) J. Am. Chem. Soc 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to provide full-length NCSM polypeptides or fragments thereof. Alternatively, such sequences may be ordered from any number of companies which specialize in production of polypeptides. Most commonly, polypeptides of the invention are produced by expressing coding nucleic acids and recovering polypeptides, e.g., as described below.

Methods for producing the polypeptides of the invention are also included. One such method comprises introducing into a population of cells any nucleic acid described herein, which is operatively linked to a regulatory sequence effective to produce the encoded polypeptide, culturing the cells in a culture medium to produce the polypeptide, and isolating the polypeptide from the cells or from the culture medium. An amount of nucleic acid sufficient to facilitate uptake by the cells (transfection) and/or expression of the polypeptide is utilized. The culture medium can be any described herein and in the Examples. Additional media are known to those of skill in the art. The nucleic acid is introduced into such cells by any delivery method described herein, including, e.g., injection, gene gun, passive uptake, etc. The nucleic acid of the invention may be part of a vector, such as a recombinant expression vector, including a DNA plasmid vector, or any vector described herein. The nucleic acid or vector comprising a nucleic acid of the invention may be prepared and formulated as described herein, above, and in the Examples below. Such a nucleic acid or expression vector may be introduced into a population of cells of a mammal in vivo, or selected cells of the mammal (e.g., tumor cells) may be removed from the mammal and the nucleic acid expression vector introduced ex vivo into the population of such cells in an amount sufficient such that uptake and expression of the encoded polypeptide results. Or, a nucleic acid or vector comprising a nucleic acid of the invention is produced using cultured cells in vitro. In one aspect, the method of producing a polypeptide of the invention comprises introducing into a population of cells a recombinant expression vector comprising any nucleic acid described herein in an amount and formula such that uptake of the vector and expression of the polypeptide will result; administering the expression vector into a mammal by any introduction/delivery format described herein; and isolating the polypeptide from the mammal or from a byproduct of the mammal.

Polypeptides of the invention can be subject to various changes, such as one or more amino acid or nucleic acid insertions, deletions, and substitutions, either conservative or non-conservative, including where, e.g., such changes might provide for certain advantages in their use, e.g., in their therapeutic or prophylactic use or administration or diagnostic application. Procedures for making variants of polypeptides by using amino acid substitutions, deletions, insertions, and additions are routine in the art. Polypeptides and variants thereof having the desired ability to induce an immune response against a mammalian EpCAM or antigenic fragment thereof (e.g., T cell proliferation/activation abilities, cytokine-inducing properties, ability to induce EpCAM-specific antibodies, and/or anti-EpCAM antibody binding properties) are readily identified by assays known to those of skill in the art and by the assays described herein. The nucleic acids of the invention can also be subject to various changes, such as one or more substitutions of one or more nucleic acids in one or more codons such that a particular codon encodes the same or a different amino acid, resulting in either a conservative or non-conservative substitution, or one or more deletions of one or more nucleic acids in the sequence. The nucleic acids can also be modified to include one or more codons that provide for optimum expression in an expression system (e.g., mammalian cell or mammalian expression system), while, if desired, said one or more codons still encode the same amino acid(s). Procedures for making variants of nucleic acids by using nucleic acid substitutions, deletions, insertions, and additions, and degenerate codons, are routine in the art, and nucleic acid variants encoding polypeptides having the desired properties described herein (e.g., an ability to induce an immune response against an mEpCAM) are readily identified using the assays described herein. Such nucleic acid changes might provide for certain advantages in their therapeutic or prophylactic use or administration, or diagnostic application. In one aspect, the nucleic acids and polypeptides can be modified in a number of ways so long as they comprise a sequence substantially identical (as defined below) to a sequence in a respective TAg-encoding nucleic acid or TAg polypeptide of the invention.

The polypeptides provided by the invention are of various sizes and composition. For example, in some aspects, the invention provides polypeptides comprising an immunogenic amino acid sequence that is about 185, 240, 265, or 315 amino acids in length. In addition, the invention also provides, polypeptides comprising a novel signal peptide sequence and/or immunogenic amino acid sequence, which signal peptide sequence and/or immunogenic amino acid sequence can be only about 20-25 amino acids in length. Immunogenic fragments of polypeptides of the invention, which can be as small as about 8, 10, 12, 15, or 20 amino acids in length also are provided. Also provided are novel polypeptide sequences that correspond to a transmembrane or a cytoplasmic domain.

Using Polypeptides

Polypeptides of the invention that have an ability to induce an immune response against a mammalian EpCAM or antigenic fragment thereof (e.g., T cell proliferation/activation abilities, cytokine-inducing properties, ability to induce EpCAM-specific antibodies, and/or anti-EpCAM antibody binding properties) are useful in a variety therapeutic or prophylactic methods described below. Polypeptides of the invention having the ability to induce the production of one or more T cell-associated cytokines in a tissue, organ, and/or host comprising T cells, when the polypeptide is administered or expressed therein in an immunogenic amount, are useful in these applications. Further, polypeptides of the invention that induce the production of interferon-gamma when administered to or expressed in a mammalian host in an amount sufficient to stimulate such production. Such polypeptides are useful in treating tumors associated with EpCAM over expression, including particular cancers, as discussed elsewhere herein. Such polypeptides are useful as vaccines to treat such tumors and/or associated metastatic diseases. Polypeptides of the invention having the ability to bind antibodies to hEpCAM are useful in diagnostic assays to detect e.g., the presence of such antibodies in human serum. Diagnostic assays are discussed in greater detail below. Nucleic acids of the invention that encodes polypeptides having such properties are similarly useful in such methods and applications, as described in greater detail below.

In another aspect, a polypeptide or antigenic fragment thereof of the invention is used to produce antibodies that have, e.g., diagnostic, therapeutic, or prophylactic uses. Antibodies to polypeptides or peptide fragments thereof of the invention may be generated by methods well known in the art. Such antibodies may include, but are not limited to, polyclonal, monoclonal, chimeric, humanized, single chain, Fab fragments and fragments produced by a Fab expression library. Antibodies, e.g., those that block receptor binding, are especially preferred for therapeutic and/or prophylactic use. Polypeptides for antibody induction do not require biological activity; however, the polypeptides or oligopeptides are antigenic. Peptides used to induce specific antibodies may have an amino acid sequence consisting of at least about 10 amino acids, preferably at least about 15 or 20 amino acids or at least about 25 or 30 amino acids. Short stretches of a polypeptide of the invention may be fused with another protein, such as keyhole limpet hemocyanin, and antibody produced against the chimeric molecule.

Methods of producing polyclonal and monoclonal antibodies are known to those of skill in the art, and many antibodies are available. See, e.g., Current Protocols in Immunology, John Colligan et al., eds., Vols. I-IV (John Wiley & Sons, Inc., NY, 1991 and 2001 Supplement); and Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; and Goding (1986) Monoclonal Antibodies: Principles and Practice (2d ed.) Academic Press, New York, N.Y.; and Kohler and Milstein (1975) Nature 256:495-497. Other suitable techniques for antibody preparation include selection of libraries of recombinant antibodies in phage or similar vectors. See Huse et al. (1989) Science 246:1275-1281; and Ward et al. (1989) Nature 341:544-546. Specific monoclonal and polyclonal antibodies and antisera will usually bind with a K_{D} of at least about 0.1 μM, preferably at least about 0.01 μM or better, and most typically and preferably 0.001 μM or better.

Detailed methods for preparation of chimeric (humanized) antibodies can be found in U.S. Pat. No. 5,482,856. Additional details on humanization and other antibody production and engineering techniques can be found in Borrebaeck (ed.) (1995) Antibody Engineering, 2^{nd} Edition Freeman and Company, NY (Borrebaeck); McCafferty et al. (1996) Antibody Engineering, A Practical Approach IRL at Oxford Press, Oxford, England (McCafferty), and Paul (1995) Antibody Engineering Protocols Humana Press, Towata, N.J. (Paul). In one useful embodiment, this invention provides for fully humanized antibodies against the polypeptides of the invention or fragments thereof. Humanized antibodies are especially desirable in applications where the antibodies are used as therapeutics and/or prophylactics in vivo in human patients. Human antibodies consist of characteristically human immunoglobulin sequences. The human antibodies of this invention can be produced in using a wide variety of methods (see, e.g., Larrick et al., U.S. Pat. No. 5,001,065, and Borrebaeck, McCafferty, and Paul, supra, for a review). In one embodiment, the human antibodies of the present invention are produced initially in trioma cells. Genes encoding the antibodies are then cloned and expressed in other cells, such as nonhuman mammalian cells. The general approach for producing human antibodies by trioma technology is described by Ostberg et al. (1983), Hybridoma 2:361-367, Ostberg, U.S. Pat. No. 4,634,664, and Engelman et al., U.S. Pat. No. 4,634,666. The antibody-producing cell lines obtained by this method are called triomas because they are descended from three cells two human and one mouse. Triomas have been found to produce antibody more stably than ordinary hybridomas made from human cells.

Additional applications and uses of the polypeptides, nucleic acids, vectors, antibodies, compositions, and vectors of the invention are discussed elsewhere herein.

Nucleic Acids of the Invention

One aspect of the invention pertains to novel isolated, recombinant, synthetic, and/or non-naturally occurring nucleic acids that are useful in a number of contexts including, e.g., the expression of at least polypeptide that induces an immune response against a mammalian EpCAM, such as hEpCAM, or an antigenic fragment thereof. In one aspect, the invention provides an isolated, recombinant, synthetic, and/or non-naturally occurring nucleic acid comprising a nucleotide sequence encoding any at least one of (or combination of) the polypeptides of the invention described above and elsewhere herein. Any nucleic acid of the invention can be characterized as isolated, recombinant synthetic, and/or non-naturally occurring, unless otherwise stated.

In one aspect, the invention provides an isolated, recombinant, synthetic or non-naturally occurring nucleic acid comprising a nucleotide sequence that has at least about 70, 75, 80, 85, 90, 91, 92, 93, 94, 95, 96, 97, 98, 99, 99.5 or 100% nucleic acid sequence identity or sequence similarity with a nucleic acid sequence that encodes a polypeptide comprising a polypeptide sequence selected from the group consisting of SEQ ID NOS:1, 4-10, 12, 13, 32, 34, 78, and 92, or a complementary nucleotide sequence thereof. In a particular aspect, such nucleic acid encodes a polypeptide comprising a sequence selected from the group consisting of SEQ ID NOS: 1, 4-10, 12, 13, 32, 34, 78, and 92. Preferably, such nucleic acids of the invention encode a polypeptide that induces an immune response against a mammalian EpCAM or an antigenic fragment thereof, or a cell or tissue expressing an mEpCAM. Typically, such nucleic acids express polypeptides that induce an immune response to EpCAM in an appropriate context (i.e., when operably linked to a suitable promoter in frame in a nucleic acid). In one aspect, such polypeptide is able to induce an immune response against hEpCAM that is at least as great as the immune response induced by hEpCAM, an hEpCAM homolog, an hEpCAM ortholog, or an antigenic fragment of any thereof, or a cell or tissue expressing hEpCAM.

Determining the level of identity of a portion of the above-described nucleic acid to its target (i.e., SEQ ID NO:19) can be accomplished through local sequence alignment techniques described elsewhere herein (e.g., using LFASTA, LALIGN, and/or by aligning sequences manually in an optimal local sequence alignment).

In another aspect, the invention provides an isolated, recombinant, synthetic or non-naturally occurring nucleic acid comprising a polynucleotide sequence that has at least about 75, 80, 81, 82, 83, 84, 85, 86, 87, 88, 90, 91, 92, 93, 94, 95, 96, 97, 98, 99, or 100% nucleic acid sequence identity to a polynucleotide sequence selected from the group consisting of SEQ ID NOS: 16, 19-23, 26-28, 33, 35, 79, and 94. Many such nucleic acids encode a polypeptide that induces an immune response against a mammalian EpCAM, preferably hEpCAM, or an antigenic fragment thereof, or a cell or tissue expressing hEpCAM. Advantageously, many such nucleic acids have the ability to induce a T cell and/or humoral immune response to hEpCAM (e.g., a T cell and B cell immune response to EpCAM-overexpressing (EpCAM^{High}) cells in a human host) when administered to a human in an effective amount. For example, nucleic acids comprising a number of nucleotide sequences having high levels of nucleic acid sequence identity (e.g., about 85-99%) to the sequence of SEQ ID NO: 19 encode polypeptides that are able to induce an immune response to EpCAM. In a particular aspect, such nucleic acid consisting essentially or consists of the nucleotide sequence of SEQ ID NO: 19, 20, or 21. Preferably, such nucleic acid encodes an hEpCAM-specific antibody response, hEpCAM-specific T cell proliferation response, and/or cytokine production; such immune response may be at least as great as that induced by hEpCAM.

In another aspect, the invention provides an isolated, recombinant or non-naturally occurring nucleic acid encoding a polypeptide that has an ability to induce, promote, and/or enhance an immune response against hEpCAM or an antigenic fragment thereof, or cell or tissue expressing hEpCAM, wherein the nucleic acid comprises one or more of the following: (a) a polynucleotide sequence that encodes a polypeptide comprising a polypeptide sequence having at least about 80, 85, 90, 95, 96, 97, 98, 99, or 100% sequence identity to a polypeptide sequence comprising amino acid residues 81-265, 82-265, 22-265, 23-265, 24-265, or 1-265 of the polypeptide sequence of SEQ ID NO:4, or a complementary polynucleotide sequence thereof; (b) a polynucleotide sequence comprising nucleotide residues 64-795, 67-795, 70-795, 241-795, 244-795, 247-795, 64-795, 67-795, 70-795, 73-795, or 1-795 of the polynucleotide sequence of SEQ ID NO: 19, or a complementary polynucleotide sequence thereof; (c) a polynucleotide sequence selected from the group consisting of SEQ ID NOS:16, 20-23, 26-28, 33, 35, and 79, or a complementary polynucleotide sequence of any thereof; and (d) a polynucleotide sequence that, but for the degeneracy of the genetic code, hybridizes under at least stringent conditions over substantially the entire length of the polynucleotide sequence of (a), (b), or (c) above. Preferably, such nucleic acid encodes an antigenic polypeptide having an ability to induce an immune response against hEpCAM or an antigenic fragment thereof.

In a particular aspect, the invention provides a nucleic acid that comprises the nucleotide sequence of SEQ ID NO: 16 or SEQ ID NO:26, each of which encodes an extracellular domain. The nucleic acid can be any of the above-described types of nucleic acids (e.g., an RNA, a single stranded (ss) cDNA, or a DNA comprising a phosphorothioate backbone). The nucleic acid can further comprise any suitable additional nucleotide sequence(s). For example, such ECD-encoding nucleic acid can further comprise the nucleotide sequence of SEQ ID NO: 17 or SEQ ID NO:2, each of which encodes a propeptide, and optionally may further comprise the nucleotide sequence of SEQ ID NO: 18, which encodes a signal peptide. These nucleotide sequences can be directly fused together, in appropriate reading frame, such that the nucleic acid comprises a nucleotide sequence that encodes an SP/PP/ECD polypeptide, such as the polypeptide comprising the polypeptide sequence of SEQ ID NO:4.

In anther aspect is provided a nucleic acid comprising a nucleotide sequence that has, or comprises a subsequence that has, at least about 75, 80, 85, 86, 87, 88, 89, 90, 91, 92, 93, 94, 95, 96, 97, 98, 99, or 100% nucleotide sequence identity to a subsequence of SEQ ID NO:21, which subsequence comprises about nucleotide residues 241-864, 244-864, 274-864, 70-864, 67-864, or 64-864 of the nucleic acid sequence of SEQ ID NO:21. Also provided is a nucleic acid that has at least about 75, 80, 85, 86, 87, 88, 89, 90, 91, 92, 93, 94, 95, 96, 97, 98, 99, or 100% nucleotide sequence identity to a subsequence of SEQ ID NO:21, said subsequence comprising at least about nucleotide residues 241-942, 244-942, 274-942, 70-942, 67-942, or 64-942 of the nucleic acid sequence of SEQ ID NO:21. Preferably, such nucleic acids encode an antigenic polypeptide that induces an immune response against a mammalian EpCAM (e.g., hEpCAM) or an antigenic fragment thereof, including e.g., an EpCAM-specific antibody response, T cell proliferation response, and/or cytokine production. Some such encoded polypeptides induce an immune response against hEpCAM or an antigenic fragment thereof that is at least as great as the immune response induced by hEpCAM or respective antigenic fragment thereof.

Also provided is a nucleic acid comprising a nucleotide sequence that has at least about 75, 80, 85, 86, 87, 88, 89, 90, 91, 92, 93, 94, 95, 96, 97, 98, 99, or 100% nucleotide sequence identity to a subsequence of SEQ ID NO:21, said subsequence comprising about nucleotide residues 1-69 (encoding a signal peptide), 70-240 (encoding a propeptide), 796-864 (encoding a TMD) and 865-942 (encoding a CD) of the sequence of SEQ ID NO:21.

In another aspect, the invention provides a nucleic acid which comprises a nucleotide sequence that encodes a polypeptide comprising an amino acid sequence having at least about 90, 91, 92, 93, 94, 95, 96, 97, 98, 99, or 100% sequence identity to a polypeptide sequence corresponding to amino acid residues 81-265, amino acid residues 82-265, amino acid residues 22-265, amino acid residues 24-265, or amino acid residues 1-265 of SEQ ID NO:4, or a complementary nucleotide sequence thereof. Some such nucleotide sequences encode a polypeptide that induces an immune response against hEpCAM or an antigenic fragment thereof, including, e.g., an EpCAM-specific antibody response, T cell proliferation response, and/or cytokine production.

In another aspect, the invention provides an RNA nucleic acid comprising a nucleotide sequence selected from the group consisting of SEQ ID NOS:16, 19-23, 26-28, 33, 35, 79, and 94, in which all of the thymine nucleotide bases in the DNA sequence are replaced or substituted with uracil nucleotide bases. In another aspect, the invention provides an RNA nucleic acid comprising a nucleotide sequence that has at least about 80, 85, 90, 95, 96, 97, 98, or 99% nucleic acid sequence identity to at least one sequence selected from the group consisting of SEQ ID NOS: 16, 19-23, 26-28, 33, 35, 79, and 94, wherein all of the thymine bases in the sequence are replaced or substituted with uracil bases and identity is calculated as if thymine residues are equivalent to uracil residues with respect to percent identity. In yet a further variation, the invention provides an RNA nucleic acid that hybridizes under at least stringent conditions over substantially the entire length of a nucleic acid comprising a nucleotide sequence having at least about 80, 85, 90, 91, 92, 93, 94, 95, 96, 97, 98, 99% or more sequence identity to a polynucleotide sequence selected from the group consisting of SEQ ID NOS:16, 19-23, 26-28, 32-35, and 79, or that would so hybridize but for the degeneracy of the genetic code.

Immune responses induced against EpCAM by polypeptides encoded by nucleic acids of the invention include the ability to induces a T cell immune proliferation or activation response against a mammalian EpCAM or an antigenic fragment thereof or a cell or tissue expressing mEpCAM; the ability to induce production of antibodies capable of specifically binding a mammalian EPCAM or an antigenic fragment thereof or a cell or tissue expressing mEpCAM; and the ability to induce or enhance production of at least one cytokine (such as an IFN or IL). Preferably, nucleic acids of the invention encode polypeptides that induce at least one such immune response that is specifically against hEpCAM or a cell or tissue expressing hEpCAM. Preferably, nucleic acids of the invention encode polypeptides that are capable of inducing an immune response against hEpCAM that is about at least as great as the immune response induced by hEpCAM or a cell or tissue expressing hEpCAM.

Many fragments of these nucleic acids will express polypeptides that induce such an immune response, which can be readily identified with reasonable experimentation. In general, such a fragment will be at least about 24 nucleotides or base pairs in length. Usually, such a fragment will be significantly larger (e.g., at least about 60 nucleotides or base pairs in length). More commonly, such a fragment will encode an amino acid sequence of at least 45 residues in length (e.g., a fragment of SEQ ID NO:4 that is at least about 50 amino acid residues, at least about 70 amino acid residues, or more in length), which amino acid sequence does not occur in EpCAM, an EpCAM homolog, or an EpCAM ortholog.

A nucleic acid of the invention can be isolated by any suitable technique, of which several are known in the art. An isolated nucleic acid of the invention (e.g., a nucleic acid that is prepared in a host cell and subsequently substantially purified by any suitable nucleic acid purification technique) can be re-introduced into a host cell or re-introduced into a cellular or other biological environment or composition wherein it is no longer the dominant nucleic acid species and is no longer separated from other nucleic acids.

Nearly any isolated or synthetic nucleic acid of the invention can be inserted in or fused to a suitable larger nucleic acid molecule (e.g., a chromosome, plasmid, a viral genome, a gene sequence, a linear expression element, a bacterial genome, or an artificial chromosome, such as a mammalian artificial chromosome (MAC), or the yeast and bacterial counterparts thereof (i.e., a YAC or a BAC) to form a recombinant nucleic acid using standard techniques. As another example, an isolated nucleic acid of the invention can be fused to smaller nucleotide sequences, such as promoter sequences, immunostimulatory sequences, and/or sequences encoding other amino acids, such as other antigen epitopes and/or linker sequences to form a recombinant nucleic acid.

A synthetic nucleic acid is typically generated by chemical synthesis techniques applied outside of the context of a host cell (e.g., a nucleic acid produced through PCR or chemical synthesis techniques, examples of which are described further herein).

Nucleic acids encoding polypeptides of the invention can have any suitable chemical composition that permits the expression of a polypeptide of the invention or other desired biological activity (e.g., hybridization with other nucleic acids). Thus, a nucleic acid of the invention can be single stranded or double stranded RNA, DNA, or combinations thereof and can include any suitable nucleotide base, base analog, and/or backbone (e.g., a backbone formed by, or including, a phosphothioate, rather than phosphodiester, linkage). Modifications to a nucleic acid are particularly tolerable in the 3rd position of an mRNA codon sequence encoding such a polypeptide. In particular aspects, at least a portion of the nucleic acid comprises a phosphorothioate backbone, incorporating at least one synthetic nucleotide analog in place of or in addition to the naturally occurring nucleotides in the nucleic acid sequence. Also or alternatively, the nucleic acid can comprise the addition of bases other than guanine, adenine, uracil, thymine, and cytosine. Such modifications can be associated with longer half-life, and thus can be desirable in nucleic acids vectors of the invention. Thus, in one aspect, the invention provides recombinant nucleic acids and nucleic acid vectors (discussed further below), which nucleic acids or vectors comprise at least one of the aforementioned modifications, or any suitable combination thereof, wherein the nucleic acid persists longer in a mammalian host than a substantially identical nucleic acid without such a modification or modifications. Examples of modified and/or non-cytosine, non-adenine, non-guanine, non-thymine nucleotides that can be incorporated in a nucleotide sequence of the invention are provided in, e.g., the MANUAL OF PATENT EXAMINING PROCEDURE § 2422 (7th Revision—2000).

It is to be understood that a nucleic acid encoding one of the polypeptides of the invention, including those described above and elsewhere herein, is not limited to a sequence that directly codes for expression or production of a polypeptide of the invention. For example, the nucleic acid can comprise a nucleotide sequence which results in a polypeptide of the invention through intein-like expression (as described in, e.g., Colson and Davis (1994) Mol. Microbiol. 12(3):959-63, Duan et al. (1997) Cell 89(4):555-64, Perler (1998) Cell 92(1):1-4, Evans et al. (1999) Biopolymers 51(5):333-42, and de Grey, Trends Biotechnol. 18(9):394-99 (2000)), or a nucleotide sequence which comprises self-splicing introns (or other self-spliced RNA transcripts), which form an intermediate recombinant polypeptide-encoding sequence (as described in, e.g., U.S. Pat. No. 6,010,884). The nucleic acid also or alternatively can comprise sequences which result in other splice modifications at the RNA level to produce an mRNA transcript encoding the polypeptide and/or at the DNA level by way of trans-splicing mechanisms prior to transcription (principles related to such mechanisms are described in, e.g., Chabot, Trends Genet. (1996) 12(11):472-78, Cooper (1997) Am. J. Hum. Genet. 61(2):259-66, and Hertel et al. (1997) Curr. Opin. Cell. Biol. 9(3):350-57). Due to the inherent degeneracy of the genetic code, several nucleic acids can code for any particular polypeptide of the invention. Thus, for example, any of the particular nucleic acids described herein can be modified by replacement of one or more codons with an equivalent codon (with respect to the amino acid called for by the codon) based on genetic code degeneracy. Further, other nucleic acid sequences that encode a polypeptide having the same or a functionally equivalent polypeptide sequence as a polypeptide sequence of the invention can also be used to synthesize, clone and express such polypeptide.

Any of the nucleic acids of the invention as described herein may be codon optimized for expression in a particular mammal (normally humans). Techniques for codon optimization are known in the art and briefly discussed elsewhere herein. Such nucleic acids can comprise additional immunogenic acid sequences of the invention as described elsewhere herein. Further, nucleic acids can be modified by truncation or one or more residues of the C-terminus portion of the sequence. Additional, a variety of stop or termination codons may be included at the end of the nucleotide sequence as further discussed below.

The polynucleotides of the invention can be in the form of RNA or in the form of DNA, and include mRNA, cRNA, synthetic RNA and DNA, and cDNA. The nucleic acids of the invention are typically DNA molecules, and usually a double stranded DNA molecules. However, single stranded DNA, single stranded RNA, double stranded RNA, and hybrid DNA/RNA nucleic acids comprising any of the nucleotide sequences of the invention also are provided.

The nucleic acids of the invention can be double-stranded or single-stranded, and if single-stranded, can be the coding strand or the non-coding (i.e., antisense or complementary) strand. In addition to a nucleotide sequence encoding a polypeptide of the invention (e.g., nucleotide sequence that comprise the coding sequence of a TAg polypeptide), the polynucleotide of the invention can comprise one or more additional coding nucleotide sequences, so as to encode, e.g., a fusion protein, a pre-protein, a prepro-protein, or the like, a heterologous transmembrane domain and/or cytoplasmic domain, targeting sequence (other than a signal sequence), or the like (more particular examples of which are discussed further herein), and/or can comprise non-coding nucleotide sequences, such as introns, terminator sequence, or 5′ and/or 3′ untranslated regions (e.g., the 5′ untranslated region of wild-type EpCAM DNA, the 3′ untranslated region of wild-type EpCAM DNA, or both), which regions can be effective for expression of the coding sequence in a suitable host, and/or control elements, such as a promoter (e.g., naturally occurring or recombinant or shuffled promoter).

In particular aspects, a nucleic acid can comprise untranslated sequences associated with wild-type (WT) mammalian EpCAM nucleic acid, e.g., WT hEpCAM DNA or RNA. For example, the nucleic acid can be linked to the polyA sequence of EPCAM (nucleotides 1486-1491 of the EPCAM sequence—see Strnad et al., supra). Alternatively or additionally, the sequence can be associated with the GC rich noncoding sequences of EpCAM (see id.) and/or EpCAM DNA introns sequences.

Such nucleic acids may be included in a vector, cell, or host environment in which TAg coding sequence is a heterologous gene.

Polynucleotides of the invention include polynucleotide sequences that encode TAg polypeptides and fragments thereof (including, e.g., all monomeric and multimeric forms of soluble TAg polypeptides and fusion proteins), polynucleotides that hybridize under at least stringent conditions to polypeptide sequences defined herein, polynucleotide sequences complementary to these polynucleotide sequences, and variants, analogs, and homologue derivatives of all of the above. A coding sequence refers to a nucleotide sequence encodes a particular polypeptide or domain, region, or fragment of said polypeptide. A coding sequence may code for a TAg polypeptide or fragment thereof having a functional property, such as the ability to induce an immune response against EpCAM.

The polynucleotides include the respective coding sequences of components of a TAg polypeptide, including, e.g., the coding sequence for each of the signal peptide, propeptide, and ECD, and, optionally, the transmembrane domain, cytoplasmic domain and variants, analogs, and homologue derivatives thereof. A coding sequence for a TAg mature domain is also included. Polynucleotide sequences can also be found in combination with typical compositional formulations of nucleic acids, including in the presence of carriers, buffers, adjuvants, excipients, and the like, as are known to those of ordinary skill in the art. Nucleotide fragments typically comprise at least about 500 nucleotide bases, usually at least about 600, 650, or 700 bases, and often 750 or more bases. The nucleotide fragments, variants, analogs, and homologue derivatives of TAg-encoding polynucleotides may have hybridize under highly stringent conditions to another TAg-encoding polynucleotide or homologue sequence described herein and/or encode amino acid sequences having at least one of the EpCAM immune response properties described herein.

Unless otherwise indicated, a particular nucleic acid sequence described herein also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acid Res. 19:5081; Ohtsuka et al. (1985) J. Biol. Chem. 260:2605-2608; Cassol et al. (1992); Rossolini et al. (1994) Mol. Cell. Probes 8:91-98).

Nucleic Acid Hybridization

As noted above, the invention includes nucleic acids that hybridize to a target nucleic acid of the invention, such as, e.g. one selected from the group consisting of SEQ ID NOS: 16, 20-23 26-28, 33, 35, 79, and 94, wherein hybridization is over substantially the entire length of the target nucleic acid. Complementary nucleic acids are also contemplated. Preferably, the hybridizing nucleic acid hybridizes to a nucleotide sequence of the invention, such as that of SEQ ID NO: 19, under at least stringent conditions, and more preferably under at least high stringency conditions. Moderately stringent, stringent, and highly stringent hybridization conditions for nucleic acid hybridization experiments are known. Examples of factors that can be combined to achieve such levels of stringency are briefly discussed herein.

Nucleic acids “hybridize” when they associate, typically in solution. Nucleic acids hybridize due to a variety of well-characterized physico-chemical forces, such as hydrogen bonding, solvent exclusion, base stacking and the like. An extensive guide to the hybridization of nucleic acids is found in Tijssen (1993) LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” (Elsevier, N.Y.) (hereinafter “Tjissen”), as well as in Ausubel, supra, Hames and Higgins (1995) GENE PROBES 1, IRL Press at Oxford University Press, Oxford, England (Hames and Higgins 1) and Hames and Higgins (1995) GENE PROBES 2, IRL Press at Oxford University Press, Oxford, England (Hames and Higgins 2) provide details on the synthesis, labeling, detection and quantification of DNA and RNA, including oligonucleotides.

An indication that two nucleic acid sequences are substantially identical is that the two molecules hybridize to each other under at least stringent conditions. The phrase “hybridizing specifically to,” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

“Stringent hybridization wash conditions” and “stringent hybridization conditions” in the context of nucleic acid hybridization experiments, such as Southern and northern hybridizations, are sequence dependent, and are different under different environmental parameters. An extensive guide to hybridization of nucleic acids is found in Tijssen (1993), supra, and in Hames and Higgins I and Hames and Higgins 2, supra.

Generally, high stringency conditions are selected such that hybridization occurs at about 5° C. or less than the thermal melting point (Tₘ) for the specific sequence at a defined ionic strength and pH. The Tₘ is the temperature (under defined ionic strength and pH) at which 50% of the test sequence hybridizes to a perfectly matched probe. In other words, the Tₘ indicates the temperature at which the nucleic acid duplex is 50% denatured under the given conditions and its represents a direct measure of the stability of the nucleic acid hybrid. Thus, the Tₘ corresponds to the temperature corresponding to the midpoint in transition from helix to random coil; it depends on length, nucleotide composition, and ionic strength for long stretches of nucleotides. Typically, under “stringent conditions,” a probe will hybridize to its target subsequence, but to no other sequences. “Very stringent conditions” are selected to be equal to the Tₘ for a particular probe.

The Tₘ of a DNA-DNA duplex can be estimated using equation (1): Tₘ (° C.)=81.5° C.+16.6 (log₁₀M)+0.41 (% G+C)−0.72 (% f)−500/n, where M is the molarity of the monovalent cations (usually Na+), (% G+C) is the percentage of guanosine (G) and cytosine (C) nucleotides, (% f) is the percentage of formalize and n is the number of nucleotide bases (i.e., length) of the hybrid. See Rapley, R. and Walker, J. M. eds., MOLECULAR BIOMETHODS HANDBOOK (1998), Humana Press, Inc., Tijssen (1993) LABORATORY TECHNIQUES IN BIOCHEMISTRY AND MOLECULAR BIOLOGY—HYBRIDIZATION WITH NUCLEIC ACID PROBES. [hereinafter Rapley and Walker]. The Tₘ of an RNA-DNA duplex can be estimated using equation (2): Tₘ (° C.)=79.8° C.+18.5 (log₁₀M)+0.58 (% G+C)−11.8(% G+C)²−0.56 (% f)−820/n, where M is the molarity of the monovalent cations (usually Na+), (% G+C) is the percentage of guanosine (G) and cytosine (C) nucleotides, (% f) is the percentage of formamide and n is the number of nucleotide bases (i.e., length) of the hybrid. Id. Equations 1 and 2 above are typically accurate only for hybrid duplexes longer than about 100-200 nucleotides. Id. The Tₘ of nucleic acid sequences shorter than 50 nucleotides can be calculated as follows: Tₘ (° C.)=4(G+C)+2(A+T), where A (adenine), C, T (thymine), and G are the numbers of the corresponding nucleotides.

In general, non-hybridized nucleic acid material desirably is removed by a series of washes, the stringency of which can be adjusted depending upon the desired results, in conducting hybridization analysis. Low stringency washing conditions (e.g., using higher salt and lower temperature) increase sensitivity, but can product nonspecific hybridization signals and high background signals. Higher stringency conditions (e.g., using lower salt and higher temperature that is closer to the hybridization temperature) lower the background signal, typically with only the specific signal remaining. Addition useful guidance concerning such hybridization techniques is provided in, e.g., Rapley and Walker, supra (in particular, with respect to such hybridization experiments, part I, chapter 2, “Overview of principles of hybridization and the strategy of nucleic acid probe assays”), Elsevier, New York, as well as in Ausubel, supra, Sambrook et al., supra, Watson et al., supra, Hames and Higgins (1995) GENE PROBES 1, IRL Press at Oxford University Press, Oxford, England, and Hames and Higgins (1995) GENE PROBES 2, IRL Press at Oxford University Press, Oxford, England.

Exemplary stringent (or regular stringency) conditions for analysis of at least two nucleic acids comprising at least 100 nucleotides include incubation in a solution or on a filter in a Southern or northern blot comprises 50% formalin (or formamide) with 1 mg of heparin at 42° C., with the hybridization being carried out overnight. A regular stringency wash can be carried out using, e.g., a solution comprising 0.2× SSC wash at about 65° C. for about 15 minutes (see Sambrook, supra, for a description of SSC buffer). Often, the regular stringency wash is preceded by a low stringency wash to remove background probe signal. A low stringency wash can be carried out in, for example, a solution comprising 2× SSC at about 40° C. for about 15 minutes. A highly stringent wash can be carried out using a solution comprising 0.15 M NaCl at about 72° C. for about 15 minutes. An example medium (regular) stringency wash, less stringent than the regular stringency wash described above, for a duplex of, e.g., more than 100 nucleotides, can be carried out in a solution comprising 1× SSC at 45° C. for 15 minutes. An example low stringency wash for a duplex of, e.g., more than 100 nucleotides, is carried out in a solution of 4-6× SSC at 40° C. for 15 minutes. For short probes (e.g., about 10 to 50 nucleotides), stringent conditions typically involve salt concentrations of less than about 1.0 M Na⁺ ion, typically about 0.01 to 1.0 M Na⁺ ion concentration (or other salts) at pH 7.0 to 8.3, and the temperature is typically at least about 30° C. Stringent conditions can also be achieved with the addition of destabilizing agents such as formamide.

Exemplary moderate stringency conditions include overnight incubation at 37° C. in a solution comprising 20% formalin (or formamide), 0.5× SSC, 50 mM sodium phosphate (pH 7.6), 5× Denhardt's solution, 10% dextran sulfate, and 20 mg/mL denatured sheared salmon sperm DNA, followed by washing the filters in 1× SSC at about 37-50° C., or substantially similar conditions, e.g., the moderately stringent conditions described in Sambrook et al., supra, and/or Ausubel, supra.

High stringency conditions are conditions that use, for example, (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 m sodium phosphate buffer at pH 6.5 with 750 mM sodium chloride, 75 mM sodium citrate at 42° C., or (3) employ 50% formamide, 5× SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at (i) 42° C. in 0.2× SSC, (ii) at 55° C. in 50% formamide and (iii) at 55° C. in 0.1× SSC (preferably in combination with EDTA).

In general, a signal to noise ratio of 2× or 2.5×-5× (or higher) than that observed for an unrelated probe in the particular hybridization assay indicates detection of a specific hybridization. Detection of at least stringent hybridization between two sequences in the context of the present invention indicates relatively strong structural similarity or homology to, e.g., the nucleic acids of the present invention.

As noted, “highly stringent” conditions are selected to be about 5° C. or less lower than the thermal melting point (Tₘ) for the specific sequence at a defined ionic strength and pH. Target sequences that are closely related or identical to the nucleotide sequence of interest (e.g., “probe”) can be identified under highly stringency conditions. Lower stringency conditions are appropriate for sequences that are less complementary. See, e.g., Rapley and Walker; Sambrook, all supra.

Comparative hybridization can be used to identify nucleic acids of the invention, and this comparative hybridization method is a preferred method of distinguishing nucleic acids of the invention. Detection of highly stringent hybridization between two nucleotide sequences in the context of the present invention indicates relatively strong structural similarity/homology to, e.g., the nucleic acids provided in the sequence listing herein. Highly stringent hybridization between two nucleotide sequences demonstrates a degree of similarity or homology of structure, nucleotide base composition, arrangement or order that is greater than that detected by stringent hybridization conditions. In particular, detection of highly stringent hybridization in the context of the present invention indicates strong structural similarity or structural homology (e.g., nucleotide structure, base composition, arrangement or order) to, e.g., the nucleic acids provided in the sequence listings herein. For example, it is desirable to identify test nucleic acids which hybridize to the exemplar nucleic acids herein under stringent conditions.

Thus, one measure of stringent hybridization is the ability to hybridize to a nucleic acid of the invention (e.g., a nucleic acid comprising a polynucleotide sequence selected from the group of SEQ ID NOS: 16-28, 33, 35, 79, and 94, or a complementary polynucleotide sequence thereof) under highly stringent conditions (or very stringent conditions, or ultra-high stringency hybridization conditions, or ultra-ultra high stringency hybridization conditions). Stringent hybridization (including, e.g., highly stringent, ultra-high stringency, or ultra-ultra high stringency hybridization conditions) and wash conditions can easily be determined empirically for any test nucleic acid.

For example, in determining highly stringent hybridization and wash conditions, the hybridization and wash conditions are gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions are gradually increased until a probe comprising one or more nucleic acid sequences selected from SEQ ID NOS:16-28, 33, 35, 79, and 94, and complementary polynucleotide sequences thereof, binds to a perfectly matched complementary target (again, a nucleic acid comprising one or more nucleic acid sequences selected from SEQ ID NOS: 16-28, 33, 35, 79, and 94, and complementary polynucleotide sequences thereof), with a signal to noise ratio that is at least 2.5×, and optionally 5× or more as high as that observed for hybridization of the probe to an unmatched target. In this case, the unmatched target may comprise a nucleic acid corresponding to, e.g., a mammalian EpCAM such as hEpCAM.

Preferably, the hybridization analysis is carried out under hybridization conditions selected such that a nucleic acid comprising a sequence that is perfectly complementary to the a disclosed reference (or known) nucleotide sequence (e.g., SEQ ID NO:19) hybridizes with the recombinant antigen-encoding sequence (e.g., a nucleotide sequence variant of the nucleic acid sequence of SEQ ID NO: 19) with at least about 5 times, preferably at least about 7 times, and more preferably at least about 10 times, higher signal-to-noise ratio than is observed in the hybridization of the perfectly complementary nucleic acid to a nucleic acid that comprises a nucleotide sequence that is at least about 80 or 90% identical to the reference nucleic acid. Such conditions can be considered indicative for specific hybridization. The above-described hybridization conditions can be adjusted, or alternative hybridization conditions selected, to achieve any desired level of stringency in selection of a hybridizing nucleic acid sequence. For example, the above-described highly stringent hybridization and wash conditions can be gradually increased (e.g., by increasing temperature, decreasing salt concentration, increasing detergent concentration and/or increasing the concentration of organic solvents, such as formalin, in the hybridization or wash), until a selected set of criteria are met. For example, the hybridization and wash conditions can be gradually increased until a desired probe, binds to a matched complementary target, with a signal-to-noise ratio that is at least about 2.5×, and optionally at least about 5× (e.g., about 10×, about 20×, about 50×, about 100×, or even about 500×), as high as the signal-to-noise ration observed from hybridization of the probe to a nucleic acid not of the invention, such as a wild-type EpCAM-encoding DNA sequence, a human EpCAM homolog DNA, and/or an EpCAM ortholog-encoding DNA.

Making and Modifying Nucleic Acids

Nucleic acids of the invention can be obtained and/or generated by application of any suitable synthesis, manipulation, and/or isolation techniques, or combinations thereof. For example, polynucleotides of the invention are typically and preferably produced through standard nucleic acid synthesis techniques, such as solid-phase synthesis techniques known in the art. In such techniques, fragments of up to about 100 bases usually are individually synthesized, then joined (e.g., by enzymatic or chemical ligation methods, or polymerase mediated recombination methods) to form essentially any desired continuous nucleic acid sequence. The synthesis of the nucleic acids of the invention can be also facilitated (or alternatively accomplished), by chemical synthesis using, e.g., the classical phosphoramidite method, which is described in, e.g., Beaucage et al. (1981) Tetrahedron Letters 22:1859-69, or the method described by Matthes et al. (1984) EMBO J. 3:801-05, e.g., as is typically practiced in automated synthetic methods. The nucleic acid of the invention also can be produced by use of an automatic DNA synthesizer. Other techniques for synthesizing nucleic acids and related principles are described in, e.g., Itakura et al., Annu Rev Biochem 53:323 (1984), Itakura et al., Science 198:1056 (1984), and Ike et al., Nucl Acid Res 11:477 (1983).

Conveniently, custom made nucleic acids can be ordered from a variety of commercial sources, such as The Midland Certified Reagent Company (mcrc@oligos.com), the Great American Gene Company (http://www.genco.com), ExpressGen Inc. (www.expressgen.com), Operon Technologies Inc. (Alameda, Calif.). Similarly, custom peptides and antibodies can be custom ordered from any of a variety of sources, e.g., PeptidoGenic (pkim@ccnet.com), HTI Bio-products, Inc. (http://www.htibio.com), and BMA Biomedicals Ltd. (U.K.), Bio. Synthesis, Inc.

Certain nucleotides of the invention may also be obtained by screening cDNA libraries (e.g., libraries generated by recombining homologous nucleic acids as in typical recursive sequence recombination methods) using oligonucleotide probes that can hybridize to or PCR-amplify polynucleotides which encode the polypeptides of the invention. Procedures for screening and isolating cDNA clones are well-known to those of skill in the art. Such techniques are described in, e.g., Berger and Kimmel, Guide to Molecular Cloning Techniques, Methods in Enzymol. Vol. 152, Acad. Press, Inc., San Diego, Calif. (“Berger”); Sambrook, supra, and CURRENT PROTOCOLS IN MOLECULAR BIOLOGY, Ausubel, supra. Some nucleic acids of the invention can be obtained by altering a naturally occurring backbone, e.g., by mutagenesis, recursive sequence recombination (e.g., shuffling), or oligonucleotide recombination. In other cases, such polynucleotides can be made in silico or through oligonucleotide recombination methods as described in the references cited herein.

Recombinant DNA techniques useful in modification of nucleic acids are well known in the art (e.g., restriction endonuclease digestion, ligation, reverse transcription and cDNA production, and PCR). Useful recombinant DNA technology techniques and principles related thereto are provided in, e.g., Mulligan (1993) Science 260:926-932, Friedman (1991) THERAPY FOR GENETIC DISEASES, Oxford University Press, Ibanez et al. (1991) EMBO J. 10:2105-10, Ibanez et al. (1992) Cell 69:329-41 (1992), and U.S. Pat. Nos. 4,440,859, 4,530,901, 4,582,800, 4,677,063, 4,678,751, 4,704,362, 4,710,463, 4,757,006, 4,766,075, and 4,810,648, and are more particularly described in Sambrook et al. (1989) MOLECULAR CLONING: A LABORATORY MANUAL, Cold Spring Harbor Press, and the third edition thereof (2001), Ausubel et al. (1994-1999), Current Protocols in Molecular Biology, Wiley Interscience Publishers (with Greene Publishing Associates for some editions), Berger and Kimmel, “Guide to Molecular Cloning Techniques” in Meth Enzymol. 152, Acad. Press, Inc. (San Diego, Calif.), and Watson et al., Recombinant DNA (2d ed.).

Substrates and Formats for Sequence Recombination and Mutagenesis

The polynucleotides of the invention and fragments thereof are optionally used as substrates for any of a variety of recombination and recursive sequence recombination reactions, in addition to their use in standard cloning methods as set forth in, e.g., Ausubel, Berger and Sambrook, e.g., to produce additional TAg polynucleotides or fragments thereof that encode TAg polypeptides and fragments thereof having with desired properties.

A variety of protocols exist for generating and identifying molecules of the invention having one of more of the properties described herein. These procedures can be used separately, and/or in combination to produce one or more variants of a nucleic acid or set of nucleic acids, as well variants of encoded proteins. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics. While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity-generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property described herein, including, e.g., an ability to induce, promote, enhance, or modulate an immune response, favorably an immune response against EpCAM, such T cell proliferation and/or activation, cytokine production (e.g., (e.g., IL-3 production and/or IFN-γ production), and/or the production of antibodies that bind (react) with EpCAM.

Descriptions of a variety of diversity generating procedures for generating modified nucleic acid sequences that encode polypeptides of the invention as described herein are found in the following publications and the references cited therein: Soong, N. et al. (2000) Nat Genet 25(4):436-439; Stemmer et al. (1999) Tumor Targeting 4:1-4; Ness et al. (1999) Nature Biotechnol. 17:893-896; Chang et al. (1999) Nature Biotechnology 0.17:793-797; Minshull and Stemmer (1999) Curr. Opin. Chemical Biol. 3:284-290; Christians et al. (1999) Nature Biotechnol. 17:259-264; Crameri et al. (1998) Nature 391:288-291; Crameri et al. (1997) Nature Biotechnol. 15:436-438; Zhang et al. (1997) Proc. Natl. Acad. Sci. USA 94:4504-4509; Patten et al. (1997) Curr. Opin. Biotechnol. 8:724-733; Crameri et al. (1996) Nature Med. 2:100-103; Crameri et al. (1996) Nature Biotechnol. 14:315-319; Gates et al. (1996) J. Mol. Biol. 255:373-386; Stemmer (1996) “Sexual PCR and Assembly PCR” In: The Encyclopedia of Molecular Biology, VCH Publishers, NY pp.447-457; Crameri and Stemmer (1995) BioTechniq. 18:194-195; Stemmer et al., (1995) Gene 164:49-53; Stemmer (1995) Science 270:1510; Stemmer (1995) Bio/Technology 13:549-553; Stemmer (1994) Nature 370:389-391; and Stemmer (1994) Proc. Natl. Acad. Sci. USA 91:10747-10751.

The term “shuffling” is used herein to indicate recombination between non-identical sequences, in some embodiments shuffling may include crossover via homologous recombination or via non-homologous recombination, such as via cre/10× and/or flp/frt systems. Shuffling can be carried out by employing a variety of different formats, including for example, in vitro and in vivo shuffling formats, in silico shuffling formats, shuffling formats that utilize either double-stranded or single-stranded templates, primer based shuffling formats, nucleic acid fragmentation-based shuffling formats, and oligonucleotide-mediated shuffling formats, all of which are based on recombination events between non-identical sequences and are described in more detail or referenced herein below, as well as other similar recombination-based formats.

DNA-based recombination can be used to generate and identify new polypeptides having (e.g., TAg polypeptides), including those having an ability to induce mEpCAM-specific immune responses as described herein.

Mutational methods of generating diversity include, for example, site-directed mutagenesis (Ling et al. (1997) Anal. Biochem. 254(2):157-178; Dale et al. (1996) Mol. Biol. 57:369-374; Smith (1985) Ann. Rev. Genet. 19:423-462; Botstein & Shortle (1985) Science 229:1193-1201; Carter (1986) Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) Proc. Natl. Acad. Sci. USA 82:488-492; Kunkel et al. (1987) Meth. Enzymol. 154, 367-382; and Bass et al. (1988) Science 242:240-245); oligonucleotide-directed mutagenesis (Methods in Enzymol. 100:468-500 (1983); Meth. Enzymol. 154:329-350 (1987); Zoller & Smith (1982) Nucl. Acids Res. 10:6487-6500; Zoller & Smith (1983) Meth. Enzymol. 100:468-500; and Zoller & Smith (1987) Meth. Enzymol. 154:329-350); phosphorothioate-modified DNA mutagenesis (Taylor et al. (1985) Nucl. Acids Res. 13:8749-8764; Taylor et al. (1985) Nucl. Acids Res. 13:8765-8787 (1985); Nakamaye & Eckstein (1986) Nucl. Acids Res. 14:9679-9698; Sayers et al. (1988) Nucl. Acids Res. 16:791-802; and Sayers et al. (1988) Nucl. Acids Res. 16:803-814); mutagenesis using gapped duplex DNA (Kramer et al. (1984) Nucl. Acids Res. 12:9441-9456; Kramer & Fritz (1987) Meth. Enzymol. 154:350-367; Kramer et al. (1988) Nucl. Acids Res. 16:7207; and Fritz et al. (1988) Nucl. Acids Res. 16:6987-6999).

Additional suitable diversity-generating methods include point mismatch repair (Kramer et al. (1984) Cell 38:879-887), mutagenesis using repair-deficient host strains (Carter et al. (1985) Nucl. Acids Res. 13:4431-4443; and Carter (1987) Meth. Enzymol. 154:382-403), deletion mutagenesis (Eghtedarzadeh & Henikoff (1986) Nucl. Acids Res. 14:5115), restriction-selection and restriction-purification (Wells et al. (1986) Phil. Trans. R. Soc. Lond. A. 317:415-423), mutagenesis by total gene synthesis (Nambiar et al. (1984) Science 223:1299-1301; Sakamar and Khorana (1988) Nucl. Acids Res. 14:6361-6372; Wells et al. (1985) Gene 34:315-323; and Grundström et al. (1985) Nucl. Acids Res. 13:3305-3316), double-strand break repair (Mandecki (1986) Proc. Natl. Acad. Sci. USA 83:7177-7181; and Arnold (1993) Curr. Opin. Biotechnol. 4:450-455). Additional details on many of the above methods can be found in Methods in Enzymology Volume 154, which also describes useful controls for trouble-shooting problems with various mutagenesis methods.

Additional site-mutagenesis techniques are described in, e.g., Edelman et al., DNA 2:183 (1983), Zoller et al., Nucl. Acids Res. 10:6487-5400 (1982), Veira et al., Meth. Enzymol. 153:3 (1987)). Other useful mutagenesis techniques include alanine scanning, or random mutagenesis, such as iterated random point mutagenesis induced by error-prone PCR, chemical mutagen exposure, or polynucleotide expression in mutator cells (see, e.g., Bornscheueret et al., Biotechnol. Bioeng. 58, 554-59 (1998), Cadwell and Joyce, PCR Methods Appl. 3(6):S136-40 (1994), Kunkel et al., Meth. Enzymol. 204:125-39 (1991), Low et al., J. Mol. Biol. 260:359-68 (1996), Taguchi et al., Appl. Environ. Microbiol. 64(2): 492-95 (1998), and Zhao et al., Nat. Biotech. 16:258-61 (1998) for discussion of such techniques). Suitable primers for PCR-based site-directed mutagenesis or related techniques can be prepared by methods described in Crea et al., Proc. Natl. Acad. Sci. USA 75:5765 (1978).

Other useful techniques for promoting sequence diversity include PCR mutagenesis techniques (as described in, e.g., Kirsch et al., Nucl. Acids Res. 26(7):1848-50 (1998), Seraphin et al., Nucl. Acids Res. 24(16):3276-7 (1996), Caldwell et al., PCR Methods U.S. Pat. No. 5,512,463), cassette mutagenesis techniques based on the methods described in Wells et al., Gene 34:315 (1985), phagemid display techniques (as described in, e.g., Soumillion et al., Appl. Biochem. Biotechnol. 47:175-89 (1994), O'Neil et al., Curr. Opin. Struct. Biol. 5(4):443-49 (1995), Dunn, Curr. Opin. Biotechnol. 7(5):547-53 (1996), and Koivunen et al., J. Nucl. Med. 40(5):883-88 (1999)), reverse translation evolution (as described in, e.g., U.S. Pat. No. 6,194,550), saturation mutagenesis described in, e.g., U.S. Pat. No. 6,171,820), PCR-based synthesis shuffling (as described in, e.g., U.S. Pat. No. 5,965,408) and recursive ensemble mutagenesis (REM) (as described in, e.g., Arkin and Yourvan, Proc. Natl. Acad. Sci. USA 89:7811-15 (1992), and Delgrave et al., Protein Eng. 6(3):327-331 (1993)). Techniques for introducing diversity into a library of homologous sequences also are provided in U.S. Pat. Nos. 6,159,687 and 6,228,639.

Further details regarding various diversity generating methods can be found in the following U.S. patents, PCT publications and applications, and European publications: U.S. Pat. Nos. 5,605,793, 5,811,238, 5,830,721, 5,834,252, 5,837,458, and Int'l Pat. Appn. Publication Nos. WO 95/22625, WO 96/33207, WO 97/20078, WO 97/35966, WO 99/41402, WO 99/41383, WO 99/41368, WO 99/23107, WO 99/21979, WO 98/31837, WO 98/27230, WO 98/27230, WO 00/00632, WO 00/09679, WO 98/42832, WO 99/29902, WO 98/41653, WO 98/41622, WO 98/42727, WO 00/18906, WO 00/04190, WO 00/42561, WO 00/42559, WO 00/42560, PCT/US00/26708, PCT/US01/06775, and European Pat. Appn. Nos. EP 752008, EP 0932670.

Several different general classes of sequence modification methods, such as mutation, recombination, etc. are applicable to the present invention and set forth, e.g., in the references above and below. That is, nucleic acids encoding polypeptides having the desired activities or properties (e.g., such as an ability to enhance an immune response against a mammalian EPCAM) can be diversified by any of the methods described herein, e.g., including various mutation and recombination methods, individually or in combination, to generate nucleic acids with a desired activity or property, including, e.g., those described herein. The following exemplify some of the different types of formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 and PCT/US99/15972.

Synthetic recombination methods can also be used in which oligonucleotides corresponding to targets of interest (e.g., EpCAM antigens) are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., WO 00/42561; PCT/US00/26708; WO 00/42560; and WO 00/42559.

In silico methods of recombination can be effected in which genetic algorithms are used in a computer to recombine sequence strings that correspond to homologous (or even non-homologous) nucleic acids. The resulting recombined sequence strings are optionally converted into nucleic acids by synthesis of nucleic acids that correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 and WO 00/42559. Extensive details regarding in silico recombination methods are found in these applications. This methodology is generally applicable to the nucleic acid sequences and polypeptide sequences of the invention.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate library-enriched sequences, which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see, e.g., U.S. Pat. No. 5,965,408 and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., comprising partially extended chains, are then denatured and re-annealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombination procedure known as “incremental truncation for the creation of hybrid enzymes” (“ITCHY”) described in Ostermeier et al. (1999) Nature Biotech 17:1205. This approach can be used to generate an initial a library of variants, which can optionally serve as a substrate for one or more in vitro or in vivo recombination methods. See also Ostermeier et al. (1999) Proc. Natl. Acad. Sci. USA 96:3562-67; Ostermeier et al. (1999), Biological and Medicinal Chemistry 7:2139-44.

Mutational methods that result in the alteration of individual nucleotides or groups of contiguous or non-contiguous nucleotides can be favorably employed to introduce nucleotide diversity. Many mutagenesis methods are found in the above-cited references; additional details regarding mutagenesis methods can be found in following, which can also be applied to the present invention. For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can include, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above. Alternatively, in vivo recombination techniques can be used. For example, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above. Other techniques that can be used for in vivo recombination and sequence diversification are described in U.S. Pat. No. 5,756,316.

Methods for generating multispecies expression libraries have been described (in addition to the reference noted above, see, e.g., U.S. Pat. Nos. 5,783,431 and 5,824,485 and their use to identify protein activities of interest has been proposed. In addition to the references noted above, see U.S. Pat. No. 5,958,672. Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

Nucleotide sequences of the present invention can be engineered by standard techniques to make additional modifications, such as, the insertion of new restriction sites, the alteration of glycosylation patterns, the alteration of PEGylation patterns, modification of the sequence based on host cell codon preference, the introduction of recombinase sites, and the introduction of splice sites.

In some applications, it is desirable to preselect or prescreen libraries (e.g., an amplified library, a cDNA library, a normalized library, etc.) or other substrate nucleic acids prior to diversification, e.g., by recombination-based mutagenesis procedures, or to otherwise bias the substrates towards nucleic acids that encode functional products. Libraries can also be biased towards nucleic acids that have specified characteristics, e.g., hybridization to a selected nucleic acid probe. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in U.S. Pat. No. 5,939,250. Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired NCSM activity as described herein using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides including proposed non-stochastic polynucleotide reassembly and site-saturation mutagenesis methods, are applicable to the present invention as well. Random or semi-random mutagenesis using doped or degenerate oligonucleotides is also described in, e.g., Arkin and Youvan (1992) Biotechnol. 10:297-300; Reidhaar-Olson et al. (1991) Meth. Enzymol. 208:564-86; Lim and Sauer (1991) J. Mol. Biol. 219:359-76; Breyer and Sauer (1989) J. Biol. Chem. 264:13355-60); and U.S. Pat. Nos. 5,830,650 and 5,798,208, and European Patent 0 527 809B1.

It will readily be appreciated that any of the above-described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

A recombinant nucleic acid produced by recombining one or more polynucleotide sequences of the invention with one or more additional nucleic acids using any of the above-described formats alone or in combination also forms a part of the invention. The one or more additional nucleic acids may include another polynucleotide of the invention; optionally, alternatively, or in addition, the one or more additional nucleic acids can include, e.g., a nucleic acid encoding a naturally-occurring mammalian EpCAM or antigenic fragment thereof (e.g., as found in GenBank or other available literature), or, e.g., any other homologous or non-homologous nucleic acid or fragments thereof (certain recombination formats noted above, notably those performed synthetically or in silico, do not require homology for recombination).

A recombinant nucleic acid produced by recombining one or more polynucleotide sequences of the invention with one or more additional nucleic acids using any of the above-described formats alone or in combination forms a part of the invention.

Polynucleotides of the invention, including those produced by the above-described recombination, mutagenesis, and standard nucleotide synthesis techniques described herein can be screened for any suitable characteristic, such as the expression of a recombinant polypeptide able to induce an immune response against a mammalian EpCAM or an antigenic fragment thereof. Polypeptides produced by such techniques and having such characteristics are an important feature of the invention. For example, the invention provides a recombinant polypeptide encoded by a recombinant polynucleotide produced by recursive sequence recombination with any nucleic acid sequence of the invention that induces an immune response against mEpCAM or an antigenic fragment thereof.

Modified Coding Sequences

Where appropriate, nucleic acids of the invention can be modified to increase or enhance expression in a particular host by modification of the sequence with respect to codon usage and/or codon context, given the particular host(s) in which expression of the nucleic acid is desired. Codons that are utilized most often in a particular host are called optimal codons, and those not utilized very often are classified as rare or low-usage codons (see, e.g., Zhang, S. P. et al. (1991) Gene 105:61-72). Codons can be substituted to reflect the preferred codon usage of the host, a process called “codon optimization” or “controlling for species codon bias.”

Optimized coding sequence comprising codons preferred by a particular prokaryotic or eukaryotic host can be used to increase the rate of translation or to produce recombinant RNA transcripts having desirable properties, such as a longer half-life, as compared with transcripts produced from a non-optimized sequence. Techniques for producing codon-optimized sequences are known (see, e.g., Murray, E. et al. (1989) Nucl. Acids Res. 17:477-508). Translation stop codons can also be modified to reflect host preference. For example, preferred stop codons for S. cerevisiae and mammals are UAA and UGA respectively. The preferred stop codon for monocotyledonous plants is UGA, whereas insects and E. coli prefer to use UAA as the stop codon (see, e.g., Dalphin, M. E. et al. (1996). Nucl. Acids Res. 24:216-218, for discussion). The arrangement of codons in context to other codons also can influence biological properties of a nucleic acid sequences, and modifications of nucleic acids to provide a codon context arrangement common for a particular host also is contemplated by the inventors. Thus, a nucleic acid sequence of the invention can comprise a codon optimized nucleotide sequence, i.e., codon frequency optimized and/or codon pair (i.e., codon context) optimized for a particular species (e.g., the polypeptide can be expressed from a polynucleotide sequence optimized for expression in humans by replacement of “rare” human codons based on codon frequency, or codon context, such as by using techniques such as those described in Buckingham et al. (1994) Biochimie 76(5):351-54 and U.S. Pat. Nos. 5,082,767, 5,786,464, and 6,114,148). For example, the invention provides a nucleic acid comprising a nucleotide sequence variant of SEQ ID NO: 19, wherein the nucleotide sequence variant differs from SEQ ID NO: 19 by the substitution of “rare” codons for a particular host with codons commonly expressed in the host, which codons encode the same amino acid residue as the substituted “rare” codons in SEQ ID NO:19.

Vectors Vector Components, and Expression Systems

The present invention also includes recombinant constructs comprising one or more of the nucleic acid sequences as broadly described above. The constructs comprise a nucleic acid vector or other vector, such as, e.g., a plasmid, a cosmid, a phage, a virus, a virus-like particle, a bacterial artificial chromosome (BAC), a yeast artificial chromosome (YAC), and the like, into which at least one nucleic acid sequence of the invention (e.g., one which encodes a polypeptide of the invention) has been inserted, in a forward or reverse orientation. Some such non-nucleic acid vectors comprise at least one polypeptide of the invention.

In one aspect, such construct further comprises regulatory sequences, including, for example, a promoter, operably linked to the nucleic acid sequence of the invention. Large numbers of suitable vectors and promoters are known to those of skill in the art, and are commercially available. In one aspect, the nucleic acid vector is an expression vector that comprises at least one nucleic acid sequence of the invention and/or which encodes on expression at least one polypeptide of the invention.

General texts that describe molecular biological techniques useful herein, including the use of vectors, promoters and many other relevant topics, include Berger, supra; Sambrook (1989), supra, and Ausubel, supra. Examples of techniques sufficient to direct persons of skill through in vitro amplification methods, including the polymerase chain reaction (PCR) the ligase chain reaction (LCR), Q∃-replicase amplification and other RNA polymerase mediated techniques (e.g., NASBA), e.g., for the production of the homologous nucleic acids of the invention are found in Berger, Sambrook, and Ausubel, all supra, as well as Mullis et al. (1987) U.S. Pat. No. 4,683,202; PCR Protocols: A Guide to Methods and Applications (Innis et al., eds.) Academic Press Inc. San Diego, Calif. (1990) (“Innis”); Arnheim &. Levinson (Oct. 1, 1990) C&EN 36-47; The Journal Of NIH Research (1991) 3:81-94; (Kwoh et al. (1989) Proc Natl Acad Sci USA 86:1173-1177; Guatelli et al. (1990) Proc Natl Acad Sci USA 87:1874-1878; Lomeli et al. (1989) J Clin Chem 35:1826-1831; Landegren et al. (1988) Science 241:1077-1080; Van Brunt (1990) Biotechnology 8:291-294; Wu and Wallace (1989) Gene 4:560-569; Barringer et al. (1990) Gene 89:117-122, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kilobases (kb) are generated. One of skill will appreciate that essentially any RNA can be converted into a double stranded DNA suitable for restriction digestion, PCR expansion and sequencing using reverse transcriptase and a polymerase. See Ausubel, Sambrook and Berger, all supra.

The present invention also provides host cells that are transduced with vectors of the invention, and the production of polypeptides of the invention by recombinant techniques. Host cells are genetically engineered (e.g., transduced, transformed or transfected) with the vectors of this invention, which may be, for example, a cloning vector or an expression vector. The engineered host cells can be cultured in conventional nutrient media modified as appropriate for activating promoters, selecting transformants, or amplifying the NCSM gene. The culture conditions, such as temperature, pH, and the like, are those previously used with the host cell selected for expression, and will be apparent to those skilled in the art and in the references cited herein, including, e.g., Freshney (1994) Culture of Animal Cells, a Manual of Basic Technique, 3rd ed., Wiley-Liss, New York and the references cited therein.

The polypeptides of the invention can also be produced in non-animal cells such as plants, yeast, fungi, bacteria and the like. In addition to Sambrook, Berger and Ausubel, details regarding cell culture are found in, e.g., Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg N.Y.); Atlas & Parks (eds.) The Handbook of Microbiological Media (1993) CRC Press, Boca Raton, Fla.

The polynucleotides of the present invention and fragments and variants thereof may be included in any one of a variety of expression vectors for expressing a polypeptide. Such vectors include chromosomal, nonchromosomal and synthetic DNA sequences, e.g., derivatives of SV40, bacterial plasmids, phage DNA, baculovirus, yeast plasmids, vectors derived from combinations of plasmids and phage DNA, viral DNA such as vaccinia, adenovirus, pox virus, fowl pox virus, pseudorabies, adeno-associated virus, retroviruses and many others. Any vector that transduces genetic material into a cell, and, if replication is desired, which is replicable and viable in the relevant host can be used.

The nucleic acid sequence in the expression vector is operatively linked to an appropriate transcription control sequence (promoter) to direct mRNA synthesis. Examples of such promoters include: LTR or SV40 promoter, E. coli lac or trp promoter, phage lambda P_{L} promoter, CMV promoter, and other promoters known to control expression of genes in prokaryotic or eukaryotic cells or their viruses. The expression vector also contains a ribosome binding site for translation initiation, and a transcription terminator. The vector optionally includes appropriate sequences for amplifying expression, e.g., an enhancer. In addition, the expression vectors optionally comprise one or more selectable marker genes to provide a phenotypic trait for selection of transformed host cells, such as dihydrofolate reductase or neomycin resistance for eukaryotic cell culture, or such as tetracycline or ampicillin resistance in E. coli.

The vector containing the appropriate DNA sequence encoding a polypeptide of the invention, as well as an appropriate promoter or control sequence, may be employed to transform an appropriate host to permit the host to express the protein. Examples of appropriate expression hosts include: bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; mammalian cells such as CHO, COS, BHK, HEK 293 or Bowes melanoma; plant cells, etc. It is understood that not all cells or cell lines need to be capable of producing fully functional NCSM polypeptides or fragments thereof; for example, antigenic fragments of NCSM polypeptide may be produced in a bacterial or other expression system. The invention is not limited by the host cells employed.

In bacterial systems, a number of expression vectors may be selected depending upon the use intended for the desired polypeptide or fragment thereof. For example, when large quantities of a particular polypeptide or fragments thereof are needed for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which nucleotide coding sequence may be ligated into the vector in-frame with sequences for the amino-terminal Met and the subsequent 7 residues of beta-galactosidase so that a hybrid protein is produced; pIN vectors (Van Heeke & Schuster (1989) J. Biol. Chem. 264:5503-5509); pET vectors (Novagen, Madison Wis.); and the like.

Similarly, in the yeast Saccharomyces cerevisiae a number of vectors containing constitutive or inducible promoters such as alpha factor, alcohol oxidase and PGH may be used for production of the poly eptides of the invention. For reviews, see Ausubel, supra, Berger, supra, and Grant et al. (1987) Meth. Enzymol. 153:516-544.

In mammalian host cells, a number of expression systems, such as viral-based systems, may be utilized. In cases where an adenovirus is used as an expression vector, a coding sequence is optionally ligated into an adenovirus transcription/translation complex consisting of the late promoter and tripartite leader sequence. Insertion in a nonessential E1 or E3 region of the viral genome results in a viable virus capable of expressing a polypeptide of interest in infected host cells (Logan and Shenk (1984) Proc. Natl. Acad. Sci. USA 81:3655-3659). In addition, transcription enhancers, such as the rous sarcoma virus (RSV) enhancer, are used to increase expression in mammalian host cells.

The skilled artisan will recognize that introduction of a start codon to the 5′ end of a particular nucleotide sequence (e.g., a fragment of the nucleotide sequence of SEQ ID NO: 19, which fragment encodes an immunogenic amino acid sequence) usually results in the addition of an N-terminal methionine to the encoded amino acid sequence when the sequence is expressed in a mammalian cell (other modifications may occur in bacterial and/or other eukaryotic cells, such as introduction of an formyl-methionine residue at a start codon). The inventors contemplate the production and use of such N-terminal methionine variants of any amino acid sequence of the invention (e.g., one of the immunogenic fragments of the sequence of SEQ ID NO:4 described elsewhere herein).

In another aspect, the invention provides a DNA that comprises at least one expression control sequence associated with and/or typically operably linked to a nucleic acid sequence of the invention. An “expression control sequence” is any nucleic acid sequence that promotes, enhances, or controls expression (typically and preferably transcription) of another nucleic acid sequence. Suitable expression control sequences include constitutive promoters, inducible promoters, repressible promoters, and enhancers.

Promoters exert a particularly important impact on the level of recombinant polypeptide expression. The nucleic acid of the invention (e.g., recombinant DNA nucleic acid) can comprise any suitable promoter. Examples of suitable promoters include the cytomegalovirus (CMV) promoter, the HIV long terminal repeat promoter, the phosphoglycerate kinase (PGK) promoter, Rous sarcoma virus (RSV) promoters, such as RSV long terminal repeat (LTR) promoters, mouse mammary tumor virus (MMTV) promoters, HSV promoters, such as the Lap2 promoter or the herpes thymidine kinase promoter (as described in, e.g., Wagner et al. (1981) Proc. Natl. Acad. Sci. 78:144-145), promoters derived from SV40 or Epstein Barr virus, adeno-associated viral (AAV) promoters, such as the p5 promoter, metallothionein promoters (e.g., the sheep metallothionein promoter or the mouse metallothionein promoter (see, e.g., Palmiter et al. (1983) Science 222:809-814), the human ubiquitin C promoter, E. coli promoters, such as the lac and trp promoters, phage lambda P_{L} promoter, and other promoters known to control expression of genes in prokaryotic or eukaryotic cells (either directly in the cell or in viruses which infect the cell). Promoters that exhibit strong constitutive baseline expression in mammals, particularly humans, such as cytomegalovirus (CMV) promoters, such as the CMV immediate-early promoter (described in, for example, U.S. Pat. No. 5,168,062), and promoters having substantial sequence identity with such a promoter, are particularly preferred. Also preferred are recombinant promoters having novel or enhanced properties, such as those described in International Patent Application WO 02/00897 (which novel promoters can be referred to as “CMV promoter variants” or “shuffled CMV promoter variants”).

The promoter can have any suitable mechanism of action. Thus, the promoter can be, for example, an “inducible” promoter, (e.g., a growth hormone promoter, metallothionein promoter, heat shock protein promoter, E1B promoter, hypoxia induced promoter, radiation inducible promoter, or adenoviral MLP promoter and tripartite leader), an inducible-repressible promoter, a developmental stage-related promoter (e.g., a globin gene promoter), or a tissue specific promoter (e.g., a smooth muscle cell α-actin promoter, myosin light-chain 1A promoter, or vascular endothelial cadherin promoter). Suitable inducible promoters include ecdysone and ecdysone-analog-inducible promoters (ecdysone-analog-inducible promoters are commercially available through Stratagene (La Jolla Calif.)). Other suitable commercially available inducible promoter systems include the inducible Tet-Off or Tet-on systems (Clontech, Palo Alto, Calif.). The inducible promoter can be any promoter that is up-and/or downregulated in response to an appropriate signal. Additional inducible promoters include arabinose-inducible promoters, a steroid-inducible promoters (e.g., a giucocorticoid-inducible promoters), as well as pH, stress, and heat-inducible promoters.

The promoter can be, and often is, a host-native promoter, or a promoter derived from a virus that infects a particular host (e.g., a human beta actin promoter, human EF1α promoter, or a promoter derived from a human AAV operably linked to the nucleic acid can be preferred), particularly where strict avoidance of gene expression silencing due to host immunological reactions to sequences that are not regularly present in the host is of concern. The polynucleotide also or alternatively can include a bidirectional promoter system (as described in, e.g., U.S. Pat. No. 5,017,478) linked to multiple nucleotide sequences of interest (e.g., a sequence encoding the polypeptide sequence of SEQ ID NO:5 or an amino acid sequence variant thereof and a second sequence encoding EpCAM).

The nucleic acid also can be operably linked to a modified or chimeric promoter sequence. The promoter sequence is “chimeric” in that it comprises at least two nucleic acid sequence portions obtained from, derived from, or based upon at least two different sources (e.g., two different regions of an organism's genome, two different organisms, or an organism combined with a synthetic sequence). Suitable promoters also include recombinant, mutated, or recursively recombined (e.g., shuffled) promoters. Minimal promoter elements, consisting essentially of a particular TATA-associated sequence, can, for example, be used alone or in combination with additional promoter elements. TATA-less promoters also can be suitable in some contexts. The promoter and/or other expression control sequences can include one or more regulatory elements have been deleted, modified, or inactivated. Preferred promoters include the promoters described in Int'l Patent Application WO 02/00897, one or more of which can be incorporated into and/or used with nucleic acids and vectors of the invention. Other shuffled and/or recombinant promoters also can be usefully incorporated into and used in the nucleic acids and vectors of the invention, e.g., to facilitate polypeptide expression.

Other suitable promoters and principles related to the selection, use, and construction of suitable promoters are provided in, e.g., Werner (1999) Mamm Genome 10(2):168-75, Walther et al. (1996) J. Mol. Med. 74(7):379-92, Novina (1996) Trends Genet. 12(9):351-55, Hart (1996) Semin. Oncol. 23(1):154-58, Gralla (1996) Curr. Opin. Genet. Dev. 6(5):526-30, Fassler et al. (1996) Methods Enzymol 273:3-29, Ayoubi et al. (1996), 10(4) FASEB J 10(4):453-60, Goldsteine et al. (1995) Biotechnol. Annu. Rev. 1:105-28, Azizkhan et al. (1993) Crit. Rev. Eukaryot. Gene Expr. 3(4):229-54, Dynan (1989) Cell 58(1):1-4, Levine (1989) Cell 59(3):405-8, and Berk et al. (1986) Annu. Rev. Genet. 20:45-79, as well as U.S. Pat. No. 6,194,191. Other suitable promoters can be identified by use of the Eukaryotic Promoter Database (release 68) (presently available at http://www.epd.isb-sib.ch/) and other, similar, databases, such as the Transcription Regulatory Regions Database (TRRD) (version 4.1) (available at http://www.bionet.nsc.ru/trrd/) and the transcription factor database (TRANSFAC) (available at http://transfac.gbf.de/TRANSFAC/index.html).

As an alternative to a promoter, particularly in RNA vectors and constructs, the nucleic acid sequence and/or vector can comprise one or more internal ribosome entry sites (IRESs), IRES-encoding sequences, or RNA sequence enhancers (Kozak consensus sequence analogs), such as the tobacco mosaic virus omega prime sequence.

The invention also provides a polynucleotide (or vector) that also or alternatively comprises an upstream activator sequence (UAS), such as a Gal4 activator sequence (as described in, e.g., U.S. Pat. No. 6,133,028) or other suitable upstream regulatory sequence (as described in, e.g., U.S. Pat. No. 6,204,060).

A polynucleotide (or vector) of the invention can include any other expression control sequences (e.g., enhancers, translation termination sequences, initiation sequences, splicing control sequences, etc.). Typically, a nucleic acid of the invention includes a Kozak consensus sequence that is functional in a mammalian cell, which can be a naturally occurring or modified sequence such as the modified Kozak consensus sequences described in U.S. Pat. No. 6,107,477. The nucleic acid can include specific initiation signals that aid in efficient translation of a coding sequence and/or fragments contained in the expression vector. These signals can include, e.g., the ATG initiation codon and adjacent sequences. In cases where a coding sequence, its initiation codon and upstream sequences are inserted into the appropriate expression vector, no additional translational control signals may be needed. However, in cases where only a coding sequence (e.g., a mature protein coding sequence), or a portion thereof, is inserted, exogenous nucleic acid transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (see, e.g., Scharf et al. (1994) Results Probl. Cell. Differ. 20:125-62; and Bittner et al. (1987) Meth. Enzymol. 153:516-544 for discussion). Suitable enhancers include, for example, the rous sarcoma virus (RSV) enhancer and the RTE enhancers described in U.S. Pat. No. 6,225,082. Initiation signals including the ATG initiation codon and adjacent sequences are desirably incorporated in the polynucleotide. In cases where a polynucleotide sequence, its initiation codon and upstream sequences are inserted into the appropriate expression vector, no additional translational control signals may be needed. However, in cases where only a coding sequence (e.g., a mature protein coding sequence), or a portion thereof, is inserted, exogenous nucleic acid transcriptional control signals including the ATG initiation codon are to be provided. The initiation codon must be in the correct reading frame to ensure transcription of the entire insert. Exogenous transcriptional elements and initiation codons can be of various origins, both natural and synthetic. The efficiency of expression can be enhanced by the inclusion of enhancers appropriate to the cell system in use (see, e.g., Scharf et al. (1994) Results Probl. Cell. Differ. 20:125-62; and Bittner et al. (1987) Meth. Enzymol. 153:516-544).

The expression level of a nucleic acid of the invention (or a corresponding polypeptide for comparative purposes) can be assessed by any suitable technique. Examples of such techniques include Northern Blot analysis (discussed in, e.g., McMaster et al. (1997) Proc. Natl. Acad. Sci. USA 74:4835-38 (1977) and Sambrook, infra), reverse transcriptase-polymerase chain reaction (RT-PCR) (as described in, e.g., U.S. Pat. No. 5,601,820 and Zaheer et al. (1995) Neurochem. Res. 20:1457-63, and in situ hybridization techniques (as described in, e.g., U.S. Pat. Nos. 5,750,340 and 5,506,098). Quantification of proteins also can be accomplished by the Lowry assay and other classification protein quantification assays (see, e.g., Bradford (1976) Anal. Biochem. 72:248-254 and Lowry et al. (1951) J. Biol. Chem. 193:265). Western blot analysis of recombinant polypeptides of the invention obtained from the lysate of cells transfected with polynucleotides encoding such recombinant polypeptides is another suitable technique for assessing levels of recombinant polypeptide expression.

A nucleic acid of the invention (e.g., DNA) may also comprise a ribosome binding site for translation initiation and a transcription-terminating region. A suitable transcription-terminating region is, for example, a polyadenylation sequence that facilitates cleavage and polyadenylation of the RNA transcript produced from the DNA nucleic acid. Any suitable polyadenylation sequence can be used, including a synthetic optimized sequence, as well as the polyadenylation sequence of BGH (Bovine Growth Hormone), human growth hormone gene, polyoma virus, TK (Thymidine Kinase), EBV (Epstein Barr Virus), rabbit beta globin, and the papillomaviruses, including human papillomaviruses and BPV (Bovine Papilloma Virus). Suitable polyadenylation (polyA) sequences also include the SV40 (human Sarcoma Virus-40) polyadenylation sequence and the BGH polyA sequence, which is particularly preferred. Such polyA sequences are described in, e.g., Goodwin et al. (1998). Nucleic Acids Res 26(12):2891-8, Schek et al. (1992) Mol. Cell. Biol. 12(12):5386-93, and van den Hoff et al. (1993) Nucleic Acids Res. 21(21):4987-8. Additional principles related to selection of appropriate polyadenylation sequences are described in, e.g., Levitt et al. (1989) Genes Dev 1989 3(7):1019-1025, Jacob et al. (1990) Crit. Rev. Eukaryot. Gene Expr. 1(1):49-59, Chen et al. (1995) Nucleic Acids Res. 23(14):2614-2620, Moreira et al. (1995) EMBO J. 14(15):3809-3819, Carswell et al. (1989) Mol. Cell. Biol. 1989 9(10):4248-4258.

The polynucleotide can further comprise site-specific recombination sites, which can be used to modulate transcription of the polynucleotide, as described in, e.g., U.S. Pat. Nos. 4,959,317, 5,801,030 and 6,063,627, European Patent Application 0 987 326 and Int'l Patent Application Publ. No. WO 97/09439.

In one aspect, a nucleic acid of the invention comprises a T7 RNA polymerase promoter operably linked to the nucleic acid sequence, facilitating the synthesis of single stranded RNAs from the nucleic acid sequence. T7 and T7-derived sequences are known, as are expression systems using T7 (see, e.g., Tabor and Richardson (1986) Proc. Natl. Acad. Sci. USA 82:1074, Studier and Moffat (1986) J. Mol. Biol. 189:113, and Davanloo et al. (1964) Proc. Natl. Acad. Sci. USA 81:2035). In one aspect, for example, nucleic acids comprising a T7 RNA polymerase and a polynucleotide sequence encoding at least one recombinant polypeptide of the invention are provided.

The nucleic acids of the invention can be positioned in and/or administered to a host or host cell in the form of a suitable delivery vehicle (i.e., a vector). The vector can be any suitable vector, including chromosomal, non-chromosomal, and synthetic nucleic acid vectors (a nucleic acid sequence comprising any combination of the above described expression cassette elements and/or other transfection-facilitating and/or expression-promoting sequence elements). Examples of such vectors include viruses, bacterial plasmids, phages, cosmids, phagemids, derivatives of SV40, baculovirus, yeast plasmids, vectors derived from combinations of plasmids and phage DNA, and viral nucleic acid (RNA or DNA) vectors, polylysine, and bacterial cells.

In one aspect, the invention provides a naked DNA or RNA vector, including, for example, a linear expression element (as described in, e.g., Sykes and Johnston (1997) Nat Biotech 17:355-59), a compacted nucleic acid vector (as described in, e.g., U.S. Pat. No. 6,077,835 and/or Int'l Patent Appn WO 00/70087), a plasmid vector such as pBR322, pUC 19/18, or pUC 118/119, a “midge” minimal-sized nucleic acid vector (as described in, e.g., Schakowski et al. (2001) Mol. Ther. 3:793-800) or as a precipitated nucleic acid vector construct, such as a CaPO₄ precipitated construct (as described in, e.g., Int'l Patent Appn WO 00/46147, Benvenisty and Reshef (1986) Proc. Natl. Acad. Sci. USA 83:9551-55, Wigler et al. (1978), Cell 14:725, and Coraro and Pearson (1981) Somatic Cell Genetics 7:603), comprising a nucleic acid of the invention. For example, the invention provides a naked DNA plasmid comprising SEQ ID NO: 19 operably linked to a CMV promoter or CMV promoter variant and a suitable polyadenylation sequence. Naked nucleotide vectors and the usage thereof are known in the art (see, e.g., U.S. Pat. Nos. 5,589,466 and 5,973,972).

The vector typically is an expression vector that is suitable for expression in a bacterial system or other system (e.g., as opposed to a vector designed for replicating the nucleic acid sequence without expression, which can be referred to as a cloning vector). For example, in one aspect the invention provides a bacterial expression vector comprising a nucleic acid sequence of the invention. Suitable vectors include, for example, vectors which direct high level expression of fusion proteins that are readily purified (e.g., multifunctional E. coli cloning and expression vectors such as BLUESCRIPT (Stratagene), pIN vectors (Van Heeke & Schuster, J. Biol. Chem. 264:5503-5509 (1989); pET vectors (Novagen, Madison Wis.); and the like). While such bacterial expression vectors can be useful in expressing particular polypeptides of the invention, glycoproteins of the invention are preferably expressed in eukaryotic cells and, as such, the invention also provides eukaryotic expression vectors.

The expression vector also or alternatively can be a vector suitable for expression of the nucleic acid of the invention in a yeast cell. Any vector suitable for expression in a yeast system can be employed. Suitable vectors for use in, e.g., Saccharomyces cerevisiae include, for example, vectors comprising constitutive or inducible promoters such as alpha factor, alcohol oxidase and PGH (reviewed in: Ausubel, supra, Berger, supra, and Grant et al., Meth. Enzymol. 153:516-544 (1987)).

Usually the expression vector will be a vector suitable for expression of the nucleic acid in an animal cell, such as an insect cell (e.g., a SF-9 cell) or a mammalian cell (e.g., a CHO cell, 293 cell, HeLa cell, human fibroblast cell, or similar well-characterized cell). Suitable mammalian expression vectors are known in the art (see, e.g., Kaufman, Mol. Biotechnol. 16(2):151-160 (2000), Van Craenenbroeck, Eur. J. Biochem. 267(18):5665-5678 (2000), Makrides, Protein Expr. Purif. 17(2):183-202 (1999), and Yarranton, Curr. Opin. Biotechnol. 3(5):506-511 (1992)). Suitable insect cell plasmid expression vectors also are known (see, e.g., Braun, Biotechniques 26(6):1038-1040, 1042 (1999)).

An expression vector typically can be propagated in a host cell. The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, gene or vaccine gun, injection, or other common techniques (see, e.g., Davis et al., BASIC METHODS IN MOLECULAR BIOLOGY (1986) for a description of in vivo, ex vivo, and in vitro methods). Cells comprising these and other vectors of the invention form an important part of the invention.

The expression vector can also comprises nucleotides encoding a secretion/localization sequence, which targets polypeptide expression to a desired cellular compartment, membrane, or organelle, or which directs polypeptide secretion to the periplasmic space or into the cell culture media. Such sequences are known in the art, and include secretion leader or signal peptides, organelle targeting sequences (e.g., nuclear localization sequences, ER retention signals, mitochondrial transit sequences, chloroplast transit sequences), membrane localization/anchor sequences (e.g., stop transfer sequences, GPI anchor sequences), and the like.

In addition, the expression vectors of the invention optionally comprise one or more selectable marker genes to provide a phenotypic trait for selection of transformed host cells, such as dihydrofolate reductase resistance, neomycin resistance, G418 resistance, puromycin resistance, and/or blasticidin resistance for eukaryotic cell culture, or such as tetracycline or ampicillin resistance in E. coli.

Furthermore, a nucleic acid of the invention can comprise an origin of replication useful for propagation in a microorganism. The bacterial origin of replication (Ori) utilized is preferably one that does not adversely affect gene expression in mammalian cells. Examples of useful origin of replication sequences include the fl phage ori, RK2 oriV, pUC ori, and the pSC101 ori. Preferred original of replication sequences include the ColEI ori and the p15 (available from plasmid pACYC177, New England Biolab, Inc.), alternatively another low copy ori sequence (similar to p15) can be desirable in some contexts. The nucleic acid in this respect desirably acts as a shuttle vector, able to replicate and/or be expressed in both eukaryotic and prokaryotic hosts (e.g., a vector comprising an origin of replication sequences recognized in both eukaryotes and prokaryotes).

Additional nucleic acids provided by the invention include cosmids. Any suitable cosmid vector can be used to replicate, transfer, and express the nucleic acid sequence of the invention. Typically, a cosmid comprises a bacterial oriV, an antibiotic selection marker, a cloning site, and either one or two cos sites derived from bacteriophage lambda. The cosmid can be a shuttle cosmid or mammalian cosmid, comprising a SV40 oriV and, desirably, suitable mammalian selection marker(s). Cosmid vectors are further described in, e.g., Hohn et al. (1988) Biotechnology 10:113-27.

The present invention also includes recombinant constructs comprising one or more of the nucleic acids of the invention. The constructs comprise a vector, such as, a plasmid, a cosmid, a phage, a virus, a bacterial artificial chromosome (BAC), a yeast artificial chromosome (YAC), and the like, into which a nucleic acid sequence of the invention has been inserted, in a forward or reverse orientation.

In one aspect of the invention, delivery of a recombinant DNA sequence of the invention can be accomplished with a naked DNA plasmid or plasmid associated with one or more transfection-enhancing agents, as discussed further herein. The plasmid DNA vector can have any suitable combination of features. In some aspects, preferred plasmid DNA vectors comprise a strong promoter/enhancer region (e.g., human CMV, RSV, SV40, SL3-3, MMTV, or HIV LTR promoter), an effective poly(A) termination sequence, an origin of replication for plasmid product in E. coli, an antibiotic resistance gene as selectable marker, and a convenient cloning site (e.g., a polylinker). A particular plasmid vector for delivery of the nucleic acid of the invention in this respect is the vector pMaxVax 10.1, the construction and features of which are described in Example 3. Optionally, such a plasmid vector includes at least one immunostimulatory sequence (ISS) and/or at least one gene encoding a suitable cytokine adjuvant (e.g., a GM-CSF sequence, IL-2 sequence, or both), as further described elsewhere herein.

In another aspect, the invention provides a non-nucleic acid vector comprising at least one nucleic acid or polypeptide of the invention. Such a non-nucleic acid vector includes, e.g., a recombinant virus, a viral nucleic acid-protein conjugate (which, with recombinant viral particles, may sometimes be referred to as a viral vector), or a cell, such as recombinant (and usually attenuated) Salmonella, Listeria, and Bacillus Calmette-Guérin (BCG) bacterial cells. Thus, for example, the invention provides a viral vector comprising a nucleic acid of the sequence of the invention. Any suitable viral vector can be used in this respect, and several are known in the art. A viral vector can comprise any number of viral polynucleotides, alone (a viral nucleic acid vector) or, more commonly, in combination with one or more (typically two, three, or more) viral proteins, which facilitate delivery, replication, and/or expression of the nucleic acid of the invention in a desired host cell. The viral vector can be a polynucleotide comprising all or part of a viral genome, a viral protein/nucleic acid conjugate, a virus-like particle (VLP), a vector similar to those described in U.S. Pat. No. 5,849,586 and International Patent Application WO 97/04748, or an intact virus particle comprising viral nucleic acids and the nucleic acid of the invention. A viral particle viral vector (i.e., a recombinant virus) can comprise a wild-type viral particle or a modified viral particle, particular examples of which are discussed below.

The viral vector can be a vector that requires the presence of another vector or wild-type virus for replication and/or expression (i.e., a helper-dependent virus), such as an adenoviral vector amplicon. Typically, such viral vectors consist essentially of a wild-type viral particle, or a viral particle modified in its protein and/or nucleic acid content to increase transgene capacity or aid in transfection and/or expression of the nucleic acid (examples of such vectors include the herpes virus/AAV amplicons).

Preferably, though not necessarily, the viral vector particle is derived from, is based on, comprises, or consists of, a virus that normally infects animals, preferably vertebrates, such as mammals and, especially, humans. Suitable viral vector particles in this respect, include, for example, adenoviral vector particles (including any virus of or derived from a virus of the adenoviridae), adeno-associated viral vector particles (AAV vector particles) or other parvoviruses and parvoviral vector particles, papillomaviral vector particles, flaviviral vectors, picomaviral vectors, alphaviral vectors, herpes viral vectors, pox virus vectors, retroviral vectors, including lentiviral vectors. Examples of such viruses and viral vectors are provided in, e.g., Fields Virology, supra, Fields et al., eds., VIROLOGY, Raven Press, Ltd., New York (3rd ed., 1996 and 4th ed., 2001), ENCYCLOPEDIA OF VIROLOGY, R. G. Webster et al., eds., Academic Press (2nd ed., 1999), FUNDAMENTAL VIROLOGY, Fields et al., eds., Lippincott-Raven (3rd ed., 1995), Levine, “Viruses,” Scientific American Library No. 37 (1992), MEDICAL VIROLOGY, D. O. White et al., eds., Academic Press (2nd ed. 1994), and INTRODUCTION TO MODERN VIROLOGY, Dimock, N. J. et al., eds., Blackwell Scientific Publications, Ltd. (1994).

Viral vectors that can be employed with polynucleotides of the invention and the methods described herein include adeno-associated vectors, which are reviewed in, e.g., Carter (1992) Curr. Opinion Biotech. 3:533-539 (1992) and Muzcyzka (1992) Curr. Top. Microbiol. Immunol. 158:97-129 (1992). Additional types and aspects of AAV vectors are described in, e.g., Buschacher et al., Blood 5(8):2499-504, Carter, Contrib. Microbiol. 4:85-86 (2000), Smith-Arica, Curr. Cardiol. Rep. 3(1):41-49 (2001), Taj, J. Biomed. Sci. 7(4):279-91 (2000), Vigna et al., J. Gene Med. 2(5):308-16 (2000), Klimatcheva et al., Front. Biosci. 4:D481-96 (1999), Lever et al., Biochem. Soc. Trans. 27(6):841-47 (1999), Snyder, J. Gene Med. 1(3):166-75 (1999), Gerich et al., Knee Surg. Sports Traumatol. Arthrosc. 5(2):118-23 (1998), and During, Adv. Drug Deliv. Review 27(1):83-94 (1997), and U.S. Pat. Nos. 4,797,368, 5,139,941, 5,173,414, 5,614,404, 5,658,785, 5,858,775, and 5,994,136, as well as other references discussed elsewhere herein). Adeno-associated viral vectors can be constructed and/or purified using the methods set forth, for example, in U.S. Pat. No. 4,797,368 and Laughlin et al., Gene 23:65-73 (1983).

Another type of viral vector that can be employed with polynucleotides and methods of the invention is a papillomaviral vector. Suitable papillorriaviral vectors are known in the art and described in, e.g., Hewson (1999) Mol Med Today 5(1):8, Stephens (1987) Biochem J 248(1):1-1.1, and U.S. Pat. No. 5,719,054. Particularly preferred papillomaviral vectors are provided in, e.g., International Patent Application WO 99/21979.

Alphavirus vectors can be gene delivery vectors in other contexts. Alphavirus vectors are known in the art and described in, e.g., Carter (1992) Curr Opinion Biotech 3:533-539, Muzcyzka (1992) Curr. Top. Microbiol. Immunol. 158:97-129, Schlesinger Expert Opin. Biol. Ther. (2001) 1(2):177-91, Polo et al., Dev. Biol. (Basel). 2000;104:181-5, Wahlfors et al., Gene Ther. (2000) 7(6):472-80, Colombage et al., Virology. (1998) 250(1):151-63, and Int'l Patent Appn Publ. Nos. WO 01/81609, WO 00/39318, WO 01/81553, WO 95/07994, and WO 92/10578.

Another advantageous group of viral vectors are the herpes viral vectors. Examples of herpes viral vectors are described in, e.g., Lachmann et al., Curr. Opin. Mol. Ther. (1999) 1(5):622-32, Fraefel et al., Adv. Virus Res. (2000) 55:425-51, Huard et al., Neuromuscul. Disord. (1997);7(5):299-313, Glorioso et al., Annu. Rev. Microbiol. (1.995) 49:675-710, Latchman, Mol. Biotechnol. (1994) 2(2):179-95, and Frenkel et al., Gene Ther. (1994) Suppl 1:S40-6, as well as U.S. Pat. Nos. 6,261,552 and 5,599,691.

Retroviral vectors, including lentiviral vectors, also can be advantageous gene delivery vehicles in particular contexts. There are numerous retroviral vectors known in the art. Examples of retroviral vectors are described in, e.g., Miller, Curr Top Microbiol. Immunol. (1992)158:1-24; Salmons and Gunzburg (1993) Human. Gene Ther. 4:129-141; Miller et al. (1994) Meth. Enzymol. 217:581-599, Weber et al., Curr. Opin. Mol. Ther. (2001) 3(5):439-53, Hu et al., Pharmacol. Rev. (2000) 52(4):493-511, Kim et al., Adv. Virus Res. (2000) 55:545-63, Palu et al., Rev. Med. Virol. (2000) 10(3):185-202, and Takeuchi et al., Adv. Exp. Med. Biol. (2000) 465:23-35, as well as U.S. Pat. Nos. 6,326,195, 5,888,502, 5,580,766, and 5,672,510.

Baculovirus vectors are another advantageous group of viral vectors, particularly for the production of polypeptides of the invention. The production and use of baculovirus vectors is known (see, e.g., Kost, Curr. Opin. Biotechnol. 10(5):428-433 (1999) and Jones, Curr. Opin. Biotechnol. 7(5):512-516 (1996)). Where the vector is used for therapeutic uses (e.g., to induce an immune response against EpCAM-overexpressing cells) the vector will be selected such that it is able to adequately infect (or in the case of nucleic acid vectors transfect or transform) target cells in which the desired therapeutic effect is desired. For example, in methods wherein an immune response against micrometastatic cancer cells (e.g., breast cancer cells) that overexpress EpCAM is sought, a viral vector should be selected that can adequately infect cells in the vicinity of such cancerous cells (e.g., epithelial cells in nearby and/or associated tissues).

Adenoviral vectors also can be suitable viral vectors for gene transfer. Adenoviral vectors are well known in the art and described in, e.g., Graham et al. (1995) Mol. Biotechnol. 33(3):207-220, Stephenson (1998) Clin. Diagn. Virol. 10(2-3):187-94, Jacobs (1993) Clin Sci (Lond). 85(2):117-22, U.S. Pat. Nos. 5,922,576, 5,965,358 and 6,168,941 and International Patent Applications WO 98/22588, WO 98/56937, WO 99/15686, WO 99/54441, and WO 00/32754. Adenoviral vectors, herpes viral vectors, and Sindbis viral vectors, useful in the practice of the invention and suitable for organismal in vivo transduction and expression of nucleic acids of the invention, are generally described in, e.g., Jolly (1994) Cancer Gene Therapy 1:51-64, Latchman (1994) Molec. Biotechnol. 2:179-195, and Johanning et al. (1995) Nucl. Acids Res. 23:1495-1501.

Other suitable viral vectors for transduction and expression include pox viral vectors. Examples of such vectors are discussed in, e.g., Berencsi et al., J. Infect. Dis. (2001) 183(8):1171-9; Rosenwirth et al., Vaccine (2001)19(13-14):1661-70; Kittlesen et al., J. Immunol. (2000) 164(8):4204-11; Brown et al., Gene Ther. (2000).7(19):1680-9; Kanesa-thasan et al., Vaccine (2000) 19(4-5):483-91; Sten (2000) Drug 60(2):249-71. Vaccinia virus vectors (e.g., Modified Vaccinia Ankara (MVA) vectors and MVA-derived vectors) are particularly advantageous pox virus vectors in some contexts, as are fowl pox virus vectors, canary pox virus vectors, and other avipox virus vectors. Examples of such vaccinia virus vectors and uses thereof are provided in, e.g., Venugopal et al. (1994) Res. Vet. Sci. 57(2):188-193, Moss (1994) Dev. Biol. Stand. 82:55-63 (1994), Weisz et al. (1994) Mol. Cell. Biol. 43:137-159, Mahr and Payne (1992) Immunobiology 184(2-3):126-146, Hruby (1990) Clin. Microbiol. Rev. 3(2):153-170, and Int'l Patent Appn Publ. Nos. WO 92/07944, WO 98/13500, and WO 89/08716. Related canary pox, avipox, and fowl pox viruses also are known in the art (see, e.g., Ratliff et al., Acta Urol Belg. (1996) 64(2):85 and Paoletti, Proc. Natl. Acad. Sci. USA (1996) 93(21):11349-53).

In some aspects, it is preferred that the virus vector is replication-deficient in a host cell. AAV vectors, which are naturally replication-deficient in the absence of complementing adenoviruses or at least adenovirus gene products (provided by, e.g., a helper virus, plasmid, or complementation cell), are preferred in this respect. By “replication-deficient” is meant that the viral vector comprises a genome that lacks at least one replication-essential gene function. A deficiency in a gene, gene function, or gene or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient viral vector. The essential gene functions of the viral vector particle vary with the type of viral vector particle at issue. Examples of replication-deficient viral vector particles are described in, e.g., Marconi et al., Proc. Natl. Acad. Sci. USA 93(21):11319-20 (1996), Johnson and Friedmann, Methods Cell Biol. 43 (pt. A):211-30 (1994), Timiryasova et al., J. Gene Med. 3(5):468-77 (2001), Burton et al., Stem Cells 19(5):358-77 (2001), Kim et al., Virology 282(1)154-67 (2001), Jones et al., Virology 278(1):137-50 (2000), Gill et al., J. Med. Virol. 62(2):127-39 (2000), Chen and Engleman, J. Virol. 74(17):8188-93 (2000), Marconi et al., Gene Ther. 6(5):904-12 (1999), Krisky et al., Gene Ther. 5(11):1517-30 (1998), Bieniasz et al., Virology 235(1):65-72 (1997), Strayer et al., Biotechniques 22(3):447-50 (1997), Wyatt et al., Vaccine 14(15):1451-8 (1996), and Penciolelli et al., J. Virol. 61(2):579-83 (1987). Other replication-deficient vectors are based on simple MuLV vectors. See, e.g., Miller et al. (1990) Mol Cell Biol 10:4239 (1990); Kolberg (1992) J NIH Res 4:43, and Cometta et al. (1991) Hum Gene Ther 2:215). Canary pox vectors are advantageous in infecting human cells but being naturally incapable of replication therein (i.e., without genetic modification).

The basic construction of recombinant viral vectors is well understood in the art and involves using standard molecular biological techniques such as those described in, e.g., Sambrook et al., MOLECULAR CLONING: A LABORATORY MANUAL (Cold Spring Harbor Press 1989) and the third edition thereof (2001), Ausubel et al., CURRENT PROTOCOLS IN MOLECULAR BIOLOGY (Wiley Interscience Publishers 1995), and Watson et al., RECOMBINANT DNA, (2d ed.), and several of the other references mentioned herein. For example, adenoviral vectors can be constructed and/or purified using the methods set forth, for example, in Graham et al., Mol. Biotechnol. 33(3):207-220 (1995), U.S. Pat. No. 5,965,358, Donthine et al., Gene Ther. 7(20):1707-14 (2000), and other references described herein. Adeno-associated viral vectors can be constructed and/or purified using the methods set forth, for example, in U.S. Pat. No. 4,797,368 and Laughlin et al., Gene 23:65-73 (1983). Similar techniques are known in the art with respect to other viral vectors, particularly with respect to herpes viral vectors (see e.g., Lachman et al., Curr. Opin. Mol. Ther. 1(5):622-32 (1999)), lentiviral vectors, and other retroviral vectors. In general, the viral vector comprises an insertion of the nucleic acid (for example, a wild-type adenoviral vector can comprise an insertion of up to 3 KB without deletion), or, more typically, comprises one or more deletions of the virus genome to accommodate insertion of the nucleic acid and additional nucleic acids, as desired, and to prevent replication in host cells.

In one aspect, the viral vector desirably is a targeted viral vector, comprising a restricted or expanded tropism as compared to a wild-type viral particle of similar type. Targeting is typically accomplished by modification of capsid and/or envelope proteins of the virus particle. Examples of targeted virus vectors and related principles are described in, e.g., International Patent Applications WO 92/06180, WO 94/10323, WO 97/38723, and WO 01/28569, and WO 00/11201, Engelstadter et al., Gene Ther., 8(15), 1202-6 (2001), van Beusechem et al., Gene Ther. 7(22):1940-6 (2000), Boerger et al., Proc. Natl. Acad. Sci. USA 96(17):9867-72 (1999), Bartlett et al., Nat. Biotechnol. 17(2):181-6 (1999), Girod et al., Nat. Med. 5(9):1052-56 (as modified by the erratum in Nat. Med. 5(12):1438) (1999), J. Gene Med. (1999) 1(5):300-11, Karavanas et al., Crit. Rev. Oncoi. Hematol. (1998) 28(1):7-30, Wickham et al., J. Virol. 71(10):7663-9 (1997), Cripe et al., Cancer Res. 61(7):2953-60 (2001), van Deutekom et al., J. Gene Med. 1(6):393-9 (1999), McDonald et al., J. Gene Med. 1(2):103-10 (1999), Peng, Curr. Opin. Biotechnol. (1999) 10(5):454-7, Staba et al., Cancer Gene Ther. 7(1):13-9 (2000), Kibbe et al., Arch. Surg. 135(2):191-7 (2000), Harari et al., Gene Ther. 6(5):801-7 (2000), and Bouri et al., Hum Gene Ther. 10(10):1633-40 (1999), and Laquerre et al., J. Virbl. 72(12):9683-97 (1997), Buchholz, Curr. Opin. Mol. Ther. (1999) 1(5):613-21, U.S. Pat. Nos. 6,261,554, 5,962,274,5,695,991, and 6,251,654, and European Patent Applications 1 002 119 and 1 038 967. Particular targeted vectors and techniques for producing such vectors are provided in International Patent Application WO 99/23107.

Viral vectors comprising a nucleic acid of the invention and that target cancer cells (i.e., selectively infect cancer cells) are an important feature of the invention. Several types of the above-described virus particles can be targeted by modification of surface (membrane and/or capsid proteins), including recombinant adenoviruses, Newcastle disease viruses, and herpes viruses. Techniques for preparing viral vectors that target cancer cells are known (see, e.g., Galanis et al., Crit. Rev. Oncol. Hematol. 38(3):177-92 (2001)). Non-viral vectors (e.g., naked nucleic acid vectors), targeted to cancer cells (e.g., by folate targeting) also are useful delivery systems in therapeutic method of the invention (see, e.g., Ward, Curr Opin. Mol. Ther. 2(2):182-187 (2000) for a description of such vectors). Other DNA-protein conjugates that adequately target cancer cells also can be used (see, e.g., Cristiano, Front Biosci (1998) 3:D1161-70).

A viral vector particle comprising a nucleic acid can be a chimeric viral vector particle (i.e., a virus encoded by the combination of two or more viral genomes). Examples of chimeric viral vector particles are described in, e.g., Reynolds et al., Mol. Med. Today 5(1):25-31 (1999), Boursnell et al., Gene 13:311-317 (1991), Dobbe et al., Virology 288(2): 283-94 (2001), Grene et al., AIDS Res. Human. Retroviruses 13(1), 41-51 (1997), Reimann et al., J. Virol. 70(10):6922-8 (1996), Li et al., J. Virol. 67(11):6659-66 (1993), Dong et al., J. Virol. 66(12):7374-82 (1992), Wahlfors, Hum. Gene Ther. (1999) 10(7):1197-206, Reynolds et al., Mol. Med. Today 5(1):25-31 (1999), Boursnell et al., Gene 13:311-317 (1991) and U.S. Pat. Nos. 5,877,011, 6,183,753, 6,146,643, and 6,025,341.

As indicated above, non-viral vectors of the invention also can be associated with molecules that target the vector to a particular region in the host (e.g., a particular organ, tissue, and/or cell type). For example, a nucleotide can be conjugated to a targeting protein, such as a viral protein that binds a receptor or a protein that binds a receptor of a particular target (e.g., by a modification of the techniques provided in Wu and Wu, J. Biol. Chem. 263(29):14621-24 (1988)). Targeted cationic lipid compositions also are known in the art (see, e.g., U.S. Pat. No. 6,120,799). Other techniques for targeting genetic constructs are provided in International Patent Application WO 99/41402.

One aspect of the invention relates to host cells containing any of the above-described nucleic acids, vectors, or other constructs of the invention. Cells provided by the invention can be described as “recombinant” cells, in that they comprise, express, and/or are modified by transformation, transfection, and/or infection with at least one nucleic acid, vector, antibody, and/or nucleotide sequence of the invention.

The host cell can be a eukaryotic cell, such as a mammalian cell, a yeast cell, or a plant cell, or the host cell can be a prokaryotic cell, such as a bacterial cell. Introduction of the construct into the host cell can be effected by calcium phosphate transfection, DEAE-Dextran mediated transfection, electroporation, gene or vaccine gun, injection, or other common techniques (see, e.g., Davis, L., Dibner, M., and Battey, I. (1986) BASIC METHODS IN MOLECULAR BIOLOGY).

A host cell strain is optionally chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing that cleaves a “pre” or a “prepro” form of the protein may also be important for correct insertion, folding and/or function of the polypeptide, as discussed above, which in the case of many of the immunogenic amino acid sequences of the invention can be cell type-dependent. Different host cells such as E. coli, Bacillus sp., yeast, or mammalian cells, such as CHO, HeLa, BHK, MDCK, HEK 293, WI38, etc. have specific cellular machinery and characteristic mechanisms for such post-translational activities and may be chosen to ensure the correct modification and processing of the introduced foreign protein.

A nucleic acid of the invention can be inserted into an appropriate host cell (in culture or in a host organism) to permit the host to express the protein. Any suitable host cell can be used transformed/transduced by the nucleic acids of the invention. Examples of appropriate expression hosts include: bacterial cells, such as E. coli, Streptomyces, Bacillus sp., and Salmonella typhimurium; fungal cells, such as Saccharomyces cerevisiae, Pichia pastoris, and Neurospora crassa; insect cells such as Drosophila and Spodoptera frugiperda; mammalian cells such as Vero cells, HeLa cells, CHO cells, COS cells, W138 cells, NIH-3T3 cells (and other fibroblast cells, such as MRC-5 cells), MDCK cells, KB cells, SW-13 cells, MCF7 cells, BHK cells, HEK-293 cells, Bowes melanoma cells, and plant cells, etc. For example, a nucleic acid of the invention can be transformed into dicot plant cells by way of a Ti or Ri plasmid in a suitable bacterial vector (e.g., an Agrobacterium tumefaciens bacterial vector), which cells can be in a live plant, an explant, suitable protoplast cells, or other appropriate plant culture. Dicot cells are typically transformed by PEG and/or CaPO₄-mediate transfection and other known techniques (see generally Potrykus, Ciba Found Symp. 154:198-212 (1990)). Techniques for ensuring appropriate glycosylation have been developed with mammalian antibodies (i.e., so-called “plantbodies,” which can generally be applied to polypeptides and antibodies of the invention (with the recognition that some minor differences in glycosylation, such as fructose linkages, will be present in such polypeptides) (see, e.g., Ma et al., Nature Med. 4:601-606 (1998), Cabanes-Macheteau et al. (1999) Glycobiology. 9(4):365-72, Chargelegue et al. (2000), Transgenic Res. 9:187-94, and Khoudi et al. (1999) Biotechnology Bioeng. 64:135-43). It is understood that not all cells or cell lines need to be capable of producing fully functional polypeptides or fragments thereof; for example, antigenic fragments of the polypeptide may be produced in a bacterial or other non-glycosylating and/or non-proteolytic cleaving expression system. Additional examples of suitable host cells are described, for example, in U.S. Pat. No. 5,994,106 and International Patent Application WO 95/34671.

The present invention also provides host cells that are transduced, transformed or transfected with at least one nucleic acid or vector of the invention. As discussed above, a vector of the invention typically comprises a nucleic acid of the invention. Host cells are genetically engineered (e.g., transduced, transformed, infected, or transfected) with the vectors of the invention, which may be, for example, a cloning vector or an expression vector. The vector may be, for example, in the form of a plasmid, a viral particle, a phage, attenuated bacteria, or any other suitable type of vector. Host cells suitable for transduction and/or infection with viral vectors of the invention for production of the recombinant polypeptides of the invention and/or for replication of the viral vector of the invention include the above-described cells.

Examples of cells that have been demonstrated as suitable for packaging of viral vector particles are described in, e.g., Inoue et al., J. Virol. 72(9):7024-31 (1998), Polo et al., Proc. Natl. Acad. Sci. 96(8):4598-603 (1999), Farson et al., J. Gene Med. 1(3):195-209 (1999), Sheridan et al., Mol. Ther. 2(3):262-75 (2000), Chen et al., Gene Ther. 8(9):697-703 (2001), and Pizzaro et al., Gene Ther. 8(10):737-745 (2001). For replication-deficient viral vectors, such as AAV vectors, complementing cell lines, or cell lines transformed with helper viruses, or cell lines transformed with plasmids encoding essential genes, are necessary for replication of the viral vector.

The engineered host cells can be cultured in conventional nutrient media modified as appropriate for activating promoters, selecting transformants, or amplifying the gene of interest. The culture conditions, such as temperature, pH, and the like, are those previously used with the host cell selected for expression, and will be apparent to those skilled in the art and in the references cited herein, including, e.g., ANIMAL CELL TECHNOLOGY, Rhiel et al., eds., (Kluwer Academic Publishers 1999), Chaubard et al., Genetic Eng. News 20(18) (2000), Hu et al., ASM News 59:65-68 (1993), Hu et al., Biotechnol. Prog. 1:209-215 (1985), Martin et al., Biotechnol. (1987), Freshney, CULTURE OF ANIMAL CELLS: A MANUAL OF BASIC TECHNIQUE, 4th ed., (Wiley, 0.2000), Mather, INTRODUCTION TO CELL AND TISSUE CULTURE: THEORY AND TECHNIQUE, (Plenum Press, 1998), Freshney, CULTURE OF IMMORTALIZED CELLS, 3rd ed., (John Wiley & Sons, 1996), CELL CULTURE: ESSENTIAL TECHNIQUES, Doyle et al., eds. (John Wiley & Sons 1998), and GENERAL TECHNIQUES OF CELL CULTURE, Harrison et al., eds., (Cambridge Univ. Press 1997). The nucleic acid also can be contained, replicated, and/or expressed in plant cells. Techniques related to the culture of plant cells are described in, e.g., Payne et al. (1992) PLANT CELL AND TISSUE CULTURE IN LIQUID SYSTEMS John Wiley & Sons, Inc. New York, N.Y.; Gamborg and Phillips (eds.) (1995) PLANT CELL, TISSUE AND ORGAN CULTURE: FUNDAMENTAL METHODS SPRINGER LAB MANUAL, Springer-Verlag (Berlin Heidelber N.Y.) and Plant Molecular Biology (1993) R. R. D. Croy (ed.) Bios Scientific Publishers, Oxford, U.K. ISBN 0 12 198370 6. Cell culture media in general are set forth in Atlas and Parks (eds.) THE HANDBOOK OF MICROBIOLOGICAL MEDIA (1993) CRC Press, Boca Raton, Fla.

For long-term, high-yield production of recombinant proteins, stable expression systems can be used. For example, cell lines that stably express a polypeptide of the invention can be transduced with expression vectors comprising viral origins of replication and/or endogenous expression elements and a selectable marker gene. Following the introduction of the vector, cells in the cell line may be allowed to grow for 1-2 days in an enriched media before they are switched to selective media. The purpose of the selectable marker is to confer resistance to selection, and its presence allows growth and recovery of cells that successfully express the introduced sequences. For example, resistant clumps of stably transformed cells can be proliferated using tissue culture techniques appropriate to the cell type.

Host cells transformed with an expression vector and/or polynucleotide are optionally cultured under conditions suitable for the expression and recovery of the encoded protein from cell culture. The polypeptide or fragment thereof produced by such a recombinant cell may be secreted, membrane-bound, or contained intracellularly, depending on the sequence and/or the vector used. Expression vectors comprising polynucleotides encoding mature polypeptides of the invention can be designed with signal sequences that direct secretion of the mature polypeptides through a prokaryotic or eukaryotic cell membrane. Principles related to such signal sequences are discussed elsewhere herein.

Cell-free transcription/translation systems can also be employed to produce recombinant polypeptides of the invention or fragments thereof using DNAs and/or RNAs of the present invention or fragments thereof. Several such systems are commercially available. A general guide to in vitro transcription and translation protocols is found in Tymms (1995) IN VITRO TRANSCRIPTION AND TRANSLATION PROTOCOLS: METHODS IN MOLECULAR BIOLOGY, Volume 37, Garland Publishing, NY.

Additional Aspects

The invention further provides a nucleic acid comprising a first nucleotide sequence encoding at least one polypeptide of the invention and a second nucleotide sequence that is an immunostimulatory sequence, e.g., a sequence according to the sequence pattern N₁CGN₂)ₓ, wherein N₁ is, 5′ to 3′, any two purines, any purine and a guanine, or any three nucleotides; N₂ is, 5′ to 3′, any two purines, any guanine and any purine, or any three nucleotides; and x is any number greater than 0. Immunomodulatory sequences are known in the art, and described in, e.g., Wagner et al. (2000) Springer Semin Immunopathol 22(1-2):147-52, Van Uden et al. (2000) Springer Semin Immunopathol 22(1-2): 1-9, and Pisetsky (1999) Immunol Res 19(1):35-46, as well as U.S. Pat. Nos. 6,207,646, 6,194,388, 6,008,200, 6,239,116, and 6,218,371. Other immunostimulating unmethylated CpG motifs in immunostimulatory sequences are known, and it is recognized that particular motifs are effective in particular host and/or host cells.

In another aspect, the invention provides a nucleic acid that comprises a first polynucleotide sequence that encodes at least one recombinant polypeptide of the invention and further comprises a second polynucleotide sequence that encodes at least one protein adjuvant. Such nucleic acid may be an expression vector. Alternatively, the invention provides two nucleic acids that are administered separately, with the first nucleic acid comprising a polynucleotide sequence that encodes at least one recombinant polypeptide of the invention, and the second nucleic acid comprising a polynucleotide sequence that encodes a protein adjuvant. Each such nucleic acid may be an expression vector. Preferably, the adjuvant is a cytokine that promotes the immune response induced by at least immunogenic recombinant polypeptide of the invention (e.g., a polypeptide comprising a sequence having at least about 96, 97, 98, 99, or 100% sequence identity with a polypeptide sequence selected from the group of SEQ ID NOS:1, 4-10, 12-14, 32, 34, 78, and 92), which have the ability to induce at least one type of immune response against mEpCAM or hEpCAM or an antigenic fragment thereof (including, e.g., the ability to induce production of antibodies that specifically bind hEpCAM or an antigenic or immunogenic fragment thereof, the ability to induce T cell proliferation and/or activation, and/or the ability to induce production of one or more cytokines (e.g., including IL and/or IFN). Preferably, the cytokine is a granulocyte macrophage colony stimulating factor (a GM-CSF, e.g., a human GM-CSF) an interferon (e.g., human interferon (IFN) alpha, IFN-beta, IFN-γ), an Interleukin (e.g., an IL-2, IL-12, IL-15, IL-18, etc.), or a peptide comprising an amino acid sequence that is at least substantially identical (e.g., having at least about 75%, 80%, 85%, 86%, 87%, 88% or 89%, preferably at least about 90%, 91%, 92%, 93%, or 94%, and more preferably at least about 95% (e.g., about 87-95%), 96% 97%, 98%, 99%, 99.5% or more sequence identity) to the sequence of at least one such cytokine. Genes encoding such cytokines are known. Human GM-CSF sequences are described in, e.g., Wong et al. (1985) Science 228:810, Cantrell et al. (1985) Proc Natl Acad Sci 82:6250, and Kawasaki et al. (1985) Science 230:291. Desirably, in one embodiment, such a nucleic acid expresses an amount of GM-CSF or a functional analog thereof that detectably stimulates the mobilization and differentiation of dendritic cells (DCs) and/or T-cells, increases antigen presentation, and/or increases monocytes activity, such that the immune response induced by the immunogenic recombinant polypeptide of the invention is increased. Suitable interferon genes, such as IFN-γ genes also are known (see, e.g., Taya et al. (1982), Embo J. 1:953-958, Cerretti et al. (1986) J. Immunol. 136(12):4561, and Wang et al. (1992) Sci. China. B. 35(1):84-91). Desirably, the IFN, such as the IFN-γ, is expressed from the nucleic acid in an amount that increases the immune response of the immunogenic recombinant polypetpide of the invention (e.g., by enhancing a T cell immune response induced by the immunogenic polypeptide). Advantageous IFN-homologs and IFN-related molecules that can be co-expressed or co-administered with a polynucleotide and/or polypeptide of the invention are described in, e.g., International Patent Applications WO 01/25438 and WO 01/36001. Co-administration (which herein includes both simultaneous and serial administration) of about 1 to 5 to about 10 μg of a GM-CSF-encoding plasmid with about 5 to about 50 μg of a plasmid encoding one of the polypeptides of the invention is expected to be effective or useful for enhancing the antibody response in a mouse model. In another aspect, co-administration of about 1 μg to about 1 mg, 10 μg to about 500 μg, 100 μg to about 250 μg, 10 μg to about 100 μg of a GM-CSF-encoding plasmid with, respectively, an amount of 5 μg to about 5 mg, 50 μg to about 2.5 mg, 500 μg to about 1 mg, 50 μg to about 1 mg of a plasmid encoding one of the polypeptides of the invention may be effective for enhancing the antibody response in a mouse model.

Nucleic Acids Encoding TAg Polypeptides and/or Costimulators

In another aspect, the invention provides a nucleic acid comprising a first nucleotide sequence that encodes an immunogenic polypeptide of the invention (e.g., a polynucleotide sequence having at least about 95, 96, 97, 98, 99, or 100% nucleic acid sequence identity to a polynucleotide sequence selected from the group of SEQ ID NOS: 16, 19-23, 26-28, 33, 35, 79, and 94, or a polypeptide comprising a polypeptide sequence having at least about 96, 97, 98, 99, or 100% amino acid sequence identity with a polypeptide sequence selected from the group of SEQ ID NOS:1, 4-10, 12-14, 32, 34, 78, and 92) and a second nucleotide sequence that encodes a costimulatory polypeptide. Such nucleic acid may be an expression vector. Alternatively, the first and second nucleotide sequences may comprise part of two separate nucleic acids or vectors (instead of one nucleic acid or vector comprising both sequences). In one aspect, such costimulatory polypeptide induces an immune response, such as, e.g., promotes T cell activation. Measurements of T cell activation are known. Briefly, T cell activation is commonly characterized by physiological events including, e.g., T cell-associated cytokine synthesis (e.g., IFN-γ production) and induction of various activation markers such as CD25 (interleukin-2 (IL-2) receptor). CD4+ T cells recognize their immunogenic peptides in the context of MHC class II molecules, whereas CD8+ T cells recognize their immunogenic peptides in the context of MHC class I molecules.

For induction of T cell activation, cytokine synthesis, or effector function, secondary signals, such as those mediated through the CD28 receptor, can play a significant role. Two ligands for CD28 are B7-1 (CD80) and B7-2 (CD86). B7-1 and B7-2 are termed co-stimulatory polypeptides and are typically expressed on professional antigen-presenting cells (APCs).

In one aspect, the invention provides a nucleic acid comprising a first polynucleotide sequence encoding an immunogenic polypeptide of the invention (e.g., a polynucleotide sequence having at least about 95, 96, 97, 98, 99, or 100% nucleic acid sequence identity to a polynucleotide sequence selected from the group of SEQ ID NOS: 16, 19-23, 26-28, 33, 35, 79, and 94, or a polypeptide comprising a polypeptide sequence having at least about 96, 97, 98, 99, or 100% amino acid sequence identity with a polypeptide sequence selected from the group of SEQ ID NOS: 1,4-10, 12-14, 32, 34, 78, and 92), and a second polynucleotide sequence that encodes a mammalian B7-1 (e.g., human B7-1 (hB7-1) or human B7-2 (hB7-2), a functional fragment of either thereof (e.g., a fragment comprising the hB7-1 or hB7-2 extracellular domain and any other portions required for costimulation), or a variant thereof that has significant identity (e.g., at least about 95% identity) to either thereof and that promotes T cell activation). Such nucleic acid may be an expression vector. Alternatively, the first and second nucleotide sequences may comprise part of two separate nucleic acids or vectors (instead of one nucleic acid or vector comprising both sequences), but administered consecutively or together to a subject as described further herein. B7-1, which usually is expressed on activated human B-lymphocytes and macrophages, and B7-2, which is expressed on B-lymphocytes, monocytes and dendritic cells, as well as variants of such molecules, are known, and B7 molecules from several mammals have been identified (see, e.g., U.S. Pat. Nos. 5,738,852, 5,858,776, and 6,149,905, and Freeman et al., J. Immunol. 143:2714-2722 (1989)). Expression of B7-1 on human myeloma cells (Wendtner, C. et al. (1997) Gene Therapy 4(7):726-735), murine mammary tumors (Martin-Fontecha, A. et al. (2000) J. Immunol. 164(2):698-704) or murine sarcoma (Indrova et al. (1998) Intl. J. One. 12(2):387-390) is known to enhance anti-tumor immunity.

The nucleic acid or vector can also or alternatively include at least one additional different polynucleotide sequence encoding a costimulatory polypeptide. For example, the nucleic acid can comprise a third polynucleotide sequence encoding a CD40 ligand (CD40L), immunostimulatory fragment thereof, or functional variant thereof. CD40L is known to elicit an anti-tumor response and suppressor tumor progression (e.g., tumor growth) and can serve as an adjuvant in DNA vaccination.

In other aspects, the nucleic acid can comprise a third polynucleotide sequence encoding 4-1BBL. For example, the invention provides a nucleic acid comprising a first polynucleotide sequence encoding the polypeptide sequence of SEQ ID NO: 1 or an amino acid sequence variant thereof, a second polynucleotide sequence encoding a B7.1 protein or a another protein that binds CD28 receptor), and a third polynucleotide sequence that encodes a 4-1BBL or a portion thereof (a soluble receptor binding portion). Additionally or alternatively still, such nucleic acid may comprise a polynucleotide sequence that encodes O×40L (gp34) or a fragment thereof (a soluble receptor binding portion thereof).

As a further additional or alternative aspect, a nucleic acid comprising a polynucleotide sequence encoding an immunogenic polypeptide of the invention can comprise an ICOS. In other aspects, a nucleic acid comprising a polynucleotide sequence encoding an immunogenic polypeptide of the invention may further a suitable costimulatory polypeptide-encoding polynucleotide coding sequence for ICAM-1, a TRAF protein (e.g., TRAF2), or other member of the TNF/TNFR superfamily, a lymphocyte function-associated antigen (LFA-3), vascular cell adhesion molecule (VCAM-1), and suitable fragments or variants of these costimulatory polypeptides.

In one aspect, the invention provides a nucleic acid comprising a first polynucleotide sequence encoding an immunogenic polypeptide of the invention (e.g., a polynucleotide sequence having at least about 95, 96, 97, 98, 99, or 100% nucleic acid sequence identity to a polynucleotide sequence selected from the group of SEQ ID NOS: 16, 19-23, 26-28, 33, 35, 79, and 94, or a polypeptide comprising a polypeptide sequence having at least about 96, 97, 98, 99, or 100% amino acid sequence identity with a polypeptide sequence selected from the group of SEQ ID NOS:1, 4-10, 12-14, 32, 34, 78, and 92), and a second polynucleotide sequence that encodes a novel costimulatory molecule (NCSM) that binds CD28 receptor preferentially over CTLA-4 receptor; such costimulatory molecule is termed a CD28 binding protein (CD28BP). Such CD28BPs are described in Int'l Patent Application No. PCT/US01/19973, filed Jun. 22, 2001 (WO 02/00717) and Int'l Patent App. No. PCT/US02/19898, filed Jun. 21, 2002, each of which is incorporated herein by reference in its entirety for all purposes. See also Lazetic et al., J. Biol. Chem. 277:38660 (2002). An exemplary CD28BP is CD28BP-15; the polypeptide sequence of CD28BP-15 and the nucleic acid sequence encoding CD28BP-15 are shown in Int'l Patent App. No. PCT/US01/19973 (WO 02/00717) and Int'l App. No. PCT/US02/19898. The amino acid and nucleic acid sequences of CD28BP-15 are designated as SEQ ID NO:66 and SEQ ID NO:19, respectively, in PCT/US01/19973 (WO 02/00717) and PCT/US02/19898.

The nucleic acid can comprise any suitable number of such costimutatory polynucleotide sequences and/or immunostimulatory cytokine polynucleotide sequences, in any suitable combination, along with the recombinant immunogenic polypeptide-encoding sequence(s) of the invention (e.g., any of SEQ ID NOS:1, 4-10, 12-14, 32, 34, 78, and 92). These sequences can be part of a single expression cassette, but more typically and preferably are contained in separate expression cassettes (examples of which are discussed further below). In some aspects, the nucleotide sequence encoding the immunogenic polypeptide of the invention and the second nucleic acid sequence (the costimulatory polypeptide-encoding or cytokine adjuvant-encoding polynucleotide sequence) are operably linked to separate and different expression control sequences, such that they are expressed at different times and/or in response to different conditions (e.g., in response to different inducers).

In some aspects the nucleic acid comprises a first polynucleotide sequence encoding an immunogenic polypeptide of the invention (e.g., a polynucleotide sequence having at least about 95, 96, 97, 98, 99, or 100% nucleic acid sequence identity to a polynucleotide sequence selected from the group of SEQ ID NOS:16, 19-23, 26-28, 33, 3.5, 79, and 94, or a polypeptide comprising a polypeptide sequence having at least about 96, 97, 98, 99, or 100% amino acid sequence identity with a polypeptide sequence selected from the group of SEQ ID NOS:1, 4-10, 12-14, 32, 34, 78, and 92), and a second polynucleotide sequence encoding a costimulatory polypeptide (e.g., a CD28 binding protein, such as B7-1, or CD28BP-15 (see Int'l Patent App. No. PCT/US01/19973, filed Jun. 22, 2001 (WO 02/00717) and PCT/US02/19898, filed Jun. 21, 2002), which are oriented in the same orientation (read towards each other (i.e., in the same 5′-3′ direction) during translation) in the nucleic acid. In one aspect, orientation of such two polynucleotide sequences in the same orientation provides superior expression and immune response as compared to orientation of such sequences in opposite directions in a single nucleic acid.

In one particular aspect, the invention provides a multicomponent nucleic acid vector, such as a bicistronic vector. In one format, the bicistronic vector comprises: 1) a first polynucleotide sequence that encodes an immunogenic polypeptide of the invention (e.g., a polynucleotide sequence having at least about 95, 96, 97, 98, 99, or 100% nucleic acid sequence identity to a polynucleotide sequence selected from the group of SEQ ID NOS: 16, 19-23, 26-28, 33, 35, 79, and 94, or a polypeptide comprising a polypeptide sequence having at least about 96, 97, 98, 99, or 100% amino acid sequence identity with a polypeptide sequence selected from the group of SEQ ID NOS:1, 4-10, 12-14, 32, 34, 78, and 92), wherein the first nucleotide sequence is operably linked to a first promoter (e.g., CMV IE (Towne) promoter/enhancer or a chimeric CMV promoter/enhancer (e.g., a chimeric CMV promoter as described in Int'l Patent Application No. PCT/US01/20123, filed Jun. 21, 2001, published with Int'l Publ. No. WO 02/00897); and 2) a second polynucleotide sequence that encodes a co-stimulatory polypeptide (e.g., a CD28BP, such as CD28BP-15, or a WT hB7-1 or hB7-2) or an immunostimulatory cytokine (e.g., GM-CSF or TNF-α), wherein the second polynucleotide sequence is operably linked to a second promoter. The second promoter may be the same as or different from the first promoter. For example, the second promoter can be a CMV promoter/enhancer or chimeric CMV promoter/enhancer. In the context of “CMV promoters” discussed herein, it is generally understood that the term “promoter” may include both the promoter and enhancer portions of the CMV immediate/early (i.e.) or Towne promoter/enhancer sequence.

Using Nucleic Acids

Polynucleotides of the invention and fragments thereof can be used as substrates for any of a variety of recombination and recursive sequence recombination reactions described herein, in addition to their use in standard cloning methods as set forth in, e.g., Ausubel, Berger, and Sambrook, e.g., to produce additional polynucleotides or fragments thereof that encode recombinant antigens of the invention having desired properties. A variety of such reactions are known, including those developed by the inventors and their co-workers.

Polynucleotides of the invention, and nucleic acid vectors or other vectors described above comprising at least one polynucleotide of the invention, are also useful in a variety of therapeutic and/or prophylactic methods for inducing an immune response to EpCAM-associated or EpCAM-overexpressing tumors or cancers as discussed in more detail below.

The nucleic acids of the invention also can be useful for sense and anti-sense suppression of expression (e.g., to regulate expression of a nucleic acid of the invention once or when expression is no longer require or to control nucleic acid expression levels in tissues away from those in which expression of an administered nucleic acid or vector is desired). A variety of sense and anti-sense technologies are known in the art, e.g., as set forth in Lichtenstein and Nellen (1997) ANTISENSE TECHNOLOGY: A PRACTICAL APPROACH IRL Press at Oxford University, Oxford, England, and in Agrawal (1996) ANTISENSE THERAPEUTICS, Humana Press, NJ, and the references cited therein.

In this respect, the invention provides nucleic acids that comprise a nucleic acid sequence that is the substantial complement (i.e., comprises a nucleotide sequence that complements at least about 90%, preferably at least about 95, 96, 97, 98, 99%), and more preferably the complement, of any of the above-described nucleic acid sequences. Such complementary nucleic acid sequences are useful in probes, production of the nucleic acid sequences of the invention, and as antisense nucleic acids for hybridizing to nucleic acids of the invention. Short oligonucleotide sequences comprising sequences that complement the nucleic acid, e.g., of about 15, about 20, about 30, or about 50 bases (preferably at least about 12 bases), which hybridize under highly stringent conditions to a nucleic acid of the invention also are useful as probes (e.g., to determine the presence of a nucleic acid of the invention in a particular cell or tissue and/or to facilitate the purification of nucleic acids of the invention). The polynucleotides comprising complementary sequences also can be used as primers for amplification of the nucleic acids of the invention.

Additional uses of the nucleic acids and vectors of the invention are described elsewhere herein.

Antibodies

In another aspect, the invention provides novel or recombinant antibodies that are useful in a number of respects. For example, the invention provides at least one antibody induced in response to the administration or expression of at least one polypeptide of the invention (e.g., at least one polypeptide comprising a polypeptide sequence having at least about 96, 97, 98, 99, or 100% amino acid sequence identity to a polypeptide sequence selected from the group consisting of SEQ ID NOS:1, 4-10, 12-14, 32, 34, 78, and 92). In another aspect, the invention provides a population of such antibodies, expressed by antibody-producing cells (e.g., human B cells) in response to the administration and/or expression of at least one such polypeptide of the invention in an area where such polypeptide can induce such an immune response from such antibody-producing cells.

In another aspect, the invention provides at least one monoclonal antibody that binds to both a polypeptide of the invention (e.g., a polypeptide comprising or consisting essentially of SEQ ID NO:4) and mEpCAM. Such a monoclonal antibody(ies) typically is produced by a hybridoma that is generated by the fusion of an antibody-producing cell exposed to a polypeptide of the invention by administration or expression near the antibody-producing cell.

The antibodies of the invention can advantageously be characterized by the ability to detectably bind mEpCAM, such as hEpCAM, a polypeptide sequence comprising SEQ ID NO:4 or other immunogenic polypeptide sequence of the invention, or both. Desirably, antibodies of the invention are further able to facilitate an immune response against cells comprising or expressing EpCAM by targeting of antigen presenting cells (APCs), contributing to antibody-dependent cellular toxicity (ADCC), or by inducing any other suitable immunological reaction (e.g., macrophage-mediated phagocytosis).

In another aspect, the invention provides a hybridoma that expresses an antibody that binds to mEpCAM and an immunogenic polypeptide sequence of the invention (i.e., a cross-reactive antibody for mEpCAM and an immunogenic polypeptide sequence of the invention) and a method of producing such a hybridoma. Such immunogenic polypeptide sequence can comprise, e.g., a polypeptide comprising a polypeptide sequence having at least about 96, 97, 98, 99, or 100% sequence identity to a polypeptide sequence selected from the group consisting of SEQ ID NOS: 1, 4-10, 12-14, 32, 34, 78, and 92. The mEpCAM is preferably hEpCAM. The method of producing such a hybridoma includes the steps of exposing an antibody-producing cell (e.g., a spleen B cell in a mammalian host or mammalian host tissue) to a polypeptide of the invention for a suitable period of time, fusing the antibody-expressing B cell to a myeloma cell (usually a selectable “tumor partner” myeloma cell), using standard hybridoma generation techniques (e.g., PEG-induced fusion—see, e.g., METHODS IN ENZYMOLOGY: IMMUNOCHEMICAL TECHNIQUES, PART I: HYBRIDOMA TECHNOLOGY AND MONCLONAL ANTIBODIES, Langone et al. (Eds.), Academic Press (1997) and HYBRIDOMA TECHNOLOGY IN THE BIOSCIENCES AND MEDICINE, Springer, Plenum Pub. Corp. (1985) for discussion and other techniques). Advantageously, the invention provides hybridomas that express monoclonal antibodies that bind mEpCAM (preferably hEpCAM) with high optical density values (as measured in an EpCAM ELISA) and with efficient production, as is described in Example 1 in the Examples section below.

In another aspect, the invention provides a method of producing such antibodies. Such antibodies can be produced, e.g., by administering an effective amount (e.g., an antigenic amount or immunogenic amount) of at least one recombinant polypeptide of the invention or an antigenic or immunogenic fragment thereof, or an effective amount of a vector or nucleic acid encoding such at least one polypeptide, or composition comprising an effective amount of such at least one polypeptide or nucleic acid or polynucleotide encoding said at least polypeptide, to a suitable animal host or host cell. The host cell is cultured or the animal host is maintained under conditions permissive for formation of antibody-antigen complexes. Subsequently produced antibodies are recovered from the cell culture, the animal, or a byproduct of the animal (e.g., sera from a mammal). The production of antibodies can be carried out with either at least one polypeptide of the invention, or a peptide or polypeptide fragment thereof comprising at least about 10 amino acids, preferably at least about 15 amino acids (e.g., about 20 amino acids), and more preferably at least about 25 amino acids (e.g., about 30 amino acids) or more in length. Alternatively, a nucleic acid or vector can be inserted into appropriate cells, which are cultured for a sufficient time and under periods suitable for transgene expression, such that a nucleic acid sequence of the invention is expressed therein resulting in the production of antibodies that bind to the recombinant antigen encoded by the nucleic acid sequence. Antibodies thereby obtained can have diagnostic and/or prophylactic uses. Such antibodies, and compositions and pharmaceutical compositions comprising such antibodies (by use of the principles described above with respect to other compositions and pharmaceutically acceptable compositions), are features of the invention.

Antibodies produced in response to at least one polypeptide of the invention, fragment thereof, or the expression of such at least one polypeptide by a vector and/or polynucleotide of the invention can be any suitable type of antibody or antibodies. Antibodies provided by the invention include, e.g., polyclonal antibodies, monoclonal antibodies, chimeric antibodies, humanized antibodies, single chain antibodies, Fab fragments, and fragments produced by a Fab expression library. Those of skill in the art know methods of producing polyclonal and monoclonal antibodies, and many types of antibodies and methods are available. See, e.g., Current Protocols in Immunology, John Colligan et al., eds., Vols. I-IV (John Wiley & Sons, Inc., NY, 1991 and 2001 Supplement), and Harlow and Lane (1989) ANTIBODIES: A LABORATORY MANUAL, Cold Spring Harbor Press, NY, Stites et al. (eds.) BASIC AND CLINICAL IMMUNOLOGY (4th ed.) Lange Medical Publications, Los Altos, Calif., and references cited therein, Goding (1986) MONOCLONAL ANTIBODIES: PRINCIPLES AND PRACTICE (2d ed.) Academic Press, New York, N.Y., and Kohler and Milstein (1975) Nature 256:495-497. Other suitable techniques for antibody preparation include selection of libraries of recombinant antibodies in phage or similar vectors. See, Huse et al. (1989) Science 246:1275-1281; and Ward et al. (1989) Nature 341:544-546. Specific monoclonal and polyclonal antibodies and antisera will usually bind with a KD of at least about 0.1 μM, preferably at least about 0.01 μM or better, and most typically and preferably, 0.001 μM or better.

Detailed methods for preparation of chimeric (humanized) antibodies can be found in e.g., U.S. Pat. No. 5,482,856. Additional details on humanization and other antibody production and engineering techniques can be found in Borrebaeck (ed.) (1995) Antibody Engineering, 2nd Edition Freeman and Company, NY (Borrebaeck); McCafferty et al. (1996) Antibody Engineering, A Practical Approach IRL at Oxford Press, Oxford, England (McCafferty), and Paul (1995) Antibody Eng'g Protocols Humana Press, Towata, N.J. (Paul).

Humanized antibodies are especially desirable in applications where the antibodies are used as therapeutics and/or prophylactics in vivo in mammals (e.g., such as humans) and ex vivo in cells or tissues that are delivered to or transplanted into mammals (humans). Human antibodies consist of characteristically human immunoglobulin sequences. The human antibodies of this invention can be produced in using a wide variety of methods (see, e.g., Larrick et al., U.S. Pat. No. 5,001,065, and Borrebaeck McCafferty and Paul, supra, for a review). In one embodiment, the human antibodies of the present invention are produced initially in trioma cells. Genes encoding the antibodies are then cloned and expressed in other cells, such as nonhuman mammalian cells. The general approach for producing human antibodies by trioma technology is described by Ostberg et al. (1983), Hybridoma 2:361-367, Ostberg, U.S. Pat. No. 4,634,664, and Engelman et al., U.S. Pat. No. 4,634,666. The antibody-producing cell lines obtained by this method are called triomas because they are descended from three cells two human and one mouse. Triomas have been found to produce antibody more stably than ordinary hybridomas made from human cells.

Additional useful techniques for preparing antibodies are described in, e.g., Gavilodono et al., Biotechniques 29(1):128-32, 134-6, and 138 (passim) (2000), Nelson et al., Mol. Pathol. 53(3):111-7 (2000), Laurino et al., Ann. Clin. Lab. Sci. 29(3):158-66 (1999), Rapley, Mol. Biotechnol. 3(2):139-54 (1995), Zaccolo et al., Int. J. Clin. Lab. Res. 23(4):192-0.8 (1993), Morrison, Annu. Rev. Immunol. 10:239-65 (1992), “Antibodies, Annigene, and Molecular Mimiery,” Meth. Enzymol. 178 (John J. Langone, Ed. 1989), Moore, Clin. Chem. 35(9):1849-53 (1989), Rosalki et al., Clin. Chim. Acta 183(1):45-58 (1989), and Tami et al., Am. J. Hosp. Pharm. 43(11):2816-25 (1986), as well as U.S. Pat. Nos. 4,022,878, 4,350,683, and 4,022,878. A technique for producing antibodies with remarkably high binding affinities is provided in Border et al., Proc. Natl. Acad. Sci. USA 97(20):10701-05 (2000).

In another aspect, the invention provides a chimeric antibody comprising a antigen-binding fragment (or portion) of an antibody, which antibody is produced in response to the administration or expression of a polypeptide of the invention to a suitable antibody-producing cell or animal host. For example, the invention provides an antibody comprising the Fc region of a human. EpCAM antibody (e.g., KSA 1/4) and the antigen-binding portion of a mouse antibody produced in response to the expression or administration of a polypeptide of the invention (e.g., a polypeptide comprising SEQ ID NO: 1, 4, 5, or 6).

The invention also provides an antibody fusion protein, wherein an antibody of the invention is expressed as a fusion protein with an anti-tumor cytokine (e.g., TNF-α) and/or a pro-coagulant factor. In a related aspect, the invention provides conjugates of an antibody of the invention in combination with an antitumor or anticancer agent, such as a small molecule antitumor agent. The antibodies and/or antibody fragments of the invention can be used to similarly target vectors (e.g., viral vector particles) or nucleic acids to EpCAM-overexpressing cells in a tissue (e.g., an organ in a human).

In general, the polypeptides of the invention provide structural features that can be recognized, e.g., in immunological assays. The production of antisera comprising at least one antibody (for at least one antigen) that binds or specifically binds a polypeptide of the invention, and the polypeptides that are bound by such antisera, are features of the invention. Binding agents, including the novel antibodies described herein, may bind a polypeptide of the invention and/or EpCAM about 1×10² M⁻¹ to about 1×10¹⁰ M⁻¹ (i.e., about 10⁻²-10⁻¹⁰ M) or greater, including about 10⁴ to 10⁶ M⁻¹, about 10⁶ to 10⁷ M⁻¹, or about 10⁸ M⁻¹ to 10⁹ M⁻¹ or 10¹⁰ M⁻¹). Conventional hybridoma technology can be used to produce antibodies having affinities of up to about. 10⁹ M⁻¹. However, other technologies, including phage display and transgenic mice, can be used to achieve higher affinities (e.g., up to at least about 10¹² M⁻¹). In many aspects of the invention a higher binding affinity is advantageous. However, in other aspects, discussed elsewhere herein, lower affinities can be preferred. For example, antibodies with lower, but sufficient, affinity for EPCAM (e.g., an affinity of about 7×10⁷ L/mol) can be advantageous in therapeutic contexts, due to the ability of such lower-affinity antibodies to better penetrate a tumor in vivo.

In order to produce antiserum or antisera for use in an immunoassay, at least one immunogenic polypeptide (or polypeptide-encoding polynucleotide) of the invention is produced and purified as described herein. For example, a polypeptide of the invention may be produced in a mammalian cell line. Alternatively, an inbred strain of mice can immunized with the immunogenic protein(s) in combination with a standard adjuvant, such as Freund's adjuvant or alum, and a standard mouse immunization protocol (see Harlow and Lane, supra, for a standard description of antibody generation, immunoassay formats and conditions that can be used to determine specific immunoreactivity). Alternatively, at least one synthetic or recombinant polypeptide derived from at least one polypeptide sequence disclosed herein or expressed from at least one polynucleotide sequence disclosed herein can be conjugated to a carrier protein and used as an immunogen for the production of antiserum. Polyclonal antisera typically are collected and titered against the immunogenic polypeptide in an immunoassay, for example, a solid phase immunoassay with one or more of the immunogenic proteins immobilized on a solid support. In the above-described methods where novel antibodies and antisera are provided, antisera resulting from the administration of the polypeptide (or polynucleotide and/or vector) with a titer of about 10⁶ or more typically are selected, pooled and subtracted with the control co-stimulatory polypeptides to produce subtracted pooled titered polyclonal antisera.

Some antisera raised or induced by an immunizing antigen are not totally specific for their inducing antigen, but bind related (cross-reacting) antigens, either because the cross-reacting antigens share epitopes, or the epitopes are sufficiently similar in shape or structure to bind the same antibody. David Male, IMMUNOLOGY: AN ILLUSTRATED OUTLINE (Gower Medical Publishing, London & NY, 1986).

Some antibodies of the invention can cross-react with human EpCAM and one or more immunogenic polypeptide sequences of the invention (e.g., a polypeptide comprising a polypeptide sequence having at least about 96, 97, 98, 99, or 100% sequence identity to a polypeptide sequence selected from the group consisting of SEQ ID NOS:1, 4-10, 12-14, 32, 34, 78, and 92). Cross-reactivity of a population of antibodies and/or a particular antibody can be determined using standard techniques, such as competitive binding immunoassays and/or parallel binding assays, and standard calculations for determining the percent cross-reactivity. Usually, where the percent cross-reactivity is at least 5-10× as high for the test polypeptides, the test polypeptides are said to specifically bind the pooled subtracted antisera or antibody. That polypeptides, nucleic acids, recombinant cells, and vectors of the invention are able to induce the production of a population of antibodies that cross-react (i.e., bind both) hEpCAM and an immunogenic polypeptide of the invention, particular antibodies that so cross-react, or both, is an important feature of the invention. Another significant feature attendant the polypeptides, nucleic acids, vectors, and cells of the invention is the ability to induce a cross-reactive T cell-mediated immune response (e.g., a T cell proliferative immune response against an immunogenic polypeptide of the invention that also is exhibited against hEpCAM-overexpressing cells).

In yet another aspect, the invention provides anti-idiotype antibodies related to antibodies produced in response to an immunogenic polypeptide of the invention. An anti-idiotype antibody will usually bear the internal image of the Ab₁ epitope-recognition site (i.e., the image of the antigen-binding site of an antibody raised against an immunogenic polypeptide of the invention) and, as such, can often mimic the immunological properties of the portion of the antigen comprising the recognized epitope(s). Techniques for the production of anti-idiotype antibodies are known. Briefly, the invention provides a method of producing such an antibody comprising providing an Ab₁ antibody, as described above (e.g., a murine hybridoma cell monoclonal antibody to a polypeptide comprising or consisting essentially of SEQ ID NO: 12), introducing such an antibody to a tissue system or host comprising antibody-producing cells, wherein the Ab₁ antibody is foreign (e.g., to a human tissue, goat, or other mammal) to produce the anti-idiotype antibody. Alternatively, hybridomas that produce such antibodies can be generated by exposure of a suitable type of hybridoma to the antibody. Such antibodies can be subject to modification or fragmentation as described above with respect to other antibodies of the invention (e.g., the invention provides a chimeric anti-idiotype antibody, wherein the chimeric antibody comprises a human Fc fragment of a human EpCAM antibody).

In a further aspect, the invention provides an anti-anti-idiotype antibody and a method for producing the same. Anti-anti-idiotype antibodies can be produced by exposing an anti-idiotype antibody of the invention to a foreign host or host tissue comprising antibody-producing cells, and isolating resulting antibodies, or through the use of hybridomas generated from such cells (to produce monoclonal anti-anti-idiotype antibodies). Anti-anti-idiotype antibodies comprise a portion that resembles the epitope recognition sequence of an Ab₁ antibody and can be used in a manner similar to such antibodies of the invention.

Such anti-idiotype and anti-anti-idiotype antibodies of the invention are useful inasmuch as human antibodies to mouse or other non-human mammal Ab₁ antibodies do not induce production of human anti-mouse antibodies during therapeutic administration.

Methods of the Invention

Therapeutic and Prophylactic Applications

The polypeptides, nucleic acids, vectors, antibodies, cells and compositions of the invention are useful in a number of therapeutic and/or prophylactic applications, primary of which is the ability to induce an immune response(s) in a subject against human EpCAM or an antigenic fragment thereof. For example, most, if not all, of the immunogenic polypeptides of the invention (e.g., a polypeptide comprising a polypeptide sequence having at least about 96, 97, 98, 99, or 100% sequence identity to a polypeptide sequence selected from the group consisting of SEQ ID NOS:1, 4-10, 12-14, 32, 34, 78, and 92) are able to induce an immune response against hEpCAM or an antigenic fragment thereof, which immune response includes the production of antibodies capable of binding hEpCAM (or antigenic fragment thereof) by antibody-producing cells (e.g., mammalian B cells), particularly in a subject, including a mammal, including, e.g., a human. The induction and/or promotion of EpCAM-specific antibodies is an important feature of the invention.

In one aspect, the polypeptides, nucleic acids, vectors, antibodies, cells, and/or compositions of the invention are useful in therapeutic or prophylactic treatment therapies and/or vaccines for a variety of tumors and cancers, including those associated with expression or over-expression of human EpCAM. Some such polypeptides, nucleic acids, vectors, antibodies, cells, and compositions on the invention are useful in inducing specific immune responses against EpCAM, including an EpCAM-specific antibody response, a T cell proliferation or activation response (e.g., EpCAM-specific CD8+ response), and/or cytokine responses (e.g., enhanced production of cytokines, such as IFN-g and/or 1L-5). The polypeptides, nucleic acids, vectors, antibodies, cells, and compositions of the invention may also be useful in diagnostic assays as described in greater detail below.

The invention includes a method of inducing the production of antibodies that bind or specifically bind mEpCAM, preferably hEpCAM. In one aspect, such method comprises administering an effective amount of a polypeptide, nucleic acid, vector, or a combination of any thereof, to a mammalian host such that a detectable amount of antibodies that bind hEpCAM or an antigenic fragment thereof are produced therein.

In one aspect, the invention provides a method of inducing an immune response against human EpCAM or an antigenic fragment thereof in a subject, the method comprising administering to the subject an effective amount of at least immunogenic polypeptide of the invention or at least one nucleic acid encoding at least one such immunogenic polypeptide. The effective amount is typically sufficient to induce an immune response against human EpCAM. In one aspect, the immunogenic polypeptide comprises a polypeptide sequence selected from the group consisting of SEQ ID NOS:1, 4-10, 12, 13, 32, 34, 78, and 92, wherein said polypeptide is able to induce an immune response against hEpCAM that is at least as potent as the immune response induced by hEpCAM, an hEpCAM homolog, an hEpCAM ortholog, or an antigenic fragment of any thereof. In one aspect, the immunogenic polypeptide comprises a TAg-encoding extracellular domain of the invention, such as a polypeptide comprising a polypeptide sequence selected from the group consisting of SEQ ID NOS:1, 9, 12, and 92, has the ability to induce an immune response against hEpCAM at a level that is about comparable to or better than (i.e., at least as great as) an immune response induced against hEpCAM by a polypeptide comprising a polypeptide sequence that is identical or substantially identical to that of SEQ ID NO:36. In another aspect, the immunogenic polypeptide is a polypeptide sequence selected from the group consisting of SEQ ID NOS: 1, 9, 12, and 92 is at least as immunogenic in a mammalian host as a polypeptide consisting essentially of the polypeptide sequence of SEQ ID NO:36.

Such method can further comprise administering to the subject a second effective amount of at least at least one such immunogenic polypeptide or at least one nucleic acid of the invention that encodes such immunogenic polypeptide. Typically, the second effective amount is administered to the subject after the first effective amount and at a time such that the immune response to human EpCAM in the subject is enhanced.

In another aspect, the invention provides a method of inducing production of antibodies that bind human EpCAM, said method comprising administering to a subject an effective amount of: 1) at least one immunogenic polypeptide of the invention or at least one nucleic acid of the invention encoding such an immunogenic polypeptide, 2) a nucleic acid vector comprising at least one nucleic acid of the invention that encodes at least one such immunogenic polypeptide, (3) a viral vector, virus or virus-like particle (VLP) comprising at least one such immunogenic polypeptide or nucleic acid encoding such an immunogenic polypeptide of the invention, or a combination thereof, wherein the effective amount is sufficient to induce in the subject production of a detectable amount of antibodies that bind hEpCAM.

Promotion of an immune response induced by an immunogenic polypeptide, nucleic acid, vector, cell, or antibody of the invention typically results in a detectable immune response. The polypeptides, nucleic acids, vectors, cells, and antibodies of the invention also or alternatively can be associated with the induction of an immune response, as well as the increase or enhancement (quantitatively, qualitatively (by a measurable characteristic, such as antibody-antigen affinity or antibody infiltration of a tumor), or both) of an already existing immune response. The polypeptides of the invention can induce a cytotoxic (or other T-cell) immune response, a humoral (antibody-mediated) immune response, or (most desirably) both.

In another aspect, the invention provides a method of inducing or promoting an immune response against hEpCAM or an antigenic fragment thereof in a subject, the method comprising administering to the subject an effective amount of: 1) at least one immunogenic polypeptide of the invention or at least one nucleic acid of the invention encoding such an immunogenic polypeptide, 2) a nucleic acid vector comprising at least one nucleic acid of the invention that encodes at least one such immunogenic polypeptide, (3) a viral vector, virus or virus-like particle (VLP) comprising at least one such immunogenic polypeptide or nucleic acid encoding such an immunogenic polypeptide of the invention, or a combination thereof, wherein the effective amount is sufficient to induce or promote such immune response in the subject. The induced or enhanced immune response can comprise production of antibodies that bind EpCAM; T cell activation or proliferation; and/or production of at least one cytokine.

In one aspect, the immune response is a T cell mediated immune response, such as a cytotoxic (CD8+) or Th (e.g., a CD4+ (MHC Class II restricted response) immune response. As such, the invention provides methods of priming and/or stimulating CD4+ and CD8+ lymphocytes that react with EpCAM, T cell activation, and cytokine release (including, but not limited to, e.g., release of one or more tumor necrosis factors (TN F) (e.g., TNF-alpha), the production of one or more interleukins (IL) (e.g., IL-1, IL-2, IL-3, IL-4, IL-5, IL-6, IL-10, IL-12), the production of one or more interferons (IFN) (e.g., IFN-gamma, IFN-alpha, IFN-beta), or TGF from T cells, complement activation, platelet activation, enhanced and/or decreased Th1 responses, enhanced and/or decreased Th2 responses, and humoral immunological memory. Thus, for example, the invention provides a method of inducing a CD8+ T cell response in an antigen specific and MHC-restricted fashion by the administration of an immunogenic amount of a nucleic acid, polypeptide, and/or vector of the invention. An immune response induced, promoted, enhanced, and/or increased by a polypeptide, nucleic acid, cell, and/or antibody of the invention advantageously may be associated with in vivo spontaneous recognition of EpCAM (see, e.g., Mosolits et al., Cancer Immunol. Immunother. 47:315-320 (1999) for discussion of spontaneous recognition with respect to EpCAM-related tumor-associated antigens. In a more general sense, the invention provides a method of generating a specific population of lymphocytes reactive with EpCAM by administration of a nucleic acid, polypeptide, vector, cell, or antibody (e.g., an anti-idiotype antibody) of the invention.

In particularly advantageous aspects, particular polypeptides, nucleic acids, cells, vectors, and/or antibodies of the invention induce a protective immune response against EpCAM-associated cancer cells in a host (e.g., a protective immune response against breast cancer tumor development when a polypeptide, nucleic acid, and/or vector of the invention is administered to the tissue (e.g., breast) of the host when EpCAM-associated micrometastases in the tissue (e.g., breast) are detected). The induction of a protective immune response against an EpCAM-associated cancer is determined, for example, by the lack of a disease condition(s) or symptom in a mammal upon or following treatment with the polypeptide, nucleic acid, vector, cell, and/or antibody of the invention at a stage where such disease conditions would normally develop (e.g., when an EpCAM associated micrometastases are detected). In other words, the invention provides a method of restricting tumor progression, tumor growth, and/or cancer progression (e.g., the spread of a cancer, the increase in the number of cancer cells, etc.) by administration of an immunogenic amount of an immunogenic polypeptide, nucleic acid, vector, antibody, and/or cell of the invention (e.g., at least one nucleic acid comprising a polynucleotide sequence having at least about 90, 95, 96, 97, 98, 99 or 100% sequence identity to a polynucleotide sequence selected from SEQ ID NOS:16, 19-23, 26-28, 33, and 35, at least one polypeptide comprising a polypeptide sequence having at least about 96, 97, 98, 99 or 100% sequence identity selected from the group consisting of SEQ ID NOS:14-10, 12-14, 32, 78 and 92, at least one vector or cell comprising at least one such nucleic acid or polypeptide, or at least one antibody induced in response to at least one such nucleic acid, polypeptide, cell, or vector).

A cancer cell is a cell that divides and reproduces abnormally with uncontrolled growth (e.g., by exceeding the “Hayflick limit” of normal cell growth (as described in, e.g., Hayflick, Exp. Cell Res. 37:614 (1965)). “Cancer progression” refers to any event or combination of events that promote, or which are indicative of, the transition of a normal, non-neoplastic cell to a cancerous, neoplastic cell. Examples of such events include phenotypic cellular changes associated with the transformation of a normal, non-neoplastic cell to a recognized pre-neoplastic phenotype and cellular phenotypic changes that indicate transformation of a pre-neoplastic cell to a neoplastic cell. Aspects of cancer progression (also referred to herein as “cancer progression stages”) include cell crisis, immortalization and/or normal apoptotic failure, proliferation of immortalized and/or pre-neoplastic cells, transformation (i.e., changes which allow the immortalized cell to exhibit anchorage-independent, serum-independent and/or growth-factor independent, or contact inhibition-independent growth, or that are associated with cancer-indicative shape changes, round up, aneuploidy, and focus formation), proliferation of transformed cells, development of metastatic potential, migration and metastasis (e.g., the disassociation of the cell from a location and relocation to another site), new colony formation, tumor formation, tumor growth, neotumorogenesis (formation of new tumors at a location distinguishable and not in contact with the source of the transformed cell(s)), or any combination thereof. The methods of the present invention can be used to reduce, treat, prevent, or otherwise ameliorate any suitable aspect of cancer progression. The methods of the invention are particularly useful in the reduction and/or amelioration of tumor growth and metastatic potential, as described further herein. Methods that reduce, prevent, or otherwise ameliorate such aspects of cancer progression are preferred. A particularly preferred aspect of the invention is the reduction of metastatic potential of cancer cells.

The detection of cancer progression can be achieved by any suitable technique, several examples of which are known in the art. Examples of suitable techniques include PCR and RT-PCR (e.g., of cancer cell associated genes or “markers”), biopsy, electron microscopy, positron emission tomography (PET), computed tomography, immunoscintigraphy and other scintegraphic techniques, magnetic resonance imaging (MRI), karyotyping and other chromosomal analysis, immunoassay/immunocytochemical detection techniques (e.g., differential antibody recognition), histological and/or histopathologic assays (e.g., of cell membrane changes), cell kinetic studies and cell cycle analysis, ultrasound or other sonographic detection techniques, radiological detection techniques, flow cytometry, endoscopic visualization techniques, and physical examination techniques. Examples of these and other suitable techniques are described in, e.g., Rieber et al., Cancer Res., 36(10), 3568-73 (1976), Brinkley et al., Tex. Rep. Biol. Med. 37:26-44 (1978), Baky et al., Anal. 151′ Quant. Cytol. 2(3):175-85 (1980), Laurence et al., Cancer Metastasis Rev. 2(4):351-74 (1983), Cooke et al., Gut 25(7):748-55 (1984), Kim et al., Yonsei Med. J. 26(2):167-74 (1985), Glaves, Prog. Clin. Biol. Res. 212:151-67 (1986), McCoy et al., Immunol. Ser. 53:171-87 (1990), Jacobsson et al., Med. Oncol. Tumor. Pharmacother. 8(4):253-60 (1991), Swierenga et al., IARC Sci. Publ. 165-93 (1992), Hirnle, Lymphology 27(3):111-3 (1994), Laferte et al., J. Cell Biochem. 57(1):101-19 (1995), Machiels et al., Eur. J. Cell Biochem. 66(3):282-92 (1995), Chaiwun et al., Pathology (Phila) 4(1):155-68 (1996), Jacobson et al., Ann. Oncol. 6(Suppl. 3):S3-8 (1996), Meijer et al., Eur. J. Cancer 31A(7-8):1210-11 (1995), Greenman et al., J. Clin. Endocrinol. Metab. 81(4), 1628-33 (1996), Ogunbiyi et al. Ann. Surg. Oncol. 4(8):613-20 (1997), Merritt et al., Arch. Otolaryngol. Head Neck Surg. 123(2):149-52 (1997), Bobardieri et al., Q. J. Nucl. Med. 42(1):54-65 (1998), Giordano et al., J. Cell Biochem. 70(1):1-7 (1998), Siziopikou et al., Breast J. 5(4):221-29 (1999), Rasper, Surgery 126(5):827-8 (1999), von Knebel et al., Cancer Metastasis Rev. 18(1):43-64 (1999), Britton et al., Recent Results Cancer Res. 157:3-11 (2000), Caraway et al., Cancer 90(2):126-32 (2000), Castillo et al., Am. J. Neuroadiol. 21(5):948-53 (2000), Chin et al., Mayo Clin. Proc. 75(8):796-801 (2000), Kau et al., J. Ortohinolaryngol. Relat. Spe. 62(4):199-203 (2000), Krag, Cancer J. Sci. Am., 6 (Suppl. 2):S121-24 (2000), Pantel et al., Curr. Opin. Oncol. 12(1):95-101 (2000), Cook et al., Q. J. Nucl. Med. 45(1):47-52 (2001), Gambhir et al., Clin. Nucl. Med. 26(10):883-4 (2001), MacManus et al., Int. J. Radiat. Oncol. Biol. Phys. 50(2):287-93 (2001), Olilla et al., Cancer Control. 8(5):407-14 (2001), Taback et al., Recent Results Cancer Res. 158:78-92 (2001), and references cited therein. Related techniques are described in U.S. Pat. Nos. 6,294,343, 6,245,501, 6,242,186, 6,235,486, 6,232,086, 6,228,596, 6,200,765, 6,187,536, 6,080,584, 6,066,449, 6,027,905, 5,989,815, 5,939,258, 5,882,627, 5,829,437, 5,677,125, and 5,455,159 and International Patent Application Publ. Nos. WO 01/69199, WO 01/64110, WO 01/60237, WO 01/53835, WO 01/48477, WO 01/04353, WO 98/12564, WO 97/32009, WO 97/09925, and WO 96/15456.

A reduction of cancer progression can be any detectable decrease in (1) the rate of normal cells transforming to neoplastic cells (or any aspect thereof), (2) the rate of proliferation of pre-neoplastic or neoplastic cells, (3) the number of cells exhibiting a pre-neoplastic and/or neoplastic phenotype, (4) the physical area of a cell media (e.g., a cell culture, tissue, or organ (e.g., an organ in a mammalian host)) comprising pre-neoplastic and/or neoplastic cells, (5) the probability that normal cells will transform to neoplastic cells, (6) the probability that cancer cells will progress to the next aspect of cancer progression (e.g., a reduction in metastatic potential), or (7) any combination thereof. Such changes can be detected using any of the above-described techniques or suitable counterparts thereof known in the art, which are applied at a suitable time prior to the administration of the nucleic acid, polypeptide, vector, cell, and/or antibody of the invention. Times and conditions for assaying whether a reduction in cancer potential has occurred will depend on several factors including the type of cancer, type and amount of novel biological agents (biomolecules) or cells administered or expressed, and the cancer progression stage assayed for. The ordinarily skilled artisan will be able to make appropriate determinations of times and conditions for performing such assays using techniques known in the art and/or routine experimentation.

In one aspect, the invention provides therapeutic and/or prophylactic methods to reduce the cancer progression of any suitable type of cancer associated with EpCAM, EpCAM homologs, and/or EpCAM orthologs. For example, the invention provides a therapeutic method of reducing progression of a cancer in a subject in need of such treatment, said method comprising administering to the subject an effective amount of at least one nucleic acid or polypeptide of the invention, including, e.g., a nucleic acid comprising a polynucleotide sequence having at least about 90, 95, 96, 97, 98, 99, or 100% sequence identity to a polynucleotide sequence selected from the group consisting of SEQ ID NOS: 16, 19-23, 26-28, 33, 35, 79, and 94, or a polypeptide comprising a polypeptide sequence having at least about 96, 97, 98, 99, or 100% sequence identity to a polypeptide sequence selected from the group of SEQ ID NOS:1, 4-10, 12-14, 32, 34, 78, and 92, wherein the effective amount is an amount sufficient to effectively reduce progression of the cancer. Advantageously, such methods are useful in reducing cancer progression in prostate cancer cells, breast cancer cells, colon cancer cells, colorectal cancer cells, and lung cancer cells. Such methods are also useful in reducing cancer progression in both tumorigenic and non-tumorigenic cancers (e.g., non-tumor-forming hematopoietic cancers and/or dormant micrometastatic cancer cells).

In another aspects, such methods are useful in reducing tumor progression in a prostate tumor cells, breast tumor cells, colon tumor cells, colorectal tumor cells, and lung tumor cells.

In another aspect, the invention provides a therapeutic method of extending the mean or median time to recurrence of EpCAM-associated, EpCAM homolog-associated, or EpCAM-ortholog associated detectable tumor progression, cancer progression, and/or cancer/tumor-associated disease in a mammalian host, which method comprises administering a suitably immunogenic amount of a polypeptide, cell, nucleic acid, antibody, or vector of the invention to the host such that an immune response to EpCAM is generated, which immune response extends the mean or median time to recurrence of such progression or disease. Notably, immunosuppressed subjects may not be candidates for such therapy.

TAg-encoding nucleic acids (including vectors) and TAg antigens of the invention are expected to delay the occurrence of metastatic disease in subjects with EpCAM/KSA+ malignancies, such as stage II and III colon cancers. Such subjects may be undergoing surgical resection for staging and/or cure. Combination therapies that employ at least one TAg-encoding nucleic acid (e.g., a nucleic acid comprising a polynucleotide sequence having at least about 90, 95, 96, 97, 98, 99 or 100% sequence identity to a polynucleotide sequence selected from SEQ ID NOS:16, 19-23, 26-28, 33, 35, and 79, or a vector comprising at least one such nucleic acid) and/or at least one TAg antigen (e.g., a polypeptide comprising a polypeptide sequence having at least about 96, 97, 98, 99 or 100% sequence identity selected from the group consisting of SEQ ID NOS:14-10, 12-14, 32, 78 and 92) in combination with one or more costimulatory molecules, such as CD28BP-15 (discussed above and in detail below) are also expected to provide therapeutic effects for subjects when used for treating EpCAM/KSA-associated tumors, including significantly prolonging the progression of metastatic disease associated with such tumors or the median time to recurrence of such disease or tumors in subjects suffering from such disease or tumors. TAg-encoding nucleic acids (or vectors comprising such nucleic acids) and/or TAg antigens of the invention reduce the spread of malignant cells in the perioperative period for such subjects. Cytotoxic T cells and specific antibodies induced by TAg-encoding nucleic acids (including vectors) and/or TAg antigens are expected to lyse such tumor cells, thereby destroying such cells, and/or neutralize function, thereby providing anti-tumor effects (cell adhesion molecule; ligand for leukocyte-associated Ig-like receptor). Given that TAg polypeptides of the invention, administered as polypeptides or as nucleic acids that expressed such polypeptides induced or enhanced production of antibodies against human EpCAM antigen and specific CD8 T cells in at least cynomolgus monkeys, TAg polypeptides are expected to provide improvements to the therapy of colorectal cancer and to the quality and length of life of colorectal cancer patients.

In a further aspect, the invention provides a method of prolonging the survival of a human suffering from an EpCAM-associated cancer, which method comprises administering a suitably immunogenic or effective amount of at least one polypeptide, nucleic acid, vector, cell, and/or antibody of the invention to the host that induces an immune response against hEpCAM (e.g., at least one nucleic acid comprising a polynucleotide sequence having at least about 90, 95, 96, 97, 98, 99 or 100% sequence identity to a polynucleotide sequence selected from SEQ ID NOS:16, 19-23, 26-28, 33, 35, and 79, at least one polypeptide comprising a polypeptide sequence having at least about 96, 97, 98, 99 or 100% sequence identity selected from the group consisting of SEQ ID NOS:14-10, 12-14, 32, 78 and 92, at least one vector or cell comprising at least one such nucleic acid or polypeptide, or at least one antibody induced in response to at least one such nucleic acid, polypeptide, cell, or vector), such that an immune response is induced against hEpCAM (particularly against hEpCAM-associated cancer cells), which immune response prolongs the survival of the human. The amount is the amount effective in induced an immune response that prolongs survival of the human.

The invention also provides a prophylactic method of preventing (i.e., reducing the likelihood of, occurrence of, and/or time to onset of occurrence of) metastasis in a human treated for surgical cancer (e.g., colorectal cancer, breast cancer, or liver cancer). The method comprises administering to a human a therapeutic amount of at least nucleic acid, polypeptide, vector, cell, and/or antibody of the invention effective in inducing an immune response against hEpCAM (e.g., at least one nucleic acid comprising a polynucleotide sequence having at least about 90, 95, 96, 97, 98, 99 or 100% sequence identity to a polynucleotide sequence selected from SEQ ID NOS:16, 19-23, 26-28, 33, 35, and 79, at least one polypeptide comprising a polypeptide sequence having at least about 96, 97, 98, 99 or 100% sequence identity selected from the group consisting of SEQ ID NOS: 14-10, 12-14, 32, 78 and 92, at least one vector or cell comprising at least one such nucleic acid or polypeptide, or at least one antibody induced in response to at least one such nucleic acid, polypeptide, cell, or vector), such that an immune response is induced against hEpCAM in the subject for a suitable period of time and under suitable conditions such that metastasis is prevented. As with many therapeutic or prophylactic methods of the invention, this method typically is practiced by a prime-boosting administration strategy using one or more different nucleic acid(s), vector(s), polypeptide(s) and/or antibodies of the invention administered in one or more administrations in sequential format at suitable time periods for optimum treatment or enhancement of the immune response (e.g., administration of an effective amount a pMaxVax DNA vector prime followed by administration of an effective amount of one or more protein, anti-idiotype antibody, and/or viral vector particle prime boosts).

In yet another aspect, the invention provides a therapeutic method of treating, stabilizing or improving the clinical prognosis of a cancer in a human, such as a surgically treated colorectal cancer patient, which method comprises administering a suitably immunogenic or therapeutically effective amount of a polypeptide, nucleic acid, vector, cell, and/or antibody of the invention as described above to the human in need of such treatment patient, wherein said immunogenic or therapeutically effective amount is sufficient to induce an immune response in the human against hEpCAM, such that the clinical prognosis of the cancer patient is detectably treated, stabilized or improved. Preferably, the administration of such biomolecules or cells of the invention prevents the recurrence of a recognized disease state in a patient treated for an hEpCAM-associated cancer.

In a further aspect, the invention provides a therapeutic method of inducing regression of an hEpCAM-associated cancer in a human, by the administration to a human subject in need of such treatment an immunogenic or therapeutically effective amount of at least one of the TAg-encoding nucleic acids or TAg polypeptides that induces an immune response against hEpCAM (or cells or vectors comprising at least one such nucleic acid or polypeptide), wherein the immunogenic or therapeutically effective amount is sufficient to induce an immune response and/or regression of the hEpCAM-associated cancer in the human subject.

The invention also provides a therapeutic method of inducing an immune response against an mEpCAM, particularly against hEpCAM-overexpressing neoplastic cells, in a host, while also enhancing T cell activation through CD28 signaling, said method comprising co-administration to a subject in need of such treatment (e.g., having hEpCAM-overexpressing neoplastic cells) of: (1) an effective amount of at least one polypeptide, nucleic acid, cell, vector, or antibody of the invention, wherein said effective amount is sufficient to induce an immune response against hEpCAM, such that said immune response is induced; and (2) an effective amount of at least one suitable costimulatory polypeptide (or nucleic acid expressing at least one such costimulatory polypeptide), wherein said effective amount is sufficient to enhance said immune response. The costimulatory polypeptide preferably is a CD28 binding protein and most preferably a novel costimulatory molecule CD28 binding protein (“CD28BP”), such as CD28BP-15 (see, e.g., Int'l Patent App. Nos. PCT/US01/19973 (WO 02/00717) and PCT/US02/19898). Such co-administration can comprise simultaneous administration or administration in series, which series administration can comprise a period of time between administration, limited to a period shorter than the maximum time at which the co-administration of the respective molecules would not exhibit a combined, cooperative, or other associated effect with one another. Such polypeptide, nucleic acid, cell, vector, or antibody of the invention includes at least one nucleic acid comprising a polynucleotide sequence having at least about 90, 95, 96, 97, 98, 99 or 100% sequence identity to a polynucleotide sequence selected from SEQ ID NOS:16, 19-23, 26-28, 33, 35, and 79, at least one polypeptide comprising a polypeptide sequence having at least about 96, 97, 98, 99 or 100% sequence identity selected from the group consisting of SEQ ID NOS:14-10, 12-14, 32, 78 and 92, at least one vector or cell comprising at least one such nucleic acid or polypeptide, or at least one antibody induced in response to at least one such nucleic acid, polypeptide, cell, or vector.

It is to be understood that any of the methods described herein with reference to nucleic acids, vectors, cells, polypeptides, and antibodies of the invention apply equally with reference to compositions comprising such novel biological molecules, which compositions are described elsewhere herein.

Immune responses generated or induced by the polypeptides, nucleic acids, cells, antibodies, and/or vectors of the invention can be measured by any suitable technique. Examples of useful techniques in assessing humoral immune responses include flow cytometry, immunoblotting (detecting membrane-bound proteins), including dot blotting, immunohistochemistry (cell or tissue staining), enzyme immunoassays, immunoprecipitation, immunohistochemistry, RIA (radioimmunoassay), and other EIAs (enzyme immunoassays), such as ELISA (enzyme-linked immunosorbent assay including sandwich ELISA and competitive ELISA) and ELIFA (enzyme-linked immunoflow assay). ELISA assays involve the reaction of a specific first antibody with an antigen. The resulting first antibody-antigen complex is detected by using a second antibody against the first antibody; the second antibody is enzyme-labeled and an enzyme-mediated color reaction is produced by reaction with the first antibody. Suitable antibody labels for such assays include radioisotopes; enzymes, such as horseradish peroxidase (HRP) and alkaline phosphatase (AP); biotin; and fluorescent dyes, such as fluorescein or rhodamine. Both direct and indirect immunoassays can be used in this respect. HPLC and capillary electrophoresis (CE) also can be utilized in immunoassays to detect complexes of antibodies and target substances. General guidance performing such techniques and related principles are described in, e.g., Harlow and Lane (1988) ANTIBODIES, A LABORATORY MANUAL, Cold Spring Harbor Publications, New York, Hampton R et al. (1990) SEROLOGICAL METHODS A LABORATORY MANUAL, APS Press, St. Paul Minn., Stevens (1995) CLINICAL IMMUNOLOGY AND SEROLOGY A LABORATORY PERSPECTIVE, CRC press, Bjerrum (1988) HANDBOOK OF IMMUNOBLOTTING OF PROTEINS, Vol. 2, Zoa (1995) DIAGNOSTIC IMMUNOPATHOLOGY: LABORATORY PRACTICE AND CLINICAL APPLICATION, Cambridge University Press, Folds (1998) CLINICAL DIAGNOSTIC IMMUNOLOGY: PROTOCOLS IN QUALITY ASSURANCE AND STANDARDIZATION, Blackwell Science Inc., Bryant (1992) LABORATORY IMMUNOLOGY & SEROLOGY 3rd edition, W B Saunders Co., and Maddox et al. (1983) J. Exp. Med. 158:1211. Specific guidance with respect to ELISA techniques and related principles are described in, e.g., Reen (1994) Methods Mol. Biol. 32:461-6, Goldberg et al. (1993) Curr. Opin. Immunol. 5(2):278-81, Voller et al. (1982) Lab. Res. Methods Biol. Med. 5:59-81, Yolken et al. (1983) Ann. NY Acad. Sci. 420:381-90, Vaughn et al. (1999) Am. J. Trop. Med. Hyg. 60(4):693-8, and Kuno et al. J. Virol. Methods (1991) 33(1-2):101-13. Guidance with respect to Western blot techniques can found in, e.g., Ausubel et al., CURRENT PROTOCOLS IN MOLECULAR BIOLOGY (Wiley Interscience Publishers 1995). Specific exemplary applications of Western blot techniques can be found in, e.g., Churdboonchart et al. (1990) Southeast Asian J. Trop. Med. Public Health 21(4):614-20 and Dennis-Sykes et al. (1985) J. Biol. Stand 13(4):309-14. Specific guidance with respect to flow cytometry techniques is provided in, e.g., Diamond (2000) IN LIVING COLOR: PROTOCOLS IN FLOW CYTOMETRY AND CELL SORTING, Springer Verlag, Jaroszeki (1998) FLOW CYTOMETRY PROTOCOLS, 1st Ed., Shapiro (1995) PRACTICAL FLOW CYTOMETRY, 3rd edition, Rieseberg et al. (2001) Appl. Microbiol. Biotechnol. 56(3-4):350-60, Scheffold and Kern (2000) J. Clin. Immunol. 20(6):400-7, and McSharry (1994) Clin. Microbiol. Rev. (4):576-604.

Briefly, a Western blot assay may be performed by attaching a recombinant antigen, such as a recombinant polypeptide of the invention, EpCAM, EpCAM homolog, EpCAM ortholog, or other antigenic polypeptide, to a nitrocellulose paper and staining with an antibody which has a dye attached. Among the methods using a reporter enzyme is the use of a reporter-labeled antihuman antibody. The label may be an enzyme, thus providing an enzyme-linked immunosorbent assay (ELISA). It also may be a radioactive element, thus providing a radioimmunoassay (RIA).

Cytotoxic and other T cell immune responses also can be measured by any suitable technique. Examples of such techniques include ELISpot assay (particularly, IFN-γ ELISpot), intracellular cytokine staining (ICC) (particularly in combination with FACS analysis), CD8+ T cell tetramer staining/FACS, standard and modified T cell proliferation assays, chromium release CTL assay, limiting dilution analysis (LDA), and CTL killing assays. Guidance and principles related to T cell proliferation assays are described in, e.g., Plebanski and Burtles (1994) J. Immunol. Meth. 170:15, Sprent et al. (2000) Philos. Trans R. Soc. Lond. B. Biol. Sci. 355(1395):317-22, and Messele et al. (2000) Clin. Diagn. Lab. Immunol. 7(4):687-92. LDA is described in, e.g., Sharrock et al. (1990) Immunol. Today 11:281-286. ELISpot assays and related principles are described in, e.g., Czerinsky et al. (1988) J. Immunol. Methods 110:29-36, Olsson et al. (1990) J. Clin. Invest 86:981-985, Schmittel et al. (2001) J. Immunol. Methods 247(1-2):17-24, Ogg and McMichael (1999) Immunol. Lett. 66(1-3):77-80, Schmittel et al. (2001) J. Immunol. Methods 247(1-2):17-24, Kurane et al. (1989) J. Exp. Med. 170(3):763-75, Chain et al. (1987) J Immunol Methods 99(2):221-8, and Czerkinsky et al. (1988) J. Immunol. Meth. 110:29-36, as well as U.S. Pat. Nos. 5,750,356 and 6,218,132. Tetramer assays are discussed in, e.g., Skinner et al. (2000) J. Immunol. 165(2):613-7. Other T cell analytical techniques are described in Hartel et al. (1999) Scand. J. Immunol. 49(6):649-54 and Parish et al. (1983) J. Immunol. Methods 58(1-2):225-37.

T cell activation or proliferation also can be analyzed by measuring CTL activity or expression of activation antigens such as IL-2 receptor, CD69 or HLA-DR molecules. Proliferation of purified T cells can be measured in a mixed lymphocyte culture (MLC) assay. MLC assays are known in the art. Briefly, a mixed lymphocyte reaction (MLR) is performed using irradiated peripheral blood monocyte cells (PBMC) as stimulator cells and allogeneic PBMC as responders. Stimulator cells are irradiated (2500 rads) and co-cultured with allogeneic PBMC (1×10⁵ cells/well) in 96-well flat-bottomed microtiter culture plates (VWR) at 1:1 ratio for a total of 5 days. During the last 8 hours of the culture period, the cells are pulsed with 1 uCi/well of ³H-thymidine, and the cells are harvested for counting onto filter paper by a cell harvester as described above. ³H-thymidine incorporation is measured by standard techniques. Proliferation of T cells in such assays is expressed as the mean counts per minute (cpm) read for the tested wells.

ELISpot assays measure the number of T-cells secreting a specific cytokine, such as interferon-gamma or tumor necrosis factor-alpha, that serves as a marker of T-cell effectors. Cytokine-specific ELISA kits are commercially available (e.g., an IFN-γ-specific ELISPot is available through R&D Systems, Minneapolis, Minn.). ELISpot assays are further described in the Examples section.

Other techniques for assessing immune response of the polypeptide, nucleic acid, vector, cell, and/or antibody of the invention include Granzyme B assays, which measure CTL activation, CD4+ T cell proliferation assays, assays that identify specific T cells in peripheral blood, the measurement of micrometastasis in peripheral blood, and determining levels of cancer markers in a human (e.g., EpCAM expression levels). Similar methods are further discussed elsewhere herein. Delayed-type hypersensitivity reaction assays (DTH assays), which are commonly performed at the site of injection of a nucleic acid or polypeptide composition of the invention, also can be important to assessing the therapeutic usefulness of a particular composition of the invention.

In another aspect, the invention provides a polypeptide having an immunogenic polypeptide sequence of the invention (e.g., a fragment of SEQ ID NO:4 or SEQ ID NO:5 of at least about 45 amino acid residues or a polypeptide sequence that has at least about 85, 90, 95, 96, 97, 98 or 99% identity to a fragment of SEQ ID NO:4, which fragment is at least about 45 amino acids in length), which immunogenic amino acid sequence comprises at least one T cell epitope, which portion forms a peptide-MHC complex (e.g., a peptide-HLA complex) when processed in a mammalian cell with an IC₅₀ (50% inhibitory concentration) of at least about 3 μm and a DT₅₀ (time to 50% disintegration) of at least about 2 hours, and wherein the polypeptide induces an immune response against an mEpCAM.

In one aspect, such a polypeptide of the invention also or alternatively will comprise at least one (e.g., 2, 3, 4, or more) epitopes that have a Parker score of at least about 50 and/or a Rammensee score of at least about 10 (see, e.g., Trojan et al., Cancer Res., 61:4761-4765 (2001) for discussion of such measurements). Advantageously, polypeptides of the invention will comprise 2, 3, 4, or more of such T cell epitopes. Techniques for measuring the IC₅₀ and DT₅₀ of peptide-MHC complexes are known in the art (see, e.g., Ras et al., Human Immunol. 53:81-89 (1997)).

The invention also provides a method of inducing an immune response in a mammalian host against mEpCAM-associated cells (and preferably reducing the number of EpCAM-associated cells) that express a mutated gene associated with cancer progression (e.g., a mutated ras or p53 gene) or that overexpress a cancer antigen (e.g., CEA), which method comprises administering to the host possessing such cells an immunogenic amount or effective amount of at least one nucleic acid, polypeptide, cell, vector, and/or antibody of the invention that has the ability to induce such immune response against hEpCAM as described above. The immunogenic or effective amount is the amount sufficient to induce such an immune response in the host. Such cell factors also can serve as markers for measuring the reduction in the number of cancer cells in a host, brought about by the therapeutic methods of the invention. Alternatively, techniques can be employed that assess the number of EpCAM-overexpressing cells and/or that identify EpCAM-expressing cells that have neoplastic, transformed, and/or cancerous morphological and/or physiological characteristics.

For example, the invention provides a therapeutic method of inducing an immune response against hEpCAM in a human, and particularly against hEpCAM-overexpressing neoplastic or otherwise cancerous cells, comprising administering to the human in need of such treatment a first effective dose of a nucleic of the invention that induces an immune response against EpCAM (e.g., a polynucleotide sequence having at least about 90, 95, 96, 97, 98, 99 or 100% sequence identity to a polynucleotide sequence selected from SEQ ID NOS:16, 19-23, 26-28, 33, and 35), and permitting expression of the nucleic acid in the human, such that an immunogenic amount of a polypeptide of the invention is expressed in the human, thereby inducing a sufficient immune response against EpCAM and, consequently, against such EpCAM-overexpressing cells.

Such polypeptide, nucleic acid, cell, vector, or antibody of the invention includes at least one nucleic acid comprising a polynucleotide sequence having at least about 90, 95, 96, 97, 98, 99 or 100% sequence identity to a polynucleotide sequence selected from SEQ ID NOS:16, 19-23, 26-28, 33, 35, and 79 at least one polypeptide comprising a polypeptide sequence having at least about 96, 97, 98, 99 or 100% sequence identity selected from the group consisting of SEQ ID NOS: 14-10, 12-14, 32, 78 and 92, at least one vector or cell comprising at least one such nucleic acid or polypeptide, or at least one antibody induced in response to at least one such nucleic acid, polypeptide, cell, or vector.

In yet another aspect, the invention provides a method of limiting the tumor burden of a host, comprising administering at least one polypeptide, nucleic acid, cell, vector, or antibody of the invention, as described above, to such host in an effective amount such that the tumor burden is limited in the host.

The methods of the invention can be applied in vitro, in vivo, and in ex vivo contexts. For example, human colorectal cancer cells, such as cells of the cell line HT29, can serve as a useful in vitro model for assessing the immunogenicity of a polypeptide of the invention (e.g., a polypeptide comprising a polypeptide sequence having at least about 96, 97, 98, or 99% sequence identity to a sequence selected from SEQ ID NOS: 1, 4, and 5). Examples of additional suitable cancer cells for such in vitro methods are described in, e.g., the ATCC catalog, an electronic copy of which is available at http://www.atcc.org/pdf/tcl.pdf.

While a single dose of a nucleic acid, polypeptide, and/or vector of the invention can be suitable for inducing an immune response against an mEpCAM, therapeutic methods of the invention typically comprise one or more repeat administrations of the same or different nucleic acid, polypeptide, and/or vector of the invention. Thus, for example, the invention provides a therapeutic method of inducing an immune response against hEpCAM in a subject, which method comprises administering to the host in need of such treatment a first effective dose (immunogenic amount sufficient to induce an immune response) of at least one nucleic acid, polypeptide, and/or vector of the invention that is capable of inducing an immune response against hEpCAM, such that an immune response against hEpCAM is induced in the host, and subsequently introducing a second effective dose (immunogenic amount sufficient to induce an immune response) of at least one nucleic acid, polypeptide, and/or vector of the invention that is capable of inducing an immune response against hEpCAM, such that the immune response against hEpCAM is increased in the host over the first immune response without such second dose.

In some aspects, the dosage of nucleic acid, polypeptide, and/or vector in the first dose is repeated (i.e., the same vector, nucleic acid, and/or polypeptide of the invention is re-administered at a time after the first administration, such that the immune response against mEpCAM is enhanced). Alternatively, the second dose can be in a different form and/or amount than the first dose, or a different vector, nucleic acid, and/or polypeptide of the invention is administered. For example, in one aspect, administration of a naked DNA of the invention is followed by a polypeptide (e.g., a polypeptide encoded by said DNA) and/or viral vector boost (e.g., a viral vector comprising said DNA or polypeptide). More particular examples of such combined administration (or boosting) strategies are provided below. In further aspects, an additional effective dose (third effective dose), two additional doses, three additional doses, or more effective doses (e.g., a third, fourth, fifth, and sixth effective doses) of at least one nucleic acid, polypeptide, and/or vector of the invention are administered to the host, thereby increasing the resulting immune response against mEpCAM that is observed in the host. Such second, third, or further effective doses are advantageously provided to the human after the first effective dose and at a time such that the immune response to mEpCAM in the subject is enhanced as compared to if the second effective dose had not been provided.

In another aspect, the invention provides a therapeutic method of inhibiting human EpCAM:ligand interactions (including, e.g., EpCAM:EpCAM interactions, where an EpCAM molecule acts as a ligand through binding to another EpCAM molecule) in a human comprising administering to the human subject an effective amount of at least one polypeptide, nucleic acid, or vector of the invention that is capable of inducing an immune response against hEpCAM as described above, or a combination of any thereof, wherein the effective amount is an amount sufficient to detectably inhibit hEpCAM:ligand interactions, such that hEpCAM:ligand interactions are detectably inhibited in the human. In one aspect, such inhibition may result from binding of at least one polypeptide of the invention or (a polypeptide expressed from a nucleic acid or vector of the invention) to hEpCAM.

In another aspect, the invention provides a therapeutic method of inhibiting human EpCAM:ligand interactions (including, e.g., EpCAM:EpCAM interactions) in a human subject comprising administering to the human in need of such treatment at least one antibody of the invention, such as a monoclonal antibody of the invention that is induced in response to administration of a TAg-encoding nucleic acid or TAg polypeptide of the invention, in an effective amount and manner such that EpCAM:ligand interactions are detectably inhibited in the human. In one aspect, such inhibition may result from binding of at least one polypeptide of the invention or (a polypeptide expressed from a nucleic acid or vector of the invention) to hEpCAM.

In general, administration of a polypeptide, nucleic acid, and/or vector of the invention is typically employed when an immune response against a tumor is desired, whereas administration of one or more antibodies of the invention is typically used for treatment of small tumors or micrometastatic cells, tissues, and/or growths, since oncotic pressure in tumors can prevent effective circulation of antibodies in the metastatic lesion or other target area(s) in which the immune response to mEpCAM is desired.

Similarly, the invention provides a method of reducing, inhibiting, stopping, or regressing tumor progression, cancer progression, and/or neoplastic cell development and/or population growth in a subject in need of such treatment by administering an effective amount of an antibody of the invention or composition thereof to the subject. The effective amount is the amount sufficient in reducing, inhibiting, stopping, or regressing such tumor or cancer progression, or neoplastic cell development and/or population growth. Monoclonal antibodies of the invention, as described elsewhere herein, can be particularly useful in the reduction of cancer progression in a subject suffering from early stage EpCAM-associated cancers (e.g., cancer of breast, pancreas, lung, liver, rectum, colon, oral or other mucosa, and epithelial tissues, such as the gut (see, e.g., Balzar, supra). Techniques for the therapeutic administration of EpCAM antibodies can, by analogy, be applied to the novel antibodies of the invention described herein (see, e.g., Schwartzberg Critical Reviews in Oncology Hematology 40:17-24 (2001) and Clinical Cancer Research 5:399-4004 (1999) for discussion of such techniques).

Also provided are methods for inducing an immune response against EpCAM in a subject which comprise administering to the subject a population of recombinant cells of the invention that express a nucleic acid of the invention, either by an integrated or episomal nucleic acid contained therein, or a vector within such cells, in an ex vivo manner to induce an immune response to EPCAM in the host. Similarly, cells (e.g., dendritic cells) that express an immunogenic polypeptide that is associated with a transmembrane domain on the surface thereof can be administered to a subject or population of cells to induce an immune response against EpCAM.

In one aspect, a polypeptide, nucleic acid, antibody, and/or vector of the invention is administered via a composition comprising said polypeptide, nucleic acid, antibody, and/or vector and a suitable carrier or excipient. Preferably, the composition is a pharmaceutical composition and the carrier or excipient is a pharmaceutically acceptable carrier or excipient as described further herein.

An injectable, pharmaceutical composition comprising a suitable, pharmaceutically acceptable carrier (e.g., PBS) and an immunogenic amount of a polypeptide of the invention can be administered intramuscularly, intraperitoneally, subdermally, transdermally, subcutaneously, or intradermally to the host for in vivo. Alternatively, biolistic protein delivery techniques (vaccine gun delivery) can be used (examples of which are discussed elsewhere herein) for administration of a polypeptide of the invention. Any other suitable technique also can be used. Polypeptide administration can also be facilitated via liposomes (examples of which are further discussed herein).

While the following discussion is primarily directed to nucleic acids, it will be understood that it applies equally to nucleic acid vectors of the invention. A nucleic acid of the invention or composition thereof can be administered to a host by any suitable administration route. In some aspects of the invention, administration of the nucleic acid is parenteral (e.g., subcutaneous, intramuscular, or intradermal), topical, or transdermal. The nucleic acid can be introduced directly into a tissue, such as muscle, by injection using a needle or other similar device. See, e.g., Nabel et al. (1990), supra); Wolff et al. (1990) Science 247:1465-1468), Robbins (1996) Gene Therapy Protocols, Humana Press, NJ, and Joyner (1993) Gene Targeting: A Practical Approach, IRL Press, Oxford, England, and U.S. Pat. Nos. 5,580,859 and 5,589,466. Other methods such as “biolistic” or particle-mediated transformation (see, e.g., U.S. Pat. No. 4,945,050, U.S. Pat. No. 5,036,006, Sanford et al., J. Particulate Sci. Tech. 5:27-37 (1987), Yang et al., Proc. Natl. Acad. Sci. USA 87:9568-72 (1990), and Williams et al., Proc. Natl. Acad. Sci. USA 88:2726-30 (1991)). These methods are useful not only for in vivo introduction of DNA into a subject, such as a mammal, but also for ex vivo modification of cells for reintroduction into a mammal (which is discussed further elsewhere herein).

For standard gene gun administration, the vector or nucleic acid of interest is precipitated onto the surface of microscopic metal beads. The microprojectiles are accelerated with a shock wave or expanding helium gas, and penetrate tissues to a depth of several cell layers. For example, the Accel™ Gene Delivery Device manufactured by Agacetus, Inc. Middleton Wis. is suitable for use in this embodiment. The nucleic acid or vector can be administered by such techniques, e.g., intramuscularly, intradermally, subdermally, subcutaneously, and/or intraperitoneally. Additional devices and techniques related to biolistic delivery International Patent Applications WO 99/2796, WO 99/08689, WO 99/04009, and WO 98/10750, and U.S. Pat. Nos. 5,525,510, 5,630,796, 5,865,796, and 6,010,478,

The nucleic acid can be administered in association with a transfection-facilitating agent, examples of which were discussed above. The nucleic acid can be administered topically and/or by liquid particle delivery (in contrast to solid particle biolistic delivery). Examples of such nucleic acid delivery techniques, compositions, and additional constructs that can be suitable as delivery vehicles for the nucleic acids of the invention are provided in, e.g., U.S. Pat. Nos. 5,591,601, 5,593,972, 5,679,647, 5,697,901, 5,698,436, 5,739,118, 5,770,580, 5,792,751, 5,804,566, 5,811,406, 5,817,637, 5,830,876, 5,830,877, 5,846,949, 5,849,719, 5,880,103, 5,922,687, 5,981,505, 6,087,341, 6,107,095, 6,110,898, and International Patent Applications WO 98/06863, WO 98/55495, and WO 99/57275.

The choice of administration/delivery technique and the form of the antigenic polypeptide of the invention, such as a TAg antigen (or polynucleotide encoding such antigen), can influence the type of immune response observed upon administration. For example, gene gun delivery of many antigens is associated with a Th2-biased response (indicated by higher IgG1 antibody titers and comparatively low IgG2a titers). The bias of a particular immune response enables the physician or artisan to direct the immune response promoted by administration of the polypeptide and/or polynucleotide of the invention.

Alternatively, the nucleic acid can be administered to the host by way of liposome-based gene delivery. Exemplary techniques and principles related to liposome-based gene delivery is provided in, e.g., Debs and Zhu (1993) WO 93/24640; Mannino and Gould-Fogerite (1988) BioTechniques 6(7):682-691; Rose U.S. Pat. No. 5,279,833; Brigham (1991) WO 91/06309; Brigham et al. (1989) Am J Med Sci 298:278-281; Nabel et al. (1990) Science 249:1285-1288; Hazinski et al. (1991) Am J Resp Cell Molec Biol 4:206-209; and Wang and Huang (1987) Proc. Natl. Acad. Sci. USA 84:7851-7855), and Felgner et al. (1987) Proc. Natl. Acad. Sci. USA 84:7413-7414). Suitable liposome pharmaceutically acceptable compositions that can be used to deliver the nucleic acid are further described elsewhere herein.

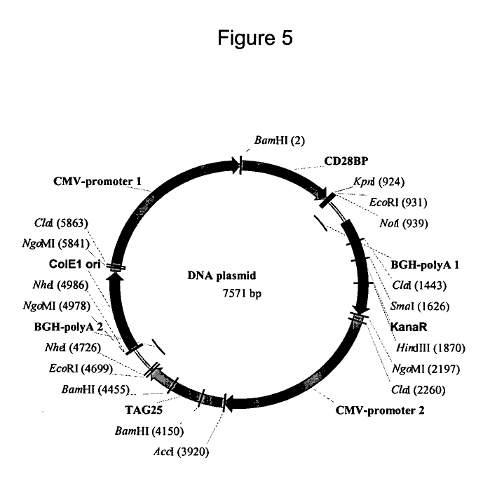

Any immunogenic amount of nucleic acid can be used in the methods of the invention. Typically, where the nucleic acid is administered by injection, about 50 micrograms (μg) to 10 mg, about 1 mg to 8, about 2 mg to about mg, about 100 μg to about 2.5 mg, typically about 500 μg to about 2 mg or about 800 μg to about 1.5 mg, and often about 2 mg or about 1 mg is administered. In one exemplary application, to induce an immune response against hEpCAM or EpCAM-overexpressing cells, e.g., a pharmaceutical comprising PBS and 10 mg of a bicistronic DNA vector encoding TAg-25 (SEQ ID NO:4) and CD28BP-15 polypeptides is administered by injection to a human subject in need of treatment (e.g., a human having an EpCAM-associated tumor or EpCAM-expressing cancer). An exemplary vector is shown in FIG. 5. Alternatively, two separate vectors are administered by injection: (1) 5 mg of a monocistronic DNA vector encoding TAg-25 (SEQ ID NO:4); and (2) 5 mg of a monocistronic DNA vector encoding CD28BP-15 polypeptide. These vectors can be delivered in together in one composition comprising both DNA vectors and PBS or, consecutively, in two compositions, each comprising one DNA vector and PBS. If desired, following administration of the DNA vector(s), a protein boost can be administered by injection to enhance the immune response; e.g., a composition comprising PBS (or other carrier) and 500 micrograms of TAg-25 (SEQ ID NO:4) is administered.

The amount of DNA plasmid for use in the methods of the invention where administration is via a gene gun, e.g., is often from about 100 to about 1000 times less than the amount used for direct injection (e.g., via standard needle injection). Despite such sensitivity, preferably at least about 1 μg of the nucleic acid is used in such biolistic delivery techniques.

Methods of the invention are practiced with a dosage of a suitable viral vector. Any suitable viral vector in any suitable concentration of viral particles can be used. For example, the mammalian host can be administered a population of retroviral vectors (examples of which are described in, e.g., Buchscher et al. (1992) J. Virol. 66(5) 2731-2739, Johann et al. (1992) J. Virol. 66 (5):1635-11640 (1992), Sommerfelt et al., (1990) Virol. 176:58-59, Wilson et al. (1989) J. Virol. 63:2374-2378, Miller et al., J. Virol. 65:2220-2224 (1991), Wong-Staal et al., PCT/US94/05700, Rosenburg and Fauci (1993) in FUNDAMENTAL IMMUNOLOGY, THIRD EDITION Paul (ed) Raven Press, Ltd., New York and the references therein), an AAV vector (as described in, e.g., West et al. (1987) Virology 160:38-47, Kotin (1994) Human Gene Therapy 5:793-801, Muzyczka (1994) J. Clin. Invst. 94:1351, Tratschin et al. (1985) Mol. Cell. Biol. 5(11):3251-3260, U.S. Pat. Nos. 4,797,368 and 5,173,414, and International Patent Application WO 93/24641), or an adenoviral vector (as described in, e.g., Berns et al. (1995) Ann. NY Acad. Sci. 772:95-104; Ali et al. (1994) Gene Ther. 1:367-384; and Haddada et al. (1995) Curr. Top. Microbiol. Immunol. 199 (Pt 3):297-306), such that immunogenic levels of expression of the nucleic acid included in the vector thereby occurs in vivo resulting in the desired immune response. Other suitable types of viral vectors are described elsewhere herein (including alternative examples of suitable retroviral, AAV, and adenoviral vectors).

Suitable infection conditions for these and other types of viral vector particles are described in, e.g., Bachrach et al., J. Virol. 74(18):8480-6 (2000), Mackay et al., J. Virol. 19(2):620-36 (1976), and Fields VIROLOGY, supra. Additional techniques useful in the production and application of viral vectors are provided in, e.g., “Practical Molecular Virology: Viral Vectors for Gene Expression” in METHODS IN MOLECULAR BIOLOGY, vol. 8, Collins, M. Ed., (Humana Press 1991), VIRAL VECTORS: BASIC SCIENCE AND GENE THERAPY, 1st Ed. (Cid-Arregui et al., Eds.) (Eaton Publishing 2000), “Viral Expression Vectors,” in CURRENT TOPICS IN MICROBIOLOGY AND IMMUNOLOGY, Oldstone et al., Eds. (Springer-Verlag, NY, 1992), and “Viral Vectors” in CURRENT COMMUNICATIONS IN BIOTECHNOLOGY, Gluzman and Hughes, Eds. (Cold Spring Harbor Laboratory Press, 1988).

The toxicity and therapeutic efficacy of the vectors that include recombinant molecules provided by the invention can be determined using standard pharmaceutical procedures in cell cultures or experimental animals. For example., the artisan can determine the LD₅₀ (the dose lethal to 50% of the population) and the ED₅₀ (the dose therapeutically effective in 50% of the population) using procedures presented herein and those otherwise known to those of skill in the art. Nucleic acids, polypeptides, proteins, fusion proteins, transduced cells and other formulations of the present invention can be administered at a rate determined, e.g., by the LD₅₀ of the formulation, and the side-effects thereof at various concentrations, as applied to the mass and overall health of the subject. Administration can be accomplished via single or divided doses.

The viral vector can be targeted to particular tissues, cells, and/or organs. Examples of such vectors are described above. For example, the viral vector or nucleic acid vector can be used to selectively deliver the nucleic acid sequence of the invention to monocytes, dendritic cells, cells associated with dendritic cells (e.g., keratinocytes associated with Langerhans cells), T-cells, and/or B-cells. The viral vector and/or nucleic acid vectors of the invention also can be targeted to EpCAM-overexpressing cells by agents that target cancerous cells (e.g., folates), antibodies to cancer cell antigens, and/or by targeting particular types of cells that may be associated with neoplastic cells (e.g., cells of the epithelium in the lung, breast, colon, rectum, or liver). The viral vector particle of the invention can be a replication-deficient viral vector. The viral vector particle also can be modified to reduce host immune response to the viral vector, thereby achieving persistent gene expression. Such “stealth” vectors are described in, e.g., Martin, Exp. Mol. Pathol. 66(1):3-7 (1999), Croyle et al., J. Virol. 75(10):4792-801 (2001), Rollins et al., Hum. Gene Ther. 7(5):619-26 (1996), Ikeda et al., J. Virol. 74(10):4765-75 (2000), Halbert et al., J. Virol. 74(3), 1524-32 (2000), and International Patent Application WO 98/40509. Alternatively or additionally, the viral vector particles can be administered by a strategy selected to reduce host immune response to the vector particles. Strategies for reducing immune response to the viral vector particle upon administration to a host are provided in, e.g., Maione et al., Proc. Natl. Acad. Sci. USA, 98(11), 5986-91 (2001), Morral et al., Proc. Natl. Acad. Sci. USA 96(22):2816-21 (1999), Pastore et al., Hum. Gene Ther. 10(11):1773-81 (1999), Morsy et al., Proc. Natl. Acad. Sci. USA 95(14):7866-71 (1998), Joos et al., Hum. Gene Ther. 7(13):1555-66 (1996), Kass-Eisler et al., Gene Ther. 3(2): 154-62 (1996), U.S. Pat. Nos. 6,093,699, 6,211,160, 6,225,113, and U.S. Patent Application 2001-0066947A1.

Any suitable population and concentration (dosage) of viral vector particles can be used to induce the immune response in the mammalian host. For example, in adenoviral vectors, at least about 1×10⁹ particles are typically used (e.g., the method can comprises administering a composition comprising at least from about 1×10⁹ particles to about 1×10¹³ particles of an adenoviral vector particle composition in an about 1-2 mL injectable solution, per dose). When delivered to a host, the population of viral vector particles is such that the multiplicity of infection (MOI) desirably is at least from about 1 to about 100 and more preferably from at least about 5 to about 30. Considerations in viral vector particle dosing are described elsewhere herein.

The term “prime” generally refers to the administration or delivery of a polypeptide of the invention or a polynucleotide encoding such polypeptide to a cell culture or population of cells in vitro, or in vivo to a subject or ex vivo to tissue or cells of a subject. The first administration or delivery (primary contact) may not be sufficient to induce or promote a measurable response (e.g., antibody response), but may be sufficient to induce a memory response, or an enhanced secondary response.

As discussed elsewhere herein, the initial delivery or administration of a polypeptide or polynucleotide of the invention to cells or a cell culture in vitro, in vivo, or ex vivo to tissue or cells of a subject typically is followed by such one or more secondary (usually repeat) administrations of the polynucleotide and/or polypeptide. Thus, for example, initial administration of a polypeptide composition can be followed, typically at least about 7 days after the initial polypeptide administration (more typically about 14-35 days or about 2, 4 or 6 months) after initial polypeptide administration), with a first repeat administration (“prime boost”) of a substantially similar (if not identical) dose of the polypeptide, typically in a similar amount as the first administration (e.g., about 5 μg to about 0.1 mg of polypeptide in a 1-2 mL injectable solution). Desirably, a second repeat administration (or “secondary boost”) is performed with a similar, if not identical, dose of the polypeptide composition at about 2-9, preferably about 3-6 months, or about 9-18 months after the initial polypeptide administration. Additional administration strategies and doses are discussed throughout.

Alternatively, a different nucleic acid, polypeptide, vector, cell, or antibody of the invention is used to boost the immune response induced by the first dosage of a nucleic acid, polypeptide, vector, cell, or antibody of the invention. For example, administration to a subject of an initial dosage of a composition comprising a polypeptide comprising the polypeptide sequence SEQ ID NO:1, SEQ ID NO:5, SEQ ID NO:4, or a suitable immunogenic polypeptide of the invention, can advantageously be followed by administration to the subject of an immunogenic second dose of a pox virus, such as a vaccinia virus, canary pox virus, or MVA viral vector, which second dose can further be followed by a third, fourth, or even fifth boost of such a pox virus, wherein such further doses of pox virus enhance the immune response against EpCAM induced by the initial dose of the immunogenic polypeptide of the invention.

The following strategies, summarized in Table 6, provide additional and particular examples of such prime-boosting administration regimens. These strategies are particular examples and do not in any way restrict the ability to use other prime-boosting or different administration strategies, examples of which are provided elsewhere herein.

TABLE 6
Exemplary Prime-Boost Administration Strategies

| 1st Administration | | | |
|---|---|---|---|
| (Prime) | Boost 1 | Boost 2 | Boost 3 |
| DNA injection (i.m.) | DNA injection (i.m.) | DNA | Adenovirus (Ad) injection |
| | | injection | (i.m.) (e.g., injection with |
| | | (i.m.) | about 1 × 10⁹-1 × 10¹¹ PFU |
| | | | Ad vector |
| | | | comprising a heterologous |
| | | | or homologous protein or |
| | | | comprising a nucleic acid |
| | | | encoding a heterologous |
| | | | or homologous |
| | | | protein for the boost)* |
| | | | (e.g., about 1 × 10⁹-1 × 10¹¹ PFU |
| | | | Ad vector |
| | | | comprising a nucleic acid |
| | | | that encodes the |
| | | | polypeptide sequence of |
| | | | SEQ ID NO: 1) |
| DNA injection (i.m.) | Pox virus (e.g., MVA, | Repeat | |
| | canary pox, avipox, or | boost 1 if | |
| | ALVAC) boost (i.m.) | desired | |
| | (virus comprises a | | |
| | heterologous or | | |
| | homologous protein or | | |
| | comprises a nucleic acid | | |
| | encoding a heterologous | | |
| | or homologous | | |
| | protein for the boost)* | | |
| | (e.g., about 2 × 10⁷ PFU | | |
| | canary pox vector | | |
| | comprising a nucleic acid | | |
| | that encodes the | | |
| | polypeptide sequence of | | |
| | SEQ ID NO: 1) | | |
| Adenovirus (i.n. - | DNA boost (i.n.) | | |
| intranasal for | | | |
| mucosal | | | |
| immunization) | | | |
| DNA injection (i.m., | Protein boost* (i.m., i.d., | | |
| i.d. (intradermal), | i.n., or s.c.) | | |
| in., or s.c.) | | | |
| DNA injection (i.m., | Protein boost* (i.m., i.d., | Protein | Protein boost* (i.m., i.n., |
| i.d., i.n., or s.c.) | i.n., or s.c.) | boost* | i.d., or s.c.) |
| | | (im., i.d.,) | |
| | | i.n., or s.c.) | |
| Protein prime (s.c. or | Protein boost* (i.m. or | | |
| i.m.) | s.c.) | | |
| DNA prime | DNA boost | Protein | |
| | | boost* | |
| DNA prime | Protein boost* | | |
| DNA prime | Adenovirus boost | | |
| | (comprising a | | |
| | heterologous or | | |
| | homologous protein for | | |
| | the boost or comprising a | | |
| | nucleic acid encoding a | | |
| | heterologous or | | |
| | homologous | | |
| | protein)* | | |
| Liposome-associated | Protein boost* | | |
| Nucleic acid vector | | | |
| prime | | | |
| DNA (i.m. - e.g., 1 mg | DNA (i.m. - e.g., 1 to 10 mg | DNA (i.m. - | Protein boost* (0.1 to 0.5 mg) |
| pMaxVax) | pMaxVax) | e.g., 1 to | |
| | | 10 mg | |
| | | pMaxVax) | |
| DNA (i.d. - e.g., 1 mg | DNA (i.d. - e.g., 1 to 10 mg | DNA (i.d. - | Protein boost* (0.1 to 0.5 mg) |
| pMaxVax) | pMaxVax) | e.g., 1 to 10 mg | |
| | | pMaxVax) | |

*A protein boost may comprise a heterologous or homologous protein. A heterologous protein used as a protein boost is a protein comprising a polypeptide seqeunce that differs from the sequence of the protein that is encoded by the nucleic acid (e.g., DNA) used for the prime immunization (e.g., nucleic acid prime or vector prime). A homologous protein used as a protein boost is a protein comprising a polypeptide sequence that is identical to the sequence of the protein that is encoded by the nucleic acid (e.g., DNA) used for the prime immunizarion (e.g., DNA prime or DNA vector prime).

A “DNA injection” in Table 6 refers to injection of a nucleic acid or nucleic acid vector of the invention. For example, a DNA injection can include injection of a monocistronic pMaxVax vector encoding SEQ ID NO:4 or bicistronic pMaxVax vector comprising a sequence encoding SEQ ID NO:4 and a second sequence encoding an immunostimulatory/anti-tumor cytokine (e.g., GM-CSF or TNF-α) or a costimulatory polypeptide (e.g., a CD28BP). A heterologous protein boost in Table 6 refers to the administration of a second polypeptide of the invention that differs from the polypeptide(s) of the invention administered in the prime administration or expressed by the DNA, plasmid, or viral vector in the prime administration. Routes of administration (e.g., s.c. (subcutaneous)) provided in Table 6 are exemplary only any suitable route of administration can be used for these or any other prime-boosting strategy described herein. The type of administration strategy can influence the type of immune response. For example, administration of a recombinant adenovirus is expected to provide very effective antibody production, whereas administration of a DNA vector (e.g., a pMaxVax vector) followed by a protein, DNA, and/or viral vector boost is expected to provide very effective T cell responses.

One method for treating or delaying occurrence of metastatic disease in subject having EpCAM/KSA-associated malignancies, such as stage II or III colon cancers, includes one or more rounds of DNA priming followed by one or more protein boosts. One round of DNA priming comprises administration to the subject in need of such treatment of a DNA vector encoding TAg-25 (or other TAg polypeptide described herein) and optionally also encoding CD28BP-15. The DNA vector is formulated in PBS at pH 7.4. Each protein boost comprises administration to the subject of TAg-25 or other TAg polypeptide of the invention. The protein is typically formulated in PBS and 1.5% alum. The dose for DNA priming typically comprises 10 mg TAg-encoding DNA; the dose for the protein boost typically comprises 500 ug TAg protein. An exemplary immunization schedule comprises two rounds of DNA-DNA-protein immunizations at four-week intervals.

Adjuvants

Any technique comprising administering a polypeptide of the invention can also include the co-administration of one or more suitable adjuvants. Examples of suitable adjuvants include Freund's emulsified oil adjuvants (complete and incomplete), alum (aluminum hydroxide and/or aluminum phosphate), lipopolysaccharides (e.g., bacterial LPS), liposomes (including dried liposomes and cytokine-containing (e.g., IFN-γ-containing and/or GM-CSF-containing) liposomes), endotoxins, cytokines (such as, e.g., IL-12) costimulatory molecules (such as, e.g., B7-1 (CD80) and/or B7-2 (CD86), calcium phosphate and calcium compound microparticles (see, e.g., International Patent Application Pub. No. WO 00/46147), mycobacterial adjuvants, Arlacel A, mineral oil, emulsified peanut oil adjuvant (adjuvant 65), Bordetella pertussis products/toxins, Cholera toxins, non-ionic block polymer surfactants, Corynebacterium granulosum derived P40 component, fatty acids, aliphatic amines, paraffinic and vegetable oils, beryllium, and immunostimulating complexes (ISCOMs—reviewed in, e.g., Höglund et al. “ISCOMs and immunostimulation with viral antigens” in SUBCELLULAR BIOCHEMISTRY (Ed. Harris, J. R.) Plenum, New York, 1989, pp. 39-68), Morein et al., “The ISCOM—an approach to subunit vaccines” in RECOMBINANT DNA VACClNES: RATIONALE AND STRATEGY (Ed. Isaacson, R. E.) Marcel Dekker, New York, 1992, pp. 369-386, and Morein et al., Clin. Immunotherapeutics 3:461-75 (1995)). Recently, monophosphoryl lipid A, ISCOMs with Quil-A, and Syntex adjuvant formulations (SAFs) containing the threonyl derivative or muramyl dipeptide also have been under consideration for use in human vaccines. Numerous types of adjuvants that can be suitable for co-administration or serial administration with one or more polypeptides of the invention are known in the art. Examples of such adjuvants are described in, e.g., Vogel et al., A COMPENDIUM OF VACClNE ADJUVANTS AND EXCIPIENTS (2d Ed.) (http://www.niaid.nih.gov/aidsvaccine/pdf/compendium.pdf), Bennet et al., J. Immun. Meth. 153:31-40 (1992), Bessler et al., Res. Immunol. 143(5):519-25 (1992), Woodard, Lab Animal Sci. 39(3):222-5 (1989), Vogel, AIDS Res. and Human Retroviruses 11(10):1277-1278 (1995), Leenaars et al., Vet. Immunol. Immunopath. 40:225-241 (1995), Linblak et al., Scandinavian J. Lab. Animal Sci 14:1-13 (1987), Buiting et al., Res. Immunol. 143(5):541-548 (1992), Gupta and Siber, Vaccine (14):1263-1276 (1996), and U.S. Pat. Nos. 6,340,464, 6,328,965, 6,299,884, 6,083,505, 6,080,725, 6,060,068, 5,961,970, 5,814,321, 5,747,024, 5,690,942, 5,679,356, 5,650,155, 5,585,099, 4,395,394, and 4,370,265.

Administration Formats

As indicated above, administration of a nucleic acid of the invention also is typically and preferably followed by boosting (at least a prime, preferably at least a prime and secondary boost). A “prime” is typically the first immunization. An initial nucleic acid administration can be followed by a repeat administration of the nucleic acid at least about 7 days, more typically and preferably about 14-35 days, or about 2, 4, or 6 months, after the initial nucleic acid administration. Alternatively, the initial administration of the nucleic acid can be followed by a prime boost of an immunogenic amount of polypeptide at such a time. Preferably, in such aspects, a secondary boost also is preferably performed with nucleic acid and/or polypeptide, in an amount similar to that used in the primary boost and/or the initial nucleic acid administration, at about 2-9, preferably about 3-6 months or about 9-18 months after the initial nucleic acid administration. Any number of boosting administrations of nucleic acid and/or polypeptide can be performed.

The polypeptide, nucleic acid, vector, cell, and/or antibody of the invention can be used to promote any suitable immune response to EpCAM in a subject in any suitable context. For example, at least one recombinant polypeptide, nucleic acid, and/or vector can be administered as a prophylactic in an immunogenic or antigenic amount to a mammal (preferably, a human) that has no detectable amount of EpCAM-associated cancer progression. Preferably, the polypeptide, nucleic acid, antibody, cell, vector, or combination thereof (or related composition) induces a protective immune response against EpCAM-associated cancers and, as such, can be considered a “vaccine” against such cancers.

As indicated elsewhere herein, the polynucleotides and vectors of the invention can be delivered by ex vivo delivery of cells, tissues, or organs. As such, the invention provides a method of promoting an immune response to EpCAM comprising inserting at least one nucleic acid and/or vector of the invention into a population of cells and implanting the cells in a mammal. Ex vivo administration strategies are known in the art (see, e.g., U.S. Pat. No. 5,399,346 and Crystal et al., Cancer Chemother. Pharmacol., 43(Suppl.), S90-S99 (1999)). Cells or tissues can be injected by a needle or gene gun or implanted into a mammal ex vivo. Briefly, in ex vivo techniques, a culture of cell (e.g., organ cells, cells of the skin, muscle, etc.) or target tissue is provided, or preferably removed from the host, contacted with the vector or polynucleotide composition, and then reimplanted into the host (e.g., using techniques described in or similar to those provided in). Ex vivo administration of the nucleic acid can be used to avoid undesired integration of the nucleic acid and to provide targeted delivery of the nucleic acid or vector. Such techniques can be performed with cultured tissues or synthetically generated tissue. Alternatively, cells can be provided or removed from the host, contacted (e.g., incubated with) an immunogenic amount of a polypeptide of the invention that is effective in prophylactically inducing an immune response to EpCAM when the cells are implanted or reimplanted to the host. The contacted cells are then delivered or returned to the subject to the site from which they were obtained or to another site (e.g., including those defined above) of interest in the subject to be treated. If desired, the contacted cells may be grafted onto a tissue, organ, or system site (including all described above) of interest in the subject using standard and well-known grafting techniques or, e.g., delivered to the blood or lymph system using standard delivery or transfusion techniques. Such techniques can be performed with any suitable type of cells. For example, in one aspect, activated T cells can be provided by obtaining T cells from a subject (e.g., mammal, such as a human) and administering to the T cells a sufficient amount of one or more polypeptides of the invention to activate effectively the T cells (or administering a sufficient amount of one or more nucleic acids of the invention with a promoter such that uptake of the nucleic acid into one or more such T cells occurs and sufficient expression of the nucleic acid results to produce an amount of a polypeptide effective to activate said T cells). The activated T cells are then returned to the subject. T cells can be obtained or isolated from the subject by a variety of methods known in the art, including, e.g., by deriving T cells from peripheral blood of the subject or obtaining T cells directly from a tumor of the subject. Other preferred cells for ex vivo methods include explanted lymphocytes, particularly B cells, antigen presenting cells (APCs), such as dendritic cells, and more particularly Langerhans cells, monocytes, macrophages, bone marrow aspirates, or universal donor stem cells. A preferred aspect of ex vivo administration of a polynucleotide or polynucleotide vector can be the assurance that the polynucleotide has not integrated into the genome of the cells before delivery or re-administration of the cells to a host. If desired, cells can be selected for those where uptake of the polynucleotide or vector, without integration, has occurred, using standard techniques known in the art.

In other aspects, a nucleic acid or vector of the invention is introduced into a host cell or host (e.g., a human) therapeutically by administering an immunogenic amount of a population of bacterial cells comprising the nucleic acid of the invention, wherein such administration results in expression of a recombinant polypeptide of the invention, and induction of an immune response to EpCAM in the host cell or host. Bacterial cells developed for mammalian gene delivery are known in the art and particular examples of such cells are provided elsewhere herein (e.g., attenuated BCG cells).

In another aspect, administration of a polynucleotide or vector (preferably a polynucleotide vector) of the invention is facilitated by application of electroporation to an effective number of cells or an effective tissue target, such that the nucleic acid and/or vector is taken up by the cells, and expressed therein, resulting in production of a recombinant polypeptide of the invention therein and subsequent induction of an immune response to EpCAM in the cells (e.g., a tissue and/or a tumor of a human).

In some aspects, the nucleic acid, polypeptide, and/or vector of the invention is desirably co-administered with an additional nucleic acid or additional nucleic vector comprising an additional nucleic acid that increases the immune response to EpCAM upon administration of the nucleic acid, polypeptide, and/or vector of the invention. Preferably, such a second nucleic acid comprises a sequence encoding a granulocyte-macrophage colony stimulating factor (GM-CSF), an interferon (e.g., IFN-γ), or both, examples of which are discussed elsewhere herein. Alternatively, the second nucleic acid can comprise immunostimulatory (CpG) sequences, as described elsewhere herein. GM-CSF, IFN-γ, or other polypeptide adjuvants also can be co-administered with the polypeptide, polynucleotide, and/or vector. Co-administration in this respect (and throughout unless otherwise indicated) encompasses administration before, simultaneously with, or after, the administration of the polynucleotide, polypeptide, and/or vector of the invention, at any suitable time resulting in an enhancement of an immune response.

As mentioned throughout, a particular advantageous utility of the polypeptides, nucleic acids, antibodies, cells, and vectors of the invention is the ability to induce an immune response against cells that overexpress EpCAM. Techniques for identification of EpCAM-overexpressing cells are known (see, e.g., Gastl et al., Lancet. 2000 Dec. 9;356(9246):1981-2 and Cirulli et al., J. Cell Biol. 140(6):1519-1534 (1998)). The novel biomolecules of the invention can also be used to identify such cells when combined with an appropriate label (e.g., a radionucleotide or reporter sequence, such as a GFP sequence). In therapeutic methods, the administration of an immunogenic amount of a polypeptide, nucleic acid, vector, antibody, or cell of the invention advantageously results in an at least ⅔rds decrease in the number of such cells in a subject after a suitable period of time. In some aspects, the decrease in the number of such cells can be significantly higher (e.g., an at least about 70, 80, 85, 90%, or 95% decrease in such cells).

The nucleic acids, polypeptides, antibodies, cells, and/or vectors of the invention can further be used to modulate morphoregulation of epithelial cells or other EpCAM-associated cells, such as islet cells. For example, the invention provides a method of modulating the outgrowth of endocrine cells from the ductal epithelium, comprising the administration of such a biomolecule to the appropriate cells or tissue during such outgrowth. In related aspects, such methods can be used to provide a method of regulating cell differentiation. In still another aspect, the invention provides a method of modulating epithelial cell proliferation, which method comprises administering an effective amount of a novel biomolecule of the invention to such cells under conditions in which epithelial cell proliferation is increased or inhibited. Other uses of the polypeptides, nucleic acids, vectors, cells, and antibodies of the invention include the regulation of morphogenesis in pancreas and mammary gland, modulation of cell-to-cell signaling, particularly in epithelial cells, the modulation of epithelial cell differentiation, and (by diagnostic techniques) the differentiation of cells of particular tissues types or morphology, including the identification of cancerous cells (e.g., breast micrometastic cells) or tumors. The novel polypeptides of the invention (particularly polypeptides comprising at the first cysteine-rich region of a polypeptide of the invention, if not both cysteine-rich regions, as well as typically a transmembrane domain, and a cytoplasmic domain, as described above (e.g., an EpCAM transmembrane and cytoplasmic domain portion), can be used to promote epithelial cell-to-cell adhesion in a calcium-independent manner. As such, aggregates of cells adhered to one another by such polypeptides are another feature of the invention.

In a further aspect, the invention provides a method of regulating cell adhesions comprising administering an effective amount of a nucleic acid, antibody, polypeptide, cell, and/or vector of the invention to suitable target cells, such that cell adhesions are modulated (i.e., either detectably increased or decreased). For example, in one sense the invention provides a method of inhibiting cadherin-mediated cell-to-cell adhesion comprising administering a polypeptide of the invention, nucleic acid of the invention, or vector of the invention into or near to EPCAM expressing epithelial cells that are associated with (e.g., are near to) cadherin-mediated cell adhesions.

The induction of an immune response to EpCAM-overexpressing cancerous cells is perhaps the most important utility of the polypeptides, nucleic acids, vectors, cells, and antibodies of the invention. The polypeptides, nucleic acids, cells, antibodies, vectors, and compositions of the invention can be used to induce an immune response against any suitable type of EpCAM-overexpressing cell associated with any suitable type of cancer including, e.g., hepatocellular carcinomas, cholangiocarcinomas, hepatoblastomas, squamous carcinomas, laryngeal carcinomas, colorectal adenocarcinomas, ovarian carcinomas, cervix carcinomas, renal cell carcinomas, prostrate carcinomas, lung carcinomas, bladder carcinomas and other cancers of the colon, lymphoid, gastrointestinal, stomach, colon, pancreas, liver, gall bladder, thyroid, thymus, tonsils, breast, and oral areas (including, e.g., micrometastatic cancer cells in such tissues). The novel biomolecules also can be used to treat and/or prevent (reduce the risk of) other and/or more particular cancers associated with EpCAM (as described in, e.g., Balzar et al., 1999, supra), such as Dukes' B or C colorectal carcinomas.

The reduction of cancer progression can be characterized by any suitable measurement including, e.g., a reduction in one or more markers of tumorigenicity in a subject (e.g., human), a reduction of micrometastatic tumor load, the treatment of a tumor-associated or micrometastatic disease, the reduction of total tumor burden, the presence of a disease-free state or conditions, and/or the increase in the overall survival of subjects in a particular population or that have particular conditions. Reduction of markers is a convenient measurement of the therapeutic effect of a treatment against a cancer. For example, the reduction of cytokeratin CK+ cells can be used to assess the effectiveness of a polypeptide, vector, nucleic acid, or related composition of the invention to provide a therapeutic effect against cancer in a host (see, e.g., Braun et al., Clin. Cancer Res. 5:3999-4004 (1999) for discussion of such cells and measurements in the context of related EpCAM therapies).

The invention also provides a method of reducing, inhibiting, or eliminating cancer progression in a subject, which method includes the use of radiation therapy, chemotherapy, or both, in combination with the administration of a polypeptide, nucleic acid, vector, antibody, and/or cell of the invention. In a further aspect, a polypeptide, nucleic acid, vector, antibody, and/or cell of the invention is co-administered with a therapeutic monoclonal antibody, small molecule drug, an anti-angiogenic agent (or an angiogenesis inhibitor), a targeted apoptotic agent, an anti-tumor antisense nucleic-acid (e.g., an antisense nucleic acid that blocks production of the protein kinase C alpha (PKCa) protein or other cancer cell-associated protein, such as C-raf kinase), or other anti-cancer agent, such as Gleevec, paclitaxel (Taxol), hycamtin, irinotecan, letrozole, anastrozole, capecitabine, goserelin, toremifene, docetaxel, tretinoin, gemcitabine, nilutamide, bicalutamide, a thymidine kinase, or herceptin. In another aspect, the invention provides a method of reducing cancer progression (e.g., a method of reducing tumor size) in a host, which method comprises administering a polypeptide, nucleic acid, vector, cell, and/or antibody of the invention in combination with an oncolytic virus, such as an oncolytic amount of a reovirus. Suitable anti-angiogenic agents for such combination therapies include, e.g., endostatins (or fragment thereof, such as the collagen XVIII fragment), angiotensins (or fragment thereof, such as the plasminogen fragment of human angiotensin), thrombospondins (e.g., thrombospondin-1), the 16 kDa fragment of prolactin, and vasostatin (or calreticulin)), Cartilage-derived inhibitor (CDI), CD59 complement fragment, Gro-beta, Heparinases, Heparin hexasaccharide fragment, Human chorionic gonadotropin (hCG), IFNs, Interferon inducible protein (IP-10), IL-12, Kringle 5 (plasminogen fragment), 2-Methoxyestradiol, Placental ribonuclease inhibitor, Plasminogen activator inhibitor, Platelet factor-4 (PF4), Proliferin-related protein (PRP), Retinoids, Tetrahydrocortisol-S, other anti-angiogenic C-X-C chemokines, and/or vasculostatin.

Diagnostic Applications

The invention also provides an in vivo diagnostic component that comprises a polypeptide of the invention conjugated to a detectable label. The invention provides a diagnostic assay for detecting mEpCAM by use of such labeled polypeptide. A particular use of such labeled polypeptides is the identification of EpCAM-overexpressing cells (e.g., EpCAM-associated cancer cells). Such assay comprises administering such a labeled polypeptide to a cells or tissue suspected of containing such cells and identifying what cells, if any, the labeled polypeptides bind to.

In one aspect, the invention provides a diagnostic method of screening a composition for antibodies that bind EpCAM and/or a polypeptide of the invention. Such diagnostic method is especially useful for determining if a composition contains antibodies to EpCAM. In one aspect, the method comprises contacting a sample of the composition with a polypeptide of the invention under conditions such that if the sample comprises antibodies that bind to EpCAM, at least one such antibody binds to the polypeptide of the invention to form a mixed composition. The mixed composition is then contacted with at least one affinity-molecule that binds to an anti-EpCAM antibody. Unbound affinity-molecule is then removed from the mixed composition, and the presence or absence of bound affinity molecules in the composition is detected, wherein the presence of an affinity molecule is indicative of the presence of antibodies that bind to EpCAM. This technique can be modified to provide an ELISA or other EIA for the detection of such antibodies in a particular medium.

Methods of Production and Purification

The invention further provides methods of making the polypeptides, polynucleotides, vectors, and cells of the invention. In one aspect, the invention provides a method of making a recombinant polypeptide of the invention by introducing a nucleic acid of the invention into a population of cells in a culture medium, culturing the cells in the medium (for a time and under conditions suitable for desired level of gene expression) to produce the polypeptide, and isolating the polypeptide from the cells, culture medium, or both. The polypeptide can be isolated from the cell culture by any suitable technique including, e.g., affinity chromatography of cell lysates and/or cell supernatants, Western blotting of cell lysates or cell supernatants and/or cell lysates, or other techniques known in the art. A variety of polypeptide purification methods are well known in the art, including those set forth in, e.g., Sandana (1997) BIOSEPARATION OF PROTEINS, Academic Press, Inc., Bollag et al. (1996) PROTEIN METHODS, 2^{nd} Edition Wiley-Liss, NY, Walker (1996) THE PROTEIN PROTOCOLS HANDBOOK Humana Press, NJ, Harris and Angal (1990) PROTEIN PURIFICATION APPLICATIONS: A PRACTICAL APPROACH IRL Press at Oxford, Oxford, England, Scopes (1993) PROTEIN PURIFICATION: PRINCIPLES AND PRACTICE 3^{rd} Edition Springer Verlag, N.Y., Janson and Ryden (1998) PROTEIN PURIFICATION: PRINCIPLES, HIGH RESOLUTION METHODS AND APPLICATIONS, Second Edition Wiley-VCH, NY; and Walker (1998) PROTEIN PROTOCOLS ON CD-ROM Humana Press, NJ. Cells suitable for polypeptide production are known in the art and are discussed elsewhere herein (e.g., Vero cells, 293 cells, BHK, CHO, and COS cells can be suitable). Cells can be lysed by any suitable technique including, e.g., sonication, microfluidization, physical shear, French press lysis, or detergent-based lysis.

In one aspect, the invention provides a method of purifying EpCAM, an EpCAM homolog, an EpCAM ortholog, or a polypeptide comprising an immunogenic amino acid sequence of the invention, which method comprises transforming a suitable host cell with a nucleic acid of the invention (e.g., a nucleic acid that encodes a polypeptide comprising the polypeptide sequence of SEQ ID NO: 1, SEQ ID NO:5, or SEQ ID NO:4) in the host cell (e.g., a CHO cell or 293 cell), lysing the cell by a suitable lysis technique (e.g., sonication, detergent lysis, or other appropriate technique), and subjecting the lysate to affinity purification with a chromatography column comprising a resin that includes at least one novel antibody of the invention (usually a monoclonal antibody of the invention) or antigen-binding fragment thereof, such that the lysate is enriched for the desired polypeptide (e.g., a polypeptide comprising the polypeptide sequence of SEQ ID NO: 1, SEQ ID NO:5, or SEQ ID NO:4).

In an alternative method, the invention provides a method for purifying such target polypeptides (e.g., a polypeptide comprising the polypeptide sequence of SEQ ID NO: 1), which method differs from the above-described method in that a nucleic acid comprising a nucleotide sequence encoding a fusion protein that comprises an immunogenic polypeptide of the invention (see, e.g., SEQ ID NO:4) and a suitable tag (e.g., an e-epitope/his tag), and purifying the polypeptide by immunoaffinity and/or IMAC chromatography enrichment techniques.

The invention provides a similar method of making a polypeptide of the invention comprising inserting a vector according to the invention to the cells, culturing the cells under appropriate conditions for expression of the nucleic acid from the vector, and isolating the polypeptide from the cells, culture medium, or both. The cells chosen are based on the desired processing of the polypeptide and based on the appropriate vector (e.g., E. coli cells can be preferred for bacterial plasmids, whereas 293 cells can be preferred for mammalian shuttle plasmids and/or adenoviruses, particularly E1-deficient adenoviruses).

In addition to recombinant production, the polypeptides of the invention may be produced by direct peptide synthesis using solid-phase techniques (see, e.g., Stewart et al. (1969) SOLID-PHASE PEPTIDE SYNTHESIS, WH Freeman Co, San Francisco and Merrifield J. (1963) J. Am. Chem. Soc. 85:2149-2154). Peptide synthesis may be performed using manual techniques or by automation. Automated synthesis may be achieved, for example, using Applied Biosystems 431A Peptide Synthesizer (Perkin Elmer, Foster City, Calif.) in accordance with the instructions provided by the manufacturer. For example, subsequences may be chemically synthesized separately and combined using chemical methods to produce a polypeptide of the invention or fragments thereof. Alternatively, synthesized polypeptides may be ordered from any number of companies that specialize in production of polypeptides. Most commonly, polypeptides of the invention are produced by expressing coding nucleic acids and recovering polypeptides, e.g., as described above.

In another aspect, the invention provides a method of producing a polypeptide of the invention comprising introducing a nucleic acid of the invention, a vector of the invention, or a combination thereof, into an animal, which typically and preferably is a mammal (e.g., a rat, a nonhuman primate, a bat, a marmoset, a pig, or a chicken), such that a polypeptide of the invention is expressed in the animal, and the polypeptide is isolated from the animal or from a byproduct of the animal. Isolation of the polypeptide from the animal or animal byproduct can be by any suitable technique, depending on the animal and desired recovery strategy. For example, the polypeptide can be recovered from sera of mice, monkeys, or pigs expressing the polypeptide of the invention. Transgenic animals (which preferably are mammals, such as the aforementioned mammals) comprising at least one nucleic acid of the invention are provided by the invention. The transgenic animal can have the nucleic acid integrated into its host genome (e.g., by an AAV vector, lentiviral vector, biolistic techniques performed with integration-promoting sequences, etc.) or can have the nucleic acid in maintained epichromosomally (e.g., in a non-integrating plasmid vector or by insertion in a non-integrating viral vector). Epichromosomal vectors can be engineered for more transient gene expression than integrating vectors. RNA-based vectors offer particular advantages in this respect.

Also provided is method of producing an isolated polypeptide of the invention which comprises introducing a nucleic acid encoding said polypeptide into a population of cells in a medium, which cells are permissive for expression of the nucleic acid, maintaining the cells under conditions in which the nucleic acid is expressed, and thereafter isolating the polypeptide from the medium.

Compositions

The invention further provides novel and useful compositions comprising one or more polypeptides, nucleic acids, vectors, cells, and/or antibodies of the invention, or combinations thereof, such as compositions corresponding to the above-described methods of the invention (e.g., a composition comprising a viral vector encoding a nucleic acid of the invention and an oncolytic virus and/or one or more anti-angiogenic factors). For example, in a general sense, the invention provides a composition comprising a polypeptide of the invention and a carrier, excipient, or diluent. Such compositions can comprise any suitable amount of any suitable number of polypeptides, fusion proteins, nucleic acids, vectors, and/or cells of the invention.

For example, in one embodiment, the invention provides composition that comprises an excipient or carrier and a plurality of more recombinant polypeptides of the invention (e.g., two, three, four, or more recombinant polypeptide), wherein the composition induces a humoral and/or T cell immune response(s) against EpCAM, an EpCAM homolog, and/or EpCAM ortholog in an animal, preferably in a mammal, more preferably in a primate, and most preferably in a human. Corresponding pharmaceutical compositions comprising a pharmaceutically acceptable excipient or carrier are also provided.

In another aspect, the invention provides compositions (including pharmaceutical compositions) that comprise an excipient or carrier (or pharmaceutically acceptable excipient, diluent, or carrier), an adjuvant and/or one or more other polypeptides comprising a cancer antigen and/or an immunogenic portion thereof.

By way of example, an effective amount of a polypeptide of the invention for an initial dosage is about 100-600 μg, usually about 300-500 μg (e.g., about 400 or 500 μg), which dosage is normally administered at about 0, 2, 4, and 6 weeks, e.g., through a subcutaneous injection. Such a composition preferably will comprise an adjuvant, such as an immunostimulatory cytokine. For example, such a composition can further comprise or be co-administered with about 75 μg GM-CSF. In protein administration strategies, the polypeptide of the invention is administered as a soluble polypeptide. A soluble polypeptide includes a polypeptide comprising a SP, PP, and ECD of the invention (e.g., TAg-25 (SEQ ID NO:4), TAg-21 (SEQ ID NO:13), TAg-18 (SEQ ID NO:32) and SEQ ID NO:78 (TAg-25/TAg-18 chimera); a polypeptide comprising a PP and ECD (e.g., SEQ ID NO:5); a polypeptide comprising an ECD (e.g., SEQ ID NO:1, 9, 12, or 92). A soluble polypeptide typically lacks a transmembrane and cytoplasmic domain or is not covalently bound to a cell membrane.

An effective amount of antibody of the invention will usually be about 500 mg for an initial dose to a human, which dose can be formulated in PBS and/orin an adjuvant such as Freund's incomplete adjuvant or alum. Normally, such a dose will be followed by subsequent administrations of smaller doses (e.g., about 100-400 mg) about ever 2-3 days or week for a period of months. In some situations, a period of higher initial doses over several (e.g., 5) consecutive days can be used (e.g., 5 consecutive daily doses of about 400-450 mg antibody). Additionally or alternatively about 300-500 mg can be administered every 4-6 weeks thereafter the initial dosage of antibody.

Where a composition comprising an antibody of the invention is to be administered to a subject, the composition can typically further comprise leucovorin (e.g., about 20 mg/M²), lenvamisole, and/or a fluorouracil composition (e.g., 5FU). Effective doses of a nucleic acid vector of the invention (e.g., a pMaxVax monocistronic or bicistronic vector) are normally about 1-15 mg (including, e.g., dose of about 1, 2, 5, 8, or 10 mg) and usually delivered in a concentration of about 2, 5, or 10 mg/ml. In one method, for example, the invention comprises administering a first dose of 10 mg nucleic acid vector comprising: 1) 5 mg of a TAg antigen-encoding polynucleotide sequence (e.g., the polynucleotide sequence of SEQ ID NO: 19); and 2) 5 mg of a costimulator-encoding polynucleotide sequence. Exemplary costimulators include human B7-1 protein and novel CD28BP polypeptides described in commonly assigned Int'l Patent App. PCT/US01/19973 (WO 02/00717) and Int'l Patent App. PCT/US02/19898, filed Jun. 21, 2002. A preferred costimulatory is CD28BP-15, which is described in Int'l Patent App. PCT/US01/19973 (amino acid and nucleic acid sequences of CD28BP-15 are SEQ ID NOS:66 and 19, respectively, in PCT/US01/19973 and PCT/US02/19898). In one aspect, the nucleic acid vector comprises a bicistronic pMaxVax vector encoding a TAg antigen (e.g., TAg-25) of the invention and CD28BP-15 (see FIG. 5).

The first dose followed by a second dose of the nucleic acid vector about 4 weeks after the first dose, followed by a boost of the same or heterologous immunogenic protein of the invention (e.g., about 400 or 500 μg protein in a 0.5-2 mL solution) after an additional about 4 week period, which can be further combined with administration of GM-CSF protein at days 0, 1, 2, and 3 (e.g., about 50-100 μg, most commonly about 75 μg). Two rounds of DNA-DNA-protein immunizations at 4-week intervals may be administered. Polypeptides encoded by nucleic acids of the vector typically (although not necessarily) will include a functional signal sequence, as described above. The nucleic acid vector is typically formulated in sterile, phosphate-buffered saline (PBS) at pH 7.4, and the TAg protein is typically formulated in PBS and Alum adjuvant.

The invention also provides a composition comprising at least one nucleic acid of the invention and a pharmaceutically acceptable carrier. Carriers for nucleic acid compositions include those described herein with respect to polypeptide compositions and those described above with respect to methods of using nucleic acids and nucleic acid compositions of the invention. In a more particular aspect, the invention provides a composition comprising a first nucleic acid encoding an immunogenic polypeptide of the invention (e.g., a polypeptide comprising SEQ ID NO: 1, 4, or 5) and a second nucleic acid encoding a second immunogenic polypeptide of the invention, wherein the first nucleic acid and second nucleic acid encode proteins having different amino acid sequences and each protein independently induces an immune response against hEpCAM. In more particular aspects, the invention provides a composition comprising a pool or library of such nucleic acids.

A pharmaceutical composition (or pharmaceutically acceptable composition) comprising a nucleic acid, polypeptide, vector, cell, or antibody of the invention can be any non-toxic composition that does not interfere with the immunogenicity of the nucleic acid, polypeptide, vector, cell, and/or antibody of the invention included therein. The composition can comprise one or more excipients or carriers, and the pharmaceutical composition comprises one or more pharmaceutically acceptable carriers. A wide variety of acceptable carriers, diluents, and excipients are known in the art. There are a wide variety of suitable formulations of compositions and pharmaceutical compositions of the present invention. For example, a variety of aqueous carriers can be used, e.g., buffered saline, such as phosphate-buffered saline (PBS), and the like are advantageous in injectable formulations of the polypeptide, polynucleotide, and/or vector of the invention. These solutions are preferably sterile and generally free of undesirable matter. These compositions may be sterilized by conventional, well-known sterilization techniques. The compositions may comprise pharmaceutically acceptable auxiliary substances as required to approximate physiological conditions, such as, e.g., pH adjusting and buffering agents, toxicity adjusting agents and the like, for example, sodium acetate, sodium chloride, potassium chloride, calcium chloride, sodium lactate and the like. Any suitable carrier can be used in the administration of the polynucleotide, polypeptide, and/or vector of the invention, and several carriers for administration of therapeutic proteins are known in the art.

The composition, pharmaceutical composition and/or pharmaceutically acceptable carrier also can include diluents, fillers, salts, buffers, detergents (e.g., a nonionic detergent, such as Tween-80), stabilizers, stabilizers (e.g., sugars or protein-free amino acids), preservants, tissue fixatives, solubilizers, and/or other materials suitable for inclusion in a pharmaceutically composition. Examples of suitable components of the pharmaceutical composition in this respect are described in, e.g., Berge et al., J. Pharm. Sci. 66(1):1-19 (1977), Wang and Hanson, J. Parenteral. Sci. Tech. 42:S4-S6 (1988), U.S. Pat. Nos. 6,165,779 and 6,225,289, and elsewhere herein. The pharmaceutical composition also can include preservatives, antioxidants, or other additives known to those of skill in the art. Additional pharmaceutically acceptable carriers are known in the art. Examples of additional suitable carriers are described in, e.g., Urquhart et al., Lancet 16:367 (1980), Lieberman et al., Pharmaceutical Dosage Forms—Disperse Systems (2nd ed., Vol. 3, 1998), Ansel et al., Pharmaceutical Dosage Forms & Drug Delivery Systems (7th ed. 2000), Martindale, THE EXTRA PHARMACOPEIA (31st edition), REMINGTON'S PHARMACEUTICAL SCIENCES (16th-20th editions), THE PHARMACOLOGICAL BASIS OF THERAPEUTICS, Goodman and Gilman, Eds. (9th ed. 1996), Wilson and Gisvolds, TEXTBOOK OF ORGANIC MEDICINAL AND PHARMACEUTICAL CHEMISTRY, Delgado and Remers, Eds. (10th ed. 1998), and U.S. Pat. Nos. 5,708,025 and 5,994,106. Principles of formulating pharmaceutically acceptable compositions are described in, e.g., Platt, Clin. Lab Med. 7:289-99 (1987), Aulton, PHARMACEUTICS: THE SCIENCE OF DOSAGE FORM DESIGN, Churchill Livingstone (New York) (1988), EXTEMPORANEOUS ORAL LIQUID DOSAGE PREPARATIONS, CSHP (1998), and J. Kans. Med. Soc., 70(1):30-32 (1969). Additional pharmaceutically acceptable carriers particularly suitable for administration of vectors are described in, for example, Int'l Patent Application Publ. No. WO 98/32859.

The composition or pharmaceutical composition of the invention can comprise or be in the form of a liposome. Suitable lipids for liposomal formulation include, without limitation, monoglycerides, diglycerides, sulfatides, lysolecithin, phospholipids, saponin, bile acids, and the like. Preparation of such liposomal formulations is described in, e.g., U.S. Pat. Nos. 4,837,028 and 4,737,323.

The form of the compositions or pharmaceutical composition can be dictated, at least in part, by the route of administration of the polypeptide, polynucleotide, cell, and/or vector of interest. Because numerous routes of administration are possible, the form of the pharmaceutical composition and/or components thereof can vary. For example, in transmucosal or transdermal administration, penetrants appropriate to the barrier to be permeated are preferably included in the composition. Such penetrants are generally known in the art, and include, for example, for transmucosal administration, detergents, bile salts, and fusidic acid derivatives. In contrast, in transmucosal administration can be facilitated through the use of nasal sprays or suppositories.

A common administration form for compositions, including pharmaceutical compositions, comprising the polypeptides and/or polynucleotides of the invention is by injection. Injectable pharmaceutically acceptable compositions comprise one or more suitable liquid carriers such as water, petroleum, physiological saline, bacteriostatic water, Cremophor ELTM (BASF, Parsippany, N.J.), phosphate buffered saline (PBS), or oils. Liquid pharmaceutical compositions can further include physiological saline solution, dextrose (or other saccharide solution), polyols, or glycols, such as ethylene glycol, propylene glycol, PEG, coating agents which promote proper fluidity, such as lecithin, isotonic agents, such as mannitol or sorbitol, organic esters such as ethyoleate, and absorption-delaying agents, such as aluminum monostearate and gelatins. Preferably, the injectable composition is in the form of a pyrogen-free, stable, aqueous solution. Preferably, the injectable aqueous solution comprises an isotonic vehicle such as sodium chloride, Ringer's injection solution, dextrose, lactated Ringer's injection solution, or an equivalent delivery vehicle (e.g., sodium chloride/dextrose injection solution). Formulations suitable for injection by intraarticular (in the joints), intravenous, intramuscular, intradermal, subdermal, intraperitoneal, and subcutaneous routes, include aqueous and non-aqueous, isotonic sterile injection solutions, which can include antioxidants, buffers, bacteriostats, and solutes that render the formulation isotonic with the blood of the intended recipient (e.g., PBS and/or saline solutions, such as 0.1 M NaCl), and aqueous and non-aqueous sterile suspensions that can include suspending agents, solubilizers, thickening agents, stabilizers, and preservatives.

The administration of a polypeptide, polynucleotide, or vector of the invention can be facilitated by a delivery device be formed of any suitable material. Examples of suitable matrix materials for producing non-biodegradable administration devices include hydroxapatite, bioglass, aluminates, or other ceramics. In some applications, a sequestering agent, such as carboxymethylcellulose (CMC), methylcellulose, or hydroxypropyl-methylcellulose (HPMC), can be used to bind the polypeptide, polynucleotide, or vector to the device for localized delivery.

In another aspect, a polynucleotide or vector of the invention can be formulated with one or more poloxamers, polyoxyethylene/polyoxypropylene block copolymers, or other surfactants or soap-like lipophilic substances for delivery of the polynucleotide or vector to a population of cells or tissue or skin of a subject in vivo, ex vivo, or in in vitro systems. See e.g., U.S. Pat. Nos. 6,149,922, 6,086,899, and 5,990,241.

Vectors and polynucleotides of the invention can be desirably associated with one or more transfection-enhancing agents. In some embodiments, a nucleic acid and/or nucleic acid vector of the invention typically is associated with stability-promoting salts, carriers (e.g., PEG), and/or formulations that aid in transfection (e.g., sodium phosphate salts, dextran carriers, iron oxide carriers, or biolistic delivery (“gene gun”) carriers, such as gold bead or powder carriers) (see, e.g., U.S. Pat. No. 4,945,050). Additional transfection-enhancing agents include viral particles to which the nucleic acid/nucleic acid vector can be conjugated, a calcium phosphate precipitating agent, a protease, a lipase, a bipuvicaine solution, a saponin, a lipid (preferably a charged lipid), a liposome (preferably a cationic liposome, examples of which are described elsewhere herein), a transfection facilitating peptide or protein-complex (e.g., a poly(ethylenimine), polylysine, or viral protein-nucleic acid complex), a virosome, or a modified cell or cell-like structure (e.g., a fusion cell).

Nucleic acids of the invention can also be delivered by in vivo or ex vivo electroporation methods, including, e.g., those described in U.S. Pat. Nos. 6,110,161 and 6,261,281, and Widera et al., J. Immunol. 164:4635-4640 (2000).

The composition, particularly the pharmaceutical composition, desirably comprises an amount of at least one polynucleotide, polypeptide, and/or vector in a dose sufficient to induce a protective immune response in a mammal, preferably a human, upon administration. The composition can comprise any suitable dose of the at least one polypeptide, polynucleotide, and/or vector. Proper dosage can be determined by any suitable technique. In a simple dosage testing regimen, low doses of the composition are administered to a test subject or system (e.g., an animal model, cell-free system, or whole cell assay system). Considerations in dosing for immunogenic polypeptide, polynucleotide, and/or vector compositions (as well as for gene transfer by viral vectors) are known in the art. Briefly, dosage is commonly determined by the efficacy of the particular nucleic acid, polypeptide, and/or vector, the condition of the subject, as well as the body weight and/or target area of the subject to be treated. The size of the dose is also determined by the existence, nature, and extent of any adverse side-effects that accompany the administration of any such particular polypeptide, nucleic acid, vector, formulation, composition, transduced cell, cell type, or the like in a particular subject. Principles related to dosage of therapeutic and prophylactic agents are provided in, e.g., Platt, Clin. Lab Med. 7:289-99 (1987), J. Kans. Med. Soc. 70(1):30-32 (1969), and other references described herein (e.g., Remington's, supra).

Typically, a nucleic acid composition of the invention comprises from about 1 μg to about 20 mg, about 1 μg to about 15 mg, about 1 μg to about 10 mg, about 1 mg to about 15 mg, about 1 mg to about 10 mg, about 5 mg to about 15 mg, about 5 mg to about 10 mg, about 1 μg to about 5 mg, about 1 μg to about 2 mg, about 1 μg to about 1 mg, 1 μg to about 500 μg, 1 μg to about 100 μg, 1 μg to about 50 μg, and 1 μg to about 10 μg of the nucleic acid. In one aspect, the composition to be administered to a host comprises about 1 to 15 mg, or about 2, 5, or 10 mg of a TAg nucleic acid or vector of the invention. The volume of carrier or diluent in which such nucleic acid is administered depends upon the amount of nucleic acid to be administered. For example, 2 mg nucleic acid is typically administered in a 1 mL volume of carrier or diluent. The amount of nucleic acid in the composition depends on the host to which the nucleic acid composition is to be administered, the characteristics of the nucleic acid (e.g., gene expression level as determined by the encoded peptide, codon optimization, and/or promoter profile), and the form of administration. For example, biolistic or “gene gun” delivery methods of as little as about 1 μg of nucleic acid dispersed in or on suitable particles is effective for inducing an immune response even in large mammals such as humans. In some instances, biolistic delivery of at least about 5 μg, more preferably at least about 10 μg, or more nucleic acid may be desirable. Biolistic delivery of nucleic acids is discussed further elsewhere herein.

For injection of a nucleic acid composition, a larger dose of nucleic acid typically will be desirable. In general, an injectable nucleic acid composition comprises at least about 1 μg nucleic acid, typically about 5 μg nucleic acid, more typically at least about 25 μg of nucleic acid or at least about 30 μg of nucleic acid, 50 μg of nucleic acid, usually at least about 75 μg or at least about 80 μg of the nucleic acid, preferably at least about 100 μg or at least about 150 μg nucleic acid, preferably at least about 500 μg, at least about 1 mg, at least about 2 mg nucleic acid, at least about ˜5 mg nucleic acid, at least about 10 mg, at least about 15 mg nucleic acid, or more. In some instances, the injectable nucleic acid composition may comprise about 0.25-15 mg or 1-10 mg of the nucleic acid, typically in a volume of diluent, carrier, or excipient of about 0.5-5 mL or 0.5 to 2 mL. Commonly, an injectable nucleic acid solution comprises about 0.5 mg, about 1 mg, 1.5 mg, or even about 2 mg nucleic acid, usually in a volume of about 0.25 mL, about 0.5 mL, 0.75 mL, about 1 mL, about 2 mL, or about 5 mL. In one aspect, 2, 5, or 10 mg nucleic acid is typically administered in a. 1 mL volume of carrier, diluent, or excipient (e.g., PBS or saline) at pH 7.4. However, in some instances, lower injectable doses (e.g., less than about 5 μg, such as, e.g., about 4 μg, about 3, about 2 μg, or about 1 μg) of the nucleic acid are about equally or more effective in producing an antibody response than the above-described higher doses. Following priming administration of one or more TAg nucleic acids of the invention (at, e.g., 4-week intervals), one or more TAg proteins of the invention may optionally be administered (e.g., as a protein boost) is a dose(s) ranging from about 0.1 mg to about 5 mg, including about 0.5 mg to 1 mg protein, wherein the protein is delivered as a composition that includes PBS and, if desired, an adjuvant, such as Alum, and optionally at pH 7.4. DNA and protein immunizations are typically delivered administered at 4-week intervals to a subject.

A viral vector composition of the invention can comprise any suitable number of viral vector particles. The dosage of viral vector particles or viral vector particle-encoding nucleic acid depends on the type of viral vector particle with respect to origin of vector (e.g., whether the vector is an alphaviral vector, papillomaviral vector, HSV vector, and/or an AAV vector), whether the vector is a transgene expressing or recombinant peptide displaying vector, the host, and other considerations discussed above. Generally, with respect to gene transfer vectors, the pharmaceutically acceptable composition comprises at least about 1×10⁵ viral vector particles in a volume of about 1 mL (e.g., at least about 1×10⁷ to about 1×10¹³ particles in about 1 mL). Higher dosages also can be suitable (e.g., at least about 1×10⁹, about 1×10¹⁰, about 1×10¹¹, about 1×10¹², or more particles in about 1 mL of carrier). The dose of viral vector particles will vary with the type of viral vector particle used. For example, an effective dose of vaccinia virus particles expressing a polypeptide of the invention can typically be about 2×10⁵ particle forming units (PFU) to about 2×10⁸ PFU. In contrast, a suitable dose of adenoviral particles will usually range from about 1×10⁸ PFU to about 1×10¹² PFU. The skilled artisan can determine similar appropriate doses for other viruses taking into account the principles discussed herein and the effectiveness of similar viral vector particle compositions known in the art.

Nucleic acid compositions of the invention can comprise additional nucleic acids. For example, a nucleic acid can be co-administered with a second immunostimulatory sequence or a second cytokines/adjuvant-encoding sequence (e.g., a sequence encoding an IFN-γ, IL-2, IL-18, TNF-α, and/or a GM-CSF). Examples of such sequences are described above. Nucleic acid compositions of the invention can comprise an additional nucleic acid sequence encoding, or nucleic acids of the invention can comprise an additional sequence encoding, one or more additional cancer-associated antigens, such as MUC, MUC2, MUC3, MUC4, MUC5AC, MUC5B, and MUC7, prostate-specific membrane antigen (PSMA), HER-2/neu, human chorionic gonadotropin-beta, gp75, gp100 (see, e.g., Chen et al., Proc. Natl. Acad. Sci. USA 92:8215-9; Kittlesen et al., J. Immunol. 160:2099-2106 (1998)), MART-1/Melan-A, and carcinoembryonic antigen (CEA), or epitopes thereof. Also or alternatively, a nucleic acid composition can comprise a nucleic acid encoding a costimulatory molecule (e.g., a CD28BP as described above). In other additional or alternative aspects, a nucleic acid of the invention can comprise a sequence encoding or a nucleic acid composition of the invention can comprise a nucleic acid molecule encoding a functional (non-mutated) tumor suppressor gene, such as ras or p53.

The invention also provides a composition comprising an aggregate of two or more polypeptides of the invention as particular polypeptides of the invention can form intermolecular associations. As such, the invention provides a composition comprising a population of one or more multimeric (e.g., dimeric or higher ordered multimeric) polypeptides of the invention (e.g., an oligomer of polypeptides comprising SEQ ID NO: 1, SEQ ID NO:5, or SEQ ID NO:4).

Vaccines

Whole Cell Vaccines

One aspect of the invention pertains a whole cell vaccine, which vaccine comprises a suitable cell, typically a dendritic cell or other APC, which usually is fused to a tumor cell, which whole cell vaccine expressed a polypeptide of the invention that, upon expression remains bound to the cell membrane (e.g., a polypeptide comprising or consisting essentially of SEQ ID NO:6). In a related sense, the invention provides a cell, which can be any cell suitable for ex vivo modification and administration, that comprises a nucleic acid sequence of the invention (e.g., SEQ ID NO: 19 or SEQ ID NO:21), which nucleic acid sequence is expressed from the cell to produce an immunogenic polypeptide of the invention upon administration of the cell to a host.

Tumor-Specific Vaccines

Vaccines inducing an immune response against tumor antigens provide advantages as compared to mAb treatments, because both humoral (specific Ab) and cellular (cytotoxic T cells) arms of the immune system are utilized. Because EpCAM/KSA is a tumor-associated antigen that is overexpressed on a wide variety of adenocarcinomas, it has been targeted using monoclonal antibody approaches. Such approaches have been utilized in human clinical trials' e.g., a phase III randomized multicenter trial of 1839 patients showed statistically significant improvement in survival of surgically resected stage III colon cancer patients; Fields et al., Abstract No. 508, ASCO meeting 2002. However, because another trial showed no or very limited efficacy, some therapies based on monoclonal antibodies (Abs) alone are being re-evaluated. Further, cancer vaccine development has been hampered by the immunological tolerance that prevents strong immune responses against self-antigens, such as those present on tumor cells like EpCAM.

The present invention provides a vaccine approach that induces both specific Abs and T cells against human EpCAM, and thus is expected to provide significant improvements over antibody-based therapies and conventional cancer treatments. In one aspect, the invention provides various vaccine compositions which comprise: (1) at least one novel TAg polypeptide of the invention (e.g., SEQ ID NOS: 1, 4-8) (which is novel variant of hEpCAM antigen) and/or TAg-polypeptide encoding nucleic acid (e.g., SEQ ID NOS: 16, 19-23) (e.g., or expression vector comprising such nucleic acid); and (2) optionally an adjuvant (as describd elsewhere herein) or a novel CD28 binding protein (“CD28BP”), which is a novel co-stimulatory polypeptide that displays preferential binding to human CD28 and has improved costimulatory activity over human B7.1 on T cells. (Lazetic et. al., J. Biol. Chem. 277:38660 (2002), or a nucleic acid encoding the CD28BP polypeptide (or expression vector comprising such nucleotide sequence encoding a CD28BP polpeptide). In a preferred embodiment, the CD28BP is CD28BP is CD28BP-15 (the polypeptide and nucleic acid sequences of CD28BP-15 are designated as SEQ ID NOS:66 and 19 in Int'l Patent App. PCT/US01/19973 (WO 02/00717), respectively). CD28BP-15 is also described in Lazetic et al., supra. Additional polypeptides that preferentially bind human CD28 are described in WO 02/00717. Such composition may comprise an excipient or carrier. Such a composition may be a pharmaceutical composition and the excipient or carrier may be a pharmaceutical excipient or carrier.

With such compositions, a TAg polypeptide of the invention (or nucleic acid or expression vector encoding a TAg polypeptide of the invention) is expected to stimulate a mammal's immune system to recognize the cancer cells (e.g., colon cancer cells), and the adjuvant or CD28BP polypeptide boosts or enhances the system's immune response. Such compositions are expected to allow the immune system to recognize rapidly dividing cancer (e.g., colon cancer) cells and stimulate the immune system to kill such cancer cells. The combination of a TAg polypeptide of the invention as a protein (e.g., SEQ ID NOS: 1, 4-8) or nucleic acid (e.g., SEQ ID NOS: 16, 19-23) and a CD28BP polypeptide (e.g., CD28BP-15 polypeptide (SEQ ID NOS:66) or nucleic acid (SEQ ID NO: 19) shown in WO 02/00717), when administered to a subject, is expected to augment the ability of subject's immune system to recognize and kill cancer cells.

Dosage and Administration: In one embodiment, a single dose of DNA vaccine is typically about 10 mg; a single dose of the TAg protein vaccine is typically about 500 ug. The immunization schedule may comprise two or more rounds each of DNA-DNA-Protein immunizations. Alternatively, TAg protein is administered 2-6 times, at intervals to be determined, and no TAg-encoding DNA is administered. One of skill will understand that other immunization protocols and formats can be utilized. CD28BP is optionally administered as DNA (e.g., a CD28BP-encoding DNA vector is administered) in one or two rounds of the initial DNA/DNA immunizations via injection. The TAg molecule (e.g., TAg-25) is typically administered as DNA (e.g., a TAg-25-encoding DNA vector is administered by injection) followed by a second TAg-encoding DNA injection followed by a TAg protein boost (e.g., TAg-25 protein administered by injection) in reach round of DNA/DNA/protein immunization schedule. Alternatively, TAg is delivered only as DNA (without a protein boost) or only as a protein—in either case with one or more administrations via injection.

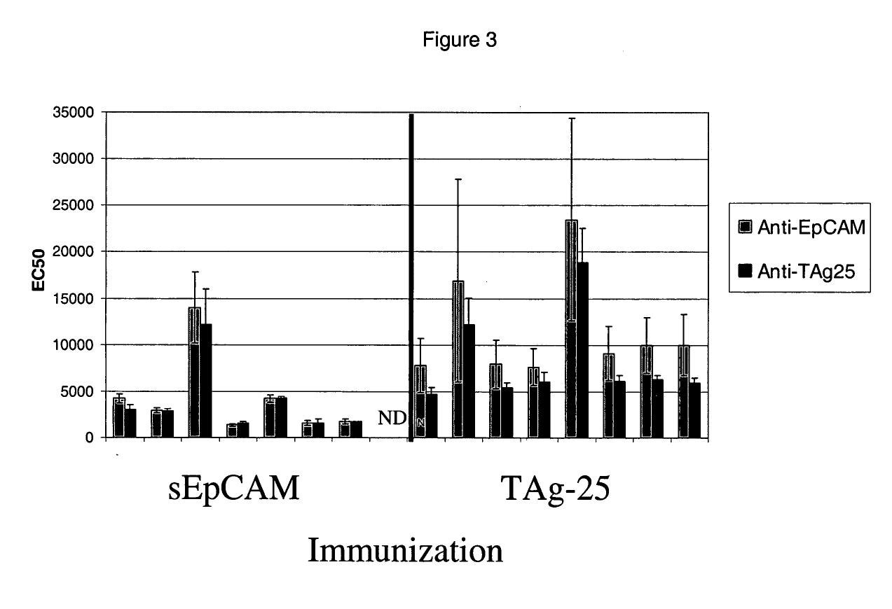

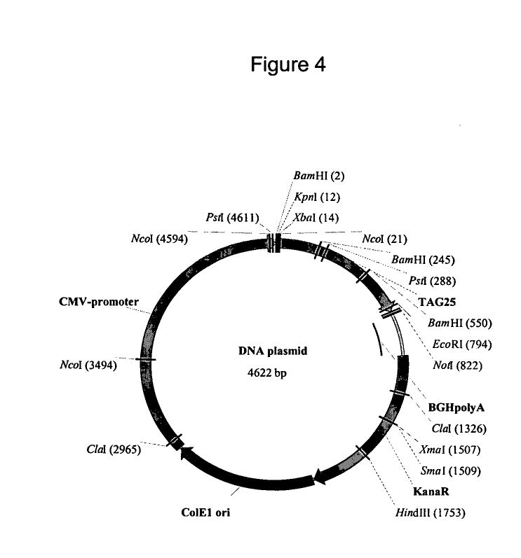

For the vaccine format including both TAg polypeptide and CD28BP, a bicistronic DNA vector encoding both TAg (e.g., TAg-25) and CD28BP (e.g., CD28BP-15) is administered first at a 10-mg dose. An exemplary vector is shown in FIGS. 3-4. Alternatively, TAg and CD28BP can be delivered in DNA format on two separate vectors; in this case, each vector is administered in a 5-mg dose. Following the first DNA immunization, a second identical DNA immunization is given using the bicistronic vector. If desired, the second DNA immunization is followed by administration of 500 ug TAg polypeptide (e.g., TAg-25). This round of immunization is optionally followed by one or more additional rounds of DNA/DNA/protein boost immunization.

Formulation: DNA is formulated in sterile, phosphate-buffered saline at a pH 7.4. TAg protein vaccine (e.g., TAg-25) is formulated in Alum adjuvant.

In an exemplary embodiment, the TAg DNA is TAg-25 (SEQ ID NO:19), and the TAg protein was TAg-25 (SEQ ID NO:4), administered via injection in two rounds each of DNA-DNA-Protein immunizations using doses noted above. In such administration format, the vaccine induced high titers of anti-EpCAM antibodies in mice and cynomolgus monkeys. TAg-25 DNA immunization of cynomolgus monkeys induced antibody responses that cross-react with human EpCAM. TAg-25 protein boost greatly augmented EpCAM-specific immune responses induced by DNA vaccine alone. TAg-25 polypeptide was at least as immunogenic as WT hEpCAM in mice and cynomolgus monkeys. Because of amino acid sequence differences between TAg-25 polypeptide and hEpCAM (which is a self antigen), improved immune responses can be expected in humans.

The vaccine composition (e.g., administered in two rounds of DNA-DNA-Protein immunizations) induced T cell immunity in mice and cynomolgus monkeys. Upon DNA immunization of cynomolgus monkeys, only the combination of TAg-25 and CD28BP was sufficient to induce EpCAM-specific IFN-γ production by CD8⁺ T cells. This IFN-γ production was not observed using either TAg-25 DNA alone or in combination with human B7.1. The TAg25 protein alone elicited high anti-EpCAM antibody (Ab) titers, but most potent responses (antibodies and CD8+ T cells) were obtained by DNA priming—protein boost approach. No side effects were observed in animals treated with the vaccine. Clinical observations of cynomolgus monkeys, analysis of serum chemistry, and assessment of immunogenicity of CD28BP in mice, monkeys and human T cells in vitro provided no evidence of harmful immunogenicity of CD28BP. CD28BP immunization of mice or cynomolgus monkeys induces antibody responses that do not cross-react with human B7.1 nor alter normal immune responses either in vivo or in vitro. The preclinical studies in mice and cynomolgus monkeys indicate the vaccine induces both humoral and cellular immune responses against the target antigen and suggests an excellent safety profile.

The invention provides methods for treating EpCAM-associated malignancies comprising administering one or more vaccine compositions of the invention. In one aspect, the method comprises administering to a subject in need of treatment an effective amount of TAg-25 DNA at two separate intervals and then administering to the subject an effective amount of TAg-25 polypeptide. The effective amount includes, but is not limited to, the respective doses. The protein and DNA vaccines are formulated as noted above.

Among other uses, the vaccine is indicated to delay the occurrence of metastatic disease in subjects with EpCAM/KSA+malignancies, such as, e.g., stage II and III colon and colorectal cancers, who are undergoing surgical resection for staging and cure. The vaccine is expected to statistically significantly prolong the median time to recurrence of the tumor. The vaccine is expected to reduce the spread of malignant cells in the peri-operative period. Cytotoxic T-cells induced by the vaccine composition are expected to kill cancer cells. In addition, antibodies induced by the vaccine composition are expected to lyse cancer cells via antibody dependent cellular cytotoxicity. This vaccine approach overcomes limitations of current cancer vaccines and is useful in breaking the immunological tolerance against EpCAM/KSA. The vaccine is useful as an adjuvant in treatment of subjects with EpCAM/KSA⁺ malignancies, including colorectal cancers.

Kits

The present invention also provides kits including one or more of the polypeptides, nucleic acids, vectors, cells, vaccines, and/or compositions of the invention. Kits of the invention optionally comprise (1) at least one polypeptide, nucleic acid, vector, cell, vaccine, or composition; (2) instructions for practicing any method described herein, including a therapeutic or prophylactic method, instructions for using any component identified in (1); (3) a container for holding said at least one such component or composition, and/or (4) packaging materials. One or more of the polypeptides, nucleic acids, vectors, cells, vaccines, and/or compositions of the invention can be packaged in packs, dispenser devices, and kits for administration to a subject, such as a mammal. Packs or dispenser devices that contain one or more unit dosage forms are provided. Typically, instructions for administration of the compounds are provided with the packaging, along with a suitable indication on the label that the compound is suitable for treatment of an indicated condition. For example, the label may state that the active compound within the packaging is useful for treating a particular EpCAM-associated tumors or diseases or conditions associated with overexpression of EpCAM.

### EXAMPLES

The following examples are illustrative and should not be construed as limiting the scope of the present invention in any way. One of ordinary skill in the art will recognize that a variety of non-critical parameters can be altered to achieve essentially similar results.

### Example 1

This example describes the generation of novel hybridomas that express antibodies that bind human EpCAM an antigenic fragment thereof (e.g., sEpCAM).

Female BALB/c mice were purchased from Taconic (Germantown, N.Y. 12526) and used for experiments at 6-8 weeks of age. All mice were housed in specific pathogen-free conditions for the course of the experiment.

The Balb/c mice received 25 μg affinity purified protein comprising SEQ ID NO:4 (hereinafter referred to as tumor-associated antigen-25 or “TAg-25”), emulsified 1:1 in Complete Freund's Adjuvant (Sigma), by subcutaneous (s.c) injection. This first administration of TAg-25 protein/adjuvant was followed by a second subcutaneous (s.c.) injection of 25 μg affinity purified TAg-25 protein emulsified 1:1 in Incomplete Freund's Adjuvant (Sigma) and a final intravenous (i.v.) administration with 25 μg affinity purified TAg-25 protein prepared in sterile phosphate buffered saline (PBS), pH 7.4. The injections of the protein compositions were given two weeks apart. Three days after the last administration, hybridomas were prepared from the spleen of the treated mice as follows.

Single cell spleen suspensions were prepared in DMEM (Gibco) supplemented with 2 mM glutamine (Gibco), 15 mM HEPES (Gibco), 5 mM Sodium Pyruvate (JRH 59-20377P), 5 mM non essential amino acids (Gibco 320-1140AG), and 20% Fetal Bovine Serum (FBS-Hyclone Lot #ALA12955). This supplemented DMEM medium is the “growth medium” used in the experiments discussed below.

The suspended spleen cells were centrifuged at 1,200 rpm for 10 minutes, resuspended in 8 ml 0.17M NH₄Cl and fused with Sp2/0 cells (ATCC # CRL-1581) as previously described in Ozato and Sachs, J. Immunol. 126:317-321 (1981). Briefly, 12 ml of 0.3% Dextran (Sigma) was added to the cell mixture and after 5 minutes at room temperature, the cells were centrifuged for 10 minutes at 1,000 rpm. The cells were then resuspended in 1 ml of PEG1500 (Roche Applied Science Cat# 0783641) at 37° C. 20 ml of serum free DMEM was slowly added to the cells and the cells were subsequently centrifuged for 10 minutes at 1,000 rpm. The cells were resuspended in selection medium (growth medium containing 2 ug/ml azaserine (Sigma, Cat# A-9666)) and 50-100 μl/well added to 96-well flat bottom plates (Costar). Plates were incubated at 37° C. for 4 days. During this 4-day period, the hybridomas were fed with growth medium as necessary.

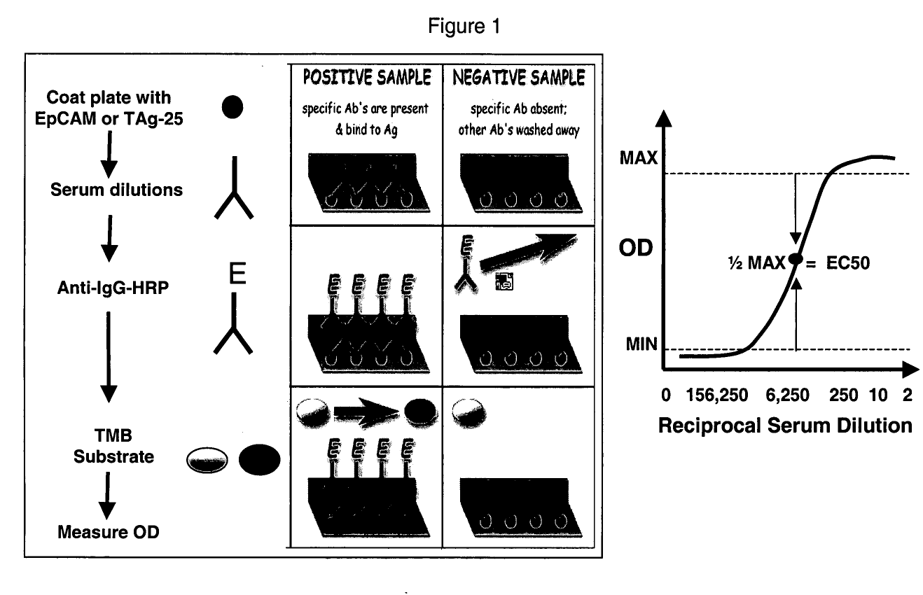

The hybridomas were plated and screened in two assays. First, the hybridomas were analyzed for their ability to generate antibodies that recognize human sEpCAM by ELISA assay. To perform the ELISA assay, 96 well ELISA plates (Nunc Maxisorb) were coated with 50 μl/well of affinity purified human sEpCAM-his-tagged fusion protein (comprising human sEpCAM fused to a histidine epitope tag (“ehis”) comprising 6 histidine residues) or human sEpCAM protein purified from baculovirus transfected insect cells (gift from Håakan Mellstedt, Cancer Centre Karolinska, Depart. Oncology, Karolinska Hospital, Stockholm, Sweden) at 0.6-1 μg/ml overnight at 4° C. Wells were then washed three times with 200 μl/well of a solution of PBS and 0.05% Tween 20 and blocked by adding 100 μl/well of a solution of PBS, 3% BSA, and 0.1% sodium azide or 5% powdered milk (diluted in a solution of PBS and 0.1% sodium azide) for 1 hour at room temperature. After washing as previously described, 50 μl/well of serum (mouse sera diluted 1/500) was added in triplicate, and the plates incubated for 2 hours at 37° C. The plates were washed as previously described, and horseradish peroxidase (HRP)-conjugated anti-murine IgG (Caltag), diluted to a concentration of 1/4000 in a solution of PBS, 0.05% Tween 20, and 0.1% BSA (100 μl/well), was added to the plates. The plates were then incubated for 1 hour at 37° C. The plates were then washed as previously described. TMB substrate (Pierce cat# 34021) was prepared according to the manufacturer's instructions and 100 μl TBS substrate/well was added to the plates until the desired color intensity (absorbance) was achieved, indicating formation of a labeled sEpCAM antigen/antibody complex. The complex concentration is determined by measuring absorbance (optical density (OD)) of the reaction substrate on each plate at 450 nm on a Spectramax 190 using Softmax Pro version 3 software (both from Molecular Devices Corp.) (FIG. 1).

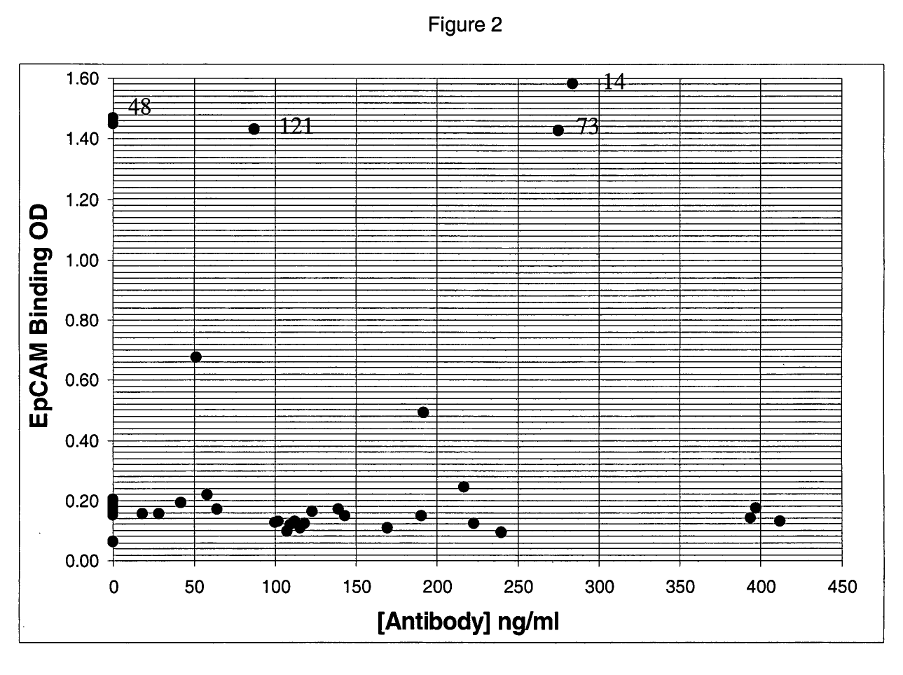

The concentration of antibodies expressed by the hybridomas was quantified through titrations made using an Easy-Titer Mouse IgG Assay kit (Pierce Cat # 23300), according to the manufacturer's instructions. The combination of information from the absorbance and titration assays allows an estimation of antibody affinity. A high ELISA OD together with low antibody concentration indicated a hybridoma secreting high affinity antibody for sEpCAM. The results of these experiments are shown in FIG. 2.

These results demonstrate that an immunogenic or antigenic polypeptide of the invention, such as a TAg antigen, can be used to generate a hybridoma that produces monoclonal antibodies that react with (e.g., bind to or specifically or selectively bind to) EpCAM or an antigenic fragment thereof, such as sEpCAM. For example, at least seven of the hybridomas generated by this method expressed monoclonal antibodies (mAbs) that specifically bound sEpCAM to produce a labeled sEpCAM-mAb complex having an OD of at least about 0.5. Moreover, two such clones produced such mAbs at concentrations of over 250 ng/ml. By evaluating this data, several hybridoma clones were selected as advantageous for the production of monoclonal antibodies that bind to human EpCAM or an antigenic fragment thereof, such as, e.g., sEpCAM or the ECD of hEpCAM. This example demonstrates that the polypeptides of the invention can be used to produce novel hybridomas that efficiently produce or express antibodies that bind to or cross-react with human EpCAM or antigenic fragments thereof, such as sEpCAM.

### Example 2

This example demonstrates the ability of an antigenic or immunogenic polypeptide of the invention to induce production of antibodies against human EpCAM or an antigenic fragment thereof in a mammalian host. Specifically, in this example, TAg-25 (SEQ ID NO:4) is shown to induce production of antibodies against sEpCAM (SEQ ID NO:40).

Six groups of eight BALB/c mice (Taconic, Germantown, N.Y. 12526) per group were injected with a protein solution comprising 10 μg purified protein (either affinity purified sEpCAM-his-tagged fusion protein, TAg-25-his-tagged fusion protein, or baculovirus cell-expressed purified sEpCAM antigen) in 100 μl of 1.5% alum, such that the animals received either 5 μg of the solution injected intramuscularly (i.m.) into each of the animal's two deltoid muscles, or 10 μg injected s.c. at the base of the animal's tail sEpCAM-his-tagged fusion protein comprises the polypeptide sequence of sEpCAM (SEQ ID NO:40) to which a histidine epitope tag comprising 6 histidine residues is fused at the C terminus of the sEpCAM polypeptide sequence. TAg-25-his-tagged fusion protein comprising the polypeptide sequence of TAg-25 (SEQ ID NO:4) to which a 6-histidine residue tag sequence is fused to the C terminus of the TAg-25 polypeptide sequence. An additional group of 4 control mice each received an administration of 10 μg bovine serum albumin (BSA) or nothing (no treatment). Treated mice received administrations of the respective protein solution on days 1, 14, and 28. Serum was collected from each of the treated and control mice at days 0, 27, 38, and 52 for antigen-specific antibody ELISA assays as described in Example 1. In this example, plates were coated with either sEpCAM or TAg-25 polypeptide at a concentration described in Example 1. Mice were sacrificed by cervical dislocation on day 83 and the spleens prepared for antigen-specific T cell assays (described elsewhere herein).

FIG. 3 shows results for two groups of eight individual mice immunized with either sEpCAM or TAg-25 polypeptide. (“ND” means “no data” for an individual mouse. The effective concentration that represents 50% of the maximum serum concentration (EC50) of antibodies generated that specifically bound either sEpCAM (SEQ ID NO:40) or TAg-25 polypeptide (SEQ ID NO:4) in these mice was determined by sEpCAM or TAg-25 antigen-specific Ab ELISA assay, respectively, as described in Example 1. As shown in FIG. 3, i.m. immunization with TAg-25 polypeptide induced production of anti-sEpCAM antibodies in 8/8 mice with EC50 values at least as great as (if not greater than) the EC50 values obtained upon i.m. administration to mice of the same amount of sEpCAM i.m. Similar results were obtained when either TAg-25 polypeptide or sEpCAM antigen was administered by s.c. route (data not shown). The EC50 values obtained in the ELISA assay using sera obtained from mice immunized with TAg-25 polypeptide were significantly higher than the background levels of sEpCAM-specific antibody present in mice receiving BSA/Alum and in the pre-bleeds from each mouse used for these studies (data not shown). These results indicate that sEpCAM-specific B cells had been selectively activated by the TAg-25 protein immunizations (data not shown). In addition, each immunization with TAg-25 polypeptide produced comparable concentrations of antibodies specific to sEpCAM or to TAg-25, suggesting that sEpCAM and TAg-25 proteins contained fully cross-reactive B cell epitopes.

The results of these experiments demonstrate that polypeptides of the invention have the ability to induce or enhance production of antibodies against EpCAM, or an antigenic fragment thereof such as sEpCAM, in a mammalian host. More particularly, the results demonstrate that Tag-25 polypeptide has an ability to induce or enhance the production of antibodies against EpCAM or an antigenic fragment thereof at concentrations comparable to, if not greater than, the concentrations of antibodies induced by EpCAM or an antigenic fragment thereof.

### Example 3

This example describes strategies for the generation of exemplary DNA vectors of the invention (e.g., pMaxVax vectors described herein), which are suitable for use in DNA immunization methodologies for inducing or promoting an immune response to EpCAM.

An exemplary monocistronic pMaxVax vector of the invention comprises, among other things: (1) a promoter for driving the expression of a transgene (or other nucleotide sequence) in a mammalian cell (including, e.g., but not limited to, a CMV promoter or a variant thereof, and shuffled, synthetic, or recombinant promoters, including those described in PCT Int'l Application No. PCT/US01/20123 (Int'l Publ. No. WO 02/00897); (2) a polylinker for cloning of one or more transgenes (or other nucleotide sequence); (3) a polyadenylation (polyA) signal sequence; and (4) a prokaryotic replication origin; and (5) antibiotic resistant gene for amplification in E. coli or other suitable cell. The construction of such a monocistronic vector is briefly described herein, although several suitable alternative techniques are available to produce such a DNA vector (e.g., applying the principles described elsewhere herein). See also the description of the pMaxVax 10.1 vector in commonly assigned International (Int'l) App. No. PCT/US01/19973 (WO 02/00717) and Int'l App. No. PCT/US02/19898.

In one embodiment, the minimal plasmid Col/Kana comprises the replication origin ColE1 and the kanamycin resistance gene (Kana^{r}). The ColE1 origin of replication (ori) mediates high copy number plasmid amplification. Alternatively, low copy number replication origins, such as p15A (from plasmid pACYC177, New England Biolabs Inc.) can be used.

To produce a monocistronic vector having these features, ColE1 ori was isolated from vector pUC19 (New England Biolabs, Inc.) by application of standard PCR techniques. To link the ColE1 origin to the Kana^{r} gene, unique NgoMIV (or “NgoMI”) and DraIII recognition sequences were added to the 5′ and 3′ PCR primers, respectively. For subsequent cloning of the mammalian transcription unit, the 5′ forward primer also was designed to include the additional restriction site NheI downstream of the NgoMIV site and EcoRV and BsrGI cloning sites upstream of the DraIII site the 3′ reverse primer. Primers were typically designed to include additional 6-8 base pairs overhang for optimal restriction digest. Typically, the ColE1 PCR reactions were performed with proof-reading polymerases, such as Tth (PE Applied Biosystems), Pfu, Pfu Turbo and Herculase (Stratagene), or Pwo (Roche), under conditions in accordance with the manufacturer's recommendations.

The ColE1 PCR product was purified with phenol/chloroform using Phase lock Gel™Tube (Eppendorf) followed by standard ethanol precipitation. The purified ColE1 PCR product was digested with the restriction enzymes NgoMIV and DraIII according to the manufacturer's recommendations (New England Biolabs, Inc.) and gel purified using the QiaExII gel extraction kit (Qiagen) according to the manufacturer's instructions. In this embodiment, the Kanamycin resistance gene (transposon Tn903) was isolated from plasmid pACYC177 (New England Biolabs, Inc.) using standard PCR techniques.

In one embodiment, the pMaxVax monocistronic vector comprises a CMV immediate early enhancer promoter (CMV IE), which can be isolated from DNA of the CMV virus, Towne strain, by standard PCR methods. The cloning sites EcoRI or EcoRV and BamHI were incorporated into the PCR forward and reverse primers. The EcoRI/EcoRV and BamHI digested CMV IE PCR fragment was cloned into pUC 19 for amplification. The CMV promoter was isolated from the amplified pUC19 plasmid by restriction digest with BamHI and BsrGI. The BsrGI site is located 168 bp downstream of the 5′ end of the CMV promoter, resulting in a 1596 bp fragment, which was isolated by standard gel purification techniques for subsequent ligation. To produce a pMaxVax monocistronic vector comprising a different promoter, a similar technique is used.

In one embodiment, a polyadenylation signal from the bovine growth hormone (BGH) gene can be used. Other polyadenylation signals which work well in mammalian cells, include, e.g., poly A signal sequences from, e.g., SV40, Herpes simplex Tk, and rabbit beta globin, and the like, and others known to those of skill in the art. For example, a BGH nucleotide sequence or fragment thereof can be isolated from pCDNA3.1 vector (Invitrogen) by standard PCR techniques using a 5′PCR forward primer which includes recognition sites for the restriction enzymes PmeI and BglII to form part of the pMaxVax vector polylinker, and a 3′ reverse primer, which includes a DraIII site for cloning to the minimal plasmid Col/Kana. Primers were prepared by standard techniques and used to amplify a BGH polyA PCR product. The BGH polyA PCR product was diluted 1:100. 1 microliter of the diluted BGH polyA PCR product was used as a template for a second PCR amplification using the same 3′ reverse primer and a second 5′ primer, which overlapped the 5′ end of the template by 20 bp, and contained another 40 bp 5′ sequence comprising BamHI, KpnI, XbaI, EcoRI, and NotI restriction sites for inclusion of these sites in the p.MaxVax10.1 vector polylinker.

The final ligation reaction to form pMaxVax monocistronic vector backbone was performed with about 20 ng each of the BsrG1 and BamHI digested CMV IE PCR product, BamHI and DraIII digested polylinker and BGH poly A PCR product, and the DraIII and BsrG1 digested minimal plasmid Col/Kana in a 50 microliter reaction with 5 microliter 10× ligase buffer and 2U ligase (Roche). Ligation, amplification, and plasmid purification were performed as described above. The plasmid was transfected into E. coli (e.g., XL1-blue-mrf (Stratagene) electro-competent bacteria) and cloned using standard techniques. For example, the transformed bacterial cells can be grown on agar plates in starter media (10 g Tryptone, 5 g Yeast Extract, 10 ng NaCl/liter DDH₂O) in selective Kanamycin medium (40 μg/ml concentration), for 5 hours, which medium is subsequently diluted 1:1000 into 200-500 mL cultures in selective LB media and thereafter grown for 14-16 hours. The bacterial cultures are spun down (pelleted) by centrifugation, and the plasmid DNA purified (Qiagen Endofree plasmid purification kit) and dissolved in endotoxin-free PBS (Sigma) at a final concentration of about 1 μg/μl).

A nucleotide sequence of the invention (SEQ ID NO: 19) encoding TAg-25, was inserted into the monocistronic pMaxVax vector by digesting the vector backbone with XbaI and NotI (which are unique restriction sites in the polylinker of the pMaxVax vector) using standard techniques, gel purifying the linearized vector, and ligating the novel cancer antigen-encoding sequence thereto. FIG. 4 shows a map of an exemplary monocistronic pMaxVax DNA vector comprising such a nucleotide sequence and other elements. Such monocistronic pMaxVax vectors are readily reproducible in E. coli and suitable for inducing an immune response in a mammal. Similar techniques were performed to add EpCAM polypeptide-encoding nucleic acids to the pMaxVax vector backbone (pMaxVax. 10.1 vector), for control experiments described herein.

Using the polylinker in the pMaxVax backbone vector, a bicistronic vector comprising a first expression cassette comprising a first nucleotide sequence encoding a costimulatory polypeptide, particularly, e.g., a cytokine or CD28BP (as described above) and a second expression cassette comprising a second nucleotide sequence of the invention (e.g., a nucleic acid comprising a polynucleotide sequence having at least about 90, 95, 96, 97, 98, 99 or 100% sequence identity to a polynucleotide sequence selected from SEQ ID NOS: 16, 19-23, 26-28, 33, 35, and 79) and can be generated. In an alternative format, the first expression cassette comprises the nucleic acid of the invention and the second expression cassette comprises the nucleotide sequence encoding the costimulatory polypeptide. For example, a pMaxVax bicistronic vector can be generated as follows. The unique restriction sites BamHI and KpnI are used to linearize the pMaxVax backbone vector and thereafter clone a first expression cassette comprising a costimulatory polypeptide-encoding nucleotide sequence operably linked to a first CMV promoter or other promoter, which expression cassette sequence was engineered to have corresponding sites at its 5′ and 3′ ends, into the backbone to form an intermediate vector. The unique restriction sites NgoMI, AccI and NheI were used to clone the second expression cassette, comprising a TAg-25-polypeptide-encoding nucleotide sequence operably linked to a second CMV promoter or other promoter in two parts (e.g., the TAg-25-encoding sequence was cloned in by the AccI and NheI sites). The resulting pMaxVax bicistronic vector is shown in FIG. 5. Bicistronic vectors of the invention can be cloned in mammalian cells (e.g., COS) E. coli cells and used as DNA vaccines in mammalian hosts. In a particular embodiment, the bicistronic vector comprises a first expression cassette comprising a first nucleotide sequence encoding CD28BP-15 (i.e., the polypeptide comprising the polypeptide sequence of SEQ ID NO:66 shown in Int'l Patent App. PCT/US01/19973) and a second expression cassette comprising a second nucleotide sequence that encodes SEQ ID NO:4 (TAg-25) of the present invention. SEQ ID NO:66 (CD28BP-15 polypeptide) of Int'l Patent App. PCT/US01/19973 is:

MGHTMKWGSLPPKRPCLWLSQLLVLTGLFYFCSGITPKSVTKRVKETVML
SCDYNTSTEELTSLRIYWQKDSKMVLAILPGKVQVWPEYKNRTITDMNDN
PRIVILALRPSDSGTYTCVIQKPVLKGAYKLEHLASVRLMIRADFPVPTI
NDLGNPSPNIRRLICSTSGGFPRPHLYWLENGEELNATNTTVSQDPGTEL
YMISSELDFNVTNNHSIVCLIKYGELSVSQIFPWSKPKQEPPIDQLPFWV
IIPVSGALVLTAVVLYCLACRHVARWKRTRRNEETVGTERLSPIYLGSAQ
SSG

### Example 4

This example demonstrates the ability of a vector comprising at least one nucleic acid of the invention to express a polypeptide that react with antibodies induced by human EpCAM or an antigenic fragment of EpCAM, such as sEpCAM.

The following four expression vectors were constructed using the methods outlined in Example 3 above: (1) a monocistronic pMaxVax expression vector encoding TAg-25 antigen (SEQ ID NO:4); (2) a monocistronic pMaxVax vector encoding sEpCAM antigen (SEQ ID NO:40); (3) a bicistronic pMaxVax vector encoding TAg-25 antigen and a costimulatory polypeptide, such as human B7-1 or CD28BP-15 (discussed above); and (4) a bicistronic pMaxVax vector encoding sEpCAM and a costimulatory polypeptide, such as human B7-1 or CD28BP-15 (discussed above). An exemplary pMaxVax monocistronic vector comprising a polynucleotide sequence (e.g., SEQ ID NO: 19) encoding TAG-25 is shown in FIG. 4 and described in Example 3 above. An exemplary pMaxVax bicistronic vector comprising both a polynucleotide sequence (e.g., SEQ ID NO: 19) encoding TAg-25 and a polynucleotide sequence encoding CD28BP-15 is shown in FIG. 5 and described in Example 3 above. The polynucleotide sequence that encodes CD28BP-15 is designated SEQ ID NO: 19 in International Patent App. PCT/US01/19973.

A pMaxVax monocistronic vector comprising a polynucleotide sequence (e.g., SEQ ID NO:93) encoding sEpCAM was similarly constructed. In addition, a pMaxVax bicistronic vector comprising both a polynucleotide sequence (e.g., SEQ ID NO:93) encoding sEpCAM and a polynucleotide sequence encoding CD28BP-15 was constructed.

Each of the pMaxVax vectors (in PBS) was transfected, respectively, into four individual HEK 293 cell cultures using Effectene reagent under conditions described by the manufacturer (Qiagen) (0.4 μg vector/one well of 6-well plate, with each well containing about 2-3×10⁵ cells). Transfected cells were cultured for 2 days under suitable cell culture conditions.

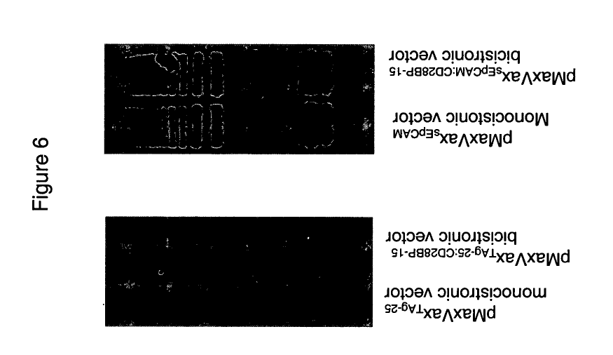

15 μl of supernatant was aspirated from each cell culture supernatant and subjected to polyacrylamide gel electrophoresis. The gel was blotted to nitrocellulose membranes using the technique described by the manufacturers (NuPage and Invitrogen, respectively). The filters were incubated with labeled sEpCAM-binding monoclonal antibody (mAb A323). The antibody-antigen incubations were performed for 1 hour at room temperature, the filters were washed 5 times for 25 minutes with PBS Buffer and 0.1% Tween 20, and the filters were further incubated with a secondary enzyme-conjugated (either a horse radish peroxidase (HRP)-conjugated or alkaline phosphatase-conjugated) anti-mouse antibody. After a 1-hour incubation at room temperature, the filters were washed and incubated with the enzyme substrates for colorimetric detection. The resulting Western blots illustrating expression and/or secretion in human 293 cells of polypeptides (e.g., polypeptides of the invention) that react with antibodies to human sEpCAM are shown in FIG. 6.

The Western blots demonstrate that a monocistronic or bicistronic vector comprising a TAg-25 nucleic acid sequence is capable of expressing a significant amount of TAg-25 polypeptide that is recognized by anti-EpCAM antibodies (A323) in mammalian cells. The intensities of the bands in the Western blots for cell cultures transfected with the monocistronic and bicistronic vectors encoding TAg-25 polypeptide are comparable to the intensities of bands resulting from cell cultures transfected with monocistronic and bicistronic vectors encoding human sEpCAM, respectively. The expression of sEpCAM produced more complex band patterns, suggesting the formation of sEpCAM multimers at levels greater than those observed with TAg-25 (see FIG. 6). The amount of sEpCAM multimers formed is expected to be small. The assay was not designed to determine whether TAg-25 polypeptide also could form multimers. Expression of CD28BP-15 was not shown by this assay, but by FACS assays (data not shown) (see, e.g., assays described in Int'l Patent App. No. PCT/US01/19973). This example demonstrates that a pMaxVax monocistronic vector that encodes a TAg antigen of the invention is capable of expressing the TAg antigen effectively in mammalian cells. This example also shows that a bicistronic vector that encodes a TAg antigen and a costimulatory polypeptide is able to express the TAg antigen effectively in mammalian cells.

### Example 5

This example demonstrates the induction of an immune response against human EpCAM or an antigenic fragment thereof by nucleic acids of the invention and vectors comprising such nucleic acids.

A first group of Balb/c mice (5 mice/group) was injected with a composition comprising 125 μg of a monocistronic pMaxVax vector encoding human sEpCAM (“pMaxVax_{sEpCAM}”) in 100 μL of sterile PBS. A second group of Balb/c mice (5 mice/group) was injected with a composition comprising 125 μg of a monocistronic vector encoding TAg-25 polypeptide (“pMaxVax_{TAg-25}”) in 100 μL of sterile PBS. To each mouse of a control group comprising 5 Balb/c mice was administered 125 μg of a pMaxVax vector that does not encode an antigen (e.g., pMaxVaxₙᵤₗₗ or “empty” vector). This vector, which served as a vector control, was identical to that shown in FIG. 4 except that no TAg-25 or sEpCAM antigen-encoding nucleotide sequence was included in the vector. Each individual mouse was injected with 65 μg intramuscularly (i.m.) into each of the two deltoid muscles for a total of 100 μg/mouse. The vector doses were administered on days 1, 20, 41, and 63, respectively. The pMaxVax_{TAg-25} vector comprised a polynucleotide sequence (e.g., SEQ ID NO: 19) that encodes TAg-25 antigen (SEQ ID NO:4). An exemplary pMaxVax_{TAg-25} vector is shown in FIG. 4. The pMaxVax_{sEpCAM} vector was identical to that shown in FIG. 4 except that a nucleotide sequence encoding sEpCAM was substituted for the nucleotide sequence encoding TAg-25 antigen.

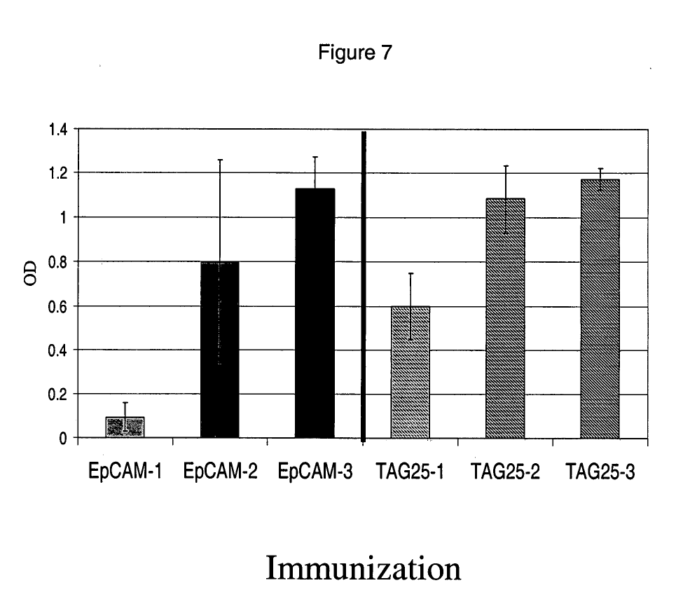

Serum was collected from each mouse at days 21, 42, and 64 for antibody ELISA assays. Mice were sacrificed by cervical dislocation on day 137 and their spleens prepared for antigen-specific T cell assays (discussed further herein). Each collected sample of mouse serum was subjected to an antigen-specific antibody ELISA assay in which the antigen was sEpCAM (1:500 dilution) as described above to determine antibody levels. The mean OD values obtained for sera pooled from each group of mice at each of the final three serum collection time points are presented in FIG. 7. Each experiment was performed in triplicate.

The resulting OD values in the ELISA for sera obtained from mice immunized with pMaxVax_{TAg-25} vector were at least as high as, if not higher than, those obtained from mice immunized with a pMaxVax_{sEpCAM} vector in all three rounds of immunization. The OD value is a reflection of antibody titer. Moreover, the OD values for sEpCAM-specific antibodies in sera obtained from mice immunized with pMaxVax_{TAg-25} vector were significantly higher than the OD values for sEpCAM-specific antibodies in sera obtained from mice immunized with the empty vector control (pMaxVaxₙᵤₗₗ) (data not shown). DNA immunization with a TAg-25 encoding DNA sequence expressed from a plasmid expression vector induced hEpCAM-specific antibodies.

This experiment demonstrates that a nucleic acid vector encoding an antigenic polypeptide of the invention can generate an immune response, particularly a humoral immune response, against human EpCAM or an antigenic fragment thereof in a mammalian host as effectively, if not more effectively, than a nucleic acid vector encoding sEpCAM.

### Example 6

This example illustrates that immunization of cynomolgus monkeys with a nucleic acid vector expressing a novel immunogenic polypeptide of the invention induces production of antibodies that specifically bind to hEpCAM or an antigenic fragment thereof and to the novel immunogenic polypeptide.

3.5 to 6.5 year-old male cynomolgus (Macaca fascicularis) monkeys ranging in weight from 2.5-6.5 kg and housed at SNBL USA, Ltd. (Everett, Wash. 98203) were selected for the following experiments.

Experimental groups of four monkeys received injections of a solution comprising a monocistronic pMaxVax_{TAg-25} vector or pMaxVax_{sEpCAM} vector in sterile PBS at pH 7.4. Specifically, each monkey was injected with 1 mg of the respective DNA vector such that animal either received an i.m. injection into the deltoid muscle at each of two sites, or an intradermal (i.d.) injection at each of 5 to 8 sites. Each immunization dose was administered on days 0, 22, 43 and 64 (for a total of 4 doses). An additional 2 control monkeys were administered a 0.9% NaCl (i.m.) solution at the same time points.

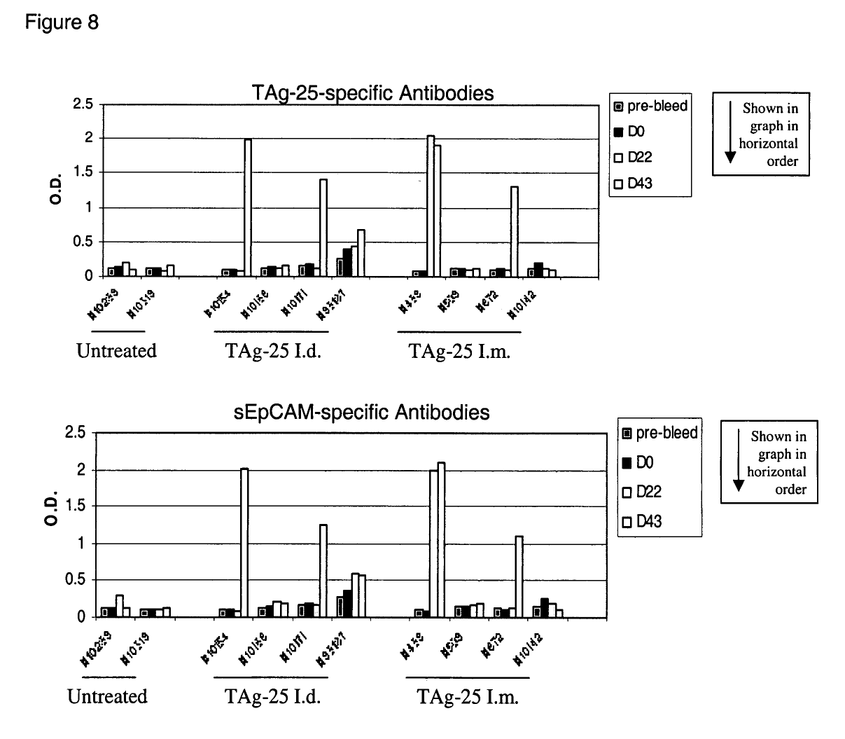

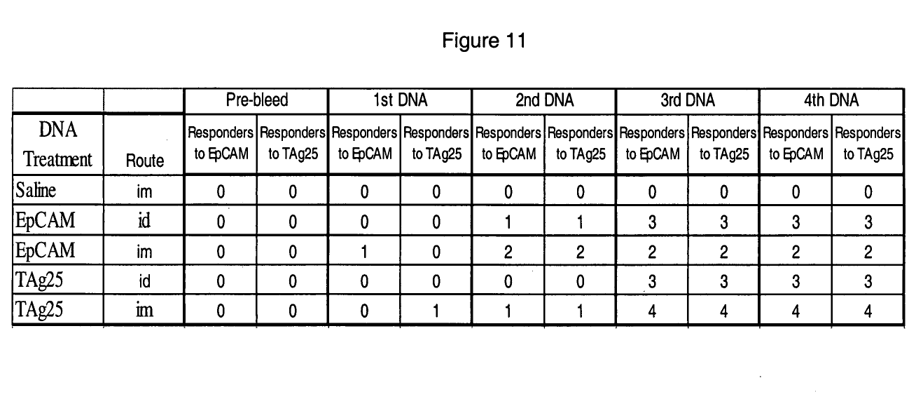

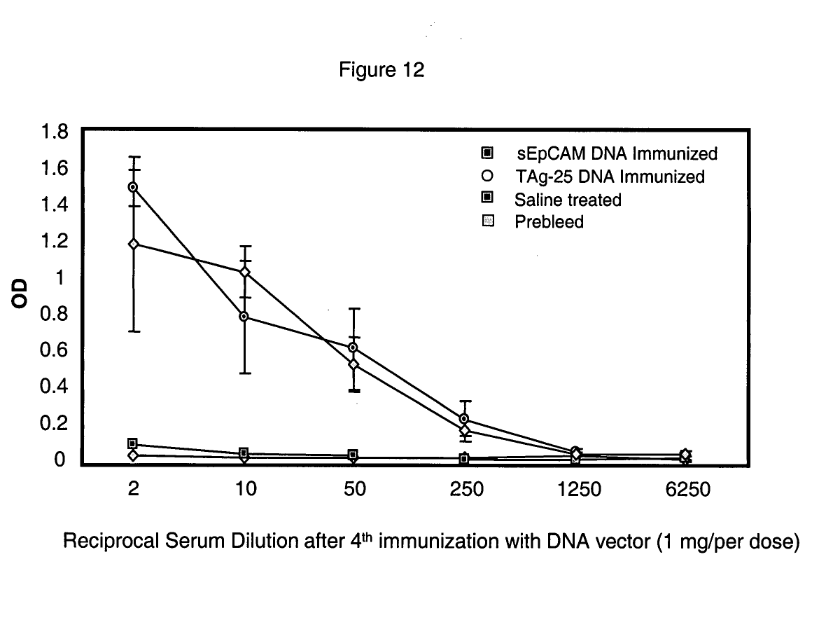

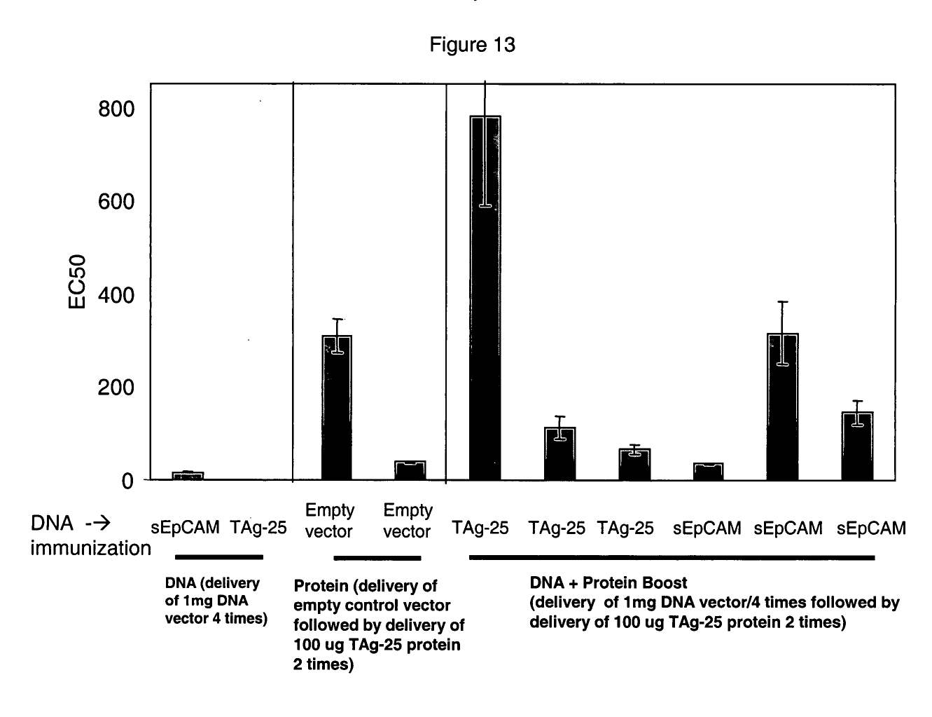

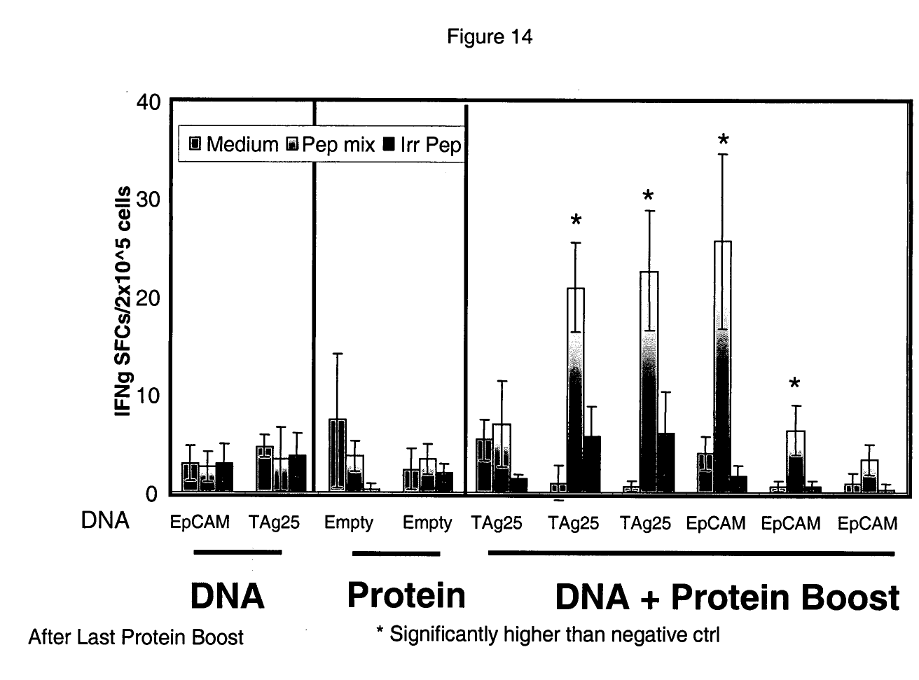

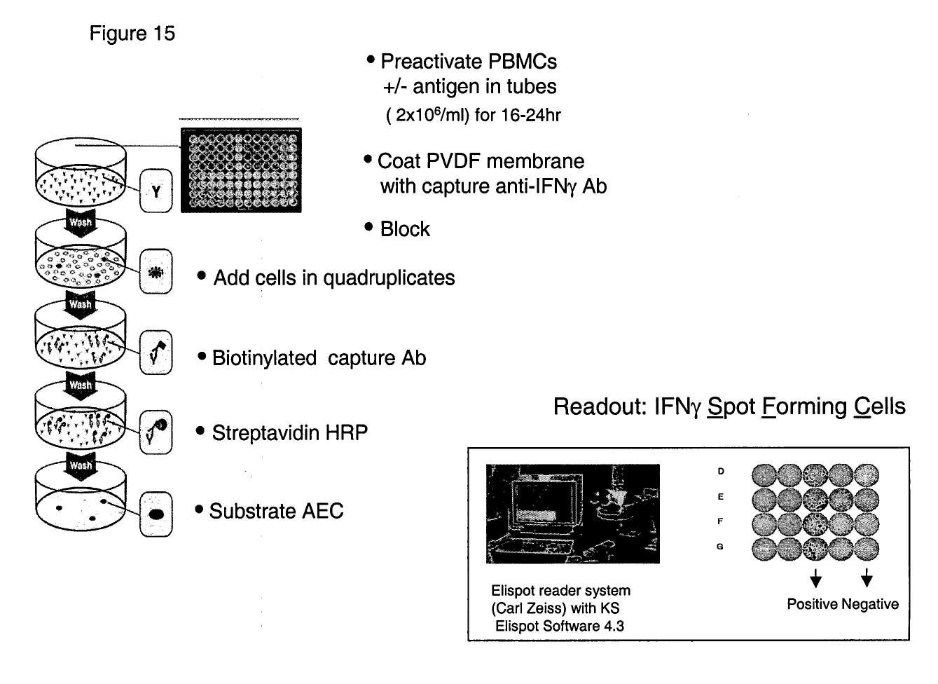

2 mL sera were collected from each monkey prior to each DNA immunization a n d 3 weeks after the last injection for antibody assays by ELISA as described in Example 1, using either sEpCAM-his-tag or TAg-25-his-tag fusion proteins to coat the ELISA plates. Optical density or EC50 values obtained from the ELISA assays were determined. Results are shown in FIGS. 8 and 11. FIG. 11 shows that the number of monkeys in each group that developed antibodies to sEpCAM increased after each of the first three DNA immunizations. A monkey was designated a responder if its obtained serum comprised antibody titer levels that produced an EC50≧5 or an OD value of ≧0.5 at a 1/10 serum dilution in the ELISA assay for either EP CAM or TAg-25. The number of animal responders is out of a total maximum of 4 for EpCAM-treated or TAg-25-treated groups or 2 for the saline-treated group sEpCAM-specific antibody responders comprised greater than half the animals per group after the third DNA immunization (i.e., at day 64) with the DNA pMaxVax vector encoding TAg-25 (the pMaxVax_{TAg-25} vector) administered by either i.m. or i.d. routes. Similar results were observed for animals immunized with the DNA pMaxVax vector encoding sEpCAM (the pMaxVax_{sEpCAM} vector) by the i.d. route, while exactly half of those monkeys given the vector encoding sEpCAM i.m. were classified as responders according to these criteria (i.e., animals whose serum demonstrated in the assay antibody levels giving an EC50≧5 or at an OD value of ≧0.5 at a 1/10 serum dilution for EpCAM or TAg-25). The fourth DNA immunization enhanced the sEpCAM-specific antibody levels beyond those detected after the third immunization in most of the responding monkeys (data not shown). These findings confirm that the TAg-25 antigen-encoding nucleic acid vector induced an sEpCAM-specific cross-reactive antibody response in at least as many non-human primates as a nucleic acid vector encoding human sEpCAM antigen. Moreover, there were as many, if not more, monkeys that developed anti-sEpCAM antibodies after the fourth immunization by i.d. or i.m. administration with the vector encoding TAg-25 antigen as there were monkeys that developed anti-sEpCAM antibodies after the 4^{th} immunization with the vector encoding sEpCAM (FIG. 1). A similar profile of responses was also observed with respect to TAg-25-antigen-specific antibodies (FIGS. 8 and 11). By comparison, sEpCAM and TAg-25-antigen-specific antibodies were not detectable under these criteria (i.e., animals whose serum demonstrated in the assay an EC50≧5 or at an OD value of ≧0.5 at a 1/10 serum dilution) in saline treated monkeys. See FIGS. 8 and 11. Expressed TAg-25 and sEpCAM antigens induced the immune responses.

The results of this experiment demonstrate that nucleic acid vectors of the invention that encode immunogenic polypeptides of the invention induce a humoral immune response in a mammal (e.g., primate). Moreover, the results confirm that such nucleic acid vectors have the ability to induce a cross-reactive antibody response in a mammal (e.g., primate) with EpCAM or an antigenic fragment thereof. Immunization with a DNA vector encoding TAg-25 induced antibodies that cross-react with hEpCAM or an antigenic fragment thereof.

### Example 7

This example demonstrates that polypeptides and nucleic acids of the invention have an ability to induce EpCAM cross-reactive T cell proliferative immune responses in mammals.

Spleens were collected from sacrificed BALB/c mice immunized with either a pMaxVax_{TAg-25} vector or empty pMaxVaxₙᵤₗₗ vector in PBS as described in Example 5, or with a solution comprising affinity purified TAg-25-his-tagged fusion protein in 1.5% alum or bovine serum albumin in 1.5% alum as described in Example 2. The BSA solution served as a negative control. In addition, several additional untreated mice were included for comparative purposes; these mice were not immunized with any DNA vector or protein. The spleens were dissociated into a single-cell suspension in 3 mL of RPMI 1640 medium supplemented with 10% FBS, 1 mM glutamine, 10 mM HEPES, 100 U/ml penicillin, 100 μg/ml streptomycin, 1 mM sodium pyruvate, and 0.05 mM β-2 mercaptoethanol (all Gibco BRL). This medium is henceforth referred to as cRPMI.

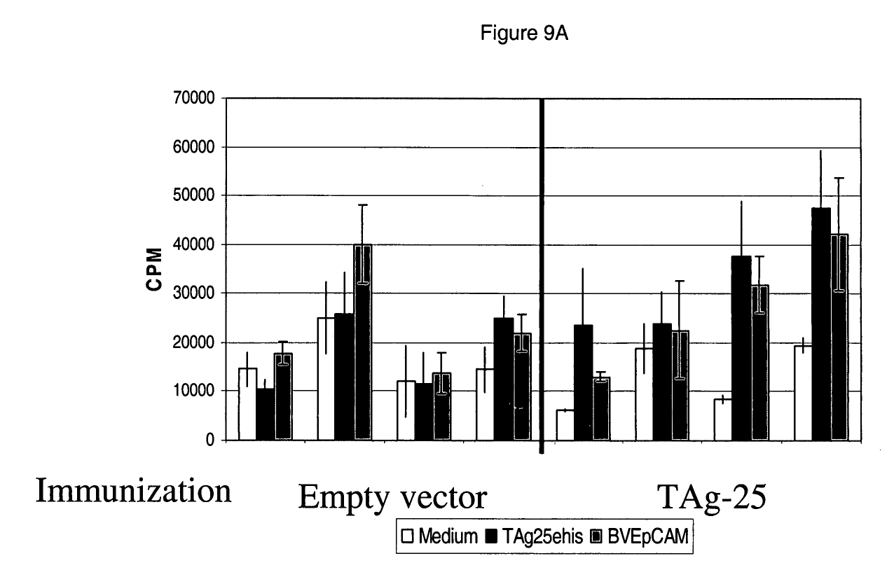

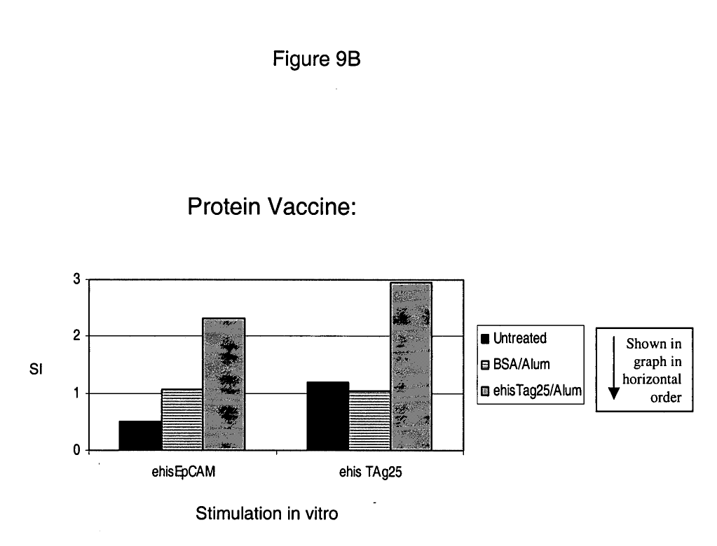

Splenic red blood cells were lysed using a solution of ammonium chloride/sodium bicarbonate 0.8%/0.08% in water. Live lymphocytes from DNA-immunized mice were counted and resuspended at 1×10⁵ cells per well in 250 μl aliquots in U-bottom 96 well plates (Costar) in cRPMI and cultured either alone or with 10 μg/ml of either Baculovirus (BV)-expressed EpCAM antigen or affinity purified TAg-25-his-tagged fusion protein (FIG. 9A). Live lymphocytes from protein-immunized mice were cultured at the same concentration and volume in U-bottom 96 well plates in cRPMI and restimulated with sEpCAM-his-tagged fusion protein or TAg-25-his-tagged fusion protein at 10 μg/ml (FIG. 9B).

The lymphocytes were cultured for 5 days at 37° C. Lymphocyte proliferation was assessed by addition of 1 μCi/well [³H]-thymidine during the last 8 hours of the culture period. Cell bound DNA was harvested on filter mats by a Tom-tech 96 harvester and [³H]-thymidine incorporation was measured on a Betaplate counter according to the manufacturer's protocol (Microbeta, Wallac). Simulation indices (SI) were calculated by dividing the mean proliferative response to a given antigen by the mean proliferative response of the same cells restimulated in the absence of the antigen. Results for the mice immunized with the pMaxVax_{TAg-25} DNA vector or the empty pMaxVaxₙᵤₗₗ DNA vector and restimulated with either TAg-25-his-tagged fusion protein or BV-expressed sEpCAM are shown in FIG. 9A. “Medium” refers to cRPMI described above. Results for the mice immunized with TAg-25-his-tagged fusion protein/alum solution, mice immunized with the BSA/alum solution, and untreated mice, each of which was restimulated with either TAg-25-his-tagged fusion protein or sEpCAM-his-tagged fusion protein are provided in FIG. 9B.

As can be seen in FIG. 9A, in 3 of 4 mice immunized with pMaxVax_{TAg-25} vector (which encodes TAg-25 polyeptide), a T cell proliferative immune response against sEpCAM antigen was induced. The level of proliferation induced when lymphocytes were restimulated with BV-expressed sEpCAM antigen was comparable to that observed when such lymphocytes were restimulated with TAg-25-his-tagged fusion protein. Moreover, the levels of sEpCAM-specific T cell proliferative responses observed for lymphocytes from mice immunized with TAg-25-polypeptide encoding DNA expression vector were significantly higher than for mice immunized with an empty vector, indicating that the observed human EpCAM-specific T cell proliferative immune response was induced or enhanced by immunization with the TAg-25-encoding vector.

Likewise, the sEpCAM-specific T cell proliferative responses induced by immunizing animals with TAg-25 polypeptide in 1.5% alum were significantly higher than the T cell proliferative responses induced in mice immunized with BSA in 1.5% alum, and for untreated control mice (FIG. 9B), indicating that the induced or enhanced T cell proliferation was attributable to the immunogenicity of the TAg-25 polypeptide. Immunization with TAg-25 protein induced T cell proliferative responses that cross-reacted with human sEpCAM. These results confirm that specific immunity to human EpCAM or an antigenic fragment thereof, such as sEpCAM, is induced via administration to a subject of either TAg-25-encoding polynucleotide (SEQ ID NO: 19) or TAg-25 polypeptide (SEQ ID NO:4). TAg-25 encoding polynucleotide may be administered via a DNA vector, such as a pMaxVax plasmid vector.

### Example 8

This example demonstrates the induction of T cell-associated cytokine production in cells immunized with a vector encoding an immunogenic polypeptide of the invention.

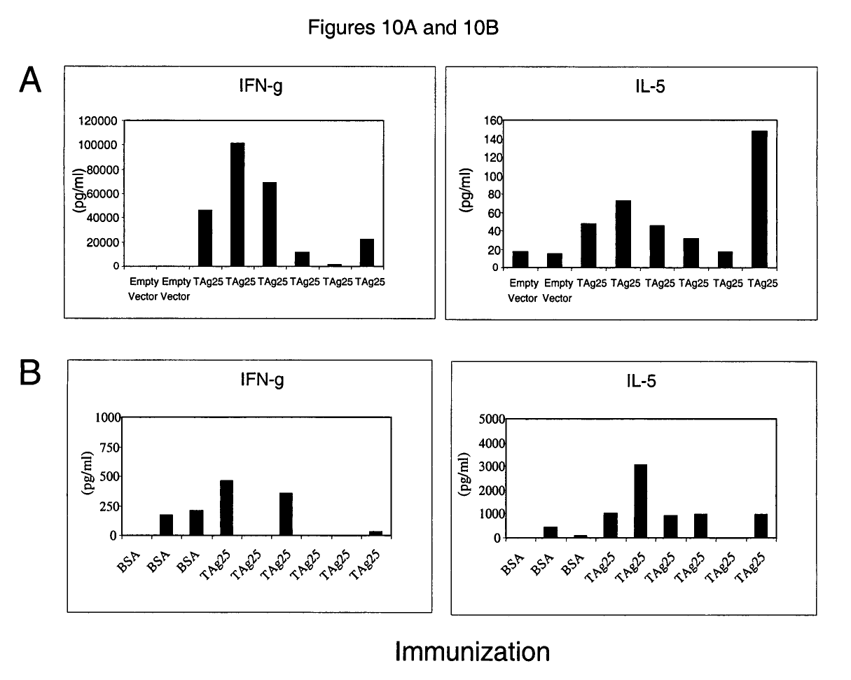

Lymphocytes prepared from sacrificed BALB/c mice immunized with pMaxVax_{TAg-25} or pMaxVaxₙᵤₗₗ in PBS as described in Example 5, or with a solution comprising affinity purified TAg-25-his-tagged fusion protein in 1.5% alum or BSA in 1.5% alum as described in Example 2, were restimulated with an sEpCAM-his-fusion protein or TAg-25-his-tagged fusion protein after five days of culturing as described in Example 8. Supernatant from these cultures was collected and subjected to a two-site sandwich ELISA assay for the detection of IFN-γ or IL-5. Such assay is well known in the art (see, e.g., Slade, S. J., Immunobiol. 179:353 (1989); Abrams, J., Curr. Protocols in Immunol. 13:6.1 (1995)). The assay was designed with a cytokine sensitivity of 125 pg/ml (1 U/ml=0.1 ng/ml). Cytokine (IFN-γ; IL-5) concentrations in the supernatant of each such restimulated lymphocyte cultures are shown in FIGS. 10A-10B.

As shown in FIG. 10A, supernatant obtained from lymphocyte cultures obtained from mice immunized with a pMaxVax_{TAg-25} vector and restimulated with an sEpCAM-his-tagged fusion protein or TAg-25-his-tagged fusion protein contained a significant concentration of IFN-γ (e.g., about 50 ng/mL) and considerably less IL-5 (e.g., about 60 pg/mL under the same restimulation conditions). In contrast, the cells of mice that received empty DNA vector control (pMaxVaxₙᵤₗₗ) failed to produce detectable concentrations of antigen-specific IFN-γ and also produced significantly lower IL-5 concentrations than did the cells obtained from mice that had been immunized with a pMaxVax_{TAg-25} vector when those cells were cultured in the presence of sEpCAM-his-tagged fusion protein.

FIG. 10B shows the cytokine concentration of supernatants harvested from lymphocyte cultures obtained from mice immunized with a solution of affinity purified TAg-25-his-tagged fusion protein in 1.5% alum or BSA in 1.5% alum and restimulated with sEpCAM-his-tagged fusion protein. The induced sEpCAM-specific IL-5 concentrations, but not IFN-γ concentrations, observed in supernatants obtained from mice immunized with TAg-25-his-tagged fusion protein were significantly higher than respective IL-5 or IFN-γ concentrations in supernatants obtained from mice receiving BSA. These results indicate that immunization of a mammal either with a DNA vector encoding TAg-25 polypeptide or with a TAg-25 polypeptide (SEQ ID NO:4) induced or enhanced production of lymphocyte-associated cytokines when such lymphocytes were restimulated with sEpCAM antigen. TAg-25 antigen induced cytokine-specific responses to EpCAM whether delivered in protein or DNA format. Administration of a DNA vector encoding TAg-25 polypeptide (e.g., a DNA vector comprising the polynucleotide sequence of SEQ ID NO: 19) favored production of sEpCAM-specific IFN-γ, while administration of aTAg-25 polypeptide favored sEpCAM-specific IL-5 production. Importantly, these results demonstrate that these effector cell immune responses induced by such nucleic acids and polypeptides of the invention are cross-reactive against EpCAM (or an antigenic fragment thereof) in mammalian lymphocytes.

Previous in vitro studies have suggested that IFN-γ may be essential at early stages of culture for differentiation of precursor CD8+ T cells into lytic effector cells (see, e.g., Ostankovitch, M. et al., Int. J. Cancer 72:987 (1997); Stuhler, G. et al., Proc. Natl. Acad. Sci. USA 94:622 (1997)). A Th1-polarized environment in vivo also has been suggested to favor the cytokine-dependent proliferation of cytotoxic T lymphocytes (see, e.g., Keene, J. A. et al., J. Exp. Med. 155:768 (1982)) or to alter antigen-presenting cells so that they become stimulatory for CD8+ T cells (see, e.g., Guerder, S. et al., J. Exp. Med. 176:553 (1992)). Given that CD8+ T cells are believed important for immune responses to tumor cells, the production of IFN-γ in these restimulated lymphocytes suggests that immunization with a nucleic acid encoding TAg-25, or a vector comprising such nucleic acid, creates an environment favorable to the activation of protective CD8+ T lymphocytes.

### Example 9

This example demonstrates the production of a mouse antibody for use in therapeutic and/or prophylactic methods of the invention (e.g., fro treatment of cancers expressing hEpCAM), methods for detecting EpCAM, and methods for affinity purification of a soluble TAg-25 and/or human EpCAM or antigenic fragment thereof. Determination of a variable heavy chain coding sequence for a mouse anti-TAg-25 mAb (1-121.1) is described.

Application of 1-121.1 mAb for purification of TAg-25/EpCAM. Evaluation of mouse monoclonal antibody, 1-121.1, as a reagent for affinity purification of TAg-25 was determined by isolating the antibody from conditioned medium of suspension cultures of hybridoma cell line, 1-121.1, and coupling the anti-TAg-25 mAb to pre-activated CNBr-sepharose beads. These antibody-coupled beads were packed under pressure into a glass chromatography column, which was subsequently used to capture previously purified TAg-25-his-tagged fusion protein captured by a HiTrap anti-e epitope affinity column (Pharmacia, Piscataway, N.J.).

Purification of anti-TAg-25 mAb was accomplished by passing conditioned medium from suspension cultures of hybridoma cell line, 1-121.1, over a 5 ml Hi-Trap protein-G affinity column (Pharmacia, Piscataway, N.J.). The binding and elution was done according to the manufacturer's protocol. The antibody was buffered exchanged into 0.1M bicarbonate buffer pH 8.3.

Approximately 10 mg of purified 1-121.1 mAb was coupled to 250 mg of CNBr-sepharose beads (Pharmacia, Piscataway, N.J.) according to the manufacturer's instructions. The anti-TAg-25 coupled sepharose beads were packed into a glass chromatography column up to an operating pressure of 0.3 mPA. Following the manufacturer's instructions, 400 ug of TAg-25-his-tagged fusion protein was loaded onto the affinity column and eluted with 0.1 M glycine at pH 2.7. The TAg-25-his-tagged fusion protein was eluted as a single peak and determined to be predominantly of the 42 kDa form (data not shown).

Cloning of the antigen binding domains of anti-TAg-25 monoclonal antibody. Hybridoma cell line, 1-121.1, secretes a mouse antibody that binds TAg-25 and EpCAM. The light and heavy chains belong to lambda and IgG1 isotypes, respectively. PCR primers were designed according to the N-terminal amino acid sequence of either the V_{H} or V_{L} domain of the 1-121.1 mAb using standard techniques and the corresponding CH or CL region of the antibody chains (Sequences of Immunological Interests, Kabat, V1-3, NIH). The sequences of the primers are as follows:

```
VH domain of the 1-121.1 mAb (HVC1.121.1F)
                                            E V K L L E S G G
(5′ ATTCTGCA-GATATC- GAG GTG AAG CTG CTG GAG TCT GGC GG 3′)
              EcoRV C C  C C  A
                                          A
CR domain of the 1-121.mAb (Kabat) (HVC1.121.1R)
Shown is the reverse complement of the nucleotide coding sequence for: AKTTPPSV
(5′ ATAGTTTA-GCGGCCGC-GAC AGA TGG GGG TGT CGT TTT GGC 3′)
                              NotI
CL domain of the 1-121.mAb (Kabat) (LVC1.121.1R)
Shown is the reverse complement of the nucleotide coding sequence for: FPPSSEEL
(5′ ATAGTTTA-GCGGCCGC-GAG CTC TTC AGA GGA AGG TGG AAA 3′)
```

Total RNA was isolated from approximately 4×10⁷ cells of hybridoma cell line, 1-121.1 and subsequently converted to poly (A) mRNA using a commercial RNA isolation kit (Stratagene, La Jolla, Calif.). The mRNA was primed with an oligo d(T) primer and first strand cDNA was generated according to the manufacturer's instructions (ProStar First Strand RT-PCR kit, Stratagene, La Jolla, Calif.). The nucleotide sequence encoding the heavy chain variable region of 1-121.1 was determined using PCR using primers, (HVC1.121.1F) and (HVC1.121.1R), under standard PCR conditions. The PCR product was digested with EcoRV and NotI and subcloned into pCDNA3.1(+). The nucleotide sequence of the variable heavy chain domain was determined by DNA sequencing as follows:

(SEQ ID NO:94)

GAGGTGAAGCTGCTGGAGTCCGGAGGTGGCCTGGTGCAGCCTGGAGGATC
CCTGAAACTCTCCTGTGCAGCCTCAGGATTCGATTTTAGTAGATACTGGA
TGAGTTGGGTCCGGCAGGCTCCAGGGAAAGGGCTAGAATGGATTGGAGAT
ATTAATCTAGAAAGCAATACGATAAACTATACGCCATCTCTAAAGGATAA
ATTCATCATCTCCAGAGACAACGCCAAAAATACGCTGTACCTGCAAATGA
ACAAAGTGAGATCTGAGGACACAGCCCTTTATTACTGTGCAAGAGGGGCC
TATACTATGGACTACTGGGGTCAAGGAACCTCAGTCACCGTCTCCTCAGC
CAAAACGACACCCCCATCTGTC

The amino acid sequence of the variable heavy chain domain is:

(SEQ ID NO:95)

EVKLLESGGGLVQPGGSLKLSCAASGFDFSRYWMSWVRQAPGKGLEWIGD
INLESNTINYTPSLKDKFIISRDNAKNTLYLQMNKVRSEDTALYYCARGA
YTMDYWGQGTSVTVSSAKTTPPSVAA

The invention includes isolated or recombinant nucleic acid having at least about 0.90, 95, 96, 97, 98, 99 or more % sequence identity to the polynucleotide sequence of SEQ ID NO:94 and isolated or recombinant polypeptides having at least about 90, 95, 96, 97, 98, 99 or more % sequence identity to the polypeptide sequence of SEQ ID NO:95. These antibodies are useful in therapeutic and/or prophylactic methods for treating EpCAM-associated diseases as discussed above.

### Example 10

This example provides a strategy for assessing the safety and immunogenicity of nucleic acids of the invention. Sixty monkeys divided into twelve groups of five animals each, divided into 11 treatment groups and 1 control group, are used to study the safety and effectiveness of the immunogenic nucleic acids of the invention and immune responses induced by such nucleic acids administered optionally with a costimulatory molecule (e.g., DNA vector encoding CD28 binding protein, such as CD28BP-15). The administration strategies for the 11 experimental treatment groups are set forth in Table 7.

TABLE 7
Experimental Design for Safety/Immunogenicity Study

| Group | Route | Treatment |
|---|---|---|
| 1 | i.m. | 2 mg pMaxVaxₙᵤₗₗ - administered on days 0, 21, 42, and 63 |
| | | 10 μg hepatitis B surface antigen (HBsAg) polypeptide |
| | | vaccine (or variant thereof) - administered on days 91 and 119 |
| | | 100 μg TAg-25 protein - administered on days 105 and 133 |
| 2 | i.d. | 2 mg pMaxVaxₙᵤₗₗ - administered on days 0, 21, 42, and 63 |
| 3 | i.m. | 2 mg pMaxVax_{TAg-25} (monocistronic vector) - administered on days 0, |
| | | 21, 42, and 63 |
| | | 10 μg HBsAg - administered on days 91, 119 |
| | | 100 μg TAg-25 protein - administered on days 105 and days 133 |
| 4 | i.d. | 2 mg pMaxVax_{TAg-25} - administered on days 0, 21, 42, and 63 |
| 5 | i.m. | 2 mg pMaxVax_{TAg-21:hB7-1} (bicistronic vector) - administered on days 0, |
| | | 21, 42, and 63 |
| | | 10 μg HBsAg - administered on days 91, 119 |
| | | 100 μg TAg-25 protein - administered on days 105 and days 133 |
| 6 | i.d. | 2 mg pMaxVax_{TAg-21:hB7-1} - administered on days 0, 21, 42, and 63 |
| 7 | i.m. | 2 mg pMaxVax_{TAg-25:CD28BP-15} - administered on days 0, 21, 42, and 63 |
| | | 10 μg HBsAg - administered on days 91, 119 |
| | | 100 μg TAg-25 protein - administered on days 105 and days 133 |
| 8 | i.d. | 2 mg pMaxVax_{TAg-25:CD28BP-15} - administered on days 0, 21, 42, and 63 |
| 9 | i.m. | 1 mg pMaxVax_{TAg-25} and 1 mg pMaxVaxₙᵤₗₗ (i.e., 1 mg of |
| | | two monocistronic pMaxVax vector DNA per dose) - |
| | | administered on days 0, 21, 42, and 63 |
| 10 | i.m. | 1 mg pMaxVax_{TAg-25} and 1 mg pMaxVax_{hB7-1} - |
| | | administered on days 0, 21, 42, and 63 |
| 11 | i.m. | 1 mg pMaxVax_{TAg-25} and 1 mg pMaxVax_{CD28BP-15} - |
| | | administered on days 0, 21, 42, and 63 |

“CD28BP” refers to the synthetic or recombinant CD28-binding polypeptide, or nucleic acid encoding such polypeptide, as described in commonly assigned Int'l Patent App. No. PCT/US01/19973 (Int'l Publ. No. WO 02/00717) and Int'l Patent App. No. PCT/US02/19898. A CD28BP polypeptide is an immunomodulatory polypeptide. An exemplary CD28BP polypeptide is CD28BP-15, which comprises the polypeptide sequence of SEQ ID NO:66 shown in Int'l Patent App. Nos. US01/19973 and US02/19898. An exemplary nucleic acid encoding CD28BP-15 comprises the polynucleotide sequence of SEQ ID NO: 19 shown in Int'l Patent App. Nos. PCT/US01/19973 and PCT/US02/19898. Other immunomodulatory polypeptides and nucleic acids encoding such polypeptides, including, e.g., those CD28BP polypeptides described in these international applications can also be employed in methods of the invention.

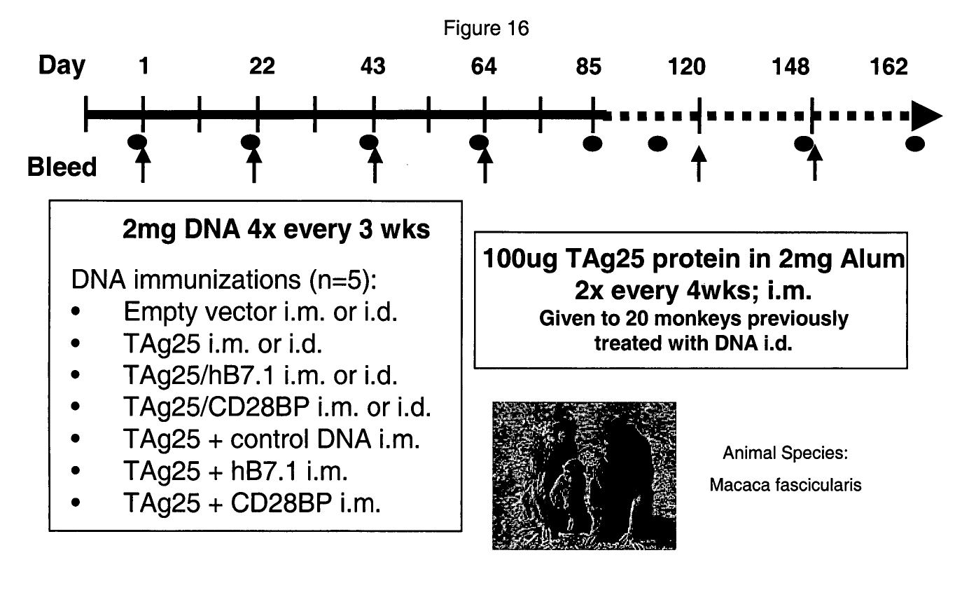

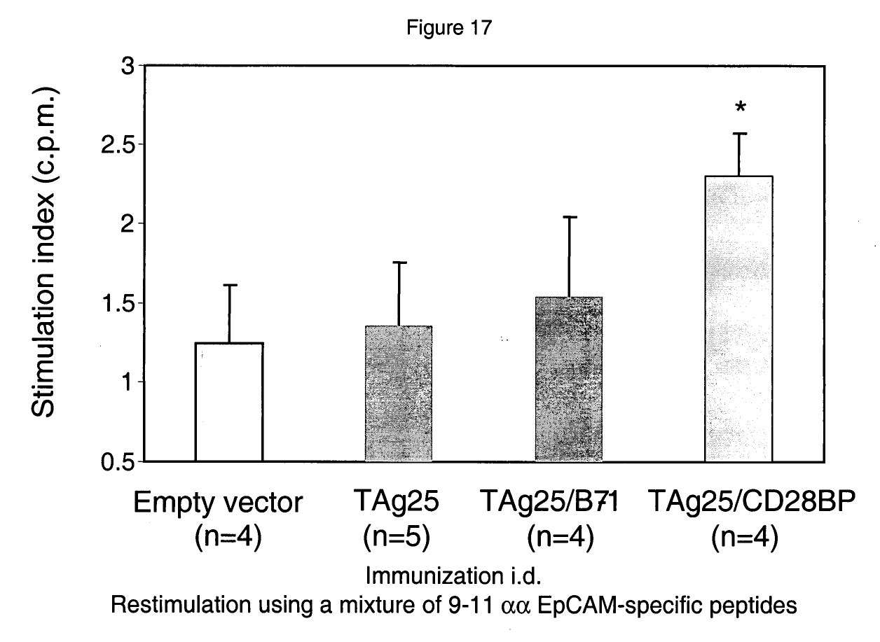

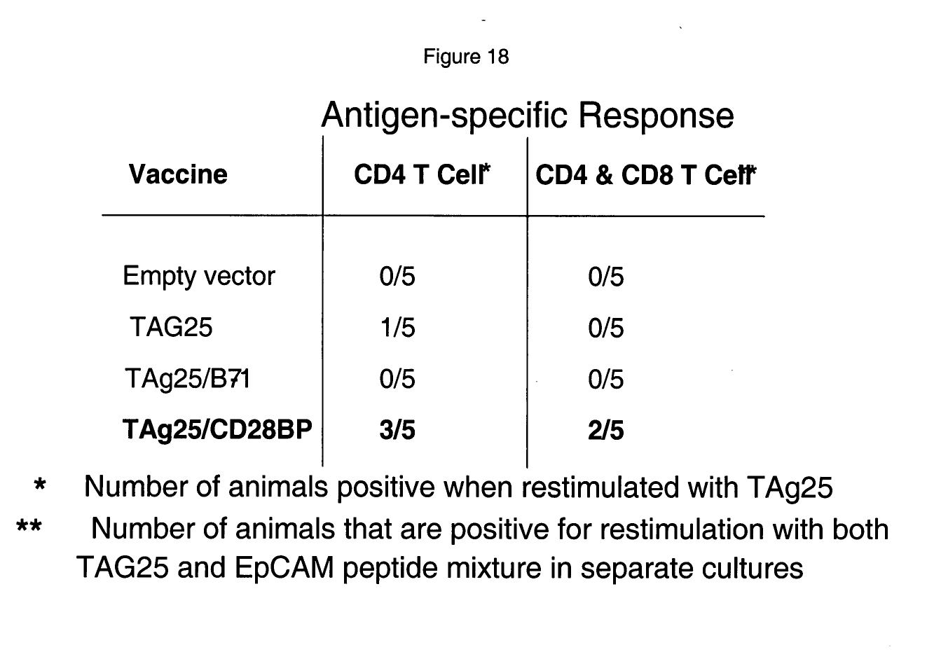

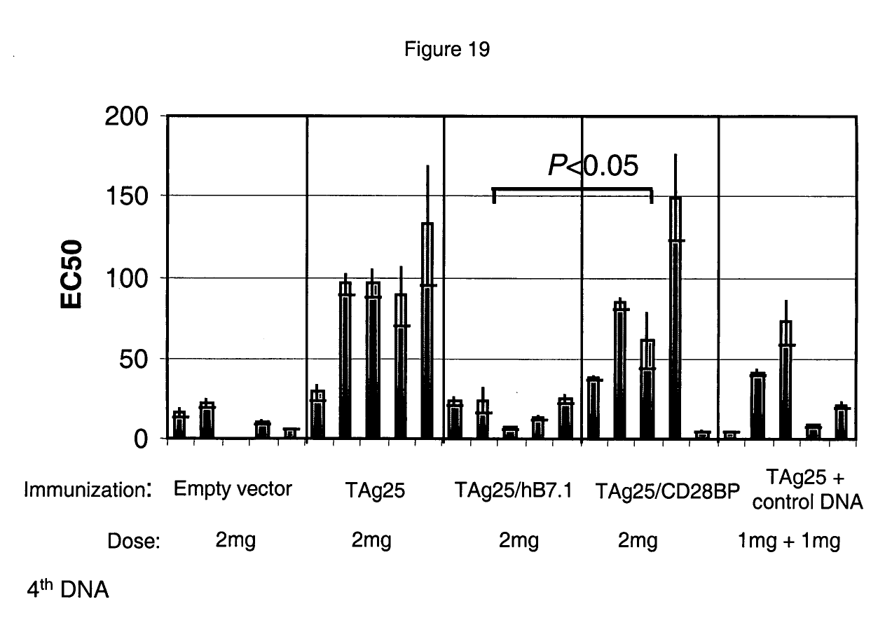

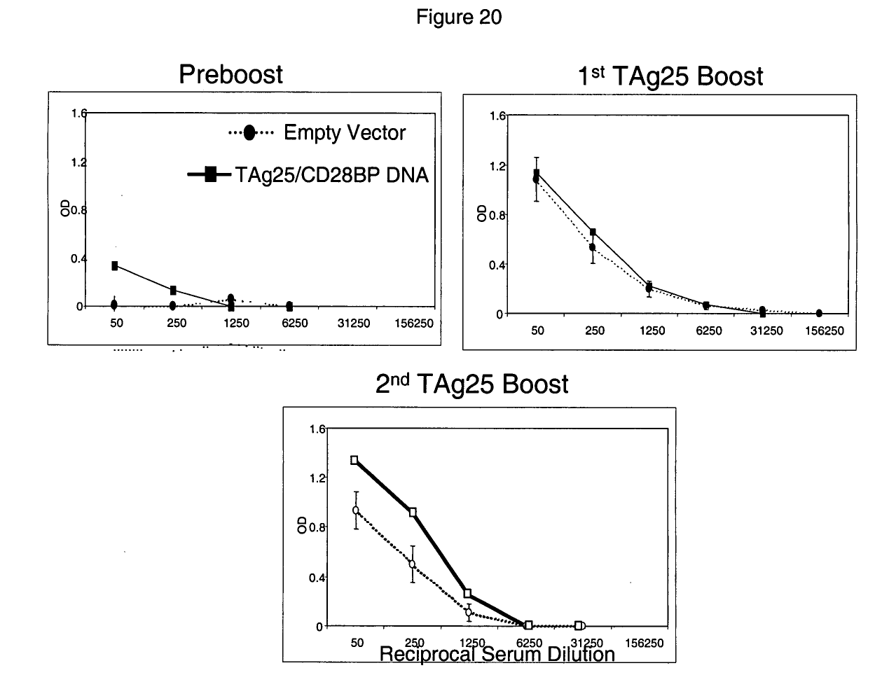

The animals are assessed for clinical findings, clinical abnormalities, sEpCAM antigen and/or EpCAM IgG ELISA titers, T cell proliferation, T cell effector function (cytokine production), percent inhibition of in vitro T cells by sera from vaccinated monkeys, blood tests, serum chemistries, and urinalysis. See data and results shown in FIGS. 16-20.

The results of the experiments demonstrate, in part, that nucleic acids of the invention are capable of inducing humoral and T cell-associated immune responses in primates without jeopardizing the safety of the primates by such treatment. As such, the results of these experiments demonstrate the effectiveness of such nucleic acid vaccines of the invention as vaccines against EpCAM associated cancers in mammals, including non-human primates and humans.

Immunization i.d. of monkeys with 2 mg of a bicistronic DNA plasmid vector encoding TAg-25 and a costimulatory polypeptide (e.g., CD28BP-15) in PBS, in four separate immunizations, induced production of a high titer of antibodies against human sEpCAM and a potent EpCAM-specific CD8+ T cell proliferative response (data not shown). An exemplary bicistronic vector is pMaxVax_{Tag-25:CD28BP-15}.

While the foregoing invention has been described in some detail for purposes of clarity and understanding, it will be clear to one skilled in the art from a reading of this disclosure that various changes in form and detail can be made without departing from the true scope of the invention. It is understood that the examples and embodiments described herein are for illustrative purposes only and that various modifications or changes in light thereof will be suggested to persons skilled in the art and are to be included within the spirit and purview of this application and scope of the appended claims. For example, all the techniques and apparatus described above may be used in various combinations. All references, including publications, patent applications, and patents, and other documents, cited herein are incorporated herein by reference in their entirety for all purposes to the same extent as if each individual reference were individually and specifically indicated to be incorporated herein by reference in its entirety for all purposes and/or were set forth in its entirety herein.

All amino acid or nucleotide sequences of one of the aforementioned sequence patterns are to be considered individually disclosed herein. Thus, for example, an amino acid sequence pattern of three residues, where a “Xaa” represents one of the amino acid positions in the pattern represents a disclosure of twenty different amino acid sequences (i.e., one sequence for each naturally occurring amino acid residue that could be present in the Xaa position).

1-138. (canceled)

139. An isolated or recombinant polypeptide comprising a polypeptide sequence that has at least 96% sequence identity to a polypeptide sequence comprising amino acid residues 81-265 of SEQ ID NO:4, wherein said isolated or recombinant polypeptide has an ability to induce an immune response against human epithelial cell adhesion molecule (EpCAM) or an antigenic fragment of human EpCAM.

140. The polypeptide of claim 139, wherein the polypeptide comprises a polypeptide sequence that has at least about 96% sequence identity to a polypeptide sequence comprising amino acid residues 24-265 of SEQ ID NO:4.

141. The polypeptide of claim 139, wherein the polypeptide comprises a polypeptide sequence that has at least about 96% sequence identity to the polypeptide sequence of SEQ ID NO:4.

142. The polypeptide of claim 139, wherein the polypeptide comprises amino acid residues 81-265 of SEQ ID NO:4.

143. The polypeptide of claim 140, wherein the polypeptide comprises amino acid residues 24-265 of SEQ ID NO:4.

144. The polypeptide of claim 141, wherein the polypeptide comprises the polypeptide sequence of SEQ ID NO:4.

145. The polypeptide of claim 139, wherein the polypeptide has an ability to induce production of antibodies against human EpCAM or an antigenic fragment thereof.

146. The polypeptide of claim 139, wherein the polypeptide induces a T cell response against human EpCAM.

147. The polypeptide of claim 146, wherein the polypeptide T cell proliferation response against human EpCAM.

148. The polypeptide of claim 139, wherein the polypeptide induces production of at least one cytokine.

149. The polypeptide of claim 139, wherein the at least one cytokine is interferon-gamma.

150. The polypeptide of claim 139, wherein the polypeptide is glycosylated and/or pegylated.

151. The polypeptide of claim 139, wherein the immune response comprises the production of antibodies that bind human EpCAM, proliferation of T cells, and production or one or more cytokines.

152. The polypeptide of claim 140, wherein the polypeptide has an ability to induce production of antibodies against human EpCAM or an antigenic fragment thereof.

153. The polypeptide of claim 140, wherein the polypeptide induces a T cell response against human EpCAM.

154. The polypeptide of claim 140, wherein the polypeptide induces production of at least one cytokine.

155. The polypeptide of claim 140, wherein the immune response comprises the production of antibodies that bind human EpCAM, proliferation of T cells, and production or one or more cytokines.

156. The polypeptide of claim 141, wherein the immune response comprises at least one of an ability to induce production of antibodies against human EpCAM or an antigenic fragment thereof, induce a T cell response against human EpCAM, or induce production of at least one cytokine.

157. A composition comprising the polypeptide of claim 139 and a carrier, diluent, or excipient.

158. The composition of claim 157, wherein the composition further comprises at least one adjuvant, immunomodulatory polypeptide, or cytokine, or any combination thereof.


SEQUENCE LISTING (95 sequences)

SEQ ID NO: 1 - protein, 185 aa, Artificial Sequence
  TAg-25 fragment comprising extra-cellular domain (ECD)
RIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLK

SEQ ID NO: 2 - protein, 57 aa, Artificial Sequence
  TAg-25 fragment comprising propeptide (PP)
QEECVCENYKLAVNCFVNNNRECQCTSVGAQNTVICSKLAAKCLVMKAEMNGSKLGR

SEQ ID NO: 3 - protein, 23 aa, Artificial Sequence
  TAg-25 fragment comprising signal peptide (SP)
MAPPQALALGLLLAAATATFAAA

SEQ ID NO: 4 - protein, 265 aa, Artificial Sequence
  TAg-25 polypeptide (which comprises signal peptide+propeptide+ ECD)
MAPPQALALGLLLAAATATFAAAQEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLK

SEQ ID NO: 5 - protein, 242 aa, Artificial Sequence
  TAg-25 fragment comprising propeptide+ ECD
QEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLK

SEQ ID NO: 6 - protein, 314 aa, Artificial Sequence
  TAg-25 full-length/membrane-bound form, which comprises N- to C-terminus signal peptide+propeptide+ECD+TMD+CD
MAPPQALALGLLLAAATATFAAAQEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLKAGVIAVIVVVVMAVVAGIVVLVISRKKRMAKYEKAEIKEMGEMHRELNA

SEQ ID NO: 7 - protein, 234 aa, Artificial Sequence
  Mature Tag-25 polypeptide, which comprises N- to C-terminus ECD+TMD+CD
RIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLKAGVIAVIVVVVMAVVAGIVVLVISRKKRMAKYEKAEIKEMGEMHRELNA

SEQ ID NO: 8 - protein, 208 aa, Artificial Sequence
  Tag-25 fragment comprising ECD+TMD
RIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLKAGVIAVIVVVVMAVVAGIVVLVI

SEQ ID NO: 9 - protein, 63 aa, Artificial Sequence
  TAg-18 fragment comprising ECD
QNDVDIADVAHYFEKDVKGESLFHSSKKMDLRVNGEQLDLDPGQTLIYYVDRNAPEFSMQALK

SEQ ID NO: 10 - protein, 115 aa, Artificial Sequence
  TAg-18   ECD+TMD+CD
QNDVDIADVAHYFEKDVKGESLFHSSKKMDLRVNGEQLDLDPGQTLIYYVDRNAPEFSMQALKAGVCAVIVVVMIAVVAGIVVLVISRKKRMAKYEKAEIKEMGRMHRELNASVL

SEQ ID NO: 11 - protein, 29 aa, Artificial Sequence
  TAg-18 CD-like sequence
SRKKRMAKYEKAEIKEMGRMHRELNASVL

SEQ ID NO: 12 - protein, 186 aa, Artificial Sequence
  TAg-21 fragment comprising ECD
RIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSSKKMDLRVNGELLDLDPGQTLIYYVDEKAPEFSMQGLK

SEQ ID NO: 13 - protein, 266 aa, Artificial Sequence
  TAg-21 polypeptide (comprising SP+PP+ECD)
MAPPQALAFGLLLAAATATFAAAQEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLRTALQKEITTRYQLDPKFITSILYENNVITIDLMQNSSQKTQNDVDIADVAHYFEKDVKGESLFHSSKKMDLRVNGEQLDLDPGQTLIYYVDRNAPEFSMQALK

SEQ ID NO: 14 - protein, 289 aa, Artificial Sequence
  TAg-21 extended polypeptide comprising SP+PP+ECD+TMD
MAPPQALAFGLLLAAATATFAAAQEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLRTALQKEITTRYQLDPKFITSILYENNVITIDLMQNSSQKTQNDVDIADVAHYFEKDVKGESLFHSSKKMDLRVNGEQLDLDPGQTLIYYVDRNAPEFSMQALKAGIIAVIVVVMIAVVAGIVVLVI

SEQ ID NO: 15 - protein, 23 aa, Artificial Sequence
  Tag-21 TMD
AGIIAVIVVVMIAVVAGIVVLVI

SEQ ID NO: 16 - DNA, 555 bp, Artificial Sequence
  DNA encoding TAg-25 fragment comprising ECD domain
aggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaag

SEQ ID NO: 17 - DNA, 171 bp, Artificial Sequence
  DNA encoding TAg-25 fragment comprising propeptide
caggaggagtgtgtgtgcgaaaactacaagctcgctgtcaactgtttcgtcaacaataatagagaatgccagtgcacttctgtgggagcacagaatacagtgatctgtagcaaactggctgcaaagtgtctggtcatgaaggccgaaatgaacggatccaagctcgggcgg

SEQ ID NO: 18 - DNA, 69 bp, Artificial Sequence
  DNA encoding TAg-25 fragment comprising SP
atggcaccccctcaagcactggcactgggtctgctgctggccgccgctaccgccactttcgccgcagca

SEQ ID NO: 19 - DNA, 795 bp, Artificial Sequence
  DNA encoding TAg-25 polypeptide (which comprises signal peptide+propeptide+ ECD)
atggcaccccctcaagcactggcactgggtctgctgctggccgccgctaccgccactttcgccgcagcacaggaggagtgtgtgtgcgaaaactacaagctcgctgtcaactgtttcgtcaacaataatagagaatgccagtgcacttctgtgggagcacagaatacagtgatctgtagcaaactggctgcaaagtgtctggtcatgaaggccgaaatgaacggatccaagctcgggcggaggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaag

SEQ ID NO: 20 - DNA, 726 bp, Artificial Sequence
  DNA encoding TAg-25 fragment comprising PP+ECD
caggaggagtgtgtgtgcgaaaactacaagctcgctgtcaactgtttcgtcaacaataatagagaatgccagtgcacttctgtgggagcacagaatacagtgatctgtagcaaactggctgcaaagtgtctggtcatgaaggccgaaatgaacggatccaagctcgggcggaggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaag

SEQ ID NO: 21 - DNA, 942 bp, Artificial Sequence
  DNA encoding TAg-25 full-length/membrane bound form which comprises SP+PP+ECD+TMD+CD
atggcaccccctcaagcactggcactgggtctgctgctggccgccgctaccgccactttcgccgcagcacaggaggagtgtgtgtgcgaaaactacaagctcgctgtcaactgtttcgtcaacaataatagagaatgccagtgcacttctgtgggagcacagaatacagtgatctgtagcaaactggctgcaaagtgtctggtcatgaaggccgaaatgaacggatccaagctcgggcggaggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaaggctggtgttattgctgttattgtggttgtggtgatggcagttgttgctggaattgttgtgctggttatttccagaaagaagagaatggcaaagtatgagaaggctgagataaaggagatgggtgagatgcatagggaactcaatgca

SEQ ID NO: 22 - DNA, 702 bp, Artificial Sequence
  DNA encoding TAg-25 Mature domain (which comprises ECD+TMD+CD of a full-length or membrane bound form of TAg-25).
aggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaaggctggtgttattgctgttattgtggttgtggtgatggcagttgttgctggaattgttgtgctggttatttccagaaagaagagaatggcaaagtatgagaaggctgagataaaggagatgggtgagatgcatagggaactcaatgca

SEQ ID NO: 23 - DNA, 624 bp, Artificial Sequence
  DNA encoding TAg-25 fragment comprising ECD+TMD
aggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcaagacgacgtggacatcgctgatgtggcctactattttgagaaggacgttaagggggaatcactgttccattcaaagaaaatggatctgagggttaatggcgagctgctggacctggacccagggcaaaccctgatctattatgtggacgagaaggctccagaattctctatgcaaggcctgaaggctggtgttattgctgttattgtggttgtggtgatggcagttgttgctggaattgttgtgctggttatt

SEQ ID NO: 24 - DNA, 69 bp, Artificial Sequence
  DNA sequence encoding TAg-21 fragment comprising SP
atggcacctccccaggcactggcatttggactgctgctggctgcagcaaccgccacattcgctgctgcc

SEQ ID NO: 25 - DNA, 171 bp, Artificial Sequence
  DNA encoding TAg-21 fragment comprising propeptide
caggaggagtgtgtgtgtgagaactataaactggctgtcaattgttttgttaataacaatagggagtgccaatgtactagcgtgggagcccaaaacactgtcatttgctccaaactcgccgccaaatgtctcgtcatgaaagctgaaatgaatggtagcaaactgggacgg

SEQ ID NO: 26 - DNA, 558 bp, Artificial Sequence
  DNA encoding Tag-21 fragment comprising ECD
aggattaagcccgaaggggccctccagaacaatgacggactctacgatccagactgcgacgagagcgggctgttcaaggctaagcagtgcaatggcaccgccacctgttggtgtgtgaataccgctggagtgcggcggacagacaaagacactgagatcacctgtagcgagagggtgcgcacttattggatcatcattgaactgaaacacaaggaacgcgaatccccatatgattccaagagcctgaggaccgccctccagaaagagatcactactagatatcagctggaccccaaattcatcaccagcattctgtacgagaacaatgtcattacaatcgatctgatgcaaaacagcagccagaagacccagaatgacgtggacatcgccgatgtggcccattattttgagaaagatgtcaagggggaatcactgttccacagctccaagaagatggacctgagagtgaacggtgaacaactcgacctcgatcctgggcagacactgatctactatgtcgacaggaatgcccctgaattcagcatgcaggccctgaag

SEQ ID NO: 27 - DNA, 867 bp, Artificial Sequence
  DNA encoding TAg-21 extended polypeptide comprising SP+PP+ECD+TMD
atggcacctccccaggcactggcatttggactgctgctggctgcagcaaccgccacattcgctgctgcccaggaggagtgtgtgtgtgagaactataaactggctgtcaattgttttgttaataacaatagggagtgccaatgtactagcgtgggagcccaaaacactgtcatttgctccaaactcgccgccaaatgtctcgtcatgaaagctgaaatgaatggtagcaaactgggacggaggattaagcccgaaggggccctccagaacaatgacggactctacgatccagactgcgacgagagcgggctgttcaaggctaagcagtgcaatggcaccgccacctgttggtgtgtgaataccgctggagtgcggcggacagacaaagacactgagatcacctgtagcgagagggtgcgcacttattggatcatcattgaactgaaacacaaggaacgcgaatccccatatgattccaagagcctgaggaccgccctccagaaagagatcactactagatatcagctggaccccaaattcatcaccagcattctgtacgagaacaatgtcattacaatcgatctgatgcaaaacagcagccagaagacccagaatgacgtggacatcgccgatgtggcccattattttgagaaagatgtcaagggggaatcactgttccacagctccaagaagatggacctgagagtgaacggtgaacaactcgacctcgatcctgggcagacactgatctactatgtcgacaggaatgcccctgaattcagcatgcaggccctgaaggccggtatcatcgccgtgatcgtggttgttatgatcgccgttgtggccggcatcgtcgtgctggtgatc

SEQ ID NO: 28 - DNA, 345 bp, Artificial Sequence
  DNA sequence encoding TAg-18 alternative fragment comprising ECD+TMD+CD
cagaatgatgtggacatagctgatgtggctcattattttgaaaaagatgttaaaggtgaatccttgtttcattcttctaagaaaatggacctgagagtaaatggagaacaactggatctggatcctggtcaaactttaatttattatgttgatagaaatgcacctgaattttcaatgcaggctctaaaagctggtgtttgtgctgttattgtggttgtgatgatagcagttgttgctggaattgttgtgctggttatttccagaaagaagagaatggcaaagtatgagaaggctgagataaaggagatgggtaggatgcatagggaactcaatgcatcagtccta

SEQ ID NO: 29 - DNA, 552 bp, Artificial Sequence
  DNA sequence encoding human EpCAM fragment comprising hEpCAM ECD
gcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaa

SEQ ID NO: 30 - DNA, 63 bp, Artificial Sequence
  DNA encoding hEpCAM fragment comprising signal peptide
atggcgcccccgcaggtcctcgcgttcgggcttctgcttgccgcggcgacggcgacttttgcc

SEQ ID NO: 31 - DNA, 180 bp, Artificial Sequence
  DNA encoding hEpCAM fragment comprising propeptide
gcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaaga

SEQ ID NO: 32 - protein, 266 aa, Artificial Sequence
  TAg-18 chimeric polypeptide (comprising SP+PP+ECD)
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAHYFEKDVKGESLFHSSKKMDLRVNGEQLDLDPGQTLIYYVDRNAPEFSMQALK

SEQ ID NO: 33 - DNA, 798 bp, Artificial Sequence
  DNA encoding TAg-18 chimeric polypeptide comprising SP+PP+ECD
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggctcattattttgaaaaagatgttaaaggtgaatccttgtttcattcttctaagaaaatggacctgagagtaaatggagaacaactggatctggatcctggtcaaactttaatttattatgttgatagaaatgcacctgaattttcaatgcaggctctaaaa

SEQ ID NO: 34 - protein, 318 aa, Artificial Sequence
  TAg-18 chimera extended polypeptide which comprises SP+PP+ECD+TMD+CD
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAHYFEKDVKGESLFHSSKKMDLRVNGEQLDLDPGQTLIYYVDRNAPEFSMQALKAGVCAVIVVVMIAVVAGIVVLVISRKKRMAKYEKAEIKEMGEMHRELNASVL

SEQ ID NO: 35 - DNA, 957 bp, Artificial Sequence
  DNA sequence encoding TAg-18 chimera (comprising SP+PP+ECD+TMD+CD)
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggctcattattttgaaaaagatgttaaaggtgaatccttgtttcattcttctaagaaaatggacctgagagtaaatggagaacaactggatctggatcctggtcaaactttaatttattatgttgatagaaatgcacctgaattttcaatgcaggctctaaaagctggtgtttgtgctgttattgtggttgtgatgatagcagttgttgctggaattgttgtgctggttatttccagaaagaagagaatggcaaagtatgagaaggctgagataaaggagatgggtaggatgcatagggaactcaatgcatcagtcctataa

SEQ ID NO: 36 - protein, 185 aa, Homo sapiens
  Fragment of human EpCAM (hEpCAM) comprising hEpCAM ECD
RAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 37 - protein, 23 aa, Homo sapiens
  Fragment of hEpCAM comprising hEpCAM signal peptide
MAPPQVLAFGLLLAAATATFAAA

SEQ ID NO: 38 - protein, 57 aa, Homo sapiens
  Fragment of hEpCAM comprising hEpCAM propeptide
QEECVCENYKLAVNCFVNNNRQCQCTSVGAQNTVICSKLAAKCLVMKAEMNGSKLGR

SEQ ID NO: 39 - protein, 242 aa, Homo sapiens
  Fragment of hEpCAM comprising hEpCAM propeptide+ECD
QEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 40 - protein, 265 aa, Homo sapiens
  Fragment of hEpCAM comprising hEpCAM signal peptide+ propeptide+ECD (termed “sEpCAM”)
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLK

SEQ ID NO: 41 - protein, 314 aa, Homo sapiens [sequence omitted]

SEQ ID NO: 42 - DNA, 942 bp, Homo sapiens
  DNA encoding WT full-length/membrane-bound hEpCAM (comprising signal peptide+propeptide+ECD+TMD+CD domains)
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatggcagttgttgctggaattgttgtgctggttatttccagaaagaagagaatggcaaagtatgagaaggctgagataaaggagatgggtgagatgcatagggaactcaatgca

SEQ ID NO: 43 - protein, 234 aa, Homo sapiens
  Mature domain of hEpCAM (comprising ECD+TMD+CD)
RAKPEGALQNNDGLYDPDCDESGLFKAKQCNGTSTCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVMAVVAGIVVLVISRKKRMAKYEKAEIKEMGEMHRELNA

SEQ ID NO: 44 - DNA, 699 bp, Homo sapiens
  DNA encoding mature domain of hEpCAM (comprising ECD+TMD+CD)
gcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaagctggtgttattgctgttattgtggttgtggtgatggcagttgttgctggaattgttgtgctggttatttccagaaagaagagaatggcaaagtatgagaaggctgagataaaggagatgggtgagatgcatagggaactcaatgca

SEQ ID NO: 45 - protein, 23 aa, Artificial Sequence
  Fragment of hEpCAM comprising TMD
AGVIAVIVVVVMAVVAGIVVLVI

SEQ ID NO: 46 - protein, 26 aa, Artificial Sequence
  Fragment of hEpCAM CD
SRKKRMAKYEKAEIKEMGEMHRELNA

SEQ ID NO: 47 - protein, 9 aa, Artificial Sequence
  Epitope of hEpCAM in ECD
GLYDPDCDE

SEQ ID NO: 48 - protein, 9 aa, Artificial Sequence
  Epitope of hEpCAM in ECD
ILYENNVIT

SEQ ID NO: 49 - protein, 9 aa, Artificial Sequence
  Epitope of hEpCAM in ECD
YQLDPKFIT

SEQ ID NO: 50 - protein, 11 aa, Artificial Sequence
  Epitope of hEpCAM in ECD
YQLDPKFITSI

SEQ ID NO: 51 - protein, 19 aa, Artificial Sequence
  Epitope of hEpCAM in ECD
YQLDPKFITSILYENNVIT

SEQ ID NO: 52 - protein, 20 aa, Artificial Sequence
  Epitope of hEpCAM in ECD
YQLDPKFITSILYENNVITI

SEQ ID NO: 53 - protein, 21 aa, Artificial Sequence
  Epitope - in ECD
YQLDPKFITSILYENNVITSI

SEQ ID NO: 54 - protein, 9 aa, Artificial Sequence
  Epitope - in ECD
LDLDPGQTL

SEQ ID NO: 55 - protein, 10 aa, Artificial Sequence
  Epitope - in ECD
LLDLDPGQTL

SEQ ID NO: 56 - protein, 10 aa, Artificial Sequence
  Epitope - in ECD
QLDLDPGQTL

SEQ ID NO: 57 - protein, 10 aa, Artificial Sequence
  Epitope - in ECD
WIIIELKHKA

SEQ ID NO: 58 - protein, 10 aa, Artificial Sequence
  Epitope - in ECD
WIIIELKHKE

SEQ ID NO: 59 - protein, 9 aa, Artificial Sequence
  Epitope - in ECD
STCWCVNTA

SEQ ID NO: 60 - protein, 9 aa, Artificial Sequence
  Epitope - in ECD
ATCWCVNTA

SEQ ID NO: 61 - protein, 11 aa, Artificial Sequence
  Epitope - in ECD
YVDEKAPEFSM

SEQ ID NO: 62 - protein, 11 aa, Artificial Sequence
  Epitope - in ECD
YVDEKAPEFSN

SEQ ID NO: 63 - protein, 18 aa, Artificial Sequence
  Epitope - in ECD
QNNDGLYDPDCDESGLFD

SEQ ID NO: 64 - protein, 18 aa, Artificial Sequence
  Epitope - in ECD
QNNDGLYDPDCDESGLFK

SEQ ID NO: 65 - protein, 9 aa, Artificial Sequence
  Epitope in ECD/TMD
SMQGLKAGV

SEQ ID NO: 66 - protein, 11 aa, Artificial Sequence
  Epitope in ECD/TMD
SMQGLKAVAGV

SEQ ID NO: 67 - protein, 14 aa, Artificial Sequence
  Epitope in ECD/TMD
SMQGLKAVAGVTAV

SEQ ID NO: 68 - protein, 11 aa, Artificial Sequence
  Epitope in ECD/TMD
GLKAGVIAVIV

SEQ ID NO: 69 - protein, 9 aa, Artificial Sequence
  Epitope in ECD/TMD
GLKAGVIAV

SEQ ID NO: 70 - protein, 10 aa, Artificial Sequence
  Epitope in ECD/TMD
GLKAGVIAVI

SEQ ID NO: 71 - protein, 10 aa, Artificial Sequence
  Propeptide epitope
CVCENYKLAV

SEQ ID NO: 72 - protein, 18 aa, Artificial Sequence
  Propeptide epitope
AQNTVICSKLAAKCLVMK

SEQ ID NO: 73 - protein, 11 aa, Artificial Sequence
  Propeptide epitope
LLLAAATATFA

SEQ ID NO: 74 - protein, 9 aa, Artificial Sequence
  Epitope - in signal peptide
QVLAFGLLL

SEQ ID NO: 75 - protein, 9 aa, Artificial Sequence
  Epitope - in signal peptide
LLAATATFA

SEQ ID NO: 76 - protein, 9 aa, Artificial Sequence
  TAg-25 signal peptide epitope
QALALGLLL

SEQ ID NO: 77 - protein, 9 aa, Artificial Sequence
  Epitope in TMD
VVAGIVVLV

SEQ ID NO: 78 - protein, 266 aa, Artificial Sequence
  Tag-25/18 chimera (comprising SP+PP+ECD)
MAPPQALALGLLLAAATATFAAAQEECVCENYKLAVNCFVNNNRECQCTSVGAQNTVICSKLAAKCLVMKAEMNGSKLGRRIKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQNDVDIADVAHYFEKDVKGESLFHSSKKMDLRVNGEQLDLDPGQTLIYYVDRNAPEFSMQALK

SEQ ID NO: 79 - DNA, 798 bp, Artificial Sequence
  DNA encoding Tag-25/18 chimera comprising SP+PP+ECD
atggcaccccctcaagcactggcactgggtctgctgctggccgccgctaccgccactttcgccgcagcacaggaggagtgtgtgtgcgaaaactacaagctcgctgtcaactgtttcgtcaacaataatagagaatgccagtgcacttctgtgggagcacagaatacagtgatctgtagcaaactggctgcaaagtgtctggtcatgaaggccgaaatgaacggatccaagctcgggcggaggatcaaacctgaaggagctctgcagaacaacgatggtctctacgaccccgactgtgacgagtccggcctcttcaaggccaaacagtgtaatggcactgctacatgctggtgcgtgaacaccgctggggtgcgccggaccgataaggataccgaaattacctgttctgagagggtccggacatattggatcatcattgaactcaaacataaagagcgcgagtctccatacgattctaaatccctccatactgcactgcaaaaggaaatcactacacgctaccagctggatccaaaattcattacatccatcctctatgagaacaatgttattacaattgatctgatgcaaaatagctctcagaagactcagaatgatgtggacatagctgatgtggctcattattttgaaaaagatgttaaaggtgaatccttgtttcattcttctaagaaaatggacctgagagtaaatggagaacaactggatctggatcctggtcaaactttaatttattatgttgatagaaatgcacctgaattttcaatgcaggctctaaaa

SEQ ID NO: 80 - protein, 25 aa, Artificial Sequence
  TAg-18 TMD
AGVCAVAVIVVVMIAVVAGIVVLVI

SEQ ID NO: 81 - protein, 53 aa, Artificial Sequence
  Seq Pattern 1 (propeptide alignment)
QXXCVCXNYKLXXXCXXNXXXXCQCTSXGXQNTVICSKLAXMKAEMXXSKXGR

SEQ ID NO: 82 - protein, 53 aa, Artificial Sequence
  Seq pattern 2 - propeptide alignment with retention of epitopes
QXXCVCENYKLAVXCXXNXXXXCQCTSXGXQNTVICSKLAVMKAEMXXSKXGR

SEQ ID NO: 83 - protein, 23 aa, Artificial Sequence
  Signal peptide sequence pattern
MAXPXXLAXGLLLAXXTATXAAA

SEQ ID NO: 84 - protein, 33 aa, Artificial Sequence
  EGF-LIKE DOMAIN 1 SEQ PATTERN
CXCXXXXXXXXCXXXXXXXCXCXXXXXXXXXXC

SEQ ID NO: 85 - protein, 70 aa, Artificial Sequence
  EGF-LIKE DOMAIN 2 SEQ PATTERN
CXXXXXXXXXXXXXXXXXXXXXXXXXXXXXXXXCXXXXXXXXXXCXXXXXCXCXXXXXXXXXXXXXXXXC

SEQ ID NO: 86 - protein, 33 aa, Artificial Sequence
  EGF-LIKE DOMAIN 1 SEQ PATTERN with TAg-25 epitope
CVCENYKLAVXCXXXXXXXCXCXXXXXXXXXXC

SEQ ID NO: 87 - protein, 70 aa, Artificial Sequence
  EGF-LIKE DOMAIN 2 SEQ PATTERN with TAg-25 epitopes and RR site
CXXXXXXXXXXXXXRRXXXXXXXQNNDGLYDPDCDESGLFKXXXCXXXATCWCVNTAXXXXXXXXXXXXC

SEQ ID NO: 88 - protein, 109 aa, Artificial Sequence
  Combined EGF domains (all Cys) pattern
CXCXXXXXXXXCXXXXXXXCXCXXXXXXXXXXCXXXXXXCXXXXXXXXXXXXXXXXXXXXXXXXXXXXXXXXCXXXXXXXXXXCXXXXXCXCXXXXXXXXXXXXXXXXC

SEQ ID NO: 89 - protein, 109 aa, Artificial Sequence
  Example of 12 Cys pattern with some epitopes
CVCENYKLAVXCXXXXXXXCXCXXXXXXXXXXCXXXXXXCXXXXXXXXXXXXXRRXXXXXXXQNNDGLYDPDCDESGLFKXXXCXXXATCWCVNTAXXXXXXXXXXXXC

SEQ ID NO: 90 - protein, 55 aa, Artificial Sequence
  Mature portion of SEQ ID NO89
RXXXXXXXQNNDGLYDPDCDESGLFKXXXCXXXATCWCVNTAXXXXXXXXXXXXC

SEQ ID NO: 91 - protein, 71 aa, Artificial Sequence
  Sequence pattern - Thyroglobulin type-1 repeat motif
CXVERXXXXXXSXXXXXXXXEGALXXXXGLYXPXCDEXGXXKXXQCXXXXXXCWCVDXXGXXXXXXDXXXE

SEQ ID NO: 92 - protein, 184 aa, Artificial Sequence
  TAg-25 fragment comprising ECD N-terminal variant
IKPEGALQNNDGLYDPDCDESGLFKAKQCNGTATCWCVNTAGVRRTDKDTEITCSERVRTYWIIIELKHKERESPYDSKSLHTALQKEITTRYQLDPKFITSILYENNVITIDLMQNSSQKTQDDVDIADVAYYFEKDVKGESLFHSKKMDLRVNGELLDLDPGQTLIYYVDEKAPEFSMQGLK

SEQ ID NO: 93 - DNA, 795 bp, Artificial Sequence
  DNA encoding hEpCAM antigenic fragment comprising SP+PP+ECD (termed “sEpCAM”)
atggcgcccccgcaggtcctcgcgttcgggcttctgcttgccgcggcgacggcgacttttgccgcagctcaggaagaatgtgtctgtgaaaactacaagctggccgtaaactgctttgtgaataataatcgtcaatgccagtgtacttcagttggtgcacaaaatactgtcatttgctcaaagctggctgccaaatgtttggtgatgaaggcagaaatgaatggctcaaaacttgggagaagagcaaaacctgaaggggccctccagaacaatgatgggctttatgatcctgactgcgatgagagcgggctctttaaggccaagcagtgcaacggcacctccacgtgctggtgtgtgaacactgctggggtcagaagaacagacaaggacactgaaataacctgctctgagcgagtgagaacctactggatcatcattgaactaaaacacaaagcaagagaaaaaccttatgatagtaaaagtttgcggactgcacttcagaaggagatcacaacgcgttatcaactggatccaaaatttatcacgagtattttgtatgagaataatgttatcactattgatctggttcaaaattcttctcaaaaaactcagaatgatgtggacatagctgatgtggcttattattttgaaaaagatgttaaaggtgaatccttgtttcattctaagaaaatggacctgacagtaaatggggaacaactggatctggatcctggtcaaactttaatttattatgttgatgaaaaagcacctgaattctcaatgcagggtctaaaa

SEQ ID NO: 94 - DNA, 372 bp, Artificial Sequence
  nucleotide sequence of a mAb variable heavy chain domain
gaggtgaagctgctggagtccggaggtggcctggtgcagcctggaggatccctgaaactctcctgtgcagcctcaggattcgattttagtagatactggatgagttgggtccggcaggctccagggaaagggctagaatggattggagatattaatctagaaagcaatacgataaactatacgccatctctaaaggataaattcatcatctccagagacaacgccaaaaatacgctgtacctgcaaatgaacaaagtgagatctgaggacacagccctttattactgtgcaagaggggcctatactatggactactggggtcaaggaacctcagtcaccgtctcctcagccaaaacgacacccccatctgtc

SEQ ID NO: 95 - protein, 126 aa, Artificial Sequence
  amino acid sequence of a mAb variable heavy chain domain
EVKLLESGGGLVQPGGSLKLSCAASGFDFSRYWMSWVRQAPGKGLEWIGDINLESNTINYTPSLKDKFIISRDNAKNTLYLQMNKVRSEDTALYYCARGAYTMDYWGQGTSVTVSSAKTTPPSVAA